SEQUENCE LISTING (198 sequences)

SEQ ID NO: 1 - DNA, 4374 bp, synthetic construct
  misc_feature 1..4374: note = coFVIII-5
  source 1..4374: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 2 - DNA, 4374 bp, synthetic construct
  misc_feature 1..4374: note = coFVIII-4
  source 1..4374: mol_type = other DNA
atgcagatcgagctgagcacgtgcttcttcctgtgcctgctgaggttctgcttcagcgccaccaggaggtactacctgggcgccgtggagctgagctgggactacatgcagagcgacctgggcgagctgcccgtggacgccaggttcccccccagggtgcccaagagcttccccttcaacacgagcgtggtgtacaagaagaccctgttcgtggagttcaccgaccatctgttcaatatcgccaagcccaggcccccctggatggggctgctggggcccacgatccaggccgaggtgtacgacaccgtggtcatcaccctgaagaacatggccagccaccccgtgagcctgcacgccgtgggcgtgagctactggaaggccagcgagggcgccgagtacgacgaccagaccagccagagggagaaggaggacgacaaggtgttccccggcggcagccacacctacgtgtggcaggtgctgaaggagaatgggcccatggccagcgaccccctgtgcctgacctactcttacctgagccacgtggatctggtgaaggacctgaacagcggcctgatcggcgccctgctggtgtgcagggagggcagcctggccaaggagaagacccagaccctgcacaagttcatcctgctgttcgccgtgttcgacgagggcaagagctggcacagcgagaccaagaacagcctgatgcaggatagggacgccgccagcgccagggcctggcccaagatgcacaccgtgaacggctacgtgaacaggtctctgcccggcctgatcggctgccacaggaagagcgtgtactggcacgtgatcggcatggggaccacccccgaggtgcacagcatcttcctggagggccacacgttcctggtgaggaatcacaggcaggccagcctggagatcagcccgatcaccttcctgaccgcccagaccctgctgatggacctggggcagttcctgctgttctgccatatcagctctcaccagcacgacggcatggaggcctacgtgaaggtggatagctgccccgaggagccccagctgaggatgaagaacaacgaggaggccgaggactacgacgacgacctgaccgacagcgagatggacgtggtgaggttcgacgacgacaatagcccgagcttcatccagatcaggagcgtggccaagaagcaccccaagacctgggtgcattacatcgccgccgaggaggaggattgggactacgcccccctggtgctggcccccgacgacaggtcttacaagagccagtacctgaacaacgggccccagaggatcggcaggaagtacaagaaggtgaggttcatggcctacaccgacgagaccttcaagaccagggaggcgatccagcacgagagcgggatcctggggcccctgctgtacggcgaggtgggcgacacgctgctgatcatcttcaagaaccaggccagcaggccgtacaatatctacccccacgggatcaccgacgtgaggcccctgtactctaggaggctgcccaagggcgtgaagcacctgaaggacttccccatcctgcccggcgagatcttcaagtacaagtggaccgtgaccgtggaggacgggcccacgaagagcgaccccaggtgcctgaccaggtactacagctctttcgtgaacatggagagggacctggccagcggcctgatcgggcccctgctgatctgctacaaggagagcgtggatcagaggggcaaccagatcatgagcgacaagaggaacgtgatcctgttcagcgtgttcgacgagaataggtcttggtacctgaccgagaatatccagaggttcctgcccaaccccgccggcgtgcagctggaggatcccgagttccaggccagcaacatcatgcacagcatcaacggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttcagcggcgagaccgtgttcatgagcatggagaaccccggcctgtggatcctgggctgccataactccgacttcaggaataggggcatgaccgccctgctgaaggtgagctcttgcgacaagaacaccggcgactactacgaggatagctacgaggatatcagcgcctacctgctgagcaagaacaacgccatcgagcccaggtctttcagccagaacccccccgtgctgaagaggcaccagagggagatcaccaggacgaccctgcagagcgaccaggaggagatcgactacgacgacacgatcagcgtggagatgaagaaggaggatttcgacatctacgacgaggacgagaatcagagccccaggtctttccagaagaagaccaggcattacttcatcgccgccgtggagaggctgtgggactacggcatgagcagctctccccacgtgctgaggaatagggcccagagcggcagcgtgccccagttcaagaaggtggtgttccaggagttcaccgacggcagcttcacccagcccctgtacaggggcgagctgaacgagcacctgggcctgctggggccctacatcagggccgaggtggaggataacatcatggtgaccttcaggaatcaggccagcaggccctatagcttctatagctctctgatcagctacgaggaggatcagaggcagggcgccgagcccaggaagaacttcgtgaagcccaacgagaccaagacctacttctggaaggtgcagcaccacatggcccccacgaaggacgagttcgactgcaaggcctgggcctacttcagcgacgtggatctggagaaggacgtgcacagcggcctgatcgggcccctgctggtgtgccacaccaacaccctgaaccccgcccacggcaggcaggtgaccgtgcaggagttcgccctgttcttcaccatcttcgacgagaccaagagctggtacttcaccgagaatatggagaggaattgcagggccccctgcaatatccagatggaggacccgaccttcaaggagaattacaggttccacgccatcaacggctacatcatggacacgctgcccggcctggtcatggcccaggatcagaggatcaggtggtatctgctgagcatggggagcaacgagaatatccacagcatccacttcagcggccacgtgttcaccgtgaggaagaaggaggagtacaagatggccctgtacaatctgtaccccggcgtgttcgagaccgtggagatgctgcccagcaaggccgggatctggagggtggagtgcctgatcggcgagcacctgcacgccggcatgagcacgctgttcctggtgtactctaacaagtgccagacccccctggggatggccagcggccacatcagggacttccagatcaccgccagcggccagtacggccagtgggcccccaagctggccaggctgcactattccggaagcatcaacgcctggagcacgaaggagcccttcagctggatcaaggtggatctgctggcccccatgatcatccacgggatcaagacccagggcgccaggcagaagttcagctctctgtatatcagccagttcatcatcatgtactctctggacggcaagaagtggcagacctacaggggcaacagcaccggcacgctgatggtgttcttcggcaacgtggactctagcgggatcaagcacaatatcttcaacccccccatcatcgccaggtacatcaggctgcaccccacccattactctatcaggtctaccctgaggatggagctgatgggctgcgacctgaacagctgcagcatgcccctggggatggagagcaaggccatcagcgacgcccagatcaccgccagctcttacttcaccaacatgttcgccacctggagcccgagcaaggccaggctgcacctgcagggcaggtctaacgcctggaggccccaggtgaacaaccccaaggagtggctgcaggtggatttccagaagaccatgaaggtgaccggcgtgaccacgcagggcgtgaagagcctgctgaccagcatgtacgtgaaggagttcctgatcagctctagccaggacggccaccagtggaccctgttcttccagaacggcaaggtgaaggtgttccagggcaaccaggatagcttcacccccgtggtgaacagcctggacccccccctgctgaccaggtatctgaggatccacccccagagctgggtgcaccagatcgccctgaggatggaggtgctgggctgcgaggcccaggatctgtattga

SEQ ID NO: 3 - DNA, 4374 bp, synthetic construct
  misc_feature 1..4374: note = coFVIII-52
  source 1..4374: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 4 - DNA, 4374 bp, synthetic construct
  misc_feature 1..4374: note = coFVIII-62
  source 1..4374: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 5 - DNA, 4374 bp, synthetic construct
  misc_feature 1..4374: note = coFVIII-25
  source 1..4374: mol_type = other DNA
atgcagattgagctgagcacctgcttcttcctgtgcctgctgaggttctgcttctctgccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtcttcgacgagaacaggagctggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 6 - DNA, 4374 bp, synthetic construct
  misc_feature 1..4374: note = coFVIII-26
  source 1..4374: mol_type = other DNA
atgcagattgagctgagcacctgcttcttcctgtgcctgctgaggttctgcttctctgccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtcttcgacgagaacaggagctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 7 - DNA, 1734 bp, synthetic construct
  misc_feature 1..1734: note = coFVIII-52-NT58
  source 1..1734: mol_type = other DNA
gcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtg

SEQ ID NO: 8 - DNA, 2583 bp, synthetic construct
  misc_feature 1..2583: note = coFVIII-52-CT
  source 1..2583: mol_type = other DNA
tttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 9 - DNA, 1734 bp, synthetic construct
  misc_feature 1..1734: note = coFVIII-62-NT58
  source 1..1734: mol_type = other DNA
gccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtg

SEQ ID NO: 10 - DNA, 2583 bp, synthetic construct
  misc_feature 1..2583: note = coFVIII-62-CT
  source 1..2583: mol_type = other DNA
tttgatgaaaacagatcctggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaaccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 11 - DNA, 1734 bp, synthetic construct
  misc_feature 1..1734: note = coFVIII-25-NT58
  source 1..1734: mol_type = other DNA
gccaccaggagatactacctgggcgccgtggagctgagctgggactacatgcagtctgacctgggcgagctgccagtggacgccaggttcccccccagagtgcccaagagcttccccttcaacaccagcgtggtgtacaagaagaccctgttcgtggagttcactgaccacctgttcaacatcgccaagcccaggcccccctggatgggcctgctgggccccaccatccaggccgaggtgtacgacaccgtggtcatcaccctgaagaacatggccagccaccccgtctccctgcacgccgtgggggtgagctactggaaggcctctgagggcgccgagtacgacgaccagaccagccagagggagaaggaggacgacaaggtgttccctgggggcagccacacctacgtgtggcaggtcctgaaggagaacggccccatggcctctgaccccctgtgcctgacctacagctacctgagccacgtggacctggtgaaggacctgaactctggcctgattggggccctgctggtgtgcagggagggcagcctggccaaggagaagacccagaccctgcacaagttcatcctgctgttcgccgtgttcgacgagggcaagagctggcactctgaaaccaagaacagcctgatgcaggacagggacgccgcctctgccagggcctggcccaagatgcacaccgtcaacggctacgtcaacaggagcctgcctggcctgattggctgccacaggaagagcgtgtactggcatgtgatcggcatgggcaccacccctgaggtgcacagcatcttcctggagggccacaccttcctggtcaggaaccacaggcaggccagcctggagatcagccccatcaccttcctgaccgcccagaccctgctgatggacctgggccagttcctgctgttctgccacatctccagccaccagcacgacggcatggaggcctacgtgaaagtggacagctgccctgaggagccccagctgaggatgaagaacaacgaggaggccgaggactatgatgacgacctgaccgacagcgagatggacgtggtcaggttcgacgacgacaacagccccagcttcatccagatcaggagcgtggccaagaagcaccccaagacctgggtgcactacatcgctgctgaggaggaggactgggactatgcccccctggtgctggcccctgatgacaggagctacaagagccagtacctgaacaatggcccccagaggattggcaggaagtacaagaaagtcaggttcatggcctacactgatgaaaccttcaagaccagggaggccatccagcatgagtctggcatcctgggccccctgctgtacggggaggtgggggacaccctgctgatcatcttcaagaaccaggccagcaggccctacaacatctacccccatggcatcaccgacgtgaggcccctgtacagcaggaggctgcctaagggggtgaagcacctgaaagacttccccatcctgcctggggagatcttcaagtacaagtggactgtgactgtggaggacggccccaccaagagcgaccccaggtgcctgaccagatactacagcagcttcgtcaacatggagagggacctggcctctggcctgattggccccctgctgatctgctacaaggagtctgtggaccagaggggcaaccagatcatgagcgacaagaggaacgtgatcctgttctctgtc

SEQ ID NO: 12 - DNA, 2583 bp, synthetic construct
  misc_feature 1..2583: note = coFVIII-25-CT
  source 1..2583: mol_type = other DNA
ttcgacgagaacaggagctggtacctgactgaaaacatccagcggttcctccccaaccccgcgggcgtgcagctggaagatcctgagtttcaggcatcaaacatcatgcactccattaacggctacgtgttcgattcgctgcagctgagcgtgtgtctgcacgaagtggcctactggtacatcctgtccattggtgcccagactgacttcctgtccgtgtttttctccggctacacgttcaagcacaagatggtgtacgaggacaccctgaccctcttccctttttccggcgaaactgtgtttatgagcatggagaatcccggcctgtggatcttgggctgccacaacagcgacttccgtaacagaggaatgactgcgctgctcaaggtgtccagctgcgacaagaacaccggagactattatgaggactcatacgaggacatctccgcctacctcctgtccaagaataacgccattgaacctcggagcttcagccagaacccacccgtgcttaagagacatcaacgggagatcactaggaccaccctgcagtcagaccaggaggaaatcgactacgatgacaccatctcggtcgagatgaagaaggaggactttgacatctacgacgaagatgaaaaccagagcccgaggtcgttccaaaagaaaacccgccactactttattgctgctgtcgagcggctgtgggactacggaatgtcgtcctcgccgcacgtgctccgcaaccgagcccagagcggctcggtgccgcaattcaagaaggtcgtgttccaggagttcactgacgggagcttcactcagcctttgtaccggggagaactcaatgaacatctcggcctcctcggaccttacatcagagcagaagtggaagataacatcatggtcactttccgtaaccaagccagccgcccgtactcgttctactcctccctcatttcttacgaagaggaccagcggcagggcgcagaaccgcgcaagaacttcgtgaagcccaacgaaaccaagacctacttctggaaagtgcagcatcatatggccccgactaaggacgagtttgactgcaaagcctgggcctacttctccgatgtggacttggagaaggacgtccactccggcctcatcggtcccctgctcgtgtgccataccaataccctgaaccccgcacacggtcgccaggtcaccgtgcaggagttcgctctgttcttcactatcttcgacgaaactaagtcctggtacttcaccgagaacatggagaggaactgcagagccccctgtaacatccagatggaggacccgacgttcaaggaaaactaccggttccacgccattaacggatacatcatggatacgctgccgggtcttgtgatggcccaggatcaacggatcagatggtacttattgtcgatgggcagcaacgagaacatccactctattcacttctccggtcatgtgttcactgtgcggaagaaggaagagtacaagatggccctgtacaacctttatcccggagtgttcgaaactgtggaaatgctgccgtcgaaggccggcatttggcgcgtggagtgtttgattggagaacatctccatgcggggatgtcaaccctgttcctggtgtatagcaacaagtgccagactccgcttgggatggcgtcaggacacattagggatttccagatcactgcgtccggccagtacggccaatgggcccctaagctggcccgcctgcattactccggatccattaacgcctggtcaaccaaggagccattctcctggatcaaggtggaccttctggcccccatgattatccacggaattaagacccagggggcccggcagaagttctcctcactgtacatcagccagttcataatcatgtactccctggacggaaagaagtggcaaacctacagggggaacagcaccggcacactgatggtctttttcggaaatgtggactcctccgggattaagcataacatcttcaaccctccgattatcgctcggtacattagacttcaccctacccactacagcattcgctccaccctgcggatggaactgatgggctgcgatctgaactcgtgcagcatgccgttgggaatggagtccaaagcaatttccgacgcgcagatcaccgcctcgtcctactttaccaacatgttcgccacgtggtcaccgtccaaggcccggctgcacctccagggaagatccaacgcatggcggccacaggtcaacaaccctaaggagtggctccaggtggacttccagaaaaccatgaaggtcaccggagtcacaacccagggagtgaagtcgctgctgacttctatgtacgtcaaggagttcctgatctccagcagccaggacgggcaccagtggaccctgttcttccaaaatggaaaggtcaaggtgtttcagggcaatcaggattcattcaccccggtggtgaactcccttgatccacccctcctgacccgctaccttcgcatccacccacagtcctgggtgcaccagatcgcgctgaggatggaggtcctgggatgcgaagcccaggacctgtactga

SEQ ID NO: 13 - DNA, 1734 bp, synthetic construct; identical to SEQ ID NO: 11
  misc_feature 1..1734: note = coFVIII-26-NT58
  source 1..1734: mol_type = other DNA

SEQ ID NO: 14 - DNA, 2583 bp, synthetic construct
  misc_feature 1..2583: note = coFVIII-26-CT
  source 1..2583: mol_type = other DNA
ttcgacgagaacaggagctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 15 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - DNA, 4371 bp, synthetic construct
  misc_feature 1..4371: note = BDD-FVIII (non-optimized; "parental"), Nucleotide Sequence
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 17 - protein, 1438 aa, synthetic construct
  REGION 1..1438: note = BDD-FVIII (non-optimized; "parental"), Amino Acid Sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - DNA, 450 bp, synthetic construct
  misc_feature 1..450: note = XTEN nucleotide sequence
  source 1..450: mol_type = other DNA
ggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagc

SEQ ID NO: 19 - DNA, 4824 bp, synthetic construct
  misc_feature 1..4824: note = coFVIII-52-XTEN
  source 1..4824: mol_type = other DNA
atgcaaatcgaactgagcacctgtttcttcctctgcctgctgagattctgtttctccgcgacccgccgatactacctgggagcagtggagctctcctgggattacatgcagagcgaccttggggagctgcccgtggatgccaggttccctccccgggtgccaaagtcgtttccgttcaacacctccgtggtgtacaagaaaactctgttcgtggagttcaccgaccacctgttcaatatcgccaagcccagacctccctggatggggctgttgggacctaccatccaagcggaggtgtacgacactgtggtcatcactctgaagaacatggcctcgcatcccgtgtccctgcacgccgtgggagtgtcttactggaaagcgtccgagggggccgaatacgacgaccagacctcgcagagagaaaaggaagatgacaaggtgttcccaggaggatcgcacacctacgtgtggcaagtgttgaaggagaacggcccaatggcctccgacccgctgtgcctgacctactcgtacctgtcccacgtggacctcgtgaaggacctcaactcgggactgattggagccctgctggtctgcagggaaggctcactggcgaaagaaaagactcagaccttgcacaagttcattctgctgttcgctgtgttcgacgaggggaagtcgtggcacagcgagactaagaactccctgatgcaagatagagatgccgcctccgcccgggcctggcctaagatgcacaccgtgaacggttacgtgaaccgctccctccctggcctgattggatgccaccggaagtccgtgtactggcacgtgatcgggatggggaccacccccgaggtgcacagcatcttcctggaaggtcacacatttctcgtgcgcaaccaccggcaggcctccctggaaatcagccccattaccttcctcactgcccagactctgctgatggacctgggacagttcctgctgttctgccatatctcctcccaccaacatgacggaatggaggcatacgtgaaggtcgattcctgccctgaggaaccccagctccgcatgaagaacaatgaggaagccgaggactacgacgacgacctgacggatagcgagatggatgtggtccggttcgatgacgataacagcccttccttcatccaaattcgctcggtggcaaagaagcaccccaagacctgggtgcattacattgcggcggaagaagaggactgggattatgccccgcttgtcctcgctcctgacgaccggagctacaagagccagtacctgaacaacggtccacagaggatcggtagaaagtacaagaaggtccgcttcatggcctataccgacgaaaccttcaaaactagagaggccatccaacacgaatccggcatcctgggcccgctcttgtacggagaagtcggcgacacccttctcattatcttcaagaaccaggcttcccggccgtacaacatctatccgcatgggatcactgacgtgcgcccactgtactcgcggcgcctgcccaagggtgtcaaacacctgaaggattttccgatccttccgggagaaatcttcaagtacaagtggaccgtgaccgtggaagatggcccaactaagtctgaccctagatgcctcacccgctactactcatccttcgtcaacatggagcgcgacctggccagcggactgatcggcccgctgctgatttgctacaaggaatcagtggaccaacggggaaaccagatcatgtcggataagaggaacgtcatcctcttctccgtgtttgacgaaaaccggtcgtggtacctgaccgagaacatccagaggttcctgcccaaccctgctggggtgcagctggaggaccccgagttccaggccagcaacatcatgcacagcatcaatggctacgtgttcgacagcctgcagctgagcgtgtgcctgcacgaggtggcctactggtacatcctgagcatcggcgcccagaccgacttcctgagcgtgttcttctctggctacaccttcaagcacaagatggtgtatgaggacaccctgaccctgttccccttcagcggggagactgtcttcatgagcatggagaaccctggcctgtggatcctgggctgccacaacagcgacttcaggaacaggggcatgactgccctgctgaaagtctccagctgtgacaagaacaccggggactactacgaggacagctacgaggacatcagcgcctacctgctgagcaagaacaatgccatcgagcccaggagcttctctcagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagccccccagtgctgaagaggcaccagagggagatcaccaggaccaccctgcagtctgaccaggaggagatcgactatgatgacaccatcagcgtggagatgaagaaggaggacttcgacatctacgacgaggacgagaaccagagccccaggagcttccagaagaagaccaggcactacttcattgctgctgtggagaggctgtgggactatggcatgtccagcagcccccatgtgctgaggaacagggcccagtctggcagcgtgccccagttcaagaaagtcgtgttccaggagttcaccgacggcagcttcacccagcccctgtacagaggggagctgaacgagcacctgggcctgctgggcccctacatcagggccgaggtggaggacaacatcatggtgaccttcaggaaccaggccagcaggccctacagcttctacagcagcctgatcagctacgaggaggaccagaggcagggggctgagcccaggaagaactttgtgaagcccaatgaaaccaagacctacttctggaaggtgcagcaccacatggcccccaccaaggacgagttcgactgcaaggcctgggcctacttctctgacgtggacctggagaaggacgtgcactctggcctgattggccccctgctggtgtgccacaccaacaccctgaaccctgcccatggcaggcaggtgactgtgcaggagttcgccctgttcttcaccatcttcgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtcatggcccaggaccagaggatcaggtggtatctgctgagcatgggcagcaacgagaacatccacagcatccacttctctggccacgtgttcactgtgaggaagaaggaggagtacaagatggccctgtacaacctgtaccctggggtgttcgaaaccgtggagatgctgcccagcaaggccggcatctggagggtggagtgcctgattggggagcacctgcacgccggcatgagcaccctgttcctggtgtacagcaacaagtgccagacccccctgggcatggcctctggccacatcagggacttccagatcactgcctctggccagtacggccagtgggcccccaagctggccaggctgcactactccggaagcatcaatgcctggagcaccaaggagcccttcagctggatcaaagtggacctgctggcccccatgatcatccacggcatcaagacccagggggccaggcagaagttctccagcctgtacatcagccagttcatcatcatgtacagcctggacggcaagaagtggcagacctacaggggcaacagcaccggcaccctgatggtgttcttcggcaacgtggacagcagcggcatcaagcacaacatcttcaacccccccatcatcgccagatacatcaggctgcaccccacccactacagcatcaggagcaccctgaggatggagctgatgggctgtgacctgaacagctgcagcatgcccctgggcatggagagcaaggccatctctgacgcccagatcactgcctccagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtacgtgaaggagttcctgatctccagcagccaggacggccaccagtggaccctgttcttccagaatggcaaggtgaaggtgttccagggcaaccaggacagcttcacccctgtggtcaacagcctggacccccccctgctgaccagatacctgaggatccacccccagagctgggtgcaccagatcgccctgaggatggaggtgctgggctgtgaggcccaggacctgtactga

SEQ ID NO: 20 - DNA, 4824 bp, synthetic construct
  misc_feature 1..4824: note = coFVIII-1-XTEN
  source 1..4824: mol_type = other DNA
atgcagattgagctgtctacttgctttttcctgtgcctgctgaggttttgcttttccgctacacgaaggtattatctgggggctgtggaactgtcttgggattacatgcagagtgacctgggagagctgccagtggacgcaaggtttccccctagagtccctaagtcattccccttcaacactagcgtggtctacaagaaaacactgttcgtggagtttactgatcacctgttcaacatcgcaaagcctaggccaccctggatgggactgctggggccaacaatccaggccgaggtgtacgacaccgtggtcattacacttaagaacatggcctcacaccccgtgagcctgcatgctgtgggcgtcagctactggaaggcttccgaaggagcagagtatgacgatcagacttcccagagagaaaaagaggacgataaggtgtttcctggcggatctcatacctacgtgtggcaggtcctgaaagagaatggccctatggcctccgaccctctgtgcctgacctactcttatctgagtcacgtggacctggtcaaggatctgaacagcggcctgatcggagccctgctggtgtgcagggaaggaagcctggctaaggagaaaacccagacactgcataagttcattctgctgttcgccgtgtttgacgaagggaaatcatggcacagcgagacaaagaatagtctgatgcaggacagggatgccgcttcagccagagcttggcccaaaatgcacactgtgaacggctacgtcaatcgctcactgcctgggctgatcggctgccaccgaaagagcgtgtattggcatgtcatcgggatgggcaccacacctgaagtgcactccattttcctggagggacatacctttctggtccgcaaccaccgacaggcttccctggagatctctccaattaccttcctgacagcacagactctgctgatggacctggggcagttcctgctgttttgccacatcagctcccaccagcatgatggcatggaggcttacgtgaaagtggactcttgtcccgaggaacctcagctgcggatgaagaacaatgaggaagcagaagactatgacgatgacctgaccgactccgagatggatgtggtccgattcgatgacgataacagcccctcctttatccagattagatctgtggccaagaaacaccctaagacatgggtccattacatcgcagccgaggaagaggactgggattatgcaccactggtgctggcaccagacgatcgctcctacaaatctcagtatctgaacaatgggccacagaggattggcagaaagtacaagaaagtgcggttcatggcatataccgatgagaccttcaagactcgcgaagccatccagcacgagagcggcatcctgggaccactgctgtacggagaagtgggagacaccctgctgatcattttcaagaaccaggccagccggccttacaatatctatccacatgggattacagatgtgcgccctctgtacagcaggagactgccaaagggcgtcaaacacctgaaggacttcccaatcctgcccggagaaatcttcaagtacaagtggactgtcaccgtcgaggatggccccactaagagcgaccctcggtgcctgacccgctactattctagtttcgtgaatatggaaagagatctggcaagcggactgatcggaccactgctgatttgttacaaagagagcgtggatcagagaggcaaccagatcatgtccgacaagcggaatgtgattctgttcagtgtctttgacgaaaacaggtcatggtacctgaccgagaacatccagagattcctgcctaatccagctggggtgcagctggaagatcctgagtttcaggcatctaacatcatgcatagtattaatggctacgtgttcgacagtttgcagctgagcgtgtgcctgcacgaggtcgcttactggtatatcctgagcattggggcacagacagatttcctgagcgtgttcttttccggctacacttttaagcataaaatggtctatgaggacacactgactctgttccccttcagcggcgaaaccgtgtttatgagcatggagaatcccggactgtggattctggggtgccacaacagcgatttcagaaatcgcggaatgactgccctgctgaaagtgtcaagctgtgacaagaacaccggggactactatgaagattcatacgaggacatcagcgcatatctgctgtccaaaaacaatgccattgaaccccggtcttttagtcagaatggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagccctccagtgctgaagcggcaccagcgcgagatcacccgcactaccctgcagagtgatcaggaagagatcgactacgacgatacaatttctgtggaaatgaagaaagaggacttcgatatctatgacgaagatgagaaccagagtcctcgatcattccagaagaaaaccaggcattactttattgccgcagtggagcggctgtgggattatggcatgtcctctagtcctcacgtgctgcgaaatagggcccagtcaggaagcgtcccacagttcaagaaagtggtcttccaggagtttacagacgggtcctttactcagccactgtacaggggcgaactgaacgagcacctgggactgctggggccctatatcagagcagaagtggaggataacattatggtcaccttcagaaatcaggcctctcggccttacagtttttattcaagcctgatctcttacgaagaggaccagcgacagggagctgaaccacgaaaaaacttcgtgaagcctaatgagaccaaaacatacttttggaaggtgcagcaccatatggccccaacaaaagacgagttcgattgcaaggcatgggcctatttttctgacgtggatctggagaaggacgtgcacagtggcctgattggcccactgctggtgtgccatactaacaccctgaatccagcccacggccggcaggtcactgtccaggagttcgctctgttctttaccatctttgatgagacaaagagctggtacttcaccgaaaacatggagcgaaattgcagggctccatgtaacattcagatggaagaccccacattcaaggagaactaccgctttcatgctatcaatggatacatcatggatactctgcccgggctggtcatggcacaggaccagagaatccggtggtatctgctgagcatgggcagcaacgagaatatccactcaattcatttcagcgggcacgtgtttactgtcaggaagaaagaagagtacaagatggccctgtacaacctgtatcccggcgtgttcgaaaccgtcgagatgctgcctagcaaggccggaatctggagagtggaatgcctgattggagagcacctgcatgctgggatgtctaccctgtttctggtgtacagtaataagtgtcagacacccctgggaatggcatccgggcatatcagggatttccagattaccgcatctggacagtacggacagtgggcacctaagctggctagactgcactattccggatctatcaacgcttggtccacaaaagagcctttctcttggattaaggtggacctgctggccccaatgatcattcatggcatcaaaactcagggagctcggcagaagttctcctctctgtacatctcacagtttatcatcatgtacagcctggatgggaagaaatggcagacataccgcggcaatagcacaggaactctgatggtgttctttggcaacgtggacagcagcggaatcaagcacaacattttcaatccccctatcattgctagatacatccggctgcacccaacccattattctattcgaagtacactgaggatggaactgatgggatgcgatctgaacagttgttcaatgcccctggggatggagtccaaggcaatctctgacgcccagattaccgccagctcctacttcactaatatgtttgctacctggagcccttccaaagcaagactgcacctgcaaggccgcagcaacgcatggcgaccacaggtgaacaatcccaaggagtggttgcaggtcgattttcagaaaactatgaaggtgaccggggtcacaactcagggcgtgaaaagtctgctgacctcaatgtacgtcaaggagttcctgatctctagttcacaggacggacatcagtggacactgttctttcagaacgggaaggtgaaagtcttccagggcaatcaggattcctttacacctgtggtcaacagtctagaccctccactgctgaccagatacctgagaatccaccctcagtcctgggtgcaccagattgccctgagaatggaagtgctgggatgcgaggcccaggatctgtactga

SEQ ID NO: 21-31 - skipped (11 entries)

SEQ ID NO: 32 - DNA, 13 bp, synthetic construct
  misc_feature 1..13: note = kozak consensus sequence
  source 1..13: mol_type = other DNA
gccgccaccatgc

SEQ ID NO: 33 - protein, 32 aa, synthetic construct
  REGION 1..32: note = CTP peptide
DPRFQDSSSSKAPPPSLPSPSRLPGPSDTPIL

SEQ ID NO: 34 - protein, 28 aa, synthetic construct
  REGION 1..28: note = CTP peptide
SSSSKAPPPSLPSPSRLPGPSDTPILPQ

SEQ ID NO: 35 - protein, 11 aa, synthetic construct
  REGION 1..11: note = albumin-binding peptide core sequence
DICLPRWGCLW

SEQ ID NO: 36 - protein, 20 aa, synthetic construct
  REGION 1..20: note = PAS sequence
ASPAAPAPASPAAPAPSAPA

SEQ ID NO: 37 - protein, 20 aa, synthetic construct
  REGION 1..20: note = PAS sequence
AAPASPAPAAPSAPAPAAPS

SEQ ID NO: 38 - protein, 20 aa, synthetic construct
  REGION 1..20: note = PAS sequence
APSSPSPSAPSSPSPASPSS

SEQ ID NO: 39 - protein, 19 aa, synthetic construct
  REGION 1..19: note = PAS sequence
APSSPSPSAPSSPSPASPS

SEQ ID NO: 40 - protein, 20 aa, synthetic construct
  REGION 1..20: note = PAS sequence
SSPSAPSPSSPASPSPSSPA

SEQ ID NO: 41 - protein, 24 aa, synthetic construct
  REGION 1..24: note = PAS sequence
AASPAAPSAPPAAASPAAPSAPPA

SEQ ID NO: 42 - protein, 20 aa, synthetic construct
  REGION 1..20: note = PAS sequence
ASAAAPAAASAAASAPSAAA

SEQ ID NO: 43 - DNA, 23 bp, synthetic construct
  misc_feature 1..23: note = miR142 target
  source 1..23: mol_type = other DNA
tccataaagtaggaaacactaca

SEQ ID NO: 44 - protein, 2332 aa, Homo sapiens; identical to SEQ ID NO: 15

SEQ ID NO: 45 - protein, 4 aa, synthetic construct
  REGION 1..4: note = amino acids 233-236 of human IgG1
ELLG

SEQ ID NO: 46 - protein, 42 aa, synthetic construct
  REGION 1..42: note = XTEN AE42-4, protein sequence
GAPGSPAGSPTSTEEGTSESATPESGPGSEPATSGSETPASS

SEQ ID NO: 47 - DNA, 126 bp, synthetic construct
  misc_feature 1..126: note = XTEN AE42-4, DNA sequence
  source 1..126: mol_type = other DNA
ggcgcgccaggttctcctgctggctcccccacctcaacagaagaggggacaagcgaaagcgctacgcctgagagtggccctggctctgagccagccacctccggctctgaaacccctgcctcgagc

SEQ ID NO: 48 - protein, 144 aa, synthetic construct
  REGION 1..144: note = XTEN AE144-2A, protein sequence
TSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPG

SEQ ID NO: 49 - DNA, 450 bp, synthetic construct
  misc_feature 1..450: note = XTEN AE144-2A, DNA sequence
  source 1..450: mol_type = other DNA
ggcgcgccaaccagtacggagccgtccgaggggagcgcaccaggaagcccggctgggagcccgacttctaccgaagagggtacatctaccgaaccaagtgaaggttcagcaccaggcacctcaacagaaccctctgagggctcggcgcctggtacaagtgagtccgccaccccagaatccgggcctgggacaagcacagaaccttcggaagggagtgcccctggaacatccgaatcggcaaccccagaatcagggccaggatctgagcccgcgacttcgggctccgagacgcctgggacatccaccgagccctccgaaggatcagccccaggcaccagcacggagccctctgagggaagcgcacctggtaccagcgaaagcgcaactcccgaatcaggtcccggtacgagcgagtcggcgaccccggagagcgggccaggtgcctcgagc

SEQ ID NO: 50 - protein, 144 aa, synthetic construct
  REGION 1..144: note = XTEN AE144-3B, protein sequence
SPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPG

SEQ ID NO: 51 - DNA, 450 bp, synthetic construct
  misc_feature 1..450: note = XTEN AE144-3B, DNA sequence
  source 1..450: mol_type = other DNA
ggcgcgccaagtcccgctggaagcccaactagcaccgaagaggggacctcagagtccgccacccccgagtccggccctggctctgagcctgccactagcggctccgagactcctggcacatccgaaagcgctacacccgagagtggacccggcacctctaccgagcccagtgagggctccgcccctggaacaagcaccgagcccagcgaaggcagcgccccagggacctccacagagcccagtgaaggcagtgctcctggcaccagcaccgaaccaagcgagggctctgcacccgggacctccaccgagccaagcgaaggctctgcccctggcacttccaccgagcccagcgaaggcagcgcccctgggagccccgctggctctcccaccagcactgaggagggcacatctaccgaaccaagtgaaggctctgcaccaggtgcctcgagc

SEQ ID NO: 52 - protein, 144 aa, synthetic construct
  REGION 1..144: note = XTEN AE144-4A, protein sequence
TSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPG

SEQ ID NO: 53 - DNA, 450 bp, synthetic construct
  misc_feature 1..450: note = XTEN AE144-4A, DNA sequence
  source 1..450: mol_type = other DNA
ggcgcgccaacgtccgaaagtgctacccctgagtcaggccctggtagtgagcctgccacaagcggaagcgaaactccggggacctcagagtctgccactcccgaatcggggccaggctctgaaccggccacttcagggagcgaaacaccaggaacatcggagagcgctaccccggagagcgggccaggaactagtactgagcctagcgagggaagtgcacctggtacaagcgagtccgccacacccgagtctggccctggctctccagcgggctcacccacgagcactgaagagggctctcccgctggcagcccaacgtcgacagaagaaggatcaccagcaggctcccccacatcaacagaggagggtacatcagaatctgctactcccgagagtggacccggtacctccactgagcccagcgaggggagtgcaccaggtgcctcgagc

SEQ ID NO: 54 - protein, 144 aa, synthetic construct
  REGION 1..144: note = XTEN AE144-5A, protein sequence
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 55 - DNA, 450 bp, synthetic construct; identical to SEQ ID NO: 18
  misc_feature 1..450: note = XTEN AE144-5A, DNA sequence
  source 1..450: mol_type = other DNA

SEQ ID NO: 56 - protein, 144 aa, synthetic construct
  REGION 1..144: note = XTEN AE144-6B, protein sequence
TSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 57 - DNA, 450 bp, synthetic construct
  misc_feature 1..450: note = XTEN AE144-6B, DNA sequence
  source 1..450: mol_type = other DNA
ggcgcgccaacatctaccgagccttccgaaggctctgcccctgggacctcagaatctgcaacccctgaaagcggccctggaacctccgaaagtgccactcccgagagcggcccagggacaagcgagtcagcaacccctgagtctggacccggcagcgagcctgcaacctctggctcagagactcccggctcagaacccgctacctcaggctccgagacacccggctctcctgctgggagtcccacttccaccgaggaaggaacatccactgagcctagtgagggctctgcccctggaaccagcacagagccaagtgagggcagtgcaccaggatccgagccagcaaccagcgggtccgagactcccgggacctctgagtctgccaccccagagagcggacccggcacttcaaccgagccctccgaaggatcagcaccaggtgcctcgagc

SEQ ID NO: 58 - protein, 144 aa, synthetic construct
  REGION 1..144: note = XTEN AG144-1, protein sequence
PGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSS

SEQ ID NO: 59 - DNA, 450 bp, synthetic construct
  misc_feature 1..450: note = XTEN AG144-1, DNA sequence
  source 1..450: mol_type = other DNA
ggcgcgccacccgggtcgtccccgtcggcgtccaccggaacagggccagggtcatccccgtcagcgtcgactgggacgggacccgggacacccggttcggggactgcatcctcctcgcctggttcgtccaccccgtcaggagccacgggttcgccgggaagcagcccaagcgcatccactggtacagggcctggggcttcaccgggtacttcatccacggggtcaccgggaacgcccggatcggggacggcttcctcatcaccaggatcgtcaacaccctcgggcgcaacgggcagccccggaacccctggttcgggtacggcgtcgtcgagccccggtgcgagcccgggaacaagctcgacaggatcgcctggggcgtcacccggcacgtcgagcacaggcagccccggaacccctggatcgggaaccgcgtcgtcaagcgcctcgagc

SEQ ID NO: 60 - protein, 144 aa, synthetic construct
  REGION 1..144: note = XTEN AG144-A, protein sequence
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSP

SEQ ID NO: 61 - DNA, 450 bp, synthetic construct
  misc_feature 1..450: note = XTEN AG144-A, DNA sequence
  source 1..450: mol_type = other DNA
ggcgcgccaggtgcctcgccgggaacatcatcaactggttcacccgggtcatccccctcggcctcaaccgggacgggtcccggctcatcccccagcgccagcactggaacaggtcctggcactcctggttccggtacggcatcgtcatccccgggaagctcaacaccgtccggagcgacaggatcacctggctcgtcaccttcggcgtcaactggaacggggccaggggcctcacccggaacgtcctcgactgggtcgcctggtacgccgggatcaggaacggcctcatcctcgcctgggtcctcaacgccctcgggtgcgactggttcgccgggaactcctggctcggggacggcctcgtcgtcgcctggggcatcaccggggacgagctccacggggtcccctggagcgtcaccggggacctcctcgacaggtagcccggcctcgagc

SEQ ID NO: 62 - protein, 144 aa, synthetic construct
  REGION 1..144: note = XTEN AG144-B, protein sequence
GTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSP

SEQ ID NO: 63 - DNA, 450 bp, synthetic construct
  misc_feature 1..450: note = XTEN AG144-B, DNA sequence
  source 1..450: mol_type = other DNA
ggcgcgccaggtacaccgggcagcggcacggcttcgtcgtcacccggctcgtccacaccgtcgggagctacgggaagcccaggagcgtcaccgggaacgtcgtcaacggggtcaccgggtacgccaggtagcggcacggccagcagctcgccaggttcatcgaccccgtcgggagcgactgggtcgcccggatcaagcccgtcagcttccactggaacaggacccgggtcgtcgccgtcagcctcaacggggacaggacctggttcatcgacgccgtcaggggcgacaggctcgcccggatcgtcaacaccctcgggggcaacggggagccctggtgcgtcgcctggaacctcatccaccggaagcccgggggcctcgccgggtacgagctccacgggatcgcccggagcgtcccccggaacttcaagcacagggagccctgcctcgagc

SEQ ID NO: 64 - protein, 144 aa, synthetic construct
  REGION 1..144: note = XTEN AG144-C, protein sequence
GTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSP

SEQ ID NO: 65 - DNA, 450 bp, synthetic construct
  misc_feature 1..450: note = XTEN AG144-C, DNA sequence
  source 1..450: mol_type = other DNA
ggcgcgccaggtacacccggatcgggtacagcgtcatcgagccccggtgcgtcacctggtacgtcgagcacggggtcgccaggggcgtcccctgggacgtcctcaacaggctcgcccggtgcgtcacccggcacgtcgtccacgggttcacctggtagctccccttccgcgtccactggcaccgggcctggaactccggggagcggcacagcgagctcgtcgccgggagcatcgcctgggacatcgagcaccgggtcgccaggagcatcgcccggaacatccagcacaggaagccccggcgcgtcgcccgggacatcaagcacaggttccccgggatcgagcacgccgtccggagccactggatcaccagggagctcgacaccttccggcgcaacgggatcgcccggagccagcccgggtacgtcaagcactggctcccctgcctcgagc

SEQ ID NO: 66 - protein, 144 aa, synthetic construct
  REGION 1..144: note = XTEN AG144-F, protein sequence
GSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 67 - DNA, 450 bp, synthetic construct
  misc_feature 1..450: note = XTEN AG144-F, DNA sequence
  source 1..450: mol_type = other DNA
ggcgcgccaggctccagcccctccgcgagcacgggaaccggaccaggttcgtcaccctcagcatcaacggggacgggaccgggggcgtcaccaggaacgtcctccaccggctcgccgggtgcatcacccggaacgtcatcgaccggatcgccagggagctcgacgccatcaggcgcaacaggatcacctggctcaagccctagcgcgtcaaccggcacgggtccgggtgcctcccctggcacgtccagcaccggatcacccggatcgagcccatccgcctcaaccggaaccggacccggtacaccagggtcgggaacagcctcctcgtcaccaggctcctcaaccccctcgggagccacgggttcgcccggttcgtcaacgccttccggagcaactggtagccccggagcatcgccaggaacttcgagcacggggtcgcccgcctcgagc

SEQ ID NO: 68 - DNA, 4374 bp, synthetic construct
  misc_feature 1..4374: note = coFVIII-1 DNA Sequence
  source 1..4374: mol_type = other DNA
atgcagattgagctgtctacttgctttttcctgtgcctgctgaggttttgcttttccgctacacgaaggtattatctgggggctgtggaactgtcttgggattacatgcagagtgacctgggagagctgccagtggacgcaaggtttccccctagagtccctaagtcattccccttcaacactagcgtggtctacaagaaaacactgttcgtggagtttactgatcacctgttcaacatcgcaaagcctaggccaccctggatgggactgctggggccaacaatccaggccgaggtgtacgacaccgtggtcattacacttaagaacatggcctcacaccccgtgagcctgcatgctgtgggcgtcagctactggaaggcttccgaaggagcagagtatgacgatcagacttcccagagagaaaaagaggacgataaggtgtttcctggcggatctcatacctacgtgtggcaggtcctgaaagagaatggccctatggcctccgaccctctgtgcctgacctactcttatctgagtcacgtggacctggtcaaggatctgaacagcggcctgatcggagccctgctggtgtgcagggaaggaagcctggctaaggagaaaacccagacactgcataagttcattctgctgttcgccgtgtttgacgaagggaaatcatggcacagcgagacaaagaatagtctgatgcaggacagggatgccgcttcagccagagcttggcccaaaatgcacactgtgaacggctacgtcaatcgctcactgcctgggctgatcggctgccaccgaaagagcgtgtattggcatgtcatcgggatgggcaccacacctgaagtgcactccattttcctggagggacatacctttctggtccgcaaccaccgacaggcttccctggagatctctccaattaccttcctgacagcacagactctgctgatggacctggggcagttcctgctgttttgccacatcagctcccaccagcatgatggcatggaggcttacgtgaaagtggactcttgtcccgaggaacctcagctgcggatgaagaacaatgaggaagcagaagactatgacgatgacctgaccgactccgagatggatgtggtccgattcgatgacgataacagcccctcctttatccagattagatctgtggccaagaaacaccctaagacatgggtccattacatcgcagccgaggaagaggactgggattatgcaccactggtgctggcaccagacgatcgctcctacaaatctcagtatctgaacaatgggccacagaggattggcagaaagtacaagaaagtgcggttcatggcatataccgatgagaccttcaagactcgcgaagccatccagcacgagagcggcatcctgggaccactgctgtacggagaagtgggagacaccctgctgatcattttcaagaaccaggccagccggccttacaatatctatccacatgggattacagatgtgcgccctctgtacagcaggagactgccaaagggcgtcaaacacctgaaggacttcccaatcctgcccggagaaatcttcaagtacaagtggactgtcaccgtcgaggatggccccactaagagcgaccctcggtgcctgacccgctactattctagtttcgtgaatatggaaagagatctggcaagcggactgatcggaccactgctgatttgttacaaagagagcgtggatcagagaggcaaccagatcatgtccgacaagcggaatgtgattctgttcagtgtctttgacgaaaacaggtcatggtacctgaccgagaacatccagagattcctgcctaatccagctggggtgcagctggaagatcctgagtttcaggcatctaacatcatgcatagtattaatggctacgtgttcgacagtttgcagctgagcgtgtgcctgcacgaggtcgcttactggtatatcctgagcattggggcacagacagatttcctgagcgtgttcttttccggctacacttttaagcataaaatggtctatgaggacacactgactctgttccccttcagcggcgaaaccgtgtttatgagcatggagaatcccggactgtggattctggggtgccacaacagcgatttcagaaatcgcggaatgactgccctgctgaaagtgtcaagctgtgacaagaacaccggggactactatgaagattcatacgaggacatcagcgcatatctgctgtccaaaaacaatgccattgaaccccggtcttttagtcagaatcctccagtgctgaagaggcaccagagggagatcacccgcactaccctgcagagtgatcaggaagagatcgactacgacgatacaatttctgtggaaatgaagaaagaggacttcgatatctatgacgaagatgagaaccagagtcctcgatcattccagaagaaaaccaggcattactttattgccgcagtggagcggctgtgggattatggcatgtcctctagtcctcacgtgctgcgaaatagggcccagtcaggaagcgtcccacagttcaagaaagtggtcttccaggagtttacagacgggtcctttactcagccactgtacaggggcgaactgaacgagcacctgggactgctggggccctatatcagagcagaagtggaggataacattatggtcaccttcagaaatcaggcctctcggccttacagtttttattcaagcctgatctcttacgaagaggaccagcgacagggagctgaaccacgaaaaaacttcgtgaagcctaatgagaccaaaacatacttttggaaggtgcagcaccatatggccccaacaaaagacgagttcgattgcaaggcatgggcctatttttctgacgtggatctggagaaggacgtgcacagtggcctgattggcccactgctggtgtgccatactaacaccctgaatccagcccacggccggcaggtcactgtccaggagttcgctctgttctttaccatctttgatgagacaaagagctggtacttcaccgaaaacatggagcgaaattgcagggctccatgtaacattcagatggaagaccccacattcaaggagaactaccgctttcatgctatcaatggatacatcatggatactctgcccgggctggtcatggcacaggaccagagaatccggtggtatctgctgagcatgggcagcaacgagaatatccactcaattcatttcagcgggcacgtgtttactgtcaggaagaaagaagagtacaagatggccctgtacaacctgtatcccggcgtgttcgaaaccgtcgagatgctgcctagcaaggccggaatctggagagtggaatgcctgattggagagcacctgcatgctgggatgtctaccctgtttctggtgtacagtaataagtgtcagacacccctgggaatggcatccgggcatatcagggatttccagattaccgcatctggacagtacggacagtgggcacctaagctggctagactgcactattccggatctatcaacgcttggtccacaaaagagcctttctcttggattaaggtggacctgctggccccaatgatcattcatggcatcaaaactcagggagctcggcagaagttctcctctctgtacatctcacagtttatcatcatgtacagcctggatgggaagaaatggcagacataccgcggcaatagcacaggaactctgatggtgttctttggcaacgtggacagcagcggaatcaagcacaacattttcaatccccctatcattgctagatacatccggctgcacccaacccattattctattcgaagtacactgaggatggaactgatgggatgcgatctgaacagttgttcaatgcccctggggatggagtccaaggcaatctctgacgcccagattaccgccagctcctacttcactaatatgtttgctacctggagcccttccaaagcaagactgcacctgcaaggccgcagcaacgcatggcgaccacaggtgaacaatcccaaggagtggttgcaggtcgattttcagaaaactatgaaggtgaccggggtcacaactcagggcgtgaaaagtctgctgacctcaatgtacgtcaaggagttcctgatctctagttcacaggacggacatcagtggacactgttctttcagaacgggaaggtgaaagtcttccagggcaatcaggattcctttacacctgtggtcaacagtctagaccctccactgctgaccagatacctgagaatccaccctcagtcctgggtgcaccagattgccctgagaatggaagtgctgggatgcgaggcccaggatctgtactga

SEQ ID NO: 69 - DNA, 577 bp, synthetic construct
  misc_feature 1..577: note = ET Promoter, DNA sequence
  source 1..577: mol_type = other DNA
ctcgaggtcaattcacgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgccaccatgg

SEQ ID NO: 70 - DNA, 4374 bp, synthetic construct; identical to SEQ ID NO: 1
  misc_feature 1..4374: note = coFVIII-5
  source 1..4374: mol_type = other DNA

SEQ ID NO: 71 - DNA, 4374 bp, synthetic construct
  misc_feature 1..4374: note = coFVIII-6
  source 1..4374: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 72 - DNA, 4824 bp, synthetic construct
  misc_feature 1..4824: note = coFVIII-6-XTEN
  source 1..4824: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcagctgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 73 - protein, 12 aa, synthetic construct
  REGION 1..12: note = AD motif
GESPGGSSGSES

SEQ ID NO: 74 - protein, 12 aa, synthetic construct
  REGION 1..12: note = AD motif
GSEGSSGPGESS

SEQ ID NO: 75 - protein, 12 aa, synthetic construct
  REGION 1..12: note = AD motif
GSSESGSSEGGP

SEQ ID NO: 76 - protein, 12 aa, synthetic construct
  REGION 1..12: note = AD motif
GSGGEPSESGSS

SEQ ID NO: 77 - protein, 12 aa, synthetic construct
  REGION 1..12: note = AE, AM motif
GSPAGSPTSTEE

SEQ ID NO: 78 - protein, 12 aa, synthetic construct
  REGION 1..12: note = AE, AM, AQ motif
GSEPATSGSETP

SEQ ID NO: 79 - protein, 12 aa, synthetic construct
  REGION 1..12: note = AE, AM, AQ motif
GTSESATPESGP

SEQ ID NO: 80 - protein, 12 aa, synthetic construct
  REGION 1..12: note = AE, AM, AQ motif
GTSTEPSEGSAP

SEQ ID NO: 81 - protein, 12 aa, synthetic construct
  REGION 1..12: note = AF, AM motif
GSTSESPSGTAP

SEQ ID NO: 82 - protein, 12 aa, synthetic construct
  REGION 1..12: note = AF, AM motif
GTSTPESGSASP

SEQ ID NO: 83 - protein, 12 aa, synthetic construct
  REGION 1..12: note = AF, AM motif
GTSPSGESSTAP

SEQ ID NO: 84 - protein, 12 aa, synthetic construct
  REGION 1..12: note = AF, AM motif
GSTSSTAESPGP

SEQ ID NO: 85 - protein, 12 aa, synthetic construct
  REGION 1..12: note = AG, AM motif
GTPGSGTASSSP

SEQ ID NO: 86 - protein, 12 aa, synthetic construct
  REGION 1..12: note = AG, AM motif
GSSTPSGATGSP

SEQ ID NO: 87 - protein, 12 aa, synthetic construct
  REGION 1..12: note = AG, AM motif
GSSPSASTGTGP

SEQ ID NO: 88 - protein, 12 aa, synthetic construct
  REGION 1..12: note = AG, AM motif
GASPGTSSTGSP

SEQ ID NO: 89 - protein, 12 aa, synthetic construct
  REGION 1..12: note = AQ motif
GEPAGSPTSTSE

SEQ ID NO: 90 - protein, 12 aa, synthetic construct
  REGION 1..12: note = AQ motif
GTGEPSSTPASE

SEQ ID NO: 91 - protein, 12 aa, synthetic construct
  REGION 1..12: note = AQ motif
GSGPSTESAPTE

SEQ ID NO: 92 - protein, 12 aa, synthetic construct
  REGION 1..12: note = AQ motif
GSETPSGPSETA

SEQ ID NO: 93 - protein, 12 aa, synthetic construct
  REGION 1..12: note = AQ motif
GPSETSTSEPGA

SEQ ID NO: 94 - protein, 12 aa, synthetic construct
  REGION 1..12: note = AQ motif
GSPSEPTEGTSA

SEQ ID NO: 95 - protein, 12 aa, synthetic construct
  REGION 1..12: note = BC motif
GSGASEPTSTEP

SEQ ID NO: 96 - protein, 12 aa, synthetic construct
  REGION 1..12: note = BC motif
GSEPATSGTEPS

SEQ ID NO: 97 - protein, 12 aa, synthetic construct
  REGION 1..12: note = BC motif
GTSEPSTSEPGA

SEQ ID NO: 98 - protein, 12 aa, synthetic construct
  REGION 1..12: note = BC motif
GTSTEPSEPGSA

SEQ ID NO: 99 - protein, 12 aa, synthetic construct
  REGION 1..12: note = BD motif
GSTAGSETSTEA

SEQ ID NO: 100 - protein, 12 aa, synthetic construct
  REGION 1..12: note = BD motif
GSETATSGSETA

SEQ ID NO: 101 - protein, 12 aa, synthetic construct
  REGION 1..12: note = BD motif
GTSESATSESGA

SEQ ID NO: 102 - protein, 12 aa, synthetic construct
  REGION 1..12: note = BD motif
GTSTEASEGSAS

SEQ ID NO: 103 - protein, 1607 aa, synthetic construct
  REGION 1..1607: note = coFVIII-6-XTEN Protein Sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 104 - DNA, 16 bp, synthetic construct
  misc_feature 1..16: note = Rep-binding site (RBS) for AAV2
  source 1..16: mol_type = other DNA
gcgcgctcgctcgctc

SEQ ID NO: 105 - skipped

SEQ ID NO: 106 - protein, 2328 aa, synthetic construct
  REGION 1..2328: note = mature full length FVIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPKRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNISDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 107 - DNA, 7053 bp, synthetic construct
  misc_feature 1..7053: note = mature full length FVIII
  source 1..7053: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 108 - skipped

SEQ ID NO: 109 - protein, 1718 aa, synthetic construct
  REGION 1..1718: note = BIVV001 BDD-FVIII(XTEN) Sequence (no Fc)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPKRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 110 - DNA, 6526 bp, synthetic construct
  misc_feature 1..6526: note = NB15.1v32 construct (AAV-FVIIIco6XTEN-ssDNA)
  source 1..6526: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaggtaagtgccgtgtgtggttcccgcgggcctggcctctttacgggttatggcccttgcgtgccttgaattactgacactgacatccactttttctttttctccacagctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 111 - DNA, 130 bp, synthetic construct
  misc_feature 1..130: note = 5'ITR (5'-end AAV2 inverted terminal repeat)
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 112 - DNA, 142 bp, synthetic construct
  misc_feature 1..142: note = Plasmid Backbone Sequence (PBS)-1
  source 1..142: mol_type = other DNA
gcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaa

SEQ ID NO: 113 - DNA, 229 bp, synthetic construct
  misc_feature 1..229: note = TTPp (liver-specific promoter)
  source 1..229: mol_type = other DNA
acgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 114 - skipped

SEQ ID NO: 115 - DNA, 106 bp, synthetic construct
  misc_feature 1..106: note = Synthetic Intron
  source 1..106: mol_type = other DNA
gtaagtgccgtgtgtggttcccgcgggcctggcctctttacgggttatggcccttgcgtgccttgaattactgacactgacatccactttttctttttctccacag

SEQ ID NO: 116 - DNA, 11 bp, synthetic construct
  misc_feature 1..11: note = Plasmid Backbone Sequence (PBS)-3
  source 1..11: mol_type = other DNA
ctagcgccacc

SEQ ID NO: 117 - DNA, 4824 bp, synthetic construct
  misc_feature 1..4824: note = FVIIIco6XTEN (open reading frame for codon-optimized FVIII version 6 containing XTEN144)
  source 1..4824: mol_type = other DNA
atgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactga

SEQ ID NO: 118 - DNA, 450 bp, synthetic construct; identical to SEQ ID NO: 18
  misc_feature 1..450: note = XTEN144
  source 1..450: mol_type = other DNA

SEQ ID NO: 119 - DNA, 19 bp, synthetic construct
  misc_feature 1..19: note = Plasmid Backbone Sequence (PBS)-4
  source 1..19: mol_type = other DNA
atcagcctgagctcgctga

SEQ ID NO: 120 - DNA, 595 bp, synthetic construct
  misc_feature 1..595: note = WPRE (mutated woodchuck hepatitis virus post-transcriptional regulatory element)
  source 1..595: mol_type = other DNA
tcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctg

SEQ ID NO: 121 - skipped

SEQ ID NO: 122 - DNA, 211 bp, synthetic construct
  misc_feature 1..211: note = bGHpA (bovine growth hormone polyadenylation signal)
  source 1..211: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 123 - DNA, 119 bp, synthetic construct
  misc_feature 1..119: note = Plasmid Backbone Sequence (PBS)-6
  source 1..119: mol_type = other DNA
tgcggtgggctctatggcttctgaggcggaaagaacgggctcgagaagcttctagatatcctctcttaaggtagcatcgagatttaaattagggataacagggtaatggcgcgggccgc

SEQ ID NO: 124 - DNA, 130 bp, synthetic construct
  misc_feature 1..130: note = 3'ITR (3'-end AAV2 inverted terminal repeat)
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 125 - protein, 415 aa, synthetic construct
  REGION 1..415: note = Mature FIX polypeptide
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 126 - protein, 415 aa, synthetic construct
  REGION 1..415: note = Mature Padua(R338L)FIX Polypeptide
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 127 - protein, 46 aa, synthetic construct
  REGION 1..46: note = FIX Signal Polypeptide and Propeptide
MQRVNMIMAESPGLITICLLGYLLSAECTVFLDHENANKILNRPKR

SEQ ID NO: 128 - DNA, 8442 bp, synthetic construct
  misc_feature 1..8442: note = human VWF
  source 1..8442: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcrgagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgygtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagstgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtcttgctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 129 - protein, 2809 aa, synthetic construct
  REGION 1..2809: note = human VWF
  SITE 540: note = misc_feature - Xaa can be any naturally occurring amino acid
  SITE 1944: note = misc_feature - Xaa can be any naturally occurring amino acid
  SITE 2012: note = misc_feature - Xaa can be any naturally occurring amino acid
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLXEPRVEDFGNAWKLHGDCQDLQKQHSDPCALNPRMTRFSEEACAVLTSPTFEACHRAVSPLPYLRNCRYDVCSCSDGRECLCGASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPXVCTGSSTRHIVTFDGQNFKLTGSCSYVLFQNKEQDLEVILHNGACSPGARQGCMKSIEVKHSALSVEXHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 130 - protein, 36 aa, synthetic construct
  REGION 1..36: note = AE36
GSPAGSPTSTEEGTSESATPESGPGSEPATSGSETP

SEQ ID NO: 131 - protein, 42 aa, synthetic construct; identical to SEQ ID NO: 46
  REGION 1..42: note = AE42

SEQ ID NO: 132 - protein, 78 aa, synthetic construct
  REGION 1..78: note = AE72
GAPTSESATPESGPGSEPATSGSETPGTSESATPESGPGSEPATSGSETPGTSESATPESGPGTSTEPSEGSAPGASS

SEQ ID NO: 133 - protein, 78 aa, synthetic construct; identical to SEQ ID NO: 132
  REGION 1..78: note = AE78

SEQ ID NO: 134 - protein, 143 aa, synthetic construct
  REGION 1..143: note = AE144
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESAPESGPGSEPATSGSETPGTSTEPSEGSAP

SEQ ID NO: 135 - protein, 144 aa, synthetic construct; identical to SEQ ID NO: 56
  REGION 1..144: note = AE144_6B

SEQ ID NO: 136 - protein, 144 aa, synthetic construct
  REGION 1..144: note = AG144
GTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSP

SEQ ID NO: 137 - protein, 144 aa, synthetic construct; identical to SEQ ID NO: 60
  REGION 1..144: note = AG144_A

SEQ ID NO: 138 - protein, 288 aa, synthetic construct
  REGION 1..288: note = AE288
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 139 - protein, 288 aa, synthetic construct
  REGION 1..288: note = AE288_2
GSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 140 - protein, 288 aa, synthetic construct
  REGION 1..288: note = AG288
PGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGS

SEQ ID NO: 141 - protein, 576 aa, synthetic construct
  REGION 1..576: note = AE576
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 142 - protein, 576 aa, synthetic construct
  REGION 1..576: note = AG576
PGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGS

SEQ ID NO: 143 - protein, 864 aa, synthetic construct
  REGION 1..864: note = AE864
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 144 - protein, 864 aa, synthetic construct
  REGION 1..864: note = AG864
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 145 - protein, 72 aa, synthetic construct
  REGION 1..72: note = XTEN_AE72_2A_1
TSTEPSEGSAPGSPAGSPTSTEEGTSTEPSEGSAPGTSTEPSEGSAPGTSESATPESGPGTSTEPSEGSAPG

SEQ ID NO: 146 - protein, 72 aa, synthetic construct
  REGION 1..72: note = XTEN_AE72_2A_2
TSESATPESGPGSEPATSGSETPGTSTEPSEGSAPGTSTEPSEGSAPGTSESATPESGPGTSESATPESGPG

SEQ ID NO: 147 - protein, 72 aa, synthetic construct
  REGION 1..72: note = XTEN_AE72_3B_1
SPAGSPTSTEEGTSESATPESGPGSEPATSGSETPGTSESATPESGPGTSTEPSEGSAPGTSTEPSEGSAPG

SEQ ID NO: 148 - protein, 72 aa, synthetic construct
  REGION 1..72: note = XTEN_AE72_3B_2
TSTEPSEGSAPGTSTEPSEGSAPGTSTEPSEGSAPGTSTEPSEGSAPGSPAGSPTSTEEGTSTEPSEGSAPG

SEQ ID NO: 149 - protein, 72 aa, synthetic construct
  REGION 1..72: note = XTEN_AE72_4A_2
TSESATPESGPGSPAGSPTSTEEGSPAGSPTSTEEGSPAGSPTSTEEGTSESATPESGPGTSTEPSEGSAPG

SEQ ID NO: 150 - protein, 72 aa, synthetic construct
  REGION 1..72: note = XTEN_AE72_5A_2
SPAGSPTSTEEGTSESATPESGPGSEPATSGSETPGTSESATPESGPGSPAGSPTSTEEGSPAGSPTSTEEG

SEQ ID NO: 151 - protein, 72 aa, synthetic construct
  REGION 1..72: note = XTEN_AE72_6B_1
TSTEPSEGSAPGTSESATPESGPGTSESATPESGPGTSESATPESGPGSEPATSGSETPGSEPATSGSETPG

SEQ ID NO: 152 - protein, 72 aa, synthetic construct
  REGION 1..72: note = XTEN_AE72_6B_2
SPAGSPTSTEEGTSTEPSEGSAPGTSTEPSEGSAPGSEPATSGSETPGTSESATPESGPGTSTEPSEGSAPG

SEQ ID NO: 153 - protein, 72 aa, synthetic construct
  REGION 1..72: note = XTEN_AE72_1A_1
SPAGSPTSTEEGTSESATPESGPGTSTEPSEGSAPGSPAGSPTSTEEGTSTEPSEGSAPGTSTEPSEGSAPG

SEQ ID NO: 154 - protein, 72 aa, synthetic construct
  REGION 1..72: note = XTEN_AE72_1A_2
TSESATPESGPGSEPATSGSETPGSEPATSGSETPGSPAGSPTSTEEGTSESATPESGPGTSTEPSEGSAPG

SEQ ID NO: 155 - protein, 144 aa, synthetic construct
  REGION 1..144: note = XTEN_AE144_1A
SPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPG

SEQ ID NO: 156 - protein, 150 aa, synthetic construct
  REGION 1..150: note = AE150
GAPGSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESATPESGPGSEPATSGSETPGTSTEPSEGSAPASS

SEQ ID NO: 157 - protein, 150 aa, synthetic construct
  REGION 1..150: note = G150
GAPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPASS

SEQ ID NO: 158 - protein, 294 aa, synthetic construct
  REGION 1..294: note = AE294
GAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASS

SEQ ID NO: 159 - protein, 294 aa, synthetic construct
  REGION 1..294: note = AG294
GAPPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSASS

SEQ ID NO: 160 - protein, 1016 aa, synthetic construct
  REGION 1..1016: note = FIX-Linker-Albumin
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADFCYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 161 - protein, 424 aa, synthetic construct
  REGION 1..424: note = FIX
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTPVSQTSKLT

SEQ ID NO: 162 - protein, 9 aa, synthetic construct
  REGION 1..9: note = Linker
RAETVFPDV

SEQ ID NO: 163 - protein, 585 aa, synthetic construct [sequence omitted]

SEQ ID NO: 164 - protein, 763 aa, synthetic construct
  REGION 1..763: note = FIX(XTEN )-Fc
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDGPSPGSPTSTEEGTSESATPESGPGSEPATSGSETPGTSESATPESGPGTSTEPSEGSAPGTSTEPSEGSAPGASSNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 712 aa, synthetic construct
  REGION 1..712: note = FIX-FXIa-AE288
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGKLTRAETGGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 166 - protein, 944 aa, synthetic construct
  REGION 1..944: note = FIX-Fc-Fc
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRRRSGGGGSGGGGSGGGGSGGGGSRRRRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - DNA, 383 bp, synthetic construct
  misc_feature 1..383: note = Parvovirus B19 KY940273.1
  source 1..383: mol_type = other DNA
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 168 - DNA, 248 bp, synthetic construct
  misc_feature 1..248: note = Parvorius B19 d135
  source 1..248: mol_type = other DNA
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaatttt

SEQ ID NO: 169 - DNA, 129 bp, synthetic construct
  misc_feature 1..129: note = Parvovirus v1 AAV2
  source 1..129: mol_type = other DNA
cggcgcgccgcttgatcttagtggcacgtcaaccagcgctggcccagagccaaccctaattccggaagtcctcagtccgccatcttgcccgcctaccggcggcgaccggcggcatcatttggtgttctt

SEQ ID NO: 170 - DNA, 113 bp, synthetic construct
  misc_feature 1..113: note = Parvovirus V2
  source 1..113: mol_type = other DNA
ctctgggccagcttgcttggggttgccttgacactaagacaagcggcgcgccgcttgatcttagtggcacgtcaaccccaagcgctggcccagagtgtcttcttttaaatttt

SEQ ID NO: 171 - DNA, 340 bp, synthetic construct
  misc_feature 1..340: note = Parvovirus V3
  source 1..340: mol_type = other DNA
caaatcagatgccgccggtcgccgccggtaggcgggacttccggtacaagatggcggacaattacgtcatttcctgtgacgtatttcctgtgacgtacttccggtggcgggacttccggaattttggctctgggccagcttgcttggggttgccttgaccaagcgcgcgccgcttgatcaccccaagcgctggcccagagccacctaaccggaagtccccccaccggaagtgacgtcacaggaaagacgtcacaggaagtaattgtccgccatcttgtaccggaagtcccgcaccggcggcgaccggcggcatctgatttggtgtcttcttttaaatttt

SEQ ID NO: 172 - DNA, 444 bp, synthetic construct
  misc_feature 1..444: note = Parvovirus U25749.1
  source 1..444: mol_type = other DNA
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 173 - DNA, 282 bp, synthetic construct
  misc_feature 1..282: note = Parvovirus GPV AAV2 V1
  source 1..282: mol_type = other DNA
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtg

SEQ ID NO: 174 - DNA, 145 bp, synthetic construct
  misc_feature 1..145: note = Parvovirus GPV AAV2 V1
  source 1..145: mol_type = other DNA
ttgaccacgcgcatgccgcgcggtcagcccaatagttaagccgggtgaccacacgtgacaggaagcacgggatgtgcgtcaccggaagcagtgaccgggctggttcgaacgaacgaaccctccaactcaaggacaagaggatatt

SEQ ID NO: 175 - DNA, 120 bp, synthetic construct
  misc_feature 1..120: note = Parvovirus GPV V2
  source 1..120: mol_type = other DNA
cggtgacgtgtttccggctgttaggttgaccacgcgcatgccgcgcggtcagcccaatagttaagccggaaacacgtcaccgactcaaggacaagaggatattttgcgcgccaggaagtg

SEQ ID NO: 176 - DNA, 102 bp, synthetic construct
  misc_feature 1..102: note = Parvovirus GPV V3
  source 1..102: mol_type = other DNA
gggaacaatcaggggaagtgaccggtgacgtcatgtaacttgcgtcacttcccgttcgaacgaacgaacgagactcaaggacaagaggcgcgccaggaagtg

SEQ ID NO: 177 - DNA, 145 bp, synthetic construct
  misc_feature 1..145: note = Parvovirus AAV2 NC_001401.2
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 178 - DNA, 130 bp, synthetic construct; identical to SEQ ID NO: 111
  misc_feature 1..130: note = Parvovirus Gtx
  source 1..130: mol_type = other DNA

SEQ ID NO: 179 - DNA, 6762 bp, synthetic construct
  misc_feature 1..6762: note = B19-FVIII construct
  source 1..6762: mol_type = other DNA
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttgcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaggtaagtgccgtgtgtggttcccgcgggcctggcctctttacgggttatggcccttgcgtgccttgaattactgacactgacatccactttttctttttctccacagctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgcaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagag

SEQ ID NO: 180 - DNA, 248 bp, synthetic construct; identical to SEQ ID NO: 168
  misc_feature 1..248: note = 5' ITR
  source 1..248: mol_type = other DNA

SEQ ID NO: 181 - DNA, 248 bp, synthetic construct
  misc_feature 1..248: note = 3' ITR
  source 1..248: mol_type = other DNA
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagag

SEQ ID NO: 182 - DNA, 6830 bp, synthetic construct
  misc_feature 1..6830: note = Synthetic polynucleotide sequence
  source 1..6830: mol_type = other DNA
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtggcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaggtaagtgccgtgtgtggttcccgcgggcctggcctctttacgggttatggcccttgcgtgccttgaattactgacactgacatccactttttctttttctccacagctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgccacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccg

SEQ ID NO: 183 - DNA, 282 bp, synthetic construct; identical to SEQ ID NO: 173
  misc_feature 1..282: note = 5' ITR
  source 1..282: mol_type = other DNA

SEQ ID NO: 184 - DNA, 282 bp, synthetic construct
  misc_feature 1..282: note = 3' ITR
  source 1..282: mol_type = other DNA
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccg

SEQ ID NO: 185 - DNA, 383 bp, synthetic construct; identical to SEQ ID NO: 167
  misc_feature 1..383: note = 5' ITR
  source 1..383: mol_type = other DNA

SEQ ID NO: 186 - DNA, 383 bp, synthetic construct
  misc_feature 1..383: note = 3' ITR
  source 1..383: mol_type = other DNA
aaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 187 - DNA, 444 bp, synthetic construct; identical to SEQ ID NO: 172
  misc_feature 1..444: note = 5' ITR
  source 1..444: mol_type = other DNA

SEQ ID NO: 188 - DNA, 444 bp, synthetic construct
  misc_feature 1..444: note = 3' ITR
  source 1..444: mol_type = other DNA
cacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgag

SEQ ID NO: 189 - DNA, 7032 bp, synthetic construct
  misc_feature 1..7032: note = Synthetic polynucleotide sequence
  source 1..7032: mol_type = other DNA
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttgcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaggtaagtgccgtgtgtggttcccgcgggcctggcctctttacgggttatggcccttgcgtgccttgaattactgacactgacatccactttttctttttctccacagctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgcaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 190 - DNA, 6824 bp, synthetic construct
  misc_feature 1..6824: note = Synthetic polynucleotide sequence
  source 1..6824: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaatcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacaggctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 191 - DNA, 278 bp, synthetic construct
  misc_feature 1..278: note = Synthetic polynucleotide sequence
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 192 - DNA, 262 bp, synthetic construct
  misc_feature 1..262: note = Synthetic polynucleotide sequence
  source 1..262: mol_type = other DNA
gtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 193 - DNA, 130 bp, synthetic construct; identical to SEQ ID NO: 124
  misc_feature 1..130: note = 3' ITR
  source 1..130: mol_type = other DNA

SEQ ID NO: 194 - DNA, 7154 bp, synthetic construct
  misc_feature 1..7154: note = Synthetic polynucleotide sequence
  source 1..7154: mol_type = other DNA
ctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtggcggcaattcagtcgataactataacggtcctaaggtagcgatttaaatacgcgctctcttaaggtagccccgggacgcgtcaattgagatctggatccggtaccgaattcgcggccgcctcgacgactagcgtttaattaaacgcgtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaggtaagtgccgtgtgtggttcccgcgggcctggcctctttacgggttatggcccttgcgtgccttgaattactgacactgacatccactttttctttttctccacagctagcgccaccatgcagattgagctgtccacttgtttcttcctgtgcctcctgcgcttctgtttctccgccactcgccggtactaccttggagccgtggagctttcatgggactacatgcagagcgacctgggcgaactccccgtggatgccagattccccccccgcgtgccaaagtccttcccctttaacacctccgtggtgtacaagaaaaccctctttgtcgagttcactgaccacctgttcaacatcgccaagccgcgcccaccttggatgggcctcctgggaccgaccattcaagctgaagtgtacgacaccgtggtgatcaccctgaagaacatggcgtcccaccccgtgtccctgcatgcggtcggagtgtcctactggaaggcctccgaaggagctgagtacgacgaccagactagccagcgggaaaaggaggacgataaagtgttcccgggcggctcgcatacttacgtgtggcaagtcctgaaggaaaacggacctatggcatccgatcctctgtgcctgacttactcctacctttcccatgtggacctcgtgaaggacctgaacagcgggctgattggtgcacttctcgtgtgccgcgaaggttcgctcgctaaggaaaagacccagaccctccataagttcatccttttgttcgctgtgttcgatgaaggaaagtcatggcattccgaaactaagaactcgctgatgcaggaccgggatgccgcctcagcccgcgcctggcctaaaatgcatacagtcaacggatacgtgaatcggtcactgcccgggctcatcggttgtcacagaaagtccgtgtactggcacgtcatcggcatgggcactacgcctgaagtgcactccatcttcctggaagggcacaccttcctcgtgcgcaaccaccgccaggcctctctggaaatctccccgattacctttctgaccgcccagactctgctcatggacctggggcagttccttctcttctgccacatctccagccatcagcacgacggaatggaggcctacgtgaaggtggactcatgcccggaagaacctcagttgcggatgaagaacaacgaggaggccgaggactatgacgacgatttgactgactccgagatggacgtcgtgcggttcgatgacgacaacagccccagcttcatccagattcgcagcgtggccaagaagcaccccaaaacctgggtgcactacatcgcggccgaggaagaagattgggactacgccccgttggtgctggcacccgatgaccggtcgtacaagtcccagtatctgaacaatggtccgcagcggattggcagaaagtacaagaaagtgcggttcatggcgtacactgacgaaacgtttaagacccgggaggccattcaacatgagagcggcattctgggaccactgctgtacggagaggtcggcgataccctgctcatcatcttcaaaaaccaggcctcccggccttacaacatctaccctcacggaatcaccgacgtgcggccactctactcgcggcgcctgccgaagggcgtcaagcacctgaaagacttccctatcctgccgggcgaaatcttcaagtataagtggaccgtcaccgtggaggacgggcccaccaagagcgatcctaggtgtctgactcggtactactccagcttcgtgaacatggaacgggacctggcatcgggactcattggaccgctgctgatctgctacaaagagtcggtggatcaacgcggcaaccagatcatgtccgacaagcgcaacgtgatcctgttctccgtgtttgatgaaaacagatcctggtacctcactgaaaacatccagaggttcctcccaaaccccgcaggagtgcaactggaggaccctgagtttcaggcctcgaatatcatgcactcgattaacggttacgtgttcgactcgctgcaactgagcgtgtgcctccatgaagtcgcttactggtacattctgtccatcggcgcccagactgacttcctgagcgtgttcttttccggttacacctttaagcacaagatggtgtacgaagataccctgaccctgttccctttctccggcgaaacggtgttcatgtcgatggagaacccgggtctgtggattctgggatgccacaacagcgactttcggaaccgcggaatgactgccctgctgaaggtgtcctcatgcgacaagaacaccggagactactacgaggactcctacgaggatatctcagcctacctcctgtccaagaacaacgcgatcgagccgcgcagcttcagccagaacggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcccgcctgtgctgaagaggcaccagcgagaaattacccggaccaccctccaatcggatcaggaggaaatcgactacgacgacaccatctcggtggaaatgaagaaggaagatttcgatatctacgacgaggacgaaaatcagtcccctcgctcattccaaaagaaaactagacactactttatcgccgcggtggaaagactgtgggactatggaatgtcatccagccctcacgtccttcggaaccgggcccagagcggatcggtgcctcagttcaagaaagtggtgttccaggagttcaccgacggcagcttcacccagccgctgtaccggggagaactgaacgaacacctgggcctgctcggtccctacatccgcgcggaagtggaggataacatcatggtgaccttccgtaaccaagcatccagaccttactccttctattcctccctgatctcatacgaggaggaccagcgccaaggcgccgagccccgcaagaacttcgtcaagcccaacgagactaagacctacttctggaaggtccaacaccatatggccccgaccaaggatgagtttgactgcaaggcctgggcctacttctccgacgtggaccttgagaaggatgtccattccggcctgatcgggccgctgctcgtgtgtcacaccaacaccctgaacccagcgcatggacgccaggtcaccgtccaggagtttgctctgttcttcaccatttttgacgaaactaagtcctggtacttcaccgagaatatggagcgaaactgtagagcgccctgcaatatccagatggaagatccgactttcaaggagaactatagattccacgccatcaacgggtacatcatggatactctgccggggctggtcatggcccaggatcagaggattcggtggtacttgctgtcaatgggatcgaacgaaaacattcactccattcacttctccggtcacgtgttcactgtgcgcaagaaggaggagtacaagatggcgctgtacaatctgtaccccggggtgttcgaaactgtggagatgctgccgtccaaggccggcatctggagagtggagtgcctgatcggagagcacctccacgcggggatgtccaccctcttcctggtgtactcgaataagtgccagaccccgctgggcatggcctcgggccacatcagagacttccagatcacagcaagcggacaatacggccaatgggcgccgaagctggcccgcttgcactactccggatcgatcaacgcatggtccaccaaggaaccgttctcgtggattaaggtggacctcctggcccctatgattatccacggaattaagacccagggcgccaggcagaagttctcctccctgtacatctcgcaattcatcatcatgtacagcctggacgggaagaagtggcagacttacaggggaaactccaccggcaccctgatggtctttttcggcaacgtggattcctccggcattaagcacaacatcttcaacccaccgatcatagccagatatattaggctccaccccactcactactcaatccgctcaactcttcggatggaactcatggggtgcgacctgaactcctgctccatgccgttggggatggaatcaaaggctattagcgacgcccagatcaccgcgagctcctacttcactaacatgttcgccacctggagcccctccaaggccaggctgcacttgcagggacggtcaaatgcctggcggccgcaagtgaacaatccgaaggaatggcttcaagtggatttccaaaagaccatgaaagtgaccggagtcaccacccagggagtgaagtcccttctgacctcgatgtatgtgaaggagttcctgattagcagcagccaggacgggcaccagtggaccctgttcttccaaaacggaaaggtcaaggtgttccaggggaaccaggactcgttcacacccgtggtgaactccctggaccccccactgctgacgcggtacttgaggattcatcctcagtcctgggtccatcagattgcattgcgaatggaagtcctgggctgcgaggcccaggacctgtactgaatcagcctgagctcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgccacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgag

SEQ ID NO: 195 - DNA, 648 bp, synthetic construct
  misc_feature 1..648: note = Synthetic polynucleotide sequence
  source 1..648: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 196 - DNA, 1428 bp, synthetic construct
  misc_feature 1..1428: note = Synthetic polynucleotide sequence
  source 1..1428: mol_type = other DNA
atggactacaaagaccatgacggtgattataaagatcatgatatcgattacaaggatgacgatgacaaggctgctgtggttctggaaaatggcgtgctgagccggaagctgagcgacttcggacaagagacaagctacatcgaggacaacagcaaccagaatggcgccgtgtctctgatcttcagcctgaaagaagaagtgggcgccctggccaaggtgctgagactgttcgaggaaaacgagatcaatctgacccacatcgagagcagacccagcagactgaacaaggacgagtacgagttcttcacctacctggacaagcggagcaagcctgtgctgggcagcatcatcaagagcctgagaaacgacatcggcgccaccgtgcacgagctgagcagagacaaagaaaagaacaccgtgccatggttccccaggaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacaccgaggaagagagaaagacctggggcaccgtgttcagaaccctgaaggccctgtacaagacccacgcctgctacgagcacaaccacatcttcccactgctggaaaagtactgcggcttccgcgaggacaatatccctcagctcgaagacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggaattcggcctgtgcaaagagggcgacagcatcaaggcttatggcgctggactgctgtctagcttcggcgagctgcagtactgtctgagcgacaagcctaagctgctgcccctggaactggaaaagaccgcctgccaagagtacacagtgaccgagttccagcctctgtactacgtggccgagagcttcaacgacgccaaagaaaaagtgcggaccttcgccgccaccattcctcggccttttagcgtcagatacgacccctacacacagcgcgtggaagtgctggacaacacacagcagctgaagattctggccgactccatcaacagcgaagtgggcattctgtgtcacgccctgcagaagatcaagagctga

SEQ ID NO: 197 - DNA, 4146 bp, synthetic construct
  misc_feature 1..4146: note = Synthetic polynucleotide sequence
  source 1..4146: mol_type = other DNA
ctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttgcggcaattcagtcgataactataacggtcctaaggtagcgatttaaatacgcgctctcttaaggtagccccgggacgcgtcaattgagatctggatccggtaccgaattcgcggccgcctcgacgactagcgtttagtaatgagacgcacaaactaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcgcgaagccgccaccatggactacaaagaccatgacggtgattataaagatcatgatatcgattacaaggatgacgatgacaaggctgctgtggttctggaaaatggcgtgctgagccggaagctgagcgacttcggacaagagacaagctacatcgaggacaacagcaaccagaatggcgccgtgtctctgatcttcagcctgaaagaagaagtgggcgccctggccaaggtgctgagactgttcgaggaaaacgagatcaatctgacccacatcgagagcagacccagcagactgaacaaggacgagtacgagttcttcacctacctggacaagcggagcaagcctgtgctgggcagcatcatcaagagcctgagaaacgacatcggcgccaccgtgcacgagctgagcagagacaaagaaaagaacaccgtgccatggttccccaggaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacaccgaggaagagagaaagacctggggcaccgtgttcagaaccctgaaggccctgtacaagacccacgcctgctacgagcacaaccacatcttcccactgctggaaaagtactgcggcttccgcgaggacaatatccctcagctcgaagacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggaattcggcctgtgcaaagagggcgacagcatcaaggcttatggcgctggactgctgtctagcttcggcgagctgcagtactgtctgagcgacaagcctaagctgctgcccctggaactggaaaagaccgcctgccaagagtacacagtgaccgagttccagcctctgtactacgtggccgagagcttcaacgacgccaaagaaaaagtgcggaccttcgccgccaccattcctcggccttttagcgtcagatacgacccctacacacagcgcgtggaagtgctggacaacacacagcagctgaagattctggccgactccatcaacagcgaagtgggcattctgtgtcacgccctgcagaagatcaagagctgagcaagtaatgagcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgcaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagatcaagcggcgcgccgcttgtcttagtgtcaaggcaaccccaagcaagctggcccagag

SEQ ID NO: 198 - DNA, 4214 bp, synthetic construct
  misc_feature 1..4214: note = Synthetic polynucleotide sequence
  source 1..4214: mol_type = other DNA
cggtgacgtgtttccggctgttaggttgaccacgcgcatgccgcgcggtcagcccaatagttaagccggaaacacgtcaccggaagtcacatgaccggaagtcacgtgaccggaaacacgtgacaggaagcacgtgaccggaactacgtcaccggatgtgcgtcaccggaagcatgtgaccggaacttgcgtcacttccccctcccctgattggctggttcgaacgaacgaaccctccaatgagactcaaggacaagaggatattttgcgcgccaggaagtggcggcaattcagtcgataactataacggtcctaaggtagcgatttaaatacgcgctctcttaaggtagccccgggacgcgtcaattgagatctggatccggtaccgaattcgcggccgcctcgacgactagcgtttagtaatgagacgcacaaactaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcgcgaagccgccaccatggactacaaagaccatgacggtgattataaagatcatgatatcgattacaaggatgacgatgacaaggctgctgtggttctggaaaatggcgtgctgagccggaagctgagcgacttcggacaagagacaagctacatcgaggacaacagcaaccagaatggcgccgtgtctctgatcttcagcctgaaagaagaagtgggcgccctggccaaggtgctgagactgttcgaggaaaacgagatcaatctgacccacatcgagagcagacccagcagactgaacaaggacgagtacgagttcttcacctacctggacaagcggagcaagcctgtgctgggcagcatcatcaagagcctgagaaacgacatcggcgccaccgtgcacgagctgagcagagacaaagaaaagaacaccgtgccatggttccccaggaccatccaagagctggacagattcgccaaccagatcctgagctatggcgccgagctggacgctgatcaccctggctttaaggaccccgtgtaccgggccagaagaaagcagtttgccgatatcgcctacaactaccggcacggccagcctattcctcgggtcgagtacaccgaggaagagagaaagacctggggcaccgtgttcagaaccctgaaggccctgtacaagacccacgcctgctacgagcacaaccacatcttcccactgctggaaaagtactgcggcttccgcgaggacaatatccctcagctcgaagacgtgtcccagttcctgcagacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagaagcttcgcccagttcagccaagagatcggactggcttctctgggagcccctgacgagtacattgagaagctggccaccatctactggttcaccgtggaattcggcctgtgcaaagagggcgacagcatcaaggcttatggcgctggactgctgtctagcttcggcgagctgcagtactgtctgagcgacaagcctaagctgctgcccctggaactggaaaagaccgcctgccaagagtacacagtgaccgagttccagcctctgtactacgtggccgagagcttcaacgacgccaaagaaaaagtgcggaccttcgccgccaccattcctcggccttttagcgtcagatacgacccctacacacagcgcgtggaagtgctggacaacacacagcagctgaagattctggccgactccatcaacagcgaagtgggcattctgtgtcacgccctgcagaagatcaagagctgagcaagtaatgagcgctgatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgggctcgagaagcttctagatatcctctcttaaggtagcatcgagatttaaattagggataacagggtaatggcgcgggccgccacttcctggcgcgcaaaatatcctcttgtccttgagtctcattggagggttcgttcgttcgaaccagccaatcaggggagggggaagtgacgcaagttccggtcacatgcttccggtgacgcacatccggtgacgtagttccggtcacgtgcttcctgtcacgtgtttccggtcacgtgacttccggtcatgtgacttccggtgacgtgtttccggcttaactattgggctgaccgcgcggcatgcgcgtggtcaacctaacagccggaaacacgtcaccg

(19) United States
(12) Patent Application Publication
(10) Pub. No.: US 2025/0269067 A1
(43) Pub. Date: Aug. 28, 2025

(54) NUCLEIC ACID MOLECULES AND USES THEREOF FOR NON-VIRAL GENE THERAPY

(71) Applicant: BIOVERATIV THERAPEUTICS INC., Waltham, MA (US)

(72) Inventors: Tongyao LIU, Waltham, MA (US); Alexey SEREGIN, Waltham, MA (US); Robert T. PETERS, Waltham, MA (US); Jiayun LIU, Waltham, MA (US); Philip ZAKAS, Waltham, MA (US); Douglas DRAGER, Waltham, MA (US); Susannah PATARROYO-WHITE, Waltham, MA (US)

(21) Appl. No.: 19/025,334

(22) Filed: Jan. 16, 2025

Related U.S. Application Data
(62) Division of application No. 16/537,192, filed on Aug. 9, 2019.
(60) Provisional application No. 62/716,826, filed on Aug. 9, 2018.

(51) Int. Cl. A61K 48/00; A61K 9/00; A61P 7/04; C07K 14/755; C12N 7/00; C12N 15/86

(52) U.S. Cl. CPC A61K 48/0058; A61K 9/0019; A61K 48/0016; A61K 48/0033; A61K 48/0066; A61P 7/04; C07K 14/755; C12N 7/00; C12N 15/86; C12N 2750/14143; C12N 2750/14171; C12N 2750/14343; C12N 2750/14371; C12N 2830/42; C12N 2830/50

(57) ABSTRACT

The present disclosure provides nucleic acid molecules comprising a first inverted terminal repeat (ITR), a second ITR, and a genetic cassette encoding a target sequence. In some embodiments, the target sequence encodes a miRNA and/or a therapeutic protein. In certain embodiments, the therapeutic protein comprises a clotting factor, a growth factor, a hormone, a cytokine, an antibody, a fragment thereof, and a combination thereof. In some embodiments, the first ITR and/or the second ITR is an ITR of a non-adeno-associated virus (AAV). The present disclosure also provides methods of treating a metabolic disorder of the liver in a subject comprising administering to the subject the nucleic acid molecule or a polypeptide encoded thereby.

### RELATED APPLICATIONS

This application is a continuation of U.S. patent application Ser. No. 16/537,192, filed Aug. 9, 2019, which claims priority to U.S. Provisional Patent Application Ser. No. 62/716,826, filed Aug. 9, 2018, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jan. 16, 2025, is named 761239_SA9-465CON_ST26.xml and is 421,309 bytes in size.

### BACKGROUND OF THE DISCLOSURE

Gene therapy offers the potential for a lasting means of treating a variety of diseases. In the past, many gene therapy treatments typically relied on the use of viruses. There are numerous viral agents that could be selected for this purpose, each with distinct properties that would make them more or less suitable for gene therapy. Zhou et al., Adv Drug Deliv Rev. 106(Pt A):3-26, 2016. However, the undesired properties of some viral vectors, including their immunogenic profiles or their propensity to cause cancer, have resulted in clinical safety concerns and, until recently, limited their clinical use to certain applications, for example, vaccines and oncolytic strategies. Cotter et al., Front Biosci. 10:1098-105 (2005).

Adeno-associated virus (AAV) is one of the most commonly investigated gene therapy vectors. AAV is a protein shell surrounding and protecting a small, single-stranded DNA genome of approximately 4.8 kilobases (kb). Naso et al., BioDrugs, 31(4): 317-334, 2017. AAV belongs to the parvovirus family and is dependent on co-infection with other viruses, mainly adenoviruses, in order to replicate. Id. Its single-stranded genome contains three genes, Rep (Replication), Cap (Capsid), and aap (Assembly). Id. These coding sequences are flanked by inverted terminal repeats (ITRs) that are required for genome replication and packaging. Id. The two cis-acting AAV ITRs are approximately 145 nucleotides in length with interrupted palindromic sequences that can fold into T shaped hairpin structures that function as primers during initiation of DNA replication.

The use of conventional AAV as a gene delivery vector has certain drawbacks, however. One of the major drawbacks is associated with the AAV's limited viral packaging capacity of about 4.5 kb of heterologous DNA. (Dong et al., Hum Gene Ther. 7(17): 2101-12, 1996). In addition, administration of AAV vectors can induce an immune response in humans. Although AAV has been shown to be less immunogenic than some other viruses (i.e. adenovirus), the capsid proteins can trigger various components of the human immune system. See Naso et al., 2017. AAV is a common virus in the human population, and most people have been exposed to AAV, accordingly most people have already developed an immune response against the particular variants to which they had previously been exposed. This pre-existing adaptive response can include neutralizing antibodies (NAbs) and T cells that could diminish the clinical efficacy of subsequent re-infections with AAV and/or the elimination of cells that have been transduced, which may disqualify patients with pre-existing anti-AAV immunity to AVV based gene therapy treatment. Furthermore, evidence suggests that the T-shaped hairpin loops of AAV ITRs are susceptible to inhibition by host cell proteins/protein complexes that bind the T-shaped hairpin structures of AAV ITRs. See, e.g., Zhou et al., Scientific Reports 7:5432 (Jul. 14, 2017).

Thus, there exists a need in the art to efficiently and persistently express target sequences, e.g., therapeutic proteins and/or miRNAs, in in vitro and in vivo settings, while avoiding some of the unintended consequences and limitations of existing AAV vector technology.

### SUMMARY OF THE DISCLOSURE

In certain aspects, a nucleic acid molecule comprising a first inverted terminal repeat (ITR) and a second ITR flanking a genetic cassette comprising a heterologous polynucleotide sequence, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided

In certain exemplary embodiments, the first ITR comprises the nucleotide sequence set forth in SEQ ID NO: 180 and the second ITR comprises the nucleotide sequence set forth in SEQ ID NO: 181. In certain exemplary embodiments, the first ITR comprises the nucleotide sequence set forth in SEQ ID NO: 183 and the second ITR comprises the nucleotide sequence set forth in SEQ ID NO: 184. In certain exemplary embodiments, the first ITR comprises the nucleotide sequence set forth in SEQ ID NO: 185 and the second ITR comprises the nucleotide sequence set forth in SEQ ID NO: 186. In certain exemplary embodiments, the first ITR comprises the nucleotide sequence set forth in SEQ ID NO: 187 and the second ITR comprises the nucleotide sequence set forth in SEQ ID NO: 188.

In certain exemplary embodiments, the first ITR and/or the second ITR consists of a nucleotide sequence set forth in SEQ ID NO: 180, 181, 183, 184, 185, 186, 187 or 188. In certain exemplary embodiments, the first ITR and the second ITR are reverse complements of each other.

In certain exemplary embodiments, the nucleic acid molecule further comprises a promoter. In certain exemplary embodiments, the promoter is a tissue-specific promoter. In certain exemplary embodiments, the promoter drives expression of the heterologous polynucleotide sequence in an organ selected from the muscle, central nervous system (CNS), ocular, liver, heart, kidney, pancreas, lungs, skin, bladder, urinary tract, or any combination thereof. In certain exemplary embodiments, the promoter drives expression of the heterologous polynucleotide sequence in hepatocytes, endothelial cells, cardiac muscle cells, skeletal muscle cells, sinusoidal cells, afferent neurons, efferent neurons, interneurons, glial cells, astrocytes, oligodendrocytes, microglia, ependymal cells, lung epithelial cells, Schwann cells, satellite cells, photoreceptor cells, retinal ganglion cells, or any combination thereof. In certain exemplary embodiments, the promoter is positioned 5′ to the heterologous polynucleotide sequence. In certain exemplary embodiments, the promoter is selected from the group consisting of a mouse thyretin promoter (mTTR), an endogenous human factor VIII promoter (F8), a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, α1-antitrypsin (AAT), muscle creatine kinase (MCK), myosin heavy chain alpha (αMHC), myoglobin (MB), desmin (DES), SPc5-12, 2R5Sc5-12, dMCK, tMCK, and a phosphoglycerate kinase (PGK) promoter.

In certain exemplary embodiments, the heterologous polynucleotide sequence further comprises an intronic sequence. In certain exemplary embodiments, the intronic sequence is positioned 5′ to the heterologous polynucleotide sequence. In certain exemplary embodiments, the intronic sequence is positioned 3′ to the promoter. In certain exemplary embodiments, the intronic sequence comprises a synthetic intronic sequence. In certain exemplary embodiments, the intronic sequence comprises SEQ ID NO: 115 or 192.

In certain exemplary embodiments, the genetic cassette further comprises a post-transcriptional regulatory element. In certain exemplary embodiments, the post-transcriptional regulatory element is positioned 3′ to the heterologous polynucleotide sequence. In certain exemplary embodiments, the post-transcriptional regulatory element comprises a mutated woodchuck hepatitis virus post-transcriptional regulatory element (WPRE), a microRNA binding site, a DNA nuclear targeting sequence, or any combination thereof. In certain exemplary embodiments, the microRNA binding site comprises a binding site to miR142-3p.

In certain exemplary embodiments, the genetic cassette further comprises a 3′UTR poly(A) tail sequence. In certain exemplary embodiments, the 3′UTR poly(A) tail sequence is selected from the group consisting of bGH poly(A), actin poly(A), hemoglobin poly(A), and any combination thereof. In certain exemplary embodiments, the 3′UTR poly(A) tail sequence comprises bGH poly(A).

In certain exemplary embodiments, the genetic cassette further comprises an enhancer sequence. In certain exemplary embodiments, the enhancer sequence is positioned between the first ITR and the second ITR.

In certain exemplary embodiments, the nucleic acid molecule comprises from 5′ to 3′: the first ITR, the genetic cassette, and the second ITR; wherein the genetic cassette comprises a tissue-specific promoter sequence, an intronic sequence, the heterologous polynucleotide sequence, a post-transcriptional regulatory element, and a 3′UTR poly(A) tail sequence. In certain exemplary embodiments, the genetic cassette comprises from 5′ to 3′: a tissue-specific promoter sequence, an intronic sequence, the heterologous polynucleotide sequence, a post-transcriptional regulatory element, and a 3′UTR poly(A) tail sequence. In certain exemplary embodiments, the tissue specific promoter sequence comprises a TTT promoter; the intron is a synthetic intron; the post-transcriptional regulatory element comprises WPRE; and the 3′UTR poly(A) tail sequence comprises bGHpA.

In certain exemplary embodiments, the genetic cassette comprises a single stranded nucleic acid. In certain exemplary embodiments, the genetic cassette comprises a double stranded nucleic acid.

In certain exemplary embodiments, the heterologous polynucleotide sequence encodes a clotting factor, a growth factor, a hormone, a cytokine, an antibody, a fragment thereof, or any combination thereof.

In certain exemplary embodiments, the heterologous polynucleotide sequence encodes a growth factor selected from the group consisting of adrenomedullin (AM), angiopoietin (Ang), autocrine motility factor, a bone morphogenetic protein (BMP) (e.g. BMP2, BMP4, BMP5, BMP7), a ciliary neurotrophic factor family member (e.g., ciliary neurotrophic factor (CNTF), leukemia inhibitory factor (LIF), interleukin-6 (IL-6)), a colony-stimulating factor (e.g., macrophage colony-stimulating factor (m-CSF), granulocyte colony-stimulating factor (G-CSF), granulocyte macrophage colony-stimulating factor (GM-CSF)), an epidermal growth factor (EGF), an ephrin (e.g., ephrin A1, ephrin A2, ephrin A3, ephrin A4, ephrin A5, ephrin B1, ephrin B2, ephrin B3), erythropoietin (EPO), a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), foetal bovine somatotrophin (FBS), a GDNF family member (e.g., glial cell line-derived neurotrophic factor (GDNF), neurturin, persephin, artemin), growth differentiation factor-9 (GDF9), hepatocyte growth factor (HGF), hepatoma-derived growth factor (HDGF), insulin, an insulin-like growth factors (e.g., insulin-like growth factor-1 (IGF-1) or IGF-2, an interleukin (IL) (e.g., IL-1, IL-2, IL-3, IL-4, IL-5, IL-6, IL-7), keratinocyte growth factor (KGF), migration-stimulating factor (MSF), macrophage-stimulating protein (MSP or hepatocyte growth factor-like protein (HGFLP)), myostatin (GDF-8), a neuregulin (e.g., neuregulin 1 (NRG1), NRG2, NRG3, NRG4), a neurotrophin (e.g., brain-derived neurotrophic factor (BDNF), nerve growth factor (NGF), a neurotrophin-3 (NT-3), NT-4, placental growth factor (PGF), platelet-derived growth factor (PDGF), renalase (RNLS), T-cell growth factor (TCGF), thrombopoietin (TPO), a transforming growth factor (e.g., transforming growth factor alpha (TGF-α), TGF-β, tumor necrosis factor-alpha (TNF-α), and vascular endothelial growth factor (VEGF), and any combination thereof.

In certain exemplary embodiments, the heterologous polynucleotide sequence encodes a hormone.

In certain exemplary embodiments, the heterologous polynucleotide sequence encodes a cytokine.

In certain exemplary embodiments, the heterologous polynucleotide sequence encodes an antibody or a fragment thereof.

In certain exemplary embodiments, the heterologous polynucleotide sequence encodes a gene selected from dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), and any combination thereof.

In certain exemplary embodiments, the heterologous polynucleotide sequence encodes a microRNA (miRNA). In certain exemplary embodiments, the miRNA down regulates the expression of a target gene selected from SOD1, HTT, RHO, and any combination thereof

In certain exemplary embodiments, the heterologous polynucleotide sequence encodes a clotting factor selected from the group consisting of factor I (FI), factor II (FII), factor III (FIII), factor IV (FVI), factor V (FV), factor VI (FVI), factor VII (FVII), factor VIII (FVIII), factor IX (FIX), factor X (FX), factor XI (FXI), factor XII (FXII), factor XIII (FVIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), and any combination thereof.

In certain exemplary embodiments, the clotting factor is FVIII. In certain exemplary embodiments, the FVIII comprises full-length mature FVIII. In certain exemplary embodiments, the FVIII comprises an amino acid sequence at least about 70%, at least about 75%, at least about 80%, at least about 85%, at least about 90%, at least about 95%, at least about 96%, at least about 97%, at least about 98%, at least about 99%, or 100% identical to an amino acid sequence having SEQ ID NO: 106.

In certain exemplary embodiments, the FVIII comprises A1 domain, A2 domain, A3 domain, C1 domain, C2 domain, and a partial or no B domain. In certain exemplary embodiments, the FVIII comprises an amino acid sequence at least about 70%, at least about 75%, at least about 80%, at least about 85%, at least about 90%, at least about 95%, at least about 96%, at least about 97%, at least about 98%, at least about 99%, or 100% identical to the amino acid sequence of SEQ ID NO:109.

In certain exemplary embodiments, the clotting factor comprises a heterologous moiety. In certain exemplary embodiments, the heterologous moiety is selected from the group consisting of albumin or a fragment thereof, an immunoglobulin Fc region, the C-terminal peptide (CTP) of the β subunit of human chorionic gonadotropin, a PAS sequence, a HAP sequence, a transferrin or a fragment thereof, an albumin-binding moiety, a derivative thereof, or any combination thereof. In certain exemplary embodiments, the heterologous moiety is linked to the N-terminus or the C-terminus of the FVIII or inserted between two amino acids in the FVIII. In certain exemplary embodiments, the heterologous moiety is inserted between two amino acids at one or more insertion site selected from the insertion sites listed in Table 4.

In certain exemplary embodiments, the FVIII further comprises A1 domain, A2 domain, C1 domain, C2 domain, an optional B domain, and a heterologous moiety, wherein the heterologous moiety is inserted immediately downstream of amino acid 745 corresponding to mature FVIII (SEQ ID NO:106).

In certain exemplary embodiments, the FVIII further comprises an FcRn binding partner. In certain exemplary embodiments, the FcRn binding partner comprises an Fc region of an immunoglobulin constant domain.

In certain exemplary embodiments, the nucleic acid sequence encoding the FVIII is codon optimized. In certain exemplary embodiments, the nucleic acid sequence encoding the FVIII is codon optimized for expression in a human.

In certain exemplary embodiments, the nucleic acid sequence encoding the FVIII comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a nucleotide sequence of SEQ ID NO: 107.

In certain exemplary embodiments, the nucleic acid sequence encoding the FVIII comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the nucleotide sequence of SEQ ID NO: 71.

In certain exemplary embodiments, the heterologous polynucleotide sequence is codon optimized. In certain exemplary embodiments, the heterologous polynucleotide sequence is codon optimized for expression in a human.

In certain exemplary embodiments, the nucleic acid molecule is formulated with a delivery agent. In certain exemplary embodiments, the delivery agent comprises a lipid nanoparticle. In certain exemplary embodiments, the delivery agent is selected from the group consisting of liposomes, non-lipid polymeric molecules, and endosomes, and any combination thereof.

In certain exemplary embodiments, the nucleic acid molecule is formulated for intravenous, transdermal, intradermal, subcutaneous, pulmonary, or oral delivery, or any combination thereof. In certain exemplary embodiments, the nucleic acid molecule is formulated for intravenous delivery.

In certain aspects, a vector comprising a nucleic acid molecule as described herein, is provided.

In certain aspects, a host cell comprising a nucleic acid molecule as described herein, is provided.

In certain aspects, a pharmaceutical composition comprising a nucleic acid molecule or a vector as described herein, and a pharmaceutically acceptable excipient, is provided.

In certain aspects, a pharmaceutical composition comprising a host cell as described herein, and a pharmaceutically acceptable excipient, is provided.

In certain aspects, a kit, comprising a nucleic acid molecule as described herein, and instructions for administering the nucleic acid molecule to a subject in need thereof, is provided.

In certain aspects, a baculovirus system for production of a nucleic acid molecule as described herein, is provided.

In certain exemplary embodiments, a nucleic acid molecule as described herein, is produced in insect cells.

In certain aspects, a nanoparticle delivery system comprising a nucleic acid molecule as described herein, is provided.

In certain aspects, a method of producing a polypeptide, comprising culturing a host cell as described herein under suitable conditions and recovering the polypeptide, is provided.

In certain aspects, a method of producing a polypeptide with clotting activity, comprising: culturing a host cell as described herein under suitable conditions and recovering the polypeptide with clotting activity, is provided.

In certain aspects, a method of expressing a heterologous polynucleotide sequence in a subject in need thereof, comprising administering to the subject a nucleic acid molecule as described herein, a vector as described herein, or a pharmaceutical composition as described herein, is provided.

In certain aspects, a method of expressing a clotting factor in a subject in need thereof, comprising administering to the subject a nucleic acid molecule as described herein, a vector as described herein, a polypeptide as described herein, or a pharmaceutical composition as described herein, is provided.

In certain aspects, a method of treating a disease or disorder in a subject in need thereof, comprising administering to the subject a nucleic acid molecule as described herein, a vector as described herein, or a pharmaceutical composition as described herein, is provided.

In certain aspects, a method of treating a subject having a clotting factor deficiency, comprising administering to the subject a nucleic acid molecule as described herein, a vector as described herein, a polypeptide as described herein, or a pharmaceutical composition as described herein, is provided.

In certain aspects, a method of treating a clotting factor deficiency in a subject in need thereof, comprising administering to the subject a nucleic acid molecule as described herein, a vector as described herein, a polypeptide as described herein, or a pharmaceutical composition as described herein, is provided.

In certain exemplary embodiments, the nucleic acid molecule is administered intravenously, transdermally, intradermally, subcutaneously, orally, pulmonarily, or any combination thereof. In certain exemplary embodiments, the nucleic acid molecule is administered intravenously.

In certain exemplary embodiments, the method further comprising administering to the subject a second agent.

In certain exemplary embodiments, the subject is a mammal. In certain exemplary embodiments, the subject is a human.

In certain exemplary embodiments, the administration of the nucleic acid molecule to the subject results in an increased FVIII activity, relative to a FVIII activity in the subject prior to the administration, wherein the FVIII activity is increased by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, or at least about 100-fold.

In certain exemplary embodiments, the subject has a bleeding disorder. In certain exemplary embodiments, the bleeding disorder is a hemophilia. In certain exemplary embodiments, the bleeding disorder is hemophilia A.

In certain aspects, a method of treating a bleeding disorder in a subject in need thereof, comprising administering to the subject a nucleic acid molecule comprising a first inverted terminal repeat (ITR) and a second ITR flanking a genetic cassette comprising a heterologous polynucleotide sequence encoding a clotting factor, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided.

In certain aspects, a method of treating hemophilia A in a subject in need thereof, comprising administering to the subject a nucleic acid molecule comprising a first inverted terminal repeat (ITR) and a second ITR flanking a genetic cassette comprising a heterologous polynucleotide sequence encoding factor VIII (FVIII), wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided

In certain aspects, a method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a liver-associated metabolic enzyme that is deficient in the subject, wherein the first ITR and/or second ITR are an ITR of a non-adeno-associated virus (non-AAV), is provided.

In certain exemplary embodiments, the the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

In certain aspects, a method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a liver-associated metabolic enzyme that is deficient in the subject, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided.

In certain exemplary embodiments, the genetic cassette comprises a single stranded nucleic acid. In certain exemplary embodiments, the genetic cassette comprises a double stranded nucleic acid.

In certain exemplary embodiments, the metabolic disorder of the liver is selected from the group consisting of phenylketonuria (PKU), a urea cycle disease, a lysosomal storage disorder, and a glycogen storage disease. In certain exemplary embodiments, the metabolic disorder of the liver is phenylketonuria (PKU).

In certain exemplary embodiments, the nucleic acid molecule is administered intravenously, transdermally, intradermally, subcutaneously, orally, pulmonarily, or any combination thereof. In certain exemplary embodiments, the nucleic acid molecule is administered intravenously.

In certain exemplary embodiments, the method further comprising administering to the subject a second agent.

In certain exemplary embodiments, the subject is a mammal. In certain exemplary embodiments, the subject is a human.

In certain aspects, a method of treating phenylketonuria (PKU) in a subject in need thereof, comprising administering to the subject a nucleic acid molecule comprising a first inverted terminal repeat (ITR) and a second ITR flanking a genetic cassette comprising a heterologous polynucleotide sequence encoding phenylalanine hydroxylase, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided.

In certain exemplary embodiments, the genetic cassette comprises a single stranded nucleic acid. In certain exemplary embodiments, the genetic cassette comprises a double stranded nucleic acid.

In certain exemplary embodiments, the nucleic acid molecule is formulated with a delivery agent. In certain exemplary embodiments, the delivery agent comprises a lipid nanoparticle.

In certain aspects, a method of cloning a nucleic acid molecule, comprising inserting a nucleic acid molecule capable of complex secondary structures into a suitable vector, and introducing the resulting vector into a bacterial host strain comprising a disruption in the SbcCD complex, is provided

In certain exemplary embodiments, the the disruption in the SbcCD complex comprises a genetic disruption in the SbcC gene and/or SbcD gene. In certain exemplary embodiments, the disruption in the SbcCD complex comprises a genetic disruption in the SbcC gene. In certain exemplary embodiments, the disruption in the SbcCD complex comprises a genetic disruption in the SbcD gene.

In certain exemplary embodiments, the nucleic acid molecule comprises a first inverted terminal repeat (ITR) and a second ITR, wherein the first and/or second ITR is a non-adeno-associated virus (non-AAV) ITR.

In certain exemplary embodiments, the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

In certain exemplary embodiments, the nucleic acid molecule further comprises a genetic cassette, wherein the genetic cassette is flanked by the first ITR and second ITR.

In certain exemplary embodiments, the genetic cassette comprises a heterologous polynucleotide sequence.

In certain exemplary embodiments, the suitable vector is a low copy vector. In certain exemplary embodiments, the suitable vector is pBR322.

In certain exemplary embodiments, the bacterial host strain is incapable of resolving cruciform DNA structures.

In certain exemplary embodiments, the bacterial host strain is PMC103, comprising the genotype sbcC, recD, mcrA, ΔmcrBCF. In certain exemplary embodiments, the bacterial host strain is PMC107, comprising the genotype recBC, recJ, sbcBC, mcrA, ΔmcrBCF. In certain exemplary embodiments, the bacterial host strain is SURE, comprising the genotype recB, recJ, sbcC, mcrA, ΔmcrBCF, umuC, uvrC.

In certain aspects, a method of cloning a nucleic acid molecule, comprising inserting a nucleic acid molecule capable of complex secondary structures into a suitable vector, and introducing the resulting vector into a bacterial host strain comprising a disruption in the SbcCD complex, wherein the nucleic acid molecule comprises a first inverted terminal repeat (ITR) and a second ITR, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof, is provided

### BRIEF DESCRIPTION OF THE DRAWINGS

FIG. 1A-1B are schematic representations of a single strand clotting factor (e.g., FVIII) expression cassette. The locations of 5′ ITR from a non-AAV (with hairpin loop at the end of the ssDNA structure), 3′ ITR from a non-AAV (with hairpin loop), a promotor sequence (e.g., TTPp or CAGp), and a transgene sequence, e.g., FVIIIco6XTEN sequence with an XTEN144 inserted within the B domain are shown. The exemplary expression cassettes also show additional possible elements, e.g., an intron sequence, WPREmut sequence, and bGHpA sequence.

FIGS. 1C-1F are schematic representations of plasmids used to prepare single strand clotting factor expression cassettes, such as the cassette shown in FIG. 1A-1B, wherein the ITRs of the cassette are derived from AAV2 (FIG. 1C), B19 (FIG. 1D), GPV (FIG. 1E), or are the wildtype B19 ITR sequence (FIG. 1F). A plasmid construct comprising an ssFVIII expression cassette as shown here was digested with Pvull (at Pvull sites) (FIG. 1C) or Lgul (at Lgul sites) (FIGS. 1D-1F) to precisely release the sequence comprising the ITRs and expression cassette. The double stranded DNA was heat denatured at 95° C. to produce ssDNA and then incubated at 4° C. to allow for ITR structure formation.

FIG. 2A is a phylogenetic tree illustrating that relationships between various parvovirus family members. B19, AAV-2, and GPV are marked by outlined boxes.

FIG. 2B is a schematic drawing of the various cassettes, including the hairpin structures.

FIGS. 3A and 3B are alignments of the ITRs of B19 (SEQ ID NO: 167), GPV (SEQ ID NO: 172), and AAV2 (SEQ ID NO: 145) (FIG. 3A) and B19 and GPV (FIG. 3B). Gray shading shows homology.

FIGS. 4A-4C show FVIII plasma activity following single-stranded FVIII-AAV naked DNA (ssAAV-FVIII; FIG. 1C), ssDNA-B19 FVIII (FIG. 1D), or ssDNA-GPV FVIII (FIG. 1E) administration via hydrodynamic injection (HDI) in Hem A mice. FVIII Activity was measured (as a percentage of normal physiological levels in humans) in plasma samples at 24 hours, 3 days, 2 weeks, 3 weeks, 1 month, 2 months, 3 months, 4 months, 5 months, and 6 months in mice treated with a single HDI of ssDNA at 50 μg/mouse (FIG. 4C), 20 μg/mouse (FIGS. 4A and 4B), 10 μg/mouse (FIGS. 4A, 4B, and 4C), or 5 μg/mouse (FIG. 4A). An HDI of 5 μg/mouse of plasmid DNA was given as a control (FIGS. 4A, 4B, and 4C).

FIG. 5 shows FVIII activity in hemophilia A mouse plasma following a single hydrodynamic injection of equal molar amounts of single-stranded naked DNA (ssAAV-FVIII, FIG. 1A), double-stranded AAV-FVIII DNA containing the ITR sequence (dsDNA), double-stranded FVIII DNA without the ITR sequence (dsDNA No ITR), or circularized double-stranded FVIII DNA without ITR or bacterial sequences (minicircle). dsDNA was generated by enzyme cleavage of the AAV-FVIII plasmid (FIG. 2C) with Pvull but not heat denatured. dsDNA No ITR was generated by enzyme cleavage of the AAV-FVIII plasmid (FIG. 2C) with AfIII and subsequently purified. Minicircle DNA was generated by ligation of the dsDNA No ITR DNA at AfIII sites. Mouse plasma was collected over 3 months or 4 months and FVIII was determined by chromogenic activity assay.

FIG. 6 shows FVIII activity in hemophilia A mouse plasma following a hydrodynamic injection of 30 μg of single-stranded naked FVIII-DNA (FIG. 1A, FIGS. 1D-1F). Plasma was collected weekly for 7 weeks and FVIII activity was determined by chromogenic assay. After 35 days (depicted as black arrow), mice receiving FVIII-B19d135 and FVIII-GPVd162 ssDNA were re-administered 30 μg via hydrodynamic injection.

FIG. 7A is a schematic representations of a single strand murine phenylalanine hydroxylase (e.g., PAH) expression cassette. The locations of 5 ITR from a non-AAV (with hairpin loop at the end of the ssDNA structure), 3′ ITR from a non-AAV (with hairpin loop), a promotor sequence (e.g., CAGp), and a transgene sequence, e.g., 3×FLAG_mPAH sequence are shown. The exemplary expression cassettes also show additional possible elements, e.g., WPREmut sequence, and bGHpA sequence.

FIGS. 7B-7D show plasma concentrations of phenylalanine (Phe) in phenylketonuria (PKU) mice before (day 0) and after single administration of single-stranded DNA containing the murine PAH cDNA and non-AAV ITRs B19d135 or GPVd162 via hydrodynamic injection. Plasma was collected at days 3, 7, 14, 28, 42, and 56 following ssDNA administration. Residual phenylalanine levels are shown as concentration in μg/ml (FIGS. 7B-7C) or as percent prior to administration (FIG. 7D). The horizontal line depicts baseline Phe levels prior to administration.

FIG. 7E shows a Western immunoblot of liver lysates from PKU mice treated with ssDNA containing the murine PAH transgene and either B19d135 or GPVd165 ITRs. Livers were collected at day 81 post treatment and protein lysates were extracted. Each well represents a single animal. The FLAG-tagged murine PAH protein was detected using the M2 anti-FLAG antibody and a GAPDH loading control was included for comparison.

FIGS. 8A-B show FVIII activity levels in Huh7 cell supernatant following transduction with FVIII-AAV DNA (FIGS. 1A-1C) encapsulated lipid nanoparticles. Plasmid FVIII-AAV under the CAGp promoter (FIG. 1B) was encapsulated at three amine-to-phosphate (NP) ratios and applied to Huh7 cells at various concentrations determined by picogreen assay (FIG. 8A). Plasmid, double stranded linear (ds), and single-stranded (ss) AAV-FVIII under the TTPp promoter (FIG. 1A) was also encapsulated in lipid nanoparticles at two NP ratios and used to transduce Huh7 cells at various DNA concentrations (FIG. 8B). FVIII was measured by chromogenic activity assay compared to a human FACT plasma standard.

### DETAILED DESCRIPTION OF THE DISCLOSURE

The present disclosure describes plasmid-like nucleic acid molecules comprising a first inverted terminal repeat (ITR), a second ITR, and a genetic cassette, e.g., encoding a target sequence (also referred to herein as a heterologous polynucleotide sequence), e.g., a therapeutic protein or a miRNA, wherein the first ITR and/or the second ITR are an ITR of a non-adeno-associated virus (e.g., the first ITR and/or the second ITR are from a non-AAV). In some embodiments, the genetic cassette encodes a therapeutic protein, e.g., the target sequence encodes a therapeutic protein. In some embodiments, the therapeutic protein comprises a protein selected from a clotting factor, a growth factor, a hormone, a cytokine, an antibody, a fragment thereof, or a combination thereof. In some embodiments, the genetic cassette encodes dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), or any combination thereof.

In some embodiments, the therapeutic protein comprises a clotting factor. In one particular embodiment, the therapeutic protein comprises a FVIII or a FIX protein.

In some embodiments, the genetic cassette encodes a miRNA. In certain embodiments, the miRNA down regulates the expression of a target gene selected from SOD1, HTT, RHO, or any combination thereof.

In certain embodiments, the non-AAV is selected from the group consisting of a member of the viral family Parvoviridae and any combination thereof. The present disclosure is further directed to methods of expressing a therapeutic protein, e.g., a clotting factor, e.g., a FVIII, in a subject in need thereof, comprising administering to the subject a nucleic acid molecule comprising a first inverted terminal repeat (ITR), a second ITR, and a genetic cassette, e.g., encoding a therapeutic protein or an miRNA, wherein the first ITR and/or the second ITR are an ITR of a non-adeno-associated virus (non-AAV). In certain embodiments, the disclosure describes an isolated nucleic acid molecule comprising a nucleotide sequence, which has sequence homology to a nucleotide sequence selected from SEQ ID NOs: 113 and 120.

In certain embodiments, the present disclosure provides nucleic acid molecules comprising a first inverted terminal repeat (ITR) and a second ITR flanking a genetic cassette comprising a heterologous polynucleotide sequence, wherein the first and/or second ITR is derived from parvovirus B19 or goose parvovirus (GPV).

Exemplary constructs of the disclosure are illustrated in the accompanying figures and sequence listing. In order to provide a clear understanding of the specification and claims, the following definitions are provided below.

### I. Definitions

It is to be noted that the term “a” or “an” entity refers to one or more of that entity: for example, “a nucleotide sequence” is understood to represent one or more nucleotide sequences. Similarly, “a therapeutic protein” and “a miRNA” is understood to represent one or more therapeutic protein and one or more miRNA, respectively. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Also as used herein, “and/or” refers to and encompasses any and all possible combinations of one or more of the associated listed items, as well as the lack of combinations when interpreted in the alternative (“or”).

“Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, an “inverted terminal repeat” (or “ITR”) refers to a nucleic acid subsequence located at either the 5′ or 3′ end of a single stranded nucleic acid sequence, which comprises a set of nucleotides (initial sequence) followed downstream by its reverse complement, i.e., palindromic sequence. The intervening sequence of nucleotides between the initial sequence and the reverse complement can be any length including zero. In one embodiment, the ITR useful for the present disclosure comprises one or more “palindromic sequences.” An ITR can have any number of functions. In some embodiments, an ITR described herein forms a hairpin structure. In some embodiments, the ITR forms a T-shaped hairpin structure. In some embodiments, the ITR forms a non-T-shaped hairpin structure, e.g., a U-shaped hairpin structure. In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire life-span of the cell). In some embodiments, the ITR promotes the stability of the nucleic acid molecule in the nucleus of a cell. In some embodiments, the ITR promotes the retention of the nucleic acid molecule in the nucleus of a cell. In some embodiments, the ITR promotes the persistence of the nucleic acid molecule in the nucleus of a cell. In some embodiments, the ITR inhibits or prevents the degradation of the nucleic acid molecule in the nucleus of a cell.

In one embodiment, the initial sequence and/or the reverse complement comprise about 2-600 nucleotides, about 2-550 nucleotides, about 2-500 nucleotides, about 2-450 nucleotides, about 2-400 nucleotides, about 2-350 nucleotides, about 2-300 nucleotides, or about 2-250 nucleotides. In some embodiments, the initial sequence and/or the reverse complement comprise about 5-600 nucleotides, about 10-600 nucleotides, about 15-600 nucleotides, about 20-600 nucleotides, about 25-600 nucleotides, about 30-600 nucleotides, about 35-600 nucleotides, about 40-600 nucleotides, about 45-600 nucleotides, about 50-600 nucleotides, about 60-600 nucleotides, about 70-600 nucleotides, about 80-600 nucleotides, about 90-600 nucleotides, about 100-600 nucleotides, about 150-600 nucleotides, about 200-600 nucleotides, about 300-600 nucleotides, about 350-600 nucleotides, about 400-600 nucleotides, about 450-600 nucleotides, about 500-600 nucleotides, or about 550-600 nucleotides. In some embodiments, the initial sequence and/or the reverse complement comprise about 5-550 nucleotides, about 5 to 500 nucleotides, about 5-450 nucleotides, about 5 to 400 nucleotides, about 5-350 nucleotides, about 5 to 300 nucleotides, or about 5-250 nucleotides. In some embodiments, the initial sequence and/or the reverse complement comprise about 10-550 nucleotides, about 15-500 nucleotides, about 20-450 nucleotides, about 25-400 nucleotides, about 30-350 nucleotides, about 35-300 nucleotides, or about 40-250 nucleotides. In certain embodiments, the initial sequence and/or the reverse complement comprise about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, or about 600 nucleotides. In particular embodiments, the initial sequence and/or the reverse complement comprise about 400 nucleotides.

In other embodiments, the initial sequence and/or the reverse complement comprise about 2-200 nucleotides, about 5-200 nucleotides, about 10-200 nucleotides, about 20-200 nucleotides, about 30-200 nucleotides, about 40-200 nucleotides, about 50-200 nucleotides, about 60-200 nucleotides, about 70-200 nucleotides, about 80-200 nucleotides, about 90-200 nucleotides, about 100-200 nucleotides, about 125-200 nucleotides, about 150-200 nucleotides, or about 175-200 nucleotides. In other embodiments, the initial sequence and/or the reverse complement comprise about 2-150 nucleotides, about 5-150 nucleotides, about 10-150 nucleotides, about 20-150 nucleotides, about 30-150 nucleotides, about 40-150 nucleotides, about 50-150 nucleotides, about 75-150 nucleotides, about 100-150 nucleotides, or about 125-150 nucleotides. In other embodiments, the initial sequence and/or the reverse complement comprise about 2-100 nucleotides, about 5-100 nucleotides, about 10-100 nucleotides, about 20-100 nucleotides, about 30-100 nucleotides, about 40-100 nucleotides, about 50-100 nucleotides, or about 75-100 nucleotides. In other embodiments, the initial sequence and/or the reverse complement comprise about 2-50 nucleotides, about 10-50 nucleotides, about 20-50 nucleotides, about 30-50 nucleotides, about 40-50 nucleotides, about 3-30 nucleotides, about 4-20 nucleotides, or about 5-10 nucleotides. In another embodiment, the initial sequence and/or the reverse complement consist of two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides. In other embodiments, an intervening nucleotide between the initial sequence and the reverse complement is (e.g., consists of) 0 nucleotide, 1 nucleotide, two nucleotides, three nucleotides, four nucleotides, five nucleotides, six nucleotides, seven nucleotides, eight nucleotides, nine nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

Therefore, an “ITR” as used herein can fold back on itself and form a double stranded segment. For example, the sequence GATCXXXXGATC comprises an initial sequence of GATC and its complement (3′CTAG5′) when folded to form a double helix. In some embodiments, the ITR comprises a continuous palindromic sequence (e.g., GATCGATC) between the initial sequence and the reverse complement. In some embodiments, the ITR comprises an interrupted palindromic sequence (e.g., GATCXXXXGATC) between the initial sequence and the reverse complement. In some embodiments, the complementary sections of the continuous or interrupted palindromic sequence interact with each other to form a “hairpin loop” structure. As used herein, a “hairpin loop” structure results when at least two complimentary sequences on a single-stranded nucleotide molecule base-pair to form a double stranded section. In some embodiments, only a portion of the ITR forms a hairpin loop. In other embodiments, the entire ITR forms a hairpin loop.

In the present disclosure, at least one ITR is an ITR of a non-adenovirus associated virus (non-AAV). In certain embodiments, the ITR is an ITR of a non-AAV member of the viral family Parvoviridae. In some embodiments, the ITR is an ITR of a non-AAV member of the genus Dependovirus or the genus Erythrovirus. In particular embodiments, the ITR is an ITR of a goose parvovirus (GPV), a Muscovy duck parvovirus (MDPV), or an Erythrovirus parvovirus B19 (also known as parvovirus B19, primate erythroparvovirus 1, B19 virus, and Erythrovirus). In certain embodiments, one ITR of two ITRs is an ITR of an AAV. In other embodiments, one ITR of two ITRs in the construct is an ITR of an AAV serotype selected from serotype 1, 2, 3, 4, 5, 6, 7, 8, 9, 10, 11 and any combination thereof. In one particular embodiment, the ITR is derived from AAV serotype 2, e.g., an ITR of AAV serotype 2.

In certain aspects of the present disclosure, the nucleic acid molecule comprises two ITRs, a 5′ ITR and a 3′ ITR, wherein the 5′ ITR is located at the 5 terminus of the nucleic acid molecule, and the 3′ ITR is located at the 3′ terminus of the nucleic acid molecule. The 5′ ITR and the 3′ ITR can be derived from the same virus or different viruses. In certain embodiments, the 5′ ITR is derived from an AAV and the 3′ ITR is not derived from an AAV virus (e.g., a non-AAV). In some embodiments, the 3′ ITR is derived from an AAV and the 5′ ITR is not derived from an AAV virus (e.g., a non-AAV). In other embodiments, the 5′ ITR is not derived from an AAV virus (e.g., a non-AAV), and the 3′ ITR is derived from the same or a different non-AAV virus.

The term “parvovirus” as used herein encompasses the family Parvoviridae, including but not limited to autonomously-replicating parvoviruses and Dependoviruses. The autonomous parvoviruses include, for example, members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

Exemplary autonomous parvoviruses include, but are not limited to, porcine parvovirus, mice minute virus, canine parvovirus, mink entertitus virus, bovine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al. VIROLOGY, volume 2, chapter 69 (4th ed., Lippincott-Raven Publishers).

The term “non-AAV” as used herein encompasses nucleic acids, proteins, and viruses from the family Parvoviridae excluding any adeno-associated viruses (AAV) of the Parvoviridae family. “Non-AAV” includes but is not limited to autonomously-replicating members of the genera Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, and Penstyldensovirus.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers).

The term “derived from,” as used herein, refers to a component that is isolated from or made using a specified molecule or organism, or information (e.g., amino acid or nucleic acid sequence) from the specified molecule or organism. For example, a nucleic acid sequence (e.g., ITR) that is derived from a second nucleic acid sequence (e.g., ITR) can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. Mutagenesis of a polypeptide typically entails manipulation of the polynucleotide that encodes the polypeptide. In some embodiments, a nucleotide or amino acid sequence that is derived from a second nucleotide or amino acid sequence has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the second nucleotide or amino acid sequence, respectively, wherein the first nucleotide or amino acid sequence retains the biological activity of the second nucleotide or amino acid sequence. In other embodiments, an ITR derived from an ITR of a non-AAV (or AAV) is at least 90% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 80% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 70% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 60% identical to the non-AAV ITR (or AAV ITR, respectively), wherein the non-AAV (or AAV) ITR retains a functional property of the non-AAV ITR (or AAV ITR, respectively). In some embodiments, an ITR derived from an ITR of a non-AAV (or AAV) is at least 50% identical to the non-AAV ITR (or AAV ITR, respectively), wherein the non-AAV (or AAV) ITR retains a functional property of the non-AAV ITR (or AAV ITR, respectively).

In certain embodiments, an ITR derived from an ITR of a non-AAV (or AAV) comprises or consists of a fragment of the ITR of the non-AAV (or AAV). In some embodiments, the ITR derived from an ITR of a non-AAV (or AAV) comprises or consists of a fragment of the ITR of the non-AAV (or AAV), wherein the fragment comprises at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 125 nucleotides, at least about 150 nucleotides, at least about 175 nucleotides, at least about 200 nucleotides, at least about 225 nucleotides, at least about 250 nucleotides, at least about 275 nucleotides, at least about 300 nucleotides, at least about 325 nucleotides, at least about 350 nucleotides, at least about 375 nucleotides, at least about 400 nucleotides, at least about 425 nucleotides, at least about 450 nucleotides, at least about 475 nucleotides, at least about 500 nucleotides, at least about 525 nucleotides, at least about 550 nucleotides, at least about 575 nucleotides, or at least about 600 nucleotides; wherein the ITR derived from an ITR of a non-AAV (or AAV) retains a functional property of the non-AAV ITR (or AAV ITR, respectively). In certain embodiments, the ITR derived from an ITR of a non-AAV (or AAV) comprises or consists of a fragment of the ITR of the non-AAV (or AAV), wherein the fragment comprises at least about 129 nucleotides, and wherein the ITR derived from an ITR of a non-AAV (or AAV) retains a functional property of the non-AAV ITR (or AAV ITR, respectively). In certain embodiments, the ITR derived from an ITR of a non-AAV (orAAV) comprises or consists of a fragment of the ITR of the non-AAV (orAAV), wherein the fragment comprises at least about 102 nucleotides, and wherein the ITR derived from an ITR of a non-AAV (or AAV) retains a functional property of the non-AAV ITR (orAAV ITR, respectively).

In some embodiments, the ITR derived from an ITR of a non-AAV (or AAV) comprises or consists of a fragment of the ITR of the non-AAV (or AAV), wherein the fragment comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the length of the ITR of the non-AAV (orAAV).

In certain embodiments, a nucleotide or amino acid sequence that is derived from a second nucleotide or amino acid sequence has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to a homologous portion of the second nucleotide or amino acid sequence, respectively, when properly aligned, wherein the first nucleotide or amino acid sequence retains the biological activity of the second nucleotide or amino acid sequence. In other embodiments, an ITR derived from an ITR of a non-AAV (orAAV) is at least 90% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 80% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 70% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 60% identical to a homologous portion of the non-AAV ITR (or AAV ITR, respectively), when properly aligned, wherein the first nucleotide or amino acid sequence retains the biological activity of the second nucleotide or amino acid sequence. In some embodiments, an ITR derived from an ITR of a non-AAV (or AAV) is at least 50% identical to a homologous portion of the non-AAV ITR (orAAV ITR, respectively), when properly aligned, wherein the first nucleotide or amino acid sequence retains the biological activity of the second nucleotide or amino acid sequence.

A “capsid-free” or “capsid-less” vector or nucleic acid molecule refers to a vector construct free from a capsid. In some embodiments, the capsid-less vector or nucleic acid molecule does not contain sequences encoding, e.g., an AAV Rep protein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse ß-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

A polynucleotide which encodes a product, e.g., a miRNA or a gene product (e.g., a polypeptide such as a therapeutic protein), can include a promoter and/or other expression (e.g., transcription or translation) control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In some embodiments, the nucleic acid molecule comprises a tissue specific promoter. In certain embodiments, the tissue specific promoter drives expression of the therapeutic protein, e.g., the clotting factor, in the liver, e.g., in hepatocytes and/or endothelial cells. In particular, embodiments, the promoter is selected from the group consisting of a mouse thyretin promoter (mTTR), an endogenous human factor VIII promoter (F8), a human alpha-1-antitrypsin promoter (hAAT), a human albumin minimal promoter, a mouse albumin promoter, a tristetraprolin (TTP) promoter, a CASI promoter, a CAG promoter, a cytomegalovirus (CMV) promoter, a phosphoglycerate kinase (PGK) promoter and any combination thereof. In some embodiments, the promoter is selected from a liver specific promoter (e.g., α1-antitrypsin (AAT)), a muscle specific promoter (e.g., muscle creatine kinase (MCK), myosin heavy chain alpha (αMHC), myoglobin (MB), and desmin (DES)), a synthetic promoter (e.g., SPc5-12, 2R5Sc5-12, dMCK, and tMCK) and any combination thereof. In one particular embodiment, the promoter comprises a TTP promoter.

The terms “restriction endonuclease” and “restriction enzyme” are used interchangeably and refer to an enzyme that binds and cuts within a specific nucleotide sequence within double stranded DNA.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construct, which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for an FVIII variant, or FVIII binding activity for the VWF fragment) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, WI), the GCG suite of programs (Wisconsin Package Version 9.0, Genetics Computer Group (GCG), Madison, WI), BLASTP, BLASTN, BLASTX (Altschul et al., J. Mol. Biol. 215:403 (1990)), and DNASTAR (DNASTAR, Inc. 1228 S. Park St. Madison, WI 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized. For the purposes of determining percent identity between a therapeutic protein, e.g., a clotting factor, sequence of the disclosure and a reference sequence, only nucleotides in the reference sequence corresponding to nucleotides in the therapeutic protein, e.g., the clotting factor, sequence of the disclosure are used to calculate percent identity. For example, when comparing a full length FVIII nucleotide sequence containing the B domain to an optimized B domain deleted (BDD) FVIII nucleotide sequence of the disclosure, the portion of the alignment including the A1, A2, A3, C1, and C2 domain will be used to calculate percent identity. The nucleotides in the portion of the full length FVIII sequence encoding the B domain (which will result in a large “gap” in the alignment) will not be counted as a mismatch. In addition, in determining percent identity between an optimized BDD FVIII sequence of the disclosure, or a designated portion thereof (e.g., nucleotides 58-2277 and 2320-4374 of SEQ ID NO:3), and a reference sequence, percent identity will be calculated by aligning dividing the number of matched nucleotides by the total number of nucleotides in the complete sequence of the optimized BDD-FVIII sequence, or a designated portion thereof, as recited herein.

As used herein, nucleotides corresponding to nucleotides in a particular sequence of the disclosure are identified by alignment of the sequence of the disclosure to maximize the identity to a reference sequence. The number used to identify an equivalent amino acid in a reference sequence is based on the number used to identify the corresponding amino acid in the sequence of the disclosure.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the disclosure with an Ig Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

As used herein, the term “insertion site” refers to a position in a polypeptide, or fragment, variant, or derivative thereof, which is immediately upstream of the position at which a heterologous moiety can be inserted. An “insertion site” is specified as a number, the number being the number of the amino acid in a reference sequence. For example, an “insertion site” in FVIII refers to the number of the amino acid sequence in mature native FVIII (SEQ ID NO: 15) to which the insertion site corresponds, which is immediately N-terminal to the position of the insertion. For example, the phrase “a3 comprises a heterologous moiety at an insertion site which corresponds to amino acid 1656 of SEQ ID NO: 15” indicates that the heterologous moiety is located between two amino acids corresponding to amino acid 1656 and amino acid 1657 of SEQ ID NO: 15.

The phrase “immediately downstream of an amino acid” as used herein refers to position right next to the terminal carboxyl group of the amino acid. Similarly, the phrase “immediately upstream of an amino acid” refers to the position right next to the terminal amine group of the amino acid.

The terms “inserted,” “is inserted,” “inserted into” or grammatically related terms, as used herein refer to the position of a heterologous moiety in a polypeptide, e.g., a clotting factor, relative to the analogous position in the parental polypeptide. For example, in certain embodiment, “inserted” and the like refer to the position of a heterologous moiety in a recombinant FVIII polypeptide, relative to the analogous position in native mature human FVIII. As used herein the terms refer to the characteristics of the polypeptide, and do not indicate, imply or infer any methods or process by which the polypeptide was made.

As used herein, the term “half-life” refers to a biological half-life of a particular polypeptide in vivo. Half-life can be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, the therapeutic protein, e.g., the clotting factor, e.g., FVIII, and chimeric proteins comprising the same are monophasic, and thus do not have an alpha phase, but just the single beta phase. Therefore, in certain embodiments, the term half-life as used herein refers to the half-life of the polypeptide in the β-phase.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

Hemostasis, as used herein, means the stopping or slowing of bleeding or hemorrhage; or the stopping or slowing of blood flow through a blood vessel or body part.

Hemostatic disorder, as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for vWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk.

The isolated nucleic acid molecules, isolated polypeptides, or vectors comprising the isolated nucleic acid molecule of the disclosure can be used prophylactically. As used herein the term “prophylactic treatment” refers to the administration of a molecule prior to a bleeding episode. In one embodiment, the subject in need of a general hemostatic agent is undergoing, or is about to undergo, surgery. A polynucleotide, polypeptide, or vector of the disclosure can be administered prior to or after surgery as a prophylactic. The polynucleotide, polypeptide, or vector of the disclosure can be administered during or after surgery to control an acute bleeding episode. The surgery can include, but is not limited to, liver transplantation, liver resection, dental procedures, or stem cell transplantation.

The isolated nucleic acid molecules, isolated polypeptides, or vectors of the disclosure are also used for on-demand treatment. The term “on-demand treatment” refers to the administration of an isolated nucleic acid molecule, isolated polypeptide, or vector in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject can have trauma, uremia, a hereditary bleeding disorder (e.g., factor VII deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining, e.g., a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, 20 IU/dL, 25 IU/dL, 30 IU/dL, 35 IU/dL, 40 IU/dL, 45 IU/dL, 50 IU/dL, 55 IU/dL, 60 IU/dL, 65 IU/dL, 70 IU/dL, 75 IU/dL, 80 IU/dL, 85 IU/dL, 90 IU/dL, 95 IU/dL, 100 IU/dL, 105 IU/dL, 110 IU/dL, 115 IU/dL, 120 IU/dL, 125 IU/dL, 130 IU/dL, 135 IU/dL, 140 IU/dL, 145 IU/dL, or 150 IU/dL in a subject by administering an isolated nucleic acid molecule, isolated polypeptide or vector of the disclosure. In another embodiment, treating or treatment means maintaining a FVIII trough level between about 1 and about 150 IU/dL, about 1 and about 125 IU/dL, about 1 and about 100 IU/dL, about 1 and about 90 IU/dL, about 1 and about 85 IU/dL, about 1 and about 80 IU/dL, about 1 and about 75 IU/dL, about 1 and about 70 IU/dL, about 1 and about 65 IU/dL, about 1 and about 60 IU/dL, about 1 and about 55 IU/dL, about 1 and about 50 IU/dL, about 1 and about 45 IU/dL, about 1 and about 40 IU/dL, about 1 and about 35 IU/dL, about 1 and about 30 IU/dL, about 1 and about 25 IU/dL, about 25 and about 125 IU/dL, about 50 and about 100 IU/dL, about 50 and about 75 IU/dL, about 75 and about 100 IU/dL, about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% of the FVIII activity in a non-hemophiliac subject. The minimum trough level required for treatment can be measured by one or more known methods and can be adjusted (increased or decreased) for each person.

“Administering,” as used herein, means to give a pharmaceutically acceptable nucleic acid molecule, polypeptide expressed therefrom, or vector comprising the nucleic acid molecule of the disclosure to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The nucleic acid molecules, polypeptides, and vectors can be administered as part of a pharmaceutical composition comprising at least one excipient.

The term “pharmaceutically acceptable” as used herein refers to molecular entities and compositions that are physiologically tolerable and do not typically produce toxicity or an allergic or similar untoward reaction, such as gastric upset, dizziness and the like, when administered to a human. Optionally, as used herein, the term “pharmaceutically acceptable” means approved by a regulatory agency of the federal or a state government or listed in the U.S. Pharmacopeia or other generally recognized pharmacopeia for use in animals, and more particularly in humans.

As used herein, the phrase “subject in need thereof” includes subjects, such as mammalian subjects, that would benefit from administration of a nucleic acid molecule, polypeptide, or vector of the disclosure, e.g., to improve hemostasis. In one embodiment, the subjects include, but are not limited to, individuals with hemophilia. In another embodiment, the subjects include, but are not limited to, individuals who have developed an inhibitor to the therapeutic protein, e.g., the clotting factor, e.g., FVIII, and thus are in need of a bypass therapy. The subject can be an adult or a minor (e.g., under 12 years old).

As used herein, the term “therapeutic protein” refers to any polypeptide known in the art that can be administered to a subject. In some embodiments, the therapeutic protein comprises a protein selected from a clotting factor, a growth factor, an antibody, a functional fragment thereof, or a combination thereof. As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. “Clotting factor” as used herein includes an activated clotting factor, its zymogen, or an activatable clotting factor. An “activatable clotting factor” is a clotting factor in an inactive form (e.g., in its zymogen form) that is capable of being converted to an active form. The term “clotting factor” includes but is not limited to factor I (FI), factor II (FII), factor V (FV), FVII, FVIII, FIX, factor X (FX), factor XI (FXI), factor XII (FXII), factor XIII (FXIII), Von Willebrand factor (VWF), prekallikrein, high-molecular weight kininogen, fibronectin, antithrombin Ill, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), zymogens thereof, activated forms thereof, or any combination thereof.

Clotting activity, as used herein, means the ability to participate in a cascade of biochemical reactions that culminates in the formation of a fibrin clot and/or reduces the severity, duration or frequency of hemorrhage or bleeding episode.

A “growth factor,” as used herein, includes any growth factor known in the art including cytokines and hormones. In some embodiments, the growth factor is selected from adrenomedullin (AM), angiopoietin (Ang), autocrine motility factor, a bone morphogenetic protein (BMP) (e.g. BMP2, BMP4, BMP5, BMP7), a ciliary neurotrophic factor family member (e.g., ciliary neurotrophic factor (CNTF), leukemia inhibitory factor (LIF), interleukin-6 (IL-6)), a colony-stimulating factor (e.g., macrophage colony-stimulating factor (m-CSF), granulocyte colony-stimulating factor (G-CSF), granulocyte macrophage colony-stimulating factor (GM-CSF)), an epidermal growth factor (EGF), an ephrin (e.g., ephrin A1, ephrin A2, ephrin A3, ephrin A4, ephrin A5, ephrin 1, ephrin B2, ephrin E3), erythropoietin (EPO), a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), foetal bovine somatotrophin (FBS), a GDNF family member (e.g., glial cell line-derived neurotrophic factor (GDNF), neurturin, persephin, artemin), growth differentiation factor-9 (GDF9), hepatocyte growth factor (HGF), hepatoma-derived growth factor (HDGF), insulin, an insulin-like growth factors (e.g., insulin-like growth factor-1 (IGF-1) or IGF-2, an interleukin (IL) (e.g., IL-1, IL-2, IL-3, IL-4, IL-5, IL-6, IL-7), keratinocyte growth factor (KGF), migration-stimulating factor (MSF), macrophage-stimulating protein (MSP or hepatocyte growth factor-like protein (HGFLP)), myostatin (GDF-8), a neuregulin (e.g., neuregulin 1 (NRG1), NRG2, NRG3, NRG4), a neurotrophin (e.g., brain-derived neurotrophic factor (BDNF), nerve growth factor (NGF), a neurotrophin-3 (NT-3), NT-4, placental growth factor (PGF), platelet-derived growth factor (PDGF), renalase (RNLS), T-cell growth factor (TCGF), thrombopoietin (TPO), a transforming growth factor (e.g., transforming growth factor alpha (TGF-α), TGF-β, tumor necrosis factor-alpha (TNF-α), and vascular endothelial growth factor (VEGF).

In some embodiments, the therapeutic protein is encoded by a gene selected from dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), or any combination thereof.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of the therapeutic protein, e.g., the clotting factor, e.g., FVIII. In another embodiment, the heterologous nucleotide sequence encodes a polypeptide that increases the hydrodynamic radius of the therapeutic protein, e.g., the clotting factor, e.g., FVIII. In other embodiments, the heterologous nucleotide sequence encodes a polypeptide that improves one or more pharmacokinetic properties of the therapeutic protein without significantly affecting its biological activity or function (e.g., a procoagulant activity). In some embodiments, the therapeutic protein is linked or connected to the polypeptide encoded by the heterologous nucleotide sequence by a linker. Non-limiting examples of polypeptide moieties encoded by heterologous nucleotide sequences include an immunoglobulin constant region or a portion thereof, albumin or a fragment thereof, an albumin-binding moiety, a transferrin, the PAS polypeptides of U.S. Pat Application No. 20100292130, a HAP sequence, transferrin or a fragment thereof, the C-terminal peptide (CTP) of the β subunit of human chorionic gonadotropin, albumin-binding small molecule, an XTEN sequence, FcRn binding moieties (e.g., complete Fc regions or portions thereof which bind to FcRn), single chain Fc regions (ScFc regions, e.g., as described in US 2008/0260738, WO 2008/012543, or WO 2008/1439545), polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others, or two or more combinations thereof. In some embodiments, the polypeptide encoded by the heterologous nucleotide sequence is linked to a non-polypeptide moiety. Non-limiting examples of the non-polypeptide moieties include polyethylene glycol (PEG), albumin-binding small molecules, polysialic acid, hydroxyethyl starch (HES), a derivative thereof, or any combinations thereof.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence).

In one embodiment, the “Fc region” refers to the portion of a single Ig heavy chain beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an Ig constant region, depending on the Ig isotype can include the CH2, CH3, and CH4 domains, as well as the hinge region. Chimeric proteins comprising an Fc region of an Ig bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

A “reference nucleotide sequence,” when used herein as a comparison to a nucleotide sequence of the disclosure, is a polynucleotide sequence essentially identical to the nucleotide sequence of the disclosure except that sequence is not optimized. For example, the reference nucleotide sequence for a nucleic acid molecule consisting of the codon optimized BDD FVIII of SEQ ID NO: 1 and a heterologous nucleotide sequence that encodes a single chain Fc region linked to SEQ ID NO: 1 at its 3′ end is a nucleic acid molecule consisting of the original (or “parent”) BDD FVIII of SEQ ID NO: 16 and the identical heterologous nucleotide sequence that encodes a single chain Fc region linked to SEQ ID NO: 16 at its 3′ end.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, and two or more combinations thereof.

### II. Nucleic Acid Molecules

The present disclosure is directed to a plasmid-like, capsid free, nucleic acid molecule that encodes a target sequence, wherein the target sequence encodes a therapeutic protein or a gene that can modulate expression of a target protein, e.g., a miRNA. A capsid, the protein shell of a virus, encloses the genetic material of the virus. Capsids are known to aid the functions of the virion by protecting the viral genome, delivering the genome to a host, and interacting with the host. Nonetheless, the viral capsids may be a factor in limiting the packaging capacity of the vectors and/or inducing immune responses, especially when used in gene therapy.

AAV vectors have emerged as one of the more common types of gene therapy vectors. However, the presence of the capsid limits the utility of an AAV vector in gene therapy. In particular, the capsid itself can limit the size of the transgene that is included in the vector to as low as less than 4.5 kb. Various therapeutic proteins that may be useful in a gene therapy can easily exceed this size even before regulatory elements are added.

Furthermore, proteins that make up the capsid can serve as antigens that can be targeted by a subject's immune system. AAV is very common in the general population, with most people having been exposed to an AAV throughout their lives. As a result, most potential gene therapy recipients have likely already developed an immune response to an AAV, and thus are more likely to reject the therapy.

Certain aspects of the present disclosure aim to overcome these deficiencies of AAV vectors. In particular, certain aspects of the present disclosure are directed to a nucleic acid molecule, comprising a first ITR, a second ITR, and a genetic cassette, e.g., encoding a therapeutic protein and/or a miRNA. In some embodiments, the first ITR and second ITR flank a genetic cassette comprising a heterologous polynucleotide sequence. In some embodiments, the nucleic acid molecule does not comprise a gene encoding a capsid protein, a replication protein, and/or an assembly protein. In some embodiments, the genetic cassette encodes a therapeutic protein. In some embodiments, the therapeutic protein comprises a clotting factor. In some embodiments, the genetic cassette encodes a miRNA. In certain embodiments, the genetic cassette is positioned between the first ITR and the second ITR. In some embodiments, the nucleic acid molecule further comprises one or more noncoding region. In certain embodiments, the one or more non-coding region comprises a promoter sequence, an intron, a post-transcriptional regulatory element, a 3′UTR poly(A) sequence, or any combination thereof.

In one embodiment, the genetic cassette is a single stranded nucleic acid. In another embodiment, the genetic cassette is a double stranded nucleic acid.

In one embodiment, the nucleic acid molecule comprises:
(a) a first ITR that is an ITR of a non-AAV family member of Parvoviridae (e.g., a B19 or GPV ITR);
(b) a tissue specific promoter sequence, e.g., TTP promoter;
(c) an intron, e.g., a synthetic intron;
(d) a nucleotide encoding a miRNA or a therapeutic protein, e.g., a clotting factor;
(e) a post-transcriptional regulatory element, e.g., WPRE;
(f) a 3′UTR poly(A) tail sequence, e.g., bGHpA;
(g) a second ITR that is an ITR of a non-AAV family member of Parvoviridae (e.g., a B19 or GPV ITR).

In one embodiment, the nucleic acid molecule comprises:
(a) a first ITR that is an ITR of a non-AAV family member of Parvoviridae;
(b) a tissue specific promoter sequence, e.g., TTP promoter;
(c) an intron, e.g., a synthetic intron;
(d) a nucleotide encoding a miRNA, wherein the miRNA down regulates the expression of a target gene selected from SOD1, HTT, RHO, and any combination thereof;
(e) a post-transcriptional regulatory element, e.g., WPRE;
(f) a 3′UTR poly(A) tail sequence, e.g., bGHpA;
(g) a second ITR that is an ITR of a non-AAV family member of Parvoviridae

In one embodiment, the nucleic acid molecule comprises:
(a) a first ITR that is an ITR of a non-AAV family member of Parvoviridae;
(b) a tissue specific promoter sequence, e.g., TTP promoter;
(c) an intron, e.g., a synthetic intron;
(d) a nucleotide encoding dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), or any combination thereof;
(e) a post-transcriptional regulatory element, e.g., WPRE;
(f) a 3′UTR poly(A) tail sequence, e.g., bGHpA;
(g) a second ITR that is an ITR of a non-AAV family member of Parvoviridae

In one embodiment, the nucleic acid molecule comprises:
(a) a first ITR that is an ITR of an AAV, e.g., an AAV serotype 2 genome;
(b) a tissue specific promoter sequence, e.g., TTP promoter;
(c) an intron, e.g., a synthetic intron;
(d) a nucleotide encoding FVIII; wherein the nucleotide has at least 85%, at least 90%, at least 95%, at least 96%, at least 97%, at least 98%, or at least 99% sequence identity to a nucleotide sequence selected from SEQ ID NOs: 1-14 or SEQ ID NO: 71, wherein the FVIII encoded by the nucleotide retains a FVIII activity;
(e) a post-transcriptional regulatory element, e.g., WPRE;
(f) a 3′UTR poly(A) tail sequence, e.g., bGHpA; and
(g) a second ITR that is an ITR of an AAV, e.g., an AAV serotype 2 genome.

In one embodiment, the nucleic acid molecule comprises:
(a) a first ITR that is an ITR of an AAV, e.g., an AAV serotype 2 genome;
(b) a tissue specific promoter sequence, e.g., TTP promoter;
(c) an intron, e.g., a synthetic intron;
(d) a nucleotide encoding a miRNA, wherein the miRNA down regulates the expression of a target gene, e.g., SOD1, HTT, RHO, and any combination thereof;
(f) a 3′UTR poly(A) tail sequence, e.g., bGHpA; and
(g) a second ITR that is an ITR of an AAV, e.g., an AAV serotype 2 genome.

In one embodiment, the nucleic acid molecule comprises:
(a) a first ITR that is an ITR of an AAV, e.g., an AAV serotype 2 genome;
(b) a tissue specific promoter sequence, e.g., TTP promoter;
(c) an intron, e.g., a synthetic intron;
(d) a nucleotide encoding dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), or any combination thereof;
(f) a 3′UTR poly(A) tail sequence, e.g., bGHpA; and
(g) a second ITR that is an ITR of an AAV, e.g., an AAV serotype 2 genome.

In another embodiment, the nucleic acid molecule comprises:
(a) a first ITR;
(b) a tissue specific promoter sequence, e.g., TTP promoter;
(c) an intron, e.g., a synthetic intron;
(d) a nucleotide encoding a miRNA or a therapeutic protein, e.g., clotting factor;
(e) a post-transcriptional regulatory element, e.g., WPRE;
(f) a 3′UTR poly(A) tail sequence, e.g., bGHpA; and
(g) a second ITR,
wherein one of the first ITR or the second ITR is an ITR of a non-AAV family member of Parvoviridae and the other ITR is an ITR of an AAV, e.g., an AAV serotype 2 genome.

In another embodiment, the nucleic acid molecule comprises:
(a) a 5′ ITR bearing the AAV2 5′ ITR sequence set forth in SEQ ID NO: 111;
(b) a tissue specific promoter sequence, e.g., TTP promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the AAV2 3′ ITR sequence set forth in SEQ ID NO: 124.

In another embodiment, the nucleic acid molecule comprises:
(a) a 5′ ITR bearing the AAV2 5′ ITR sequence set forth in SEQ ID NO: 111;
(b) a tissue specific promoter sequence, e.g., CAG promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the AAV2 3′ ITR sequence set forth in SEQ ID NO: 193.

In another embodiment, the nucleic acid molecule comprises:
(a) a first ITR;
(b) a tissue specific promoter sequence, TTP promoter;
(c) an intron, e.g., a synthetic intron;
(d) a nucleotide encoding a miRNA or a therapeutic protein, e.g., clotting factor;
(e) a post-transcriptional regulatory element, e.g., WPRE;
(f) a 3′UTR poly(A) tail sequence, e.g., bGHpA; and
(g) a second ITR,
wherein the first ITR is a synthetic ITR, the second ITR is a synthetic ITR, or both the first ITR and the second ITR are synthetic ITRs.

In another embodiment, the nucleic acid molecule comprises:
(a) a first B19 ITR;
(b) a tissue specific promoter sequence, e.g., TTP promoter;
(c) an intron, e.g., a synthetic intron;
(d) a heterologous polynucleotide sequence encoding therapeutic protein selected from the group consisting of a clotting factor, a growth factor, a hormone, a cytokine, an antibody, a fragment thereof, and a combination thereof;
(e) a post-transcriptional regulatory element, e.g., WPRE;
(f) a 3′UTR poly(A) tail sequence, e.g., bGHpA; and/or
(g) a second B19 ITR.

In another embodiment, the nucleic acid molecule comprises:
(a) a first GPV ITR;
(b) a tissue specific promoter sequence, e.g., TTP promoter;
(c) an intron, e.g., a synthetic intron;
(d) a heterologous polynucleotide sequence encoding therapeutic protein selected from the group consisting of a clotting factor, a growth factor, a hormone, a cytokine, an antibody, a fragment thereof, and a combination thereof;
(e) a post-transcriptional regulatory element, e.g., WPRE;
(f) a 3′UTR poly(A) tail sequence, e.g., bGHpA; and/or
(g) a second GPV ITR.

In another embodiment, the nucleic acid molecule comprises:
(a) a first B19 ITR;
(b) a ubiquitous promoter sequence, e.g., CAG promoter;
(c) an intron, e.g., a synthetic intron;
(d) a heterologous polynucleotide sequence encoding therapeutic protein selected from the group consisting of a clotting factor, a growth factor, a hormone, a cytokine, an antibody, a fragment thereof, and a combination thereof;
(e) a post-transcriptional regulatory element, e.g., WPRE;
(f) a 3′UTR poly(A) tail sequence, e.g., bGHpA; and/or
(g) a second B19 ITR.

In another embodiment, the nucleic acid molecule comprises:
(a) a first GPV ITR;
(b) a ubiquitous promoter sequence, e.g., CAG promoter;
(c) an intron, e.g., a synthetic intron;
(d) a heterologous polynucleotide sequence encoding therapeutic protein selected from the group consisting of a clotting factor, a growth factor, a hormone, a cytokine, an antibody, a fragment thereof, and a combination thereof;
(e) a post-transcriptional regulatory element, e.g., WPRE;
(f) a 3′UTR poly(A) tail sequence, e.g., bGHpA; and/or
(g) a second GPV ITR.

In another embodiment, the nucleic acid molecule comprises:
(a) a first B19 ITR;
(b) a tissue specific promoter sequence, e.g., TTP promoter;
(c) an intron, e.g., a synthetic intron;
(d) a heterologous polynucleotide sequence encoding phenylalanine hydroxylase (PAH);
(e) a post-transcriptional regulatory element, e.g., WPRE;
(f) a 3′UTR poly(A) tail sequence, e.g., bGHpA; and/or
(g) a second B19 ITR.

In another embodiment, the nucleic acid molecule comprises:
(a) a first GPV ITR;
(b) a tissue specific promoter sequence, e.g., TTP promoter;
(c) an intron, e.g., a synthetic intron;
(d) a heterologous polynucleotide sequence encoding phenylalanine hydroxylase (PAH);
(e) a post-transcriptional regulatory element, e.g., WPRE;
(f) a 3′UTR poly(A) tail sequence, e.g., bGHpA; and/or
(g) a second GPV ITR.

In another embodiment, the nucleic acid molecule comprises:
(a) a 5′ ITR bearing the B19d135 5′ ITR sequence set forth in SEQ ID NO: 180;
(b) a tissue specific promoter sequence, e.g., TTP promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the B19d135 3′ ITR sequence set forth in SEQ ID NO: 181.

In another embodiment, the nucleic acid molecule comprises:
(a) a 5′ ITR bearing the GPVd162 5′ ITR sequence set forth in SEQ ID NO: 183;
(b) a tissue specific promoter sequence, e.g., TTP promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the GPVd162 3′ ITR sequence set forth in SEQ ID NO: 184.

In another embodiment, the nucleic acid molecule comprises:
(a) a 5′ ITR bearing the full length B19 5′ ITR sequence set forth in SEQ ID NO: 185;
(b) a tissue specific promoter sequence, e.g., TTP promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the full length B19 3′ ITR sequence set forth in SEQ ID NO: 186.

In another embodiment, the nucleic acid molecule comprises:
(a) a 5′ ITR bearing the full length GPV 5′ ITR sequence set forth in SEQ ID NO: 187;
(b) a tissue specific promoter sequence, e.g., TTP promoter;
(c) an intron, e.g., a synthetic intron;
(d) a heterologous polynucleotide sequence encoding FVIII, e.g., FVIIIco6XTEN;
(e) a post-transcriptional regulatory element, e.g., WPRE;
(f) a 3′UTR poly(A) tail sequence, e.g., bGHpA; and/or
(g) a 3′ ITR bearing the full length GPV 3′ ITR sequence set forth in SEQ ID NO: 188.

In another embodiment, the nucleic acid molecule comprises:
(a) a 5′ ITR bearing the B19d135 5′ ITR sequence set forth in SEQ ID NO: 180;
(b) a tissue specific promoter sequence, e.g., CAG promoter;
(c) an intron, e.g., a synthetic intron;
(d) a heterologous polynucleotide sequence encoding PAH;
(e) a post-transcriptional regulatory element, e.g., WPRE;
(f) a 3′UTR poly(A) tail sequence, e.g., bGHpA; and/or
(g) a 3′ ITR bearing the B19d135 3′ ITR sequence set forth in SEQ ID NO: 181.

In another embodiment, the nucleic acid molecule comprises:
(a) a 5′ ITR bearing the GPVd162 5′ ITR sequence set forth in SEQ ID NO: 183;
(b) a tissue specific promoter sequence, e.g., CAG promoter;
(c) an intron, e.g., a synthetic intron;
(d) a heterologous polynucleotide sequence encoding PAH;
(e) a post-transcriptional regulatory element, e.g., WPRE;
(f) a 3′UTR poly(A) tail sequence, e.g., bGHpA; and/or
(g) a 3′ ITR bearing the GPVd162 3′ ITR sequence set forth in SEQ ID NO: 184.

In another embodiment, the nucleic acid molecule comprises:
(a) a 5′ ITR bearing the full length B19 5′ ITR sequence set forth in SEQ ID NO: 185;
(b) a tissue specific promoter sequence, e.g., CAG promoter;
(c) an intron, e.g., a synthetic intron;
(d) a heterologous polynucleotide sequence encoding PAH;
(e) a post-transcriptional regulatory element, e.g., WPRE;
(f) a 3′UTR poly(A) tail sequence, e.g., bGHpA; and/or
(g) a 3′ ITR bearing the full length B19 3′ ITR sequence set forth in SEQ ID NO: 186.

In another embodiment, the nucleic acid molecule comprises:
(a) a 5′ ITR bearing the full length GPV 5′ ITR sequence set forth in SEQ ID NO: 187;
(b) a tissue specific promoter sequence, e.g., CAG promoter;
(c) an intron, e.g., a synthetic intron;
(d) a heterologous polynucleotide sequence encoding PAH;
(e) a post-transcriptional regulatory element, e.g., WPRE;
(f) a 3′UTR poly(A) tail sequence, e.g., bGHpA; and/or
(g) a 3′ ITR bearing the full length GPV 3′ ITR sequence set forth in SEQ ID NO: 188.

### A. Inverted Terminal Repeats

Certain aspects of the present disclosure are directed to a nucleic acid molecule comprising a first ITR, e.g., a 5′ ITR, and second ITR, e.g., a 3′ ITR. Typically, ITRs are involved in parvovirus (e.g., AAV) DNA replication and rescue, or excision, from prokaryotic plasmids (Samulski et al., 1983, 1987; Senapathy et al., 1984; Gottlieb and Muzyczka, 1988). In addition, ITRs appear to be the minimum sequences required for AAV proviral integration and for packaging of AAV DNA into virions (McLaughlin et al., 1988; Samulski et al., 1989). These elements are essential for efficient multiplication of a parvovirus genome. It is hypothesized that the minimal defining elements indispensable for ITR function are a Rep-binding site (e.g., RBS; GCGCGCTCGCTCGCTC (SEQ ID NO: 104) for AAV2) and a terminal resolution site (e.g., TRS; AGTTGG (SEQ ID NO: 105) for AAV2) plus a variable palindromic sequence allowing for hairpin formation. Palindromic nucleotide regions normally function together in cis as origins of DNA replication and as packaging signals for the virus. Complimentary sequences in the ITRs fold into a hairpin structure during DNA replication. In some embodiments, the ITRs fold into a hairpin T-shaped structure. In other embodiments, the ITRs fold into non-T-shaped hairpin structures, e.g., into a U-shaped hairpin structure. Data suggests that the T-shaped hairpin structures ofAAV ITRs may inhibit the expression of a transgene flanked by the ITRs. See, e.g., Zhou et al., Scientific Reports 7:5432 (Jul. 14, 2017). By utilizing an ITR that does not form T-shaped hairpin structures, this form of inhibition may be avoided. Therefore, in certain aspects, a polynucleotide comprising a non-AAV ITR has an improved transgene expression compared to a polynucleotide comprising an AAV ITR that forms a T-shaped hairpin.

In some embodiments, the ITR comprises a naturally occurring ITR, e.g. the ITR comprises all or a portion of a parvovirus ITR. In some embodiments, the ITR comprises a synthetic sequence. In one embodiment, the first ITR or the second ITR comprises a synthetic sequence. In another embodiment, each of the first ITR and the second ITR comprises a synthetic sequence. In some embodiments, the first ITR or the second ITR comprises a naturally occurring sequence. In another embodiment, each of the first ITR and the second ITR comprises a naturally occurring sequence.

In some embodiments, the ITR comprises or consists of a portion of a naturally occurring ITR, e.g., a truncated ITR. In some embodiments, the ITR comprises or consists of a fragment of a naturally occurring ITR, wherein the fragment comprises at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 125 nucleotides, at least about 150 nucleotides, at least about 175 nucleotides, at least about 200 nucleotides, at least about 225 nucleotides, at least about 250 nucleotides, at least about 275 nucleotides, at least about 300 nucleotides, at least about 325 nucleotides, at least about 350 nucleotides, at least about 375 nucleotides, at least about 400 nucleotides, at least about 425 nucleotides, at least about 450 nucleotides, at least about 475 nucleotides, at least about 500 nucleotides, at least about 525 nucleotides, at least about 550 nucleotides, at least about 575 nucleotides, or at least about 600 nucleotides; wherein the ITR retains a functional property of the naturally occurring ITR. In certain embodiments, the ITR comprises or consists of a fragment of a naturally occurring ITR, wherein the fragment comprises at least about 129 nucleotides; wherein the ITR retains a functional property of the naturally occurring ITR. In certain embodiments, the ITR comprises or consists of a fragment of a naturally occurring ITR, wherein the fragment comprises at least about 102 nucleotides; wherein the ITR retains a functional property of the naturally occurring ITR.

In some embodiments, the ITR comprises or consists of a portion of a naturally occurring ITR, wherein the fragment comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the length of the naturally occurring ITR; wherein the fragment retains a functional property of the naturally occurring ITR.

In certain embodiments, the ITR comprises or consists of a sequence that has a sequence identity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to a homologous portion of a naturally occurring ITR, when properly aligned; wherein the ITR retains a functional property of the naturally occurring ITR. In other embodiments, the ITR comprises or consists of a sequence that has a sequence identity of at least 90% to a homologous portion of a naturally occurring ITR, when properly aligned; wherein the ITR retains a functional property of the naturally occurring ITR. In some embodiments, the ITR comprises or consists of a sequence that has a sequence identity of at least 80% to a homologous portion of a naturally occurring ITR, when properly aligned; wherein the ITR retains a functional property of the naturally occurring ITR. In some embodiments, the ITR comprises or consists of a sequence that has a sequence identity of at least 70% to a homologous portion of a naturally occurring ITR, when properly aligned; wherein the ITR retains a functional property of the naturally occurring ITR. In some embodiments, the ITR comprises or consists of a sequence that has a sequence identity of at least 60% to a homologous portion of a naturally occurring ITR, when properly aligned; wherein the ITR retains a functional property of the naturally occurring ITR. In some embodiments, the ITR comprises or consists of a sequence that has a sequence identity of at least 50% to a homologous portion of a naturally occurring ITR, when properly aligned; wherein the ITR retains a functional property of the naturally occurring ITR.

In some embodiments, the ITR comprises an ITR from an AAV genome. In some embodiments, the ITR is an ITR of an AAV genome selected from AAV1, AAV2, AAV3, AAV4, AAV5, AAV6, AAV7, AAV8, AAV9, AAV10 AAV11, and any combination thereof. In a particular embodiment, the ITR is an ITR of the AAV2 genome. In another embodiment, the ITR is a synthetic sequence genetically engineered to include at its 5′ and 3′ ends ITRs derived from one or more of AAV genomes.

In some embodiments, the ITR is not derived from an AAV genome. In some embodiments, the ITR is an ITR of a non-AAV. In some embodiments, the ITR is an ITR of a non-AAV genome from the viral family Parvoviridae selected from, but not limited to, the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In certain embodiments, the ITR is derived from Erythrovirus parvovirus B19 (human virus). In another embodiment, the ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY. In some embodiments, the ITR is derived from a porcine parvovirus, e.g., porcine parvovirus U44978. In some embodiments, the ITR is derived from a mice minute virus, e.g., mice minute virus U34256. In some embodiments, the ITR is derived from a canine parvovirus, e.g., canine parvovirus M19296. In some embodiments, the ITR is derived from a mink enteritis virus, e.g., mink enteritis virus D00765. In some embodiments, the ITR is derived from a Dependoparvovirus. In one embodiment, the Dependoparvovirus is a Dependovirus Goose parvovirus (GPV) strain. In a specific embodiment, the GPV strain is attenuated, e.g., GPV strain 82-0321V. In another specific embodiment, the GPV strain is pathogenic, e.g., GPV strain B.

The first ITR and the second ITR of the nucleic acid molecule can be derived from the same genome, e.g., from the genome of the same virus, or from different genomes, e.g., from the genomes of two or more different virus genomes. In certain embodiments, the first ITR and the second ITR are derived from the same AAV genome. In a specific embodiment, the two ITRs present in the nucleic acid molecule of the invention are the same, and can in particular be AAV2 ITRs. In other embodiments, the first ITR is derived from an AAV genome and the second ITR is not derived from an AAV genome (e.g., a non-AAV genome). In other embodiments, the first ITR is not derived from an AAV genome (e.g., a non-AAV genome) and the second ITR is derived from an AAV genome. In still other embodiments, both the first ITR and the second ITR are not derived from an AAV genome (e.g., a non-AAV genome). In one particular embodiment, the first ITR and the second ITR are identical.

In some embodiments, the first ITR is derived from an AAV genome, and the second ITR is derived from a genome selected from the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus and any combination thereof. In other embodiments, the second ITR is derived from an AAV genome, and the first ITR is derived from a genome selected from the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus, and any combination thereof. In other embodiments, the first ITR and the second ITR are derived from a genome selected from the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus, and any combination thereof, wherein the first ITR and the second ITR are derived from the same genome. In other embodiments, the first ITR and the second ITR are derived from a genome selected from the group consisting of Bocavirus, Dependovirus, Erythrovirus, Amdovirus, Parvovirus, Densovirus, Iteravirus, Contravirus, Aveparvovirus, Copiparvovirus, Protoparvovirus, Tetraparvovirus, Ambidensovirus, Brevidensovirus, Hepandensovirus, Penstyldensovirus, and any combination thereof, wherein the first ITR and the second ITR are derived from the different genomes.

In some embodiments, the first ITR is derived from an AAV genome, and the second ITR is derived from Erythrovirus parvovirus B19 (human virus). In other embodiments, the second ITR is derived from an AAV genome, and the first ITR is derived from Erythrovirus parvovirus B19 (human virus).

In certain embodiments, the first ITR and/or the second ITR comprises or consists of all or a portion of an ITR derived from B19. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 167, 168, 169, 170, and 171, wherein the first ITR and/or the second ITR retains a functional property of the B19 ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 167, 168, 169, 170, and 171, wherein the first ITR and/or the second ITR is capable of forming a hairpin structure. In certain embodiments, the hairpin structure does not comprise a T-shaped hairpin.

In some embodiments, the first ITR and/or the second ITR comprises or consists of a nucleotide sequence selected from SEQ ID NOs: 167, 168, 169, 170, and 171. In some embodiments, the first ITR and/or the second ITR comprises or consists of the nucleotide sequence set forth in SEQ ID NO: 167. In some embodiments, the first ITR and/or the second ITR comprises or consists of the nucleotide sequence set forth in SEQ ID NO: 168. In some embodiments, the first ITR and/or the second ITR comprises or consists of the nucleotide sequence set forth in SEQ ID NO: 169. In some embodiments, the first ITR and/or the second ITR comprises or consists of the nucleotide sequence set forth in SEQ ID NO: 170. In some embodiments, the first ITR and/or the second ITR comprises or consists of the nucleotide sequence set forth in SEQ ID NO: 171.

TABLE 1
Sample Parvovirus ITR Sequences.

| | ITR | | Length | |
|---|---|---|---|---|
| Parvovirus | ID | Description | (nt) | Sequence |
| B19 | wt | Gene Bank: | 383 | CCAAATCAGATGCCGCCGGTCGCCGCCGGTAGGC |
| | | KY940273.1 | | GGGACTTCCGGTACAAGATGGCGGACAATTACGT |
| | | | | CATTTCCTGTGACGTCATTTCCTGTGACGTCACT |
| | | | | TCCGGTGGGCGGGACTTCCGGAATTAGGGTTGGC |
| | | | | TCTGGGCCAGCTTGCTTGGGGTTGCCTTGACACT |
| | | | | AAGACAAGCGGCGCGCCGCTTGATCTTAGTGGCA |
| | | | | CGTCAACCCCAAGCGCTGGCCCAGAGCCAACCCT |
| | | | | AATTCCGGAAGTCCCGCCCACCGGAAGTGACGTC |
| | | | | ACAGGAAATGACGTCACAGGAAATGACGTAATTG |
| | | | | TCCGCCATCTTGTACCGGAAGTCCCGCCTACCGG |
| | | | | CGGCGACCGGCGGCATCTGATTTGGTGTCTTCTT |
| | | | | TTAAATTTT (SEQ ID NO: 167) |
| | d135 | excludes first | 248 | CTCTGGGCCAGCTTGCTTGGGGTTGCCTTGACAC |
| | | 135 | | TAAGACAAGCGGCGCGCCGCTTGATCTTAGTGGC |
| | | nucleotides | | ACGTCAACCCCAAGCGCTGGCCCAGAGCCAACCC |
| | | | | TAATTCCGGAAGTCCCGCCCACCGGAAGTGACGT |
| | | | | CACAGGAAATGACGTCACAGGAAATGACGTAATT |
| | | | | GTCCGCCATCTTGTACCGGAAGTCCCGCCTACCG |
| | | | | GCGGCGACCGGCGGCATCTGATTTGGTGTCTTCT |
| | | | | TTTAAATTTT (SEQ ID NO: 168) |
| | v1 | minimal | 129 | CGGCGCGCCGCTTGATCTTAGTGGCACGTCAACC |
| | | sequence | | AGCGCTGGCCCAGAGCCAACCCTAATTCCGGAAG |
| | | based on | | TCCTCAGTCCGCCATCTTGCCCGCCTACCGGCGG |
| | | comparison | | CGACCGGCGGCATCATTTGGTGTTCTT (SEQ |
| | | with AAV2 | | ID NO: 169) |
| | v2 | excludes first | 113 | CTCTGGGCCAGCTTGCTTGGGGTTGCCTTGACAC |
| | | 135 | | TAAGACAAGCGGCGCGCCGCTTGATCTTAGTGGC |
| | | nucleotides | | ACGTCAACCCCAAGCGCTGGCCCAGAGTGTCTTC |
| | | and | | TTTTAAATTTT (SEQ ID NO: 170) |
| | | corresponding | | |
| | | complementary | | |
| | | 135 | | |
| | | nucleotides in | | |
| | | palindrome | | |
| | v3 | minimal | 340 | CAAATCAGATGCCGCCGGTCGCCGCCGGTAGGCG |
| | | sequence | | GGACTTCCGGTACAAGATGGCGGACAATTACGTC |
| | | based on | | ATTTCCTGTGACGTATTTCCTGTGACGTACTTCC |
| | | comparison | | GGTGGCGGGACTTCCGGAATTTTGGCTCTGGGCC |
| | | with GPV | | AGCTTGCTTGGGGTTGCCTTGACCAAGCGCGCGC |
| | | | | CGCTTGATCACCCCAAGCGCTGGCCCAGAGCCAC |
| | | | | CTAACCGGAAGTCCCCCCACCGGAAGTGACGTCA |
| | | | | CAGGAAAGACGTCACAGGAAGTAATTGTCCGCCA |
| | | | | TCTTGTACCGGAAGTCCCGCACCGGCGGCGACCG |
| | | | | GCGGCATCTGATTTGGTGTCTTCTTTTAAATTTT |
| | | | | (SEQ ID NO: 171) |
| GPV | wt | Gene Bank: | 444 | CTCATTGGAGGGTTCGTTCGTTCGAACCAGCCAA |
| | | U25749.1 | | TCAGGGGAGGGGGAAGTGACGCAAGTTCCGGTCA |
| | | | | CATGCTTCCGGTGACGCACATCCGGTGACGTAGT |
| | | | | TCCGGTCACGTGCTTCCTGTCACGTGTTTCCGGT |
| | | | | CACGTGACTTCCGGTCATGTGACTTCCGGTGACG |
| | | | | TGTTTCCGGCTGTTAGGTTGACCACGCGCATGCC |
| | | | | GCGCGGTCAGCCCAATAGTTAAGCCGGAAACACG |
| | | | | TCACCGGAAGTCACATGACCGGAAGTCACGTGAC |
| | | | | CGGAAACACGTGACAGGAAGCACGTGACCGGAAC |
| | | | | TACGTCACCGGATGTGCGTCACCGGAAGCATGTG |
| | | | | ACCGGAACTTGCGTCACTTCCCCCTCCCCTGATT |
| | | | | GGCTGGTTCGAACGAACGAACCCTCCAATGAGAC |
| | | | | TCAAGGACAAGAGGATATTTTGCGCGCCAGGAAG |
| | | | | TG (SEQ ID NO: 172) |
| | d162 | excludes first | 282 | CGGTGACGTGTTTCCGGCTGTTAGGTTGACCACG |
| | | 162 | | CGCATGCCGCGCGGTCAGCCCAATAGTTAAGCCG |
| | | nucleotides | | GAAACACGTCACCGGAAGTCACATGACCGGAAGT |
| | | | | CACGTGACCGGAAACACGTGACAGGAAGCACGTG |
| | | | | ACCGGAACTACGTCACCGGATGTGCGTCACCGGA |
| | | | | AGCATGTGACCGGAACTTGCGTCACTTCCCCCTC |
| | | | | CCCTGATTGGCTGGTTCGAACGAACGAACCCTCC |
| | | | | AATGAGACTCAAGGACAAGAGGATATTTTGCGCG |
| | | | | CCAGGAAGTG (SEQ ID NO: 173) |
| | v1 | minimal | 145 | TTGACCACGCGCATGCCGCGCGGTCAGCCCAATA |
| | | sequence | | GTTAAGCCGGGTGACCACACGTGACAGGAAGCAC |
| | | based on | | GGGATGTGCGTCACCGGAAGCAGTGACCGGGCTG |
| | | comparison | | GTTCGAACGAACGAACCCTCCAACTCAAGGACAA |
| | | with AAV2 | | GAGGATATT (SEQ ID NO: 174) |
| | v2 | excludes first | 120 | CGGTGACGTGTTTCCGGCTGTTAGGTTGACCACG |
| | | 162 | | CGCATGCCGCGCGGTCAGCCCAATAGTTAAGCCG |
| | | nucleotides | | GAAACACGTCACCGACTCAAGGACAAGAGGATAT |
| | | and | | TTTGCGCGCCAGGAAGTG (SEQ ID NO: |
| | | corresponding | | 175) |
| | | complementary | | |
| | | 162 | | |
| | | nucleotides in | | |
| | | palindrome | | |
| | v3 | minimal | 102 | GGGAACAATCAGGGGAAGTGACCGGTGACGTCAT |
| | | sequence | | GTAACTTGCGTCACTTCCCGTTCGAACGAACGAA |
| | | based on | | CGAGACTCAAGGACAAGAGGCGCGCCAGGAAGTG |
| | | comparison | | (SEQ ID NO: 176) |
| | | with B19 | | |
| AAV2 | wt | Gene Bank: | 145 | TTGGCCACTCCCTCTCTGCGCGCTCGCTCGCTCA |
| | | NC_001401.2 | | CTGAGGCCGGGCGACCAAAGGTCGCCCGACGCCC |
| | | | | GGGCTTTGCCCGGGCGGCCTCAGTGAGCGAGCGA |
| | | | | GCGCGCAGAGAGGGAGTGGCCAACTCCATCACTA |
| | | | | GGGGTTCCT (SEQ ID NO: 177) |
| | GTx | used in GTx | 130 | CTGCGCGCTCGCTCGCTCACTGAGGCCGCCCGGG |
| | | vectors | | CAAAGCCCGGGCGTCGGGCGACCTTTGGTCGCCC |
| | | | | GGCCTCAGTGAGCGAGCGAGCGCGCAGAGAGGGA |
| | | | | GTGGCCAACTCCATCACTAGGGGTTCCT (SEQ |
| | | | | ID NO: 178) |

In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 167. In certain embodiments, the first ITR and/or the second ITR consists of SEQ ID NO: 167. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 168. In certain embodiments, the first ITR and/or the second ITR consists of SEQ ID NO: 168. In certain embodiments, the first ITR and/or the second ITR comprises a nucleotide sequence, wherein the nucleotide sequence comprises the minimal nucleotide sequence set forth in SEQ ID NO: 169, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 167, retains a functional property of the B19 ITR from which it is derived. In some embodiments, the first ITR and/or the second ITR comprises a nucleotide sequence, wherein the nucleotide sequence comprises the minimal nucleotide sequence set forth in SEQ ID NO: 169, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 167, wherein the first ITR and/or the second ITR is capable of forming a hairpin structure. In certain embodiments, the hairpin structure does not comprise a T-shaped hairpin.

In certain embodiments, the first ITR and/or the second ITR comprises or consists of all or a portion of an ITR derived from B19. In some embodiments, the second ITR is a reverse complement of the first ITR. In some embodiments, the first ITR is a reverse complement of the second ITR. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 180, 181, 185, and 186, or a functional derivative thereof. In some embodiments, the functional derivative retains a functional property of the B19 ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 180, 181, 185, and 186, or a functional derivative thereof. In some embodiments, the functional derivative is capable of forming a hairpin structure. In certain embodiments, the hairpin structure does not comprise a T-shaped hairpin.

In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 180. In certain embodiments, the first ITR and/or the second ITR consists of SEQ ID NO: 180. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 181. In certain embodiments, the first ITR and/or the second ITR consists of SEQ ID NO: 181. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 185. In certain embodiments, the first ITR and/or the second ITR consists of SEQ ID NO: 185. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 186. In certain embodiments, the first ITR and/or the second ITR consists of SEQ ID NO: 186.

In some embodiments, the first ITR and/or the second ITR comprises or consists of a nucleotide sequence selected from SEQ ID NOs: 180, 181, 185, and 186. In some embodiments, the first ITR comprises or consists of the nucleotide sequence set forth in SEQ ID NO: 180. In some embodiments, the first ITR comprises or consists of the nucleotide sequence set forth in SEQ ID NO: 181. In some embodiments, the first ITR comprises or consists of the nucleotide sequence set forth in SEQ ID NO: 185. In some embodiments, the first ITR comprises or consists of the nucleotide sequence set forth in SEQ ID NO: 186. In some embodiments, the second ITR comprises or consists of the nucleotide sequence set forth in SEQ ID NO: 180. In some embodiments, the second ITR comprises or consists of the nucleotide sequence set forth in SEQ ID NO: 181. In some embodiments, the second ITR comprises or consists of the nucleotide sequence set forth in SEQ ID NO: 185. In some embodiments, the second ITR comprises or consists of the nucleotide sequence set forth in SEQ ID NO: 186.

In some embodiments, the first ITR comprises or consists of the nucleotide sequence set forth in SEQ ID NO:180, and the second ITR comprises or consists of the nucleotide sequence set forth in SEQ ID NO: 181. In some embodiments, the first ITR comprises or consists of the nucleotide sequence set forth in SEQ ID NO:181, and the second ITR comprises or consists of the nucleotide sequence set forth in SEQ ID NO: 180. In some embodiments, the first ITR comprises or consists of the nucleotide sequence set forth in SEQ ID NO:185, and the second ITR comprises or consists of the nucleotide sequence set forth in SEQ ID NO: 186. In some embodiments, the first ITR comprises or consists of the nucleotide sequence set forth in SEQ ID NO:186, and the second ITR comprises or consists of the nucleotide sequence set forth in SEQ ID NO: 185.

In some embodiments, the first ITR is derived from an AAV genome, and the second ITR is derived from GPV. In other embodiments, the second ITR is derived from an AAV genome, and the first ITR is derived from GPV.

In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 172. In certain embodiments, the first ITR and/or the second ITR consists of SEQ ID NO: 172. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 173. In certain embodiments, the first ITR and/or the second ITR consists of SEQ ID NO: 173. In certain embodiments, the first ITR and/or the second ITR comprises or consists of all or a portion of an ITR derived from GPV. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 172, 173, 174, 175, and 176, wherein the first ITR and/or the second ITR retains a functional property of the GPV ITR from which it is derived. In some embodiments, the first ITR and/or the second ITR comprises or consists of all or a portion of an ITR derived from GPV. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 172, 173, 174, 175, and 176, wherein the first ITR and/or the second ITR is capable of forming a hairpin structure. In certain embodiments, the hairpin structure does not comprise a T-shaped hairpin. In some embodiments, the first ITR and/or the second ITR comprises or consists of a nucleotide sequence selected from SEQ ID NOs: 172, 173, 174, 175, and 176. In some embodiments, the first ITR and/or the second ITR comprises or consists of the nucleotide sequence set forth in SEQ ID NO: 172. In some embodiments, the first ITR and/or the second ITR comprises or consists of the nucleotide sequence set forth in SEQ ID NO: 173. In some embodiments, the first ITR and/or the second ITR comprises or consists of the nucleotide sequence set forth in SEQ ID NO: 174. In some embodiments, the first ITR and/or the second ITR comprises or consists of the nucleotide sequence set forth in SEQ ID NO: 175. In some embodiments, the first ITR and/or the second ITR comprises or consists of the nucleotide sequence set forth in SEQ ID NO: 176.

In certain embodiments, the first ITR and/or the second ITR comprises a nucleotide sequence, wherein the nucleotide sequence comprises the minimal nucleotide sequence set forth in SEQ ID NO: 174, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 172, wherein the first ITR and/or the second ITR retains a functional property of the GPV ITR from which it is derived. In some embodiments, the first ITR and/or the second ITR comprises a nucleotide sequence, wherein the nucleotide sequence comprises the minimal nucleotide sequence set forth in SEQ ID NO: 174, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 172, wherein the first ITR and/or the second ITR is capable of forming a hairpin structure. In certain embodiments, the hairpin structure does not comprise a T-shaped hairpin.

In certain embodiments, the first ITR and/or the second ITR comprises a nucleotide sequence, wherein the nucleotide sequence comprises the minimal nucleotide sequence set forth in SEQ ID NO: 176, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 172, wherein the first ITR and/or the second ITR retains a functional property of the GPV ITR from which it is derived. In some embodiments, the first ITR and/or the second ITR comprises a nucleotide sequence, wherein the nucleotide sequence comprises the minimal nucleotide sequence set forth in SEQ ID NO: 176, and wherein the nucleotide sequence is a at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 172, wherein the first ITR and/or the second ITR is capable of forming a hairpin structure. In certain embodiments, the hairpin structure does not comprise a T-shaped hairpin.

In certain embodiments, the first ITR and/or the second ITR comprises or consists of all or a portion of an ITR derived from GPV. In some embodiments, the second ITR is a reverse complement of the first ITR. In some embodiments, the first ITR is a reverse complement of the second ITR. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 183, 184, 187 and 188, or a functional derivative thereof. In some embodiments, the functional derivative retains a functional property of the GPV ITR from which it is derived. In some embodiments, the first ITR and/or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence selected from SEQ ID NOs: 183, 184, 187 and 188, or a functional derivative thereof. In some embodiments, the functional derivative is capable of forming a hairpin structure. In certain embodiments, the hairpin structure does not comprise a T-shaped hairpin.

In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 183. In certain embodiments, the first ITR and/or the second ITR consists of SEQ ID NO: 183. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 184. In certain embodiments, the first ITR and/or the second ITR consists of SEQ ID NO: 184. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 187. In certain embodiments, the first ITR and/or the second ITR consists of SEQ ID NO: 187. In certain embodiments, the first ITR and/or the second ITR comprises a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to the nucleotide sequence set forth in SEQ ID NO: 188. In certain embodiments, the first ITR and/or the second ITR consists of SEQ ID NO: 188.

In some embodiments, the first ITR and/or the second ITR comprises or consists of a nucleotide sequence selected from SEQ ID NOs: 183, 184, 187 and 188. In some embodiments, the first ITR comprises or consists of the nucleotide sequence set forth in SEQ ID NO: 183. In some embodiments, the first ITR comprises or consists of the nucleotide sequence set forth in SEQ ID NO: 184. In some embodiments, the first ITR comprises or consists of the nucleotide sequence set forth in SEQ ID NO: 187. In some embodiments, the first ITR comprises or consists of the nucleotide sequence set forth in SEQ ID NO: 188. In some embodiments, the second ITR comprises or consists of the nucleotide sequence set forth in SEQ ID NO: 183. In some embodiments, the second ITR comprises or consists of the nucleotide sequence set forth in SEQ ID NO: 184. In some embodiments, the second ITR comprises or consists of the nucleotide sequence set forth in SEQ ID NO: 187. In some embodiments, the second ITR comprises or consists of the nucleotide sequence set forth in SEQ ID NO: 188.

In some embodiments, the first ITR comprises or consists of the nucleotide sequence set forth in SEQ ID NO:183, and the second ITR comprises or consists of the nucleotide sequence set forth in SEQ ID NO: 184. In some embodiments, the first ITR comprises or consists of the nucleotide sequence set forth in SEQ ID NO:184, and the second ITR comprises or consists of the nucleotide sequence set forth in SEQ ID NO: 183. In some embodiments, the first ITR comprises or consists of the nucleotide sequence set forth in SEQ ID NO:187, and the second ITR comprises or consists of the nucleotide sequence set forth in SEQ ID NO: 188. In some embodiments, the first ITR comprises or consists of the nucleotide sequence set forth in SEQ ID NO:188, and the second ITR comprises or consists of the nucleotide sequence set forth in SEQ ID NO: 187.

In certain embodiments, one of the first ITR or the second ITR comprises or consists of all or a portion of an ITR derived from AAV2. In some embodiments, the first ITR or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs: 177 or 178, wherein the first ITR and/or the second ITR retains a functional property of the AAV2 ITR from which it is derived. In some embodiments, the first ITR or the second ITR comprises or consists of a nucleotide sequence at least about 50%, at least about 55%, at least about 60%, at least about 65%, at least about 65%, at least about 70%, at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NOs: 177 or 178, wherein the first ITR and/or the second ITR is capable of forming a hairpin structure. In certain embodiments, the hairpin structure does not comprise a T-shaped hairpin. In some embodiments, the first ITR and/or the second ITR comprises or consists of a nucleotide sequence set forth in SEQ ID NOs: 177 or 178. In some embodiments, the first ITR and/or the second ITR comprises or consists of the nucleotide sequence set forth in SEQ ID NO: 177. In some embodiments, the first ITR and/or the second ITR comprises or consists of the nucleotide sequence set forth in SEQ ID NO: 178.

In some embodiments, the first ITR is derived from an AAV genome, and the second ITR is derived from a Muscovy duck parvovirus (MDPV) strain. In other embodiments, the second ITR is derived from an AAV genome, and the first ITR is derived from a Muscovy duck parvovirus (MDPV) strain. In certain embodiments, the MDPV strain is attenuated, e.g., MDPV strain FZ91-30. In other embodiments, the MDPV strain is pathogenic, e.g., MDPV strain YY.

In some embodiments, the first ITR is derived from an AAV genome, and the second ITR is derived from a Dependoparvovirus. In some embodiments, the second ITR is derived from an AAV genome, and the first ITR is derived from a Dependoparvovirus. In other embodiments, the first ITR is derived from an AAV genome, and the second ITR is derived from a Dependovirus goose parvovirus (GPV) strain. In other embodiments, the second ITR is derived from an AAV genome, and the first ITR is derived from a Dependovirus GPV strain. In certain embodiments, the GPV strain is attenuated, e.g., GPV strain 82-0321V. In other embodiments, the GPV strain is pathogenic, e.g., GPV strain B.

In certain embodiments, the first ITR is derived from an AAV genome, and the second ITR is derived from a genome selected from the group consisting of porcine parvovirus, e.g., porcine parvovirus strain U44978; mice minute virus, e.g., mice minute virus strain U34256; canine parvovirus, e.g., canine parvovirus strain M19296; mink enteritis virus, e.g., mink enteritis virus strain D00765; and any combination thereof. In other embodiments, the second ITR is derived from an AAV genome, and the first ITR is derived from a genome selected from the group consisting of porcine parvovirus, e.g., porcine parvovirus strain U44978; mice minute virus, e.g., mice minute virus strain U34256; canine parvovirus, e.g., canine parvovirus strain M19296; mink enteritis virus, e.g., mink enteritis virus strain D00765; and any combination thereof.

In another particular embodiment, the ITR is a synthetic sequence genetically engineered to include at its 5′ and 3′ ends ITRs not derived from an AAV genome. In another particular embodiment, the ITR is a synthetic sequence genetically engineered to include at its 5′ and 3′ ends ITRs derived from one or more of non-AAV genomes. The two ITRs present in the nucleic acid molecule of the invention can be the same or different non-AAV genomes. In particular, the ITRs can be derived from the same non-AAV genome. In a specific embodiment, the two ITRs present in the nucleic acid molecule of the invention are the same, and can in particular be AAV2 ITRs.

In some embodiments, the ITR sequence comprises one or more palindromic sequence. A palindromic sequence of an ITR disclosed herein includes, but is not limited to, native palindromic sequences (i.e., sequences found in nature), synthetic sequences (i.e., sequences not found in nature), such as pseudo palindromic sequences, and combinations or modified forms thereof. A “pseudo palindromic sequence” is a palindromic DNA sequence, including an imperfect palindromic sequence, which shares less than 80% including less than 70%, 60%, 50%, 40%, 30%, 20%, 10%, or 5%, or no, nucleic acid sequence identity to sequences in native AAV or non-AAV palindromic sequence which form a secondary structure. The native palindromic sequences can be obtained or derived from any genome disclosed herein. The synthetic palindromic sequence can be based on any genome disclosed herein.

The palindromic sequence can be continuous or interrupted. In some embodiments, the palindromic sequence is interrupted, wherein the palindromic sequence comprises an insertion of a second sequence. In some embodiments, the second sequence comprises a promoter, an enhancer, an integration site for an integrase (e.g., sites for Cre or Flp recombinase), an open reading frame for a gene product, or a combination thereof.

In some embodiments, the ITRs form hairpin loop structures. In one embodiment, the first ITR forms a hairpin structure. In another embodiment, the second ITR forms a hairpin structure. Still in another embodiment, both the first ITR and the second ITR form hairpin structures. In some embodiments, the first ITR and/or the second ITR does not form a T-shaped hairpin structure. In certain embodiments, the first ITR and/or the second ITR forms a non-T-shaped hairpin structure. In some embodiments, the non-T-shaped hairpin structure comprises a U-shaped hairpin structure.

In some embodiments, an ITR in a nucleic acid molecule described herein may be a transcriptionally activated ITR. A transcriptionally-activated ITR can comprise all or a portion of a wild-type ITR that has been transcriptionally activated by inclusion of at least one transcriptionally active element. Various types of transcriptionally active elements are suitable for use in this context. In some embodiments, the transcriptionally active element is a constitutive transcriptionally active element. Constitutive transcriptionally active elements provide an ongoing level of gene transcription, and are preferred when it is desired that the transgene be expressed on an ongoing basis. In other embodiments, the transcriptionally active element is an inducible transcriptionally active element. Inducible transcriptionally active elements generally exhibit low activity in the absence of an inducer (or inducing condition), and are up-regulated in the presence of the inducer (or switch to an inducing condition). Inducible transcriptionally active elements may be preferred when expression is desired only at certain times or at certain locations, or when it is desirable to titrate the level of expression using an inducing agent. Transcriptionally active elements can also be tissue-specific; that is, they exhibit activity only in certain tissues or cell types.

Transcriptionally active elements, can be incorporated into an ITR in a variety of ways. In some embodiments, a transcriptionally active element is incorporated 5′ to any portion of an ITR or 3′ to any portion of an ITR. In other embodiments, a transcriptionally active element of a transcriptionally-activated ITR lies between two ITR sequences. If the transcriptionally active element comprises two or more elements which must be spaced apart, those elements may alternate with portions of the ITR. In some embodiments, a hairpin structure of an ITR is deleted and replaced with inverted repeats of a transcriptional element. This latter arrangement would create a hairpin mimicking the deleted portion in structure. Multiple tandem transcriptionally active elements can also be present in a transcriptionally-activated ITR, and these may be adjacent or spaced apart. In addition, protein binding sites (e.g., Rep binding sites) can be introduced into transcriptionally active elements of the transcriptionally-activated ITRs. A transcriptionally active element can comprise any sequence enabling the controlled transcription of DNA by RNA polymerase to form RNA, and can comprise, for example, a transcriptionally active element, as defined below.

Transcriptionally-activated ITRs provide both transcriptional activation and ITR functions to the nucleic acid molecule in a relatively limited nucleotide sequence length which effectively maximizes the length of a transgene which can be carried and expressed from the nucleic acid molecule. Incorporation of a transcriptionally active element into an ITR can be accomplished in a variety of ways. A comparison of the ITR sequence and the sequence requirements of the transcriptionally active element can provide insight into ways to encode the element within an ITR. For example, transcriptional activity can be added to an ITR through the introduction of specific changes in the ITR sequence that replicates the functional elements of the transcriptionally active element. A number of techniques exist in the art to efficiently add, delete, and/or change particular nucleotide sequences at specific sites (see, for example, Deng and Nickoloff (1992) Anal. Biochem. 200:81-88). Another way to create transcriptionally-activated ITRs involves the introduction of a restriction site at a desired location in the ITR. In addition, multiple transcriptionally activate elements can be incorporated into a transcriptionally-activated ITR, using methods known in the art.

By way of illustration, transcriptionally-activated ITRs can be generated by inclusion of one or more transcriptionally active elements such as: TATA box, GC box, CCAAT box, Sp1 site, Inr region, CRE (cAMP regulatory element) site, ATF-1/CRE site, APBβ box, APBα box, CArG box, CCAC box, or any other element involved in transcription as known in the art.

Aspects of the present disclosure provide a method of cloning a nucleic acid molecule described herein, comprising inserting a nucleic acid molecule capable of complex secondary structures into a suitable vector, and introducing the resulting vector into a suitable bacterial host strain. As known in the art, complex secondary structures (e.g., long palindromic regions) of nucleic acids may be unstable and difficult to clone in bacterial host strains. For example, nucleic acid molecules comprising a first ITR and a second ITR (e.g., non-AAV parvoviral ITRs, e.g., B19 or GPV ITRs) of the present disclosure may be difficult to clone using conventional methodologies. Long DNA plindromes inhibit DNA replication and are unstable in the genomes of E. coli, Bacillus, Steptococcus, Streptomyces, S. cerevisiae, mice, and humans. These effects result from the formation of hairpin or cruciform structures by intrastrand base pairing. In E. coli the inhibition of DNA replication can be significantly overcome in SbcC or SbcD mutants. SbcD is the nuclease subunit, and SbcC is the ATPase subunit of the SbcCD complex. The E. coli SbcCD complex is an exonuclease complex responsible for preventing the replication of long palindromes. The SbcCD complex is a nuclear with ATP-dependent double-stranded DNA exonuclease activity and ATP-independent single-stranded DNA endonuclease activity. SbcCD may recognize DNA plaindromes and collapse replication forks by attacking hairpin structures that arise.

In certain embodiments, a suitable bacterial host strain is incapable of resolving cruciform DNA structures. In certain embodiments, a suitable bacterial host strain comprises a disruption in the SbcCD complex. In some embodiments, the disruption in the SbcCD complex comprises a genetic disruption in the SbcC gene and/or SbcD gene. In certain embodiments, the disruption in the SbcCD complex comprises a genetic disruption in the SbcC gene. Various bacterial host strains that comprise a genetic disruption in the SbcC gene are known in the art. For example, without limitation, the bacterial host strain PMC103 comprises the genotype sbcC, recD, mcrA, ΔmcrBCF; the bacterial host strain PMC107 comprises the genotype recBC, recJ, sbcBC, mcrA, ΔmcrBCF; and the bacterial host strain SURE comprises the genotype recB, recJ, sbcC, mcrA, ΔmcrBCF, umuC, uvrC. Accordingly, in some embodiments a method of cloning a nucleic acid molecule described herein comprises inserting a nucleic acid molecule capable of complex secondary structures into a suitable vector, and introducing the resulting vector into host strain PMC103, PMC107, or SURE. In certain embodiments, the method of cloning a nucleic acid molecule described herein comprises inserting a nucleic acid molecule capable of complex secondary structures into a suitable vector, and introducing the resulting vector into host strain PMC103.

Suitable vectors are known in the art and described elsewhere herein. In certain embodiments, a suitable vector for use in a cloning methodology of the present disclosure is a low copy vector. In certain embodiments, a suitable vector for use in a cloning methodology of the present disclosure is pBR322.

Accordingly, the present disclosure provides a method of cloning a nucleic acid molecule, comprising inserting a nucleic acid molecule capable of complex secondary structures into a suitable vector, and introducing the resulting vector into a bacterial host strain comprising a disruption in the SbcCD complex, wherein the nucleic acid molecule comprises a first inverted terminal repeat (ITR) and a second ITR, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

### B. Therapeutic Proteins

Certain aspects of the present disclosure are directed to a nucleic acid molecule comprising a first ITR, a second ITR, and a genetic cassette encoding a target sequence, wherein the target sequence encodes a therapeutic protein. In some embodiments, the genetic cassette encodes one therapeutic protein. In some embodiments, the genetic cassette encodes more than one therapeutic protein. In some embodiments, the genetic cassette encodes two or more copies of the same therapeutic protein. In some embodiments, the genetic cassette encodes two or more variants of the same therapeutic protein. In some embodiments, the genetic cassette encodes two or more different therapeutic proteins.

Certain embodiments of the present disclosure are directed to a nucleic acid molecule comprising a first ITR, a second ITR, and a genetic cassette encoding a therapeutic protein, wherein the therapeutic protein comprises a clotting factor. In some embodiments, the clotting factor is selected from the group consisting of FI, FII, FIII, FIV, FV, FVI, FVII, FVIII, FIX, FX, FXI, FXII, FXIII), VWF, prekallikrein, high-molecular weight kininogen, fibronectin, antithrombin Ill, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), any zymogen thereof, any active form thereof, and any combination thereof. In one embodiment, the clotting factor comprises FVIII or a variant or fragment thereof. In another embodiment, the clotting factor comprises FIX or a variant or fragment thereof. In another embodiment, the clotting factor comprises FVII or a variant or fragment thereof. In another embodiment, the clotting factor comprises VWF or a variant or fragment thereof.

### 1. Clotting Factors

In some embodiments, the nucleic acid molecule comprises a first ITR, a second ITR, and a genetic cassette encoding a target sequence, wherein the target sequence encodes a therapeutic protein, wherein the therapeutic protein comprises a factor VIII polypeptide. “Factor VIII,” abbreviated throughout the instant application as “FVIII,” as used herein, means functional FVIII polypeptide in its normal role in coagulation, unless otherwise specified. Thus, the term FVIII includes variant polypeptides that are functional. “A FVIII protein” is used interchangeably with FVIII polypeptide (or protein) or FVIII. Examples of the FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632). The full length polypeptide and polynucleotide sequences are known, as are many functional fragments, mutants and modified versions. Various FVIII amino acid and nucleotide sequences are disclosed in, e.g., US Publication Nos. 2015/0158929 A1, 2014/0308280 A1, and 2014/0370035 A1 and International Publication No. WO 2015/106052 A1. FVIII polypeptides include, e.g., full-length FVIII, full-length FVIII minus Met at the N-terminus, mature FVIII (minus the signal sequence), mature FVIII with an additional Met at the N-terminus, and/or FVIII with a full or partial deletion of the B domain. FVIII variants include B domain deletions, whether partial or full deletions.

a. FVIII and Polynucleotide Sequences Encoding the FVIII Protein

In some embodiments, the nucleic acid molecule comprises a first ITR, a second ITR, and a genetic cassette encoding a target sequence, wherein the target sequence encodes a therapeutic protein, wherein the therapeutic protein comprises a factor VIII polypeptide. “Factor VIII,” abbreviated throughout the instant application as “FVIII,” as used herein, means functional FVIII polypeptide in its normal role in coagulation, unless otherwise specified. Thus, the term FVIII includes variant polypeptides that are functional. “A FVIII protein” is used interchangeably with FVIII polypeptide (or protein) or FVIII. Examples of the FVIII functions include, but are not limited to, an ability to activate coagulation, an ability to act as a cofactor for factor IX, or an ability to form a tenase complex with factor IX in the presence of Ca²⁺ and phospholipids, which then converts Factor X to the activated form Xa. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632). The full-length polypeptide and polynucleotide sequences are known, as are many functional fragments, mutants and modified versions. Various FVIII amino acid and nucleotide sequences are disclosed in, e.g., US Publication Nos. 2015/0158929 A1, 2014/0308280 A1, and 2014/0370035 A1 and International Publication No. WO 2015/106052 A1. FVIII polypeptides include, e.g., full-length FVIII, full-length FVIII minus Met at the N-terminus, mature FVIII (minus the signal sequence), mature FVIII with an additional Met at the N-terminus, and/or FVIII with a full or partial deletion of the B domain. FVIII variants include B domain deletions, whether partial or full deletions.

The FVIII portion in the chimeric protein used herein has FVIII activity. FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is used in conjunction with prothrombin time (PT), which measures the extrinsic pathway.

ROTEM analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The chromogenic assay mechanism is based on the principles of the blood coagulation cascade, where activated FVIII accelerates the conversion of Factor X into Factor Xa in the presence of activated Factor IX, phospholipids and calcium ions. The Factor Xa activity is assessed by hydrolysis of a p-nitroanilide (pNA) substrate specific to Factor Xa. The initial rate of release of p-nitroaniline measured at 405 nM is directly proportional to the Factor Xa activity and thus to the FVIII activity in the sample.

The chromogenic assay is recommended by the FVIII and Factor IX Subcommittee of the Scientific and Standardization Committee (SSC) of the International Society on Thrombosis and Hemostatsis (ISTH). Since 1994, the chromogenic assay has also been the reference method of the European Pharmacopoeia for the assignment of FVIII concentrate potency. Thus, in one embodiment, the chimeric polypeptide comprising FVIII has FVIII activity comparable to a chimeric polypeptide comprising mature FVIII or a BDD FVIII (e.g., ADVATE®, REFACTO®, or ELOCTATE®).

In another embodiment, the chimeric protein comprising FVIII of this disclosure has a Factor Xa generation rate comparable to a chimeric protein comprising mature FVIII or a BDD FVIII (e.g., ADVATE®, REFACTO®, or ELOCTATE®).

In order to activate Factor X to Factor Xa, activated Factor IX (Factor IXa) hydrolyzes one arginine-isoleucine bond in Factor X to form Factor Xa in the presence of Ca²⁺, membrane phospholipids, and a FVIII cofactor. Therefore, the interaction of FVIII with Factor IX is critical in coagulation pathway. In certain embodiments, the chimeric polypeptide comprising FVIII can interact with Factor IXa at a rate comparable to a chimeric polypeptide comprising mature FVIII sequence or a BDD FVIII (e.g., ADVATE®, REFACTO®, or ELOCTATE®).

In addition, FVIII is bound to von Willebrand Factor while inactive in circulation. FVIII degrades rapidly when not bound to VWF and is released from VWF by the action of thrombin. In some embodiments, the chimeric polypeptide comprising FVIII binds to von Willebrand Factor at a level comparable to a chimeric polypeptide comprising mature FVIII sequence or a BDD FVIII (e.g., ADVATE®, REFACTO®, or ELOCTATE®).

FVIII can be inactivated by activated protein C in the presence of calcium and phospholipids. Activated protein C cleaves FVIII heavy chain after Arginine 336 in the A1 domain, which disrupts a Factor X substrate interaction site, and cleaves after Arginine 562 in the A2 domain, which enhances the dissociation of the A2 domain as well as disrupts an interaction site with the Factor IXa. This cleavage also bisects the A2 domain (43 kDa) and generates A2-N (18 kDa) and A2-C (25 kDa) domains. Thus, activated protein C can catalyze multiple cleavage sites in the heavy chain. In one embodiment, the chimeric polypeptide comprising FVIII is inactivated by activated Protein C at a level comparable to a chimeric polypeptide comprising mature FVIII sequence or a BDD FVIII (e.g., ADVATE®, REFACTO®, or ELOCTATE®).

In other embodiments, the chimeric protein comprising FVIII has FVIII activity in vivo comparable to a chimeric polypeptide comprising mature FVIII sequence or a BDD FVIII (e.g., ADVATE®, REFACTO®, or ELOCTATE®). In a particular embodiment, the chimeric polypeptide comprising FVIII is capable of protecting a HemA mouse at a level comparable to a chimeric polypeptide comprising mature FVIII sequence or a BDD FVIII (e.g., ADVATE®, REFACTO®, or ELOCTATE®) in a HemA mouse tail vein transection model.

A “B domain” of FVIII, as used herein, is the same as the B domain known in the art that is defined by internal amino acid sequence identity and sites of proteolytic cleavage by thrombin, e.g., residues Ser741-Arg1648 of mature human FVIII. The other human FVIII domains are defined by the following amino acid residues, relative to mature human FVIII: A1, residues Ala1-Arg372; A2, residues Ser373-Arg740; A3, residues Ser1690-Ile2032; C1, residues Arg2033-Asn2172; C2, residues Ser2173-Tyr2332 of mature FVIII. The sequence residue numbers used herein without referring to any SEQ ID Numbers correspond to the FVIII sequence without the signal peptide sequence (19 amino acids) unless otherwise indicated. The A3-C1-C2 sequence, also known as the FVIII heavy chain, includes residues Ser1690-Tyr2332. The remaining sequence, residues Glu1649-Arg1689, is usually referred to as the FVIII light chain activation peptide. The locations of the boundaries for all of the domains, including the B domains, for porcine, mouse and canine FVIII are also known in the art. In one embodiment, the B domain of FVIII is deleted (“B-domain-deleted FVIII” or “BDD FVIII”). An example of a BDD FVIII is REFACTO® (recombinant BDD FVIII). In one particular embodiment the B domain deleted FVIII variant comprises a deletion of amino acid residues 746 to 1648 of mature FVIII.

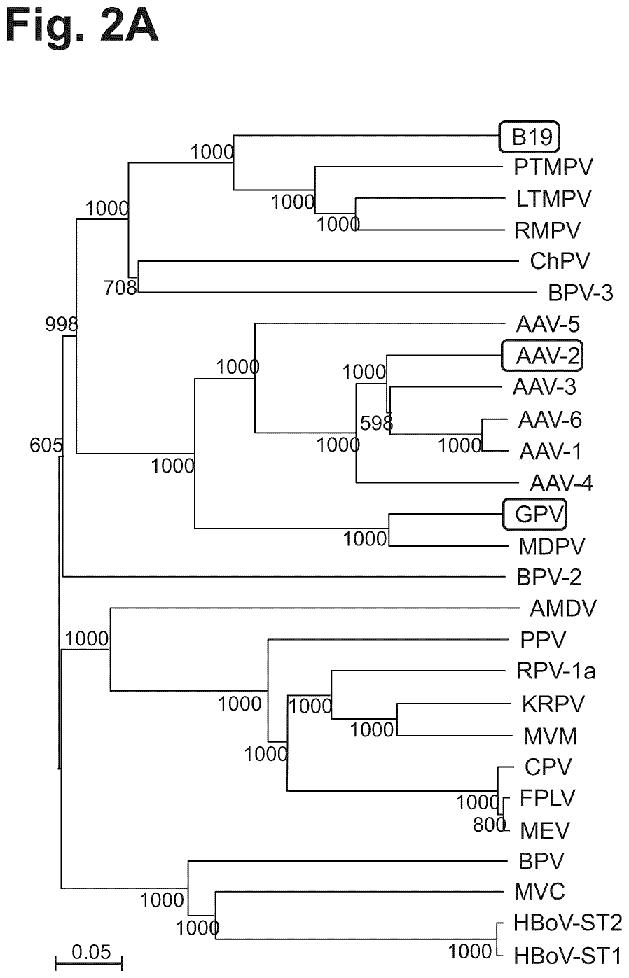

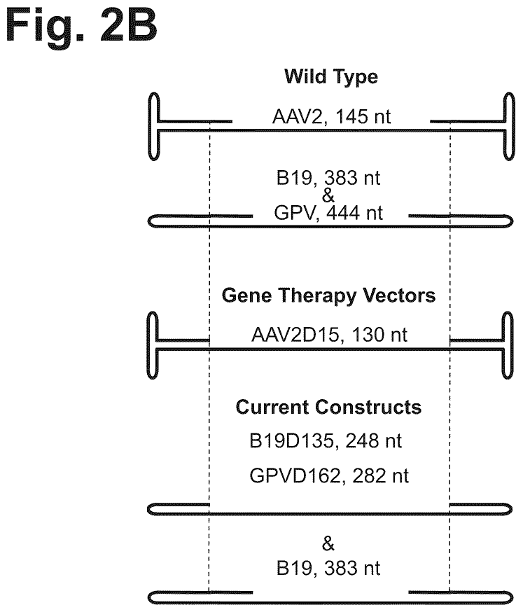

A “B-domain-deleted FVIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563 and Int'l Publ. No. WO 2015106052 A1 (PCT/US2015/010738). In some embodiments, a B-domain-deleted FVIII sequence used in the methods of the present disclosure comprises any one of the deletions disclosed at col. 4, line 4 to col. 5, line 28 and Examples 1-5 of U.S. Pat. No. 6,316,226 (also in U.S. Pat. No. 6,346,513). In another embodiment, a B-domain deleted Factor VIII is the S743/Q1638 B-domain deleted Factor VIII (SQ BDD FVIII) (e.g., Factor VIII having a deletion from amino acid 744 to amino acid 1637, e.g., Factor VIII having amino acids 1-743 and amino acids 1638-2332 of mature FVIII). In some embodiments, a B-domain-deleted FVIII used in the methods of the present disclosure has a deletion disclosed at col. 2, lines 26-51 and examples 5-8 of U.S. Pat. No. 5,789,203 (also U.S. Pat. Nos. 6,060,447, 5,595,886, and 6,228,620). In some embodiments, a B-domain-deleted Factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563. In some embodiments, a B-domain-deleted FVIII has a deletion of most of the B domain, but still contains amino-terminal sequences of the B domain that are essential for in vivo proteolytic processing of the primary translation product into two polypeptide chain, as disclosed in WO 91/09122. In some embodiments, a B-domain-deleted FVIII is constructed with a deletion of amino acids 747-1638, i.e., virtually a complete deletion of the B domain. Hoeben R. C., et al. J. Biol. Chem. 265 (13): 7318-7323 (1990). A B-domain-deleted Factor VIII may also contain a deletion of amino acids 771-1666 or amino acids 868-1562 of FVIII. Meulien P., et al. Protein Eng. 2(4): 301-6 (1988). Additional B domain deletions that are part of the invention include: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 through 1648 (Pasek (PCT application No. 88/00831)), or 816 through 1598 or 741 through 1648 (Lagner (Behring Inst. Mitt. (1988) No 82:16-25, EP 295597)). In one particular embodiment, the B-domain-deleted FVIII comprises a deletion of amino acid residues 746 to 1648 of mature FVIII. In another embodiment, the B-domain-deleted FVIII comprises a deletion of amino acid residues 745 to 1648 of mature FVIII. In some embodiments, the BDD FVIII comprises single chain FVIII that contains a deletion in amino acids 765 to 1652 corresponding to the mature full length FVIII (also known as rVIII-SingleChain and AFSTYLA®). See U.S. Pat. No. 7,041,635.

In other embodiments, BDD FVIII includes a FVIII polypeptide containing fragments of the B-domain that retain one or more N-linked glycosylation sites, e.g., residues 757, 784, 828, 900, 963, or optionally 943, which correspond to the amino acid sequence of the full-length FVIII sequence. Examples of the B-domain fragments include 226 amino acids or 163 amino acids of the B-domain as disclosed in Miao, H. Z., et al., Blood 103(a): 3412-3419 (2004), Kasuda, A, et al., J. Thromb. Haemost. 6: 1352-1359 (2008), and Pipe, S. W., et al., J. Thromb. Haemost. 9: 2235-2242 (2011) (i.e., the first 226 amino acids or 163 amino acids of the B domain are retained). In still other embodiments, BDD FVIII further comprises a point mutation at residue 309 (from Phe to Ser) to improve expression of the BDD FVIII protein. See Miao, H. Z., et al., Blood 103(a): 3412-3419 (2004). In still other embodiments, the BDD FVIII includes a FVIII polypeptide containing a portion of the B-domain, but not containing one or more furin cleavage sites (e.g., Arg1313 and Arg 1648). See Pipe, S. W., et al., J. Thromb. Haemost. 9: 2235-2242 (2011). In some embodiments, the BDD FVIII comprises single chain FVIII that contains a deletion in amino acids 765 to 1652 corresponding to the mature full length FVIII (also known as rVIII-SingleChain and AFSTYLA®). See U.S. Pat. No. 7,041,635. Each of the foregoing deletions may be made in any FVIII sequence.

A great many functional FVIII variants are known, as is discussed above and below. In addition, hundreds of nonfunctional mutations in FVIII have been identified in hemophilia patients, and it has been determined that the effect of these mutations on FVIII function is due more to where they lie within the 3-dimensional structure of FVIII than on the nature of the substitution (Cutler et al., Hum. Mutat. 19:274-8 (2002)), incorporated herein by reference in its entirety. In addition, comparisons between FVIII from humans and other species have identified conserved residues that are likely to be required for function (Cameron et al., Thromb. Haemost. 79:317-22 (1998); U.S. Pat. No. 6,251,632), incorporated herein by reference in its entirety.

In some embodiments, the FVIII polypeptide comprises a FVIII variant or fragment thereof, wherein the FVIII variant or the fragment thereof has a FVIII activity. In some embodiments, the genetic cassette encodes a full-length FVIII polypeptide. In other embodiments, the genetic cassette encodes a B domain-deleted (BDD) FVIII polypeptide, wherein all or a portion of the B domain of FVIII is deleted. In one particular embodiment, the genetic cassette encodes a polypeptide comprising an amino acid sequence having at least about 80%, at least about 85%, at least about 86%, at least about 87%, at least about 88%, at least about 89%, at least about 90%, at least about 95%, at least about 96%, at least about 97%, at least about 98%, or at least about 99% sequence identity to SEQ ID NOs: 106, 107, 109, 110, 111, or 112. In some embodiments, the genetic cassette encodes a polypeptide having the amino acid sequence of SEQ ID NO: 17 or a fragment thereof. In some embodiments, the genetic cassette encodes a polypeptide having the amino acid sequence of SEQ ID NO: 106 or a fragment thereof. In some embodiments, the genetic cassette comprises a nucleotide sequence which has at least 85%, at least 90%, at least 95%, at least 96%, at least 97%, at least 98%, or at least 99% sequence identity to SEQ ID NO: 107. In some embodiments, the genetic cassette encodes a polypeptide having the amino acid sequence of SEQ ID NO: 109 or a fragment thereof. In some embodiments, the genetic cassette comprises a nucleotide sequence which has at least 85%, at least 90%, at least 95%, at least 96%, at least 97%, at least 98%, or at least 99% sequence identity to SEQ ID NO: 16. In some embodiments, the genetic cassette comprises a nucleotide sequence which has at least 85%, at least 90%, at least 95%, at least 96%, at least 97%, at least 98%, or at least 99% sequence identity to SEQ ID NO: 109.

In some embodiments, the genetic cassette of the disclosure encodes a FVIII polypeptide comprising a signal peptide or a fragment thereof. In other embodiments, the genetic cassette encodes a FVIII polypeptide which lacks a signal peptide. In some embodiments, the signal peptide comprises amino acids 1-19 of SEQ ID NO: 17.

In some embodiments, the genetic cassette comprises a nucleotide sequence encoding a FVIII polypeptide, wherein the nucleotide sequence is codon optimized. In certain embodiments, the genetic cassette comprises a nucleotide sequence which is disclosed in International Application No. PCT/US2017/015879, which is incorporated by reference in its entirety. In some embodiments, the genetic cassette comprises a nucleotide sequence encoding a FVIII polypeptide, wherein the nucleotide sequence is codon optimized. In certain embodiments, the genetic cassette comprises a nucleotide sequence which has at least 85%, at least 90%, at least 95%, at least 96%, at least 97%, at least 98%, or at least 99% sequence identity to a nucleotide sequence selected from SEQ ID NOs: 1-14. In some embodiments, the genetic cassette comprises a nucleotide sequence which has at least 85%, at least 90%, at least 95%, at least 96%, at least 97%, at least 98%, or at least 99% sequence identity to SEQ ID NO: 71. In some embodiments, the genetic cassette comprises a nucleotide sequence which has at least 85%, at least 90%, at least 95%, at least 96%, at least 97%, at least 98%, or at least 99% sequence identity to SEQ ID NO: 19.

i. Codon Optimized Nucleotide Sequences Encoding FVIII Polypeptides

In some embodiments, a nucleic acid molecule of the present disclosure comprises a first ITR, a second ITR, and a genetic cassette encoding a target sequence, wherein the target sequence encodes a therapeutic protein, wherein the first ITR and the second ITR are derived from an AAV genome, and wherein the genetic cassette comprises a codon optimized nucleotide sequence encoding a FVIII polypeptide. In some embodiments, the codon optimized nucleotide sequence encodes a full-length FVIII polypeptide. In other embodiments, the codon optimized nucleotide sequence encodes a B domain-deleted (BDD) FVIII polypeptide, wherein all or a portion of the B domain of FVIII is deleted. In one particular embodiment, the codon optimized nucleotide sequence encodes a polypeptide comprising an amino acid sequence having at least about 80%, at least about 85%, at least about 86%, at least about 87%, at least about 88%, at least about 89%, at least about 90%, at least about 95%, at least about 96%, at least about 97%, at least about 98%, or at least about 99% sequence identity to SEQ ID NO: 17 or a fragment thereof. In one embodiment, the codon optimized nucleotide sequence encodes a polypeptide having the amino acid sequence of SEQ ID NO: 17 or a fragment thereof.

In some embodiments, the codon optimized nucleotide sequence encodes a FVIII polypeptide comprising a signal peptide or a fragment thereof. In other embodiments, the codon optimized sequence encodes a FVIII polypeptide which lacks a signal peptide. In some embodiments, the signal peptide comprises amino acids 1-19 of SEQ ID NO: 17.

In some embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 58-1791 of SEQ ID NO: 3 or (ii) nucleotides 58-1791 of SEQ ID NO: 4; and wherein the N-terminal portion and the C-terminal portion together have a FVIII polypeptide activity. In one particular embodiment, the first nucleic acid sequence has at least 85%, at least 90%, at least 95%, at least 96%, at least 97%, at least 98%, or at least 99% sequence identity to nucleotides 58-1791 of SEQ ID NO: 3. In another embodiment, the first nucleic acid sequence has at least 85%, at least 90%, at least 95%, at least 96%, at least 97%, at least 98%, or at least 99% sequence identity to nucleotides 58-1791 of SEQ ID NO: 4. In other embodiments, the first nucleotide sequence comprises nucleotides 58-1791 of SEQ ID NO: 3 or nucleotides 58-1791 of SEQ ID NO: 4.

In other embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 85%, at least 90%, at least 95%, at least 96%, at least 97%, at least 98%, or at least 99% sequence identity to (i) nucleotides 1-1791 of SEQ ID NO: 3 or (ii) nucleotides 1-1791 of SEQ ID NO: 4; and wherein the N-terminal portion and the C-terminal portion together have a FVIII polypeptide activity. In one embodiment, the first nucleotide sequence comprises nucleotides 1-1791 of SEQ ID NO: 3 or nucleotides 1-1791 of SEQ ID NO: 4. In another embodiment, the second nucleotide sequence has at least 60%, at least 70%, at least 80%, at least 90%, at least 95%, at least 96%, at least 97%, at least 98%, or at least 99% sequence identity to nucleotides 1792-4374 of SEQ ID NO: 3 or 1792-4374 of SEQ ID NO: 4. In one particular embodiment, the second nucleotide sequence comprises nucleotides 1792-4374 of SEQ ID NO: 3 or 1792-4374 of SEQ ID NO: 4. In still another embodiment, the second nucleotide sequence has at least 60%, at least 70%, at least 80%, at least 90%, at least 95%, at least 96%, at least 97%, at least 98%, or at least 99% sequence identity to nucleotides 1792-2277 and 2320-4374 of SEQ ID NO: 3 or 1792-2277 and 2320-4374 of SEQ ID NO: 4 (i.e., nucleotides 1792-4374 of SEQ ID NO: 3 or 1792-4374 of SEQ ID NO: 4 without the nucleotides encoding the B domain or B domain fragment). In one particular embodiment, the second nucleotide sequence comprises nucleotides 1792-2277 and 2320-4374 of SEQ ID NO: 3 or 1792-2277 and 2320-4374 of SEQ ID NO: 4 (i.e., nucleotides 1792-4374 of SEQ ID NO: 3 or 1792-4374 of SEQ ID NO: 4 without the nucleotides encoding the B domain or B domain fragment).

In some embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-4374 of SEQ ID NO: 5 or (ii) 1792-4374 of SEQ ID NO: 6; and wherein the N-terminal portion and the C-terminal portion together have a FVIII polypeptide activity. In certain embodiments, the second nucleic acid sequence has at least 85%, at least 90%, at least 95%, at least 96%, at least 97%, at least 98%, or at least 99% sequence identity to nucleotides 1792-4374 of SEQ ID NO: 5. In other embodiments, the second nucleic acid sequence has at least 85%, at least 90%, at least 95%, at least 96%, at least 97%, at least 98%, or at least 99% sequence identity to nucleotides 1792-4374 of SEQ ID NO: 6. In one particular embodiment, the second nucleic acid sequence comprises nucleotides 1792-4374 of SEQ ID NO: 5 or 1792-4374 of SEQ ID NO: 6. In some embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 58-1791 of SEQ ID NO: 5 or nucleotides 58-1791 of SEQ ID NO: 6. In other embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 1-1791 of SEQ ID NO: 5 or nucleotides 1-1791 of SEQ ID NO: 6.

In other embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 85%, at least 90%, at least 95%, at least 96%, at least 97%, at least 98%, or at least 99% sequence identity to (i) nucleotides 1792-2277 and 2320-4374 of SEQ ID NO: 5 (i.e., nucleotides 1792-4374 of SEQ ID NO: 5 without the nucleotides encoding the B domain or B domain fragment) or (ii) 1792-2277 and 2320-4374 of SEQ ID NO: 6 (i.e., nucleotides 1792-4374 of SEQ ID NO: 6 without the nucleotides encoding the B domain or B domain fragment); and wherein the N-terminal portion and the C-terminal portion together have a FVIII polypeptide activity. In certain embodiments, the second nucleic acid sequence has at least 85%, at least 90%, at least 95%, at least 96%, at least 97%, at least 98%, or at least 99% sequence identity to nucleotides 1792-2277 and 2320-4374 of SEQ ID NO: 5 (i.e., nucleotides 1792-4374 of SEQ ID NO: 5 without the nucleotides encoding the B domain or B domain fragment). In other embodiments, the second nucleic acid sequence has at least 85%, at least 90%, at least 95%, at least 96%, at least 97%, at least 98%, or at least 99% sequence identity to nucleotides 1792-2277 and 2320-4374 of SEQ ID NO: 6 (i.e., nucleotides 1792-4374 of SEQ ID NO: 6 without the nucleotides encoding the B domain or B domain fragment). In one particular embodiment, the second nucleic acid sequence comprises nucleotides 1792-2277 and 2320-4374 of SEQ ID NO: 5 or 1792-2277 and 2320-4374 of SEQ ID NO: 6 (i.e., nucleotides 1792-4374 of SEQ ID NO: 5 or 1792-4374 of SEQ ID NO: 6 without the nucleotides encoding the B domain or B domain fragment). In some embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 58-1791 of SEQ ID NO: 5 or nucleotides 58-1791 of SEQ ID NO: 6. In other embodiments, the first nucleic acid sequence linked to the second nucleic acid sequence listed above has at least 60%, at least 70%, at least 80%, at least 90%, at least 95%, at least 96%, at least 97%, at least 98%, or at least 99% sequence identity to nucleotides 1-1791 of SEQ ID NO: 5 or nucleotides 1-1791 of SEQ ID NO: 6.

In some embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 90%, at least 95%, at least 96%, at least 97%, at least 98%, or at least 99% sequence identity to (i) nucleotides 58-1791 of SEQ ID NO: 1, (ii) nucleotides 58-1791 of SEQ ID NO: 2, (iii) nucleotides 58-1791 of SEQ ID NO: 70, or (iv) nucleotides 58-1791 of SEQ ID NO: 71; and wherein the N-terminal portion and the C-terminal portion together have a FVIII polypeptide activity. In other embodiments, the first nucleotide sequence comprises nucleotides 58-1791 of SEQ ID NO: 1, nucleotides 58-1791 of SEQ ID NO: 2, (iii) nucleotides 58-1791 of SEQ ID NO: 70, or (iv) nucleotides 58-1791 of SEQ ID NO: 71.

In other embodiments, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least 90%, at least 95%, at least 96%, at least 97%, at least 98%, or at least 99% sequence identity to (i) nucleotides 1-1791 of SEQ ID NO: 1, (ii) nucleotides 1-1791 of SEQ ID NO: 2, (iii) nucleotides 1-1791 of SEQ ID NO: 70, or (iv) nucleotides 1-1791 of SEQ ID NO: 71; and wherein the N-terminal portion and the C-terminal portion together have a FVIII polypeptide activity. In one embodiment, the first nucleotide sequence comprises nucleotides 1-1791 of SEQ ID NO: 1, nucleotides 1-1791 of SEQ ID NO: 2, (iii) nucleotides 1-1791 of SEQ ID NO: 70, or (iv) nucleotides 1-1791 of SEQ ID NO: 71. In another embodiment, the second nucleotide sequence linked to the first nucleotide sequence has at least 60%, at least 70%, at least 80%, at least 90%, at least 95%, at least 96%, at least 97%, at least 98%, or at least 99% sequence identity to nucleotides 1792-4374 of SEQ ID NO: 1, 1792-4374 of SEQ ID NO: 2, (iii) nucleotides 1792-4374 of SEQ ID NO: 70, or (iv) nucleotides 1792-4374 of SEQ ID NO: 71. In one particular embodiment, the second nucleotide sequence linked to the first nucleotide sequence comprises (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71. In other embodiments, the second nucleotide sequence linked to the first nucleotide sequence has at least 60%, at least 70%, at least 80%, at least 90%, at least 95%, at least 96%, at least 97%, at least 98%, or at least 99% sequence identity to (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71. In one embodiment, the second nucleotide sequence comprises (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71.

In another embodiment, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 90%, at least 95%, at least 96%, at least 97%, at least 98%, or at least 99% sequence identity to (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71; and wherein the N-terminal portion and the C-terminal portion together have a FVIII polypeptide activity. In one particular embodiment, the second nucleic acid sequence comprises (i) nucleotides 1792-4374 of SEQ ID NO: 1, (ii) nucleotides 1792-4374 of SEQ ID NO: 2, (iii) nucleotides 1792-4374 of SEQ ID NO: 70, or (iv) nucleotides 1792-4374 of SEQ ID NO: 71. In some embodiments, the codon optimized sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least 90%, at least 95%, at least 96%, at least 97%, at least 98%, or at least 99% sequence identity to (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71 (i.e., nucleotides 1792-4374 of SEQ ID NO: 1, nucleotides 1792-4374 of SEQ ID NO: 2, nucleotides 1792-4374 of SEQ ID NO: 70, or nucleotides 1792-4374 of SEQ ID NO: 71 without the nucleotides encoding the B domain or B domain fragment); and wherein the N-terminal portion and the C-terminal portion together have a FVIII polypeptide activity. In one embodiment, the second nucleic acid sequence comprises (i) nucleotides 1792-2277 and 2320-4374 of SEQ ID NO: 1, (ii) nucleotides 1792-2277 and 2320-4374 of SEQ ID NO: 2, (iii) nucleotides 1792-2277 and 2320-4374 of SEQ ID NO: 70, or (iv) nucleotides 1792-2277 and 2320-4374 of SEQ ID NO: 71 (i.e., nucleotides 1792-4374 of SEQ ID NO: 1, nucleotides 1792-4374 of SEQ ID NO: 2, nucleotides 1792-4374 of SEQ ID NO: 70, or nucleotides 1792-4374 of SEQ ID NO: 71 without the nucleotides encoding the B domain or B domain fragment).

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 1. In other embodiments, the nucleotide sequence comprises a nucleic acid sequence having at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 1 (i.e., nucleotides 58-4374 of SEQ ID NO: 1 without the nucleotides encoding the B domain or B domain fragment). In other embodiments, the nucleic acid sequence has at least 90%, at least 91%, at least 92%, at least 93%, at least 94%, at least 95%, at least 96%, at least 97%, at least 98%, or at least 99% sequence identity to SEQ ID NO: 1. In other embodiments, the nucleotide sequence comprises nucleotides 58-2277 and 2320-4374 of SEQ ID NO: 1 (i.e., nucleotides 58-4374 of SEQ ID NO: 1 without the nucleotides encoding the B domain or B domain fragment) or nucleotides 58 to 4374 of SEQ ID NO: 1. In still other embodiments, the nucleotide sequence comprises nucleotides 1-2277 and 2320-4374 of SEQ ID NO: 1 (i.e., nucleotides 1-4374 of SEQ ID NO: 1 without the nucleotides encoding the B domain or B domain fragment) or nucleotides 1 to 4374 of SEQ ID NO: 1.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 94%, at least 95%, at least 96%, at least 97%, at least 98%, or at least 99% sequence identity to nucleotides 58 to 4374 of SEQ ID NO: 2. In other embodiments, the nucleotide sequence comprises a nucleic acid sequence having at least 94%, at least 95%, at least 96%, at least 97%, at least 98%, or at least 99% sequence identity to nucleotides 58-2277 and 2320-4374 of SEQ ID NO: 2. In other embodiments, the nucleic acid sequence has at least 94%, at least 95%, at least 96%, at least 97%, at least 98%, or at least 99% sequence identity to SEQ ID NO: 2. In other embodiments, the nucleotide sequence comprises nucleotides 58-2277 and 2320-4374 of SEQ ID NO: 2 (i.e., nucleotides 58-4374 of SEQ ID NO: 2 without the nucleotides encoding the B domain or B domain fragment) or nucleotides 58 to 4374 of SEQ ID NO: 2. In still other embodiments, the nucleotide sequence comprises nucleotides 1-2277 and 2320-4374 of SEQ ID NO: 2 (i.e., nucleotides 1-4374 of SEQ ID NO: 2 without the nucleotides encoding the B domain or B domain fragment) or nucleotides 1 to 4374 of SEQ ID NO: 2.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58 to 4374 of SEQ ID NO: 70. In other embodiments, the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 70 (i.e., nucleotides 58-4374 of SEQ ID NO: 70 without the nucleotides encoding the B domain or B domain fragment). In other embodiments, the nucleic acid sequence has at least 85%, at least 86%, at least 87%, at least 88%, at least 89%, at least 90%, at least 91%, at least 95%, at least 96%, at least 97%, at least 98%, or at least 99% sequence identity to SEQ ID NO: 70. In other embodiments, the nucleotide sequence comprises nucleotides 58-2277 and 2320-4374 of SEQ ID NO: 70 (i.e., nucleotides 58-4374 of SEQ ID NO: 70 without the nucleotides encoding the B domain or B domain fragment) or nucleotides 58 to 4374 of SEQ ID NO: 70. In still other embodiments, the nucleotide sequence comprises nucleotides 1-2277 and 2320-4374 of SEQ ID NO: 70 (i.e., nucleotides 1-4374 of SEQ ID NO: 70 without the nucleotides encoding the B domain or B domain fragment) or nucleotides 1 to 4374 of SEQ ID NO: 70.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58 to 4374 of SEQ ID NO: 71. In other embodiments, the nucleotide sequence comprises a nucleic acid sequence having at least 85%, at least 86%, at least 87%, at least 88%, at least 89%, at least 90%, at least 91%, at least 95%, at least 96%, at least 97%, at least 98%, or at least 99% sequence identity to nucleotides 58-2277 and 2320-4374 of SEQ ID NO: 71 (i.e., nucleotides 58-4374 of SEQ ID NO: 71 without the nucleotides encoding the B domain or B domain fragment). In other embodiments, the nucleic acid sequence has at least 85%, at least 86%, at least 87%, at least 88%, at least 89%, at least 90%, at least 91%, at least 95%, at least 96%, at least 97%, at least 98%, or at least 99% sequence identity to SEQ ID NO: 71. In other embodiments, the nucleotide sequence comprises nucleotides 58-2277 and 2320-4374 of SEQ ID NO: 71 (i.e., nucleotides 58-4374 of SEQ ID NO: 71 without the nucleotides encoding the B domain or B domain fragment) or nucleotides 58 to 4374 of SEQ ID NO: 71. In still other embodiments, the nucleotide sequence comprises nucleotides 1-2277 and 2320-4374 of SEQ ID NO: 71 (i.e., nucleotides 1-4374 of SEQ ID NO: 71 without the nucleotides encoding the B domain or B domain fragment) or nucleotides 1to 4374 of SEQ ID NO: 71.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 92%, at least 93%, at least 94%, at least 95%, at least 96%, at least 97%, at least 98%, or at least 99% sequence identity to nucleotides 58 to 4374 of SEQ ID NO: 3. In other embodiments, the nucleotide sequence comprises a nucleic acid sequence having at least 92%, at least 93%, at least 94%, at least 95%, at least 96%, at least 97%, at least 98%, or at least 99% sequence identity to nucleotides 58-2277 and 2320-4374 of SEQ ID NO: 3 (i.e., nucleotides 58-4374 of SEQ ID NO: 3 without the nucleotides encoding the B domain or B domain fragment). In certain embodiments, the nucleic acid sequence has at least 92%, at least 93%, at least 94%, at least 95%, at least 96%, at least 97%, at least 98%, or at least 99% sequence identity to SEQ ID NO: 3. In some embodiments, the nucleotide sequence comprises nucleotides 58-2277 and 2320-4374 of SEQ ID NO: 3 (i.e., nucleotides 58-4374 of SEQ ID NO: 3 without the nucleotides encoding the B domain or B domain fragment) or nucleotides 58 to 4374 of SEQ ID NO: 3. In still other embodiments, the nucleotide sequence comprises nucleotides 58-2277 and 2320-4374 of SEQ ID NO: 3 (i.e., nucleotides 1-4374 of SEQ ID NO: 3 without the nucleotides encoding the B domain or B domain fragment) or nucleotides 1 to 4374 of SEQ ID NO: 3.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 91%, at least 92%, at least 93%, at least 94%, at least 95%, at least 96%, at least 97%, at least 98%, or at least 99% sequence identity to nucleotides 58 to 4374 of SEQ ID NO: 4. In other embodiments, the nucleotide sequence comprises a nucleic acid sequence having at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 4 (i.e., nucleotides 58-4374 of SEQ ID NO: 4 without the nucleotides encoding the B domain or B domain fragment). In other embodiments, the nucleic acid sequence has at least 91%, at least 92%, at least 93%, at least 94%, at least 95%, at least 96%, at least 97%, at least 98%, or at least 99% sequence identity to SEQ ID NO: 4. In other embodiments, the nucleotide sequence comprises nucleotides 58-2277 and 2320-4374 of SEQ ID NO: 4 (i.e., nucleotides 58-4374 of SEQ ID NO: 4 without the nucleotides encoding the B domain or B domain fragment) or nucleotides 58 to 4374 of SEQ ID NO: 4. In still other embodiments, the nucleotide sequence comprises nucleotides 1-2277 and 2320-4374 of SEQ ID NO: 4 (i.e., nucleotides 1-4374 of SEQ ID NO: 4 without the nucleotides encoding the B domain or B domain fragment) or nucleotides 1 to 4374 of SEQ ID NO: 4.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 5. In other embodiments, the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 5 (i.e., nucleotides 58-4374 of SEQ ID NO: 5 without the nucleotides encoding the B domain or B domain fragment). In certain embodiments, the nucleic acid sequence has at least 89%, at least 90%, at least 91%, at least 92%, at least 93%, at least 94%, at least 95%, at least 96%, at least 97%, at least 98%, or at least 99% sequence identity to SEQ ID NO: 5. In some embodiments, the nucleotide sequence comprises nucleotides 58-2277 and 2320-4374 of SEQ ID NO: 5 (i.e., nucleotides 58-4374 of SEQ ID NO: 5 without the nucleotides encoding the B domain or B domain fragment) or nucleotides 58 to 4374 of SEQ ID NO: 5. In still other embodiments, the nucleotide sequence comprises nucleotides 1-2277 and 2320-4374 of SEQ ID NO: 5 (i.e., nucleotides 1-4374 of SEQ ID NO: 5 without the nucleotides encoding the B domain or B domain fragment) or nucleotides 1 to 4374 of SEQ ID NO: 5.

In some embodiments, the codon optimized nucleotide sequence encoding a FVIII polypeptide comprises a nucleotide sequence encoding a polypeptide with FVIII activity, wherein the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58 to 4374 of SEQ ID NO: 6. In other embodiments, the nucleotide sequence comprises a nucleic acid sequence having at least 89%, at least 90%, at least 91%, at least 92%, at least 93%, at least 94%, at least 95%, at least 96%, at least 97%, at least 98%, or at least 99% sequence identity to nucleotides 58-2277 and 2320-4374 of SEQ ID NO: 6 (i.e., nucleotides 58-4374 of SEQ ID NO: 6 without the nucleotides encoding the B domain or B domain fragment). In certain embodiments, the nucleic acid sequence has at least 89%, at least 90%, at least 91%, at least 92%, at least 93%, at least 94%, at least 95%, at least 96%, at least 97%, at least 98%, or at least 99% sequence identity to SEQ ID NO: 6. In some embodiments, the nucleotide sequence comprises nucleotides 58-2277 and 2320-4374 of SEQ ID NO: 6 (i.e., nucleotides 58-4374 of SEQ ID NO: 6 without the nucleotides encoding the B domain or B domain fragment) or nucleotides 58 to 4374 of SEQ ID NO: 6. In still other embodiments, the nucleotide sequence comprises nucleotides 1-2277 and 2320-4374 of SEQ ID NO: 6 (i.e., nucleotides 1-4374 of SEQ ID NO: 6 without the nucleotides encoding the B domain or B domain fragment) or nucleotides 1 to 4374 of SEQ ID NO: 6.

In some embodiments, the codon optimized nucleotide sequence encoding a FVIII polypeptide comprises a nucleic acid sequence encoding a signal peptide. In certain embodiments, the signal peptide is a FVIII signal peptide. In some embodiments, the nucleic acid sequence encoding a signal peptide is codon optimized. In one particular embodiment, the nucleic acid sequence encoding a signal peptide has at least 60%, at least 70%, at least 80%, at least 90%, at least 95%, at least 96%, at least 97%, at least 98%, at least 99%, or 100% sequence identity to (i) nucleotides 1 to 57 of SEQ ID NO: 1; (ii) nucleotides 1 to 57 of SEQ ID NO: 2; (iii) nucleotides 1 to 57 of SEQ ID NO: 3; (iv) nucleotides 1 to 57 of SEQ ID NO: 4; (v) nucleotides 1 to 57 of SEQ ID NO: 5; (vi) nucleotides 1 to 57 of SEQ ID NO: 6; (vii) nucleotides 1 to 57 of SEQ ID NO: 70; (viii) nucleotides 1 to 57 of SEQ ID NO: 71; or (ix) nucleotides 1 to 57 of SEQ ID NO: 68.

SEQ ID NOs: 1-6, 70, and 71 are optimized versions of SEQ ID NO: 16, the starting or “parental” or “wild-type” FVIII nucleotide sequence. SEQ ID NO: 16 encodes a B domain-deleted human FVIII. While SEQ ID NOs: 1-6, 70, and 71 are derived from a specific B domain-deleted form of FVIII (SEQ ID NO: 16), it is to be understood that the present disclosure also includes optimized versions of nucleic acids encoding other versions of FVIII. For example, other versions of FVIII can include full length FVIII, other B-domain deletions of FVIII (described herein), or other fragments of FVIII that retain FVIII activity.

In one embodiment, the genetic cassette comprises a FVIII construct, which includes a polynucleotide sequence as listed in Tables 2A-2F. In one embodiment, the genetic cassette comprises a FVIII construct, which includes a polynucleotide sequence set forth in Table 2A. In one embodiment, the genetic cassette comprises a FVIII construct, which includes a polynucleotide sequence set forth in Table 2B. In one embodiment, the genetic cassette comprises a FVIII construct, which includes a polynucleotide sequence set forth in Table 2C. In one embodiment, the genetic cassette comprises a FVIII construct, which includes a polynucleotide sequence set forth in Table 2D. In one embodiment, the genetic cassette comprises a FVIII construct, which includes a polynucleotide sequence set forth in Table 2E. In one embodiment, the genetic cassette comprises a FVIII construct, which includes a polynucleotide sequence set forth in Table 2F

In certain embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 179, 182, 189, or 194. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 179. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 182. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 189. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 194. In some embodiments, the isolated nucleic acid molecule retains the ability to express a functional FVIII protein.

TABLE 2A
Example AAV-FVIII construct (nucleotides 1-6526; SEQ ID NO: 110)

| Description | Sequence |
|---|---|
| 5′ITR (5′-end | 1 -- |
| AAV2 inverted | CTGCGCGCTCGCTCGCTCACTGAGGCCGCCCGGGCAAAGCCCGGGCGTCGGGCGACC |
| terminal repeat) | TTTGGTCGCCCGGCCTCAGTGAGCGAGCGAGCGCGCAGAGAGGGAGTGGCCAACTCC |
| (SEQ ID NO: 111) | ATCACTAGGGGTTCCT -- 130 |
| Plasmid | 131 -- |
| Backbone | GCGGCAATTCAGTCGATAACTATAACGGTCCTAAGGTAGCGATTTAAATACGCGCTC |
| Sequence (PBS)- | TCTTAAGGTAGCCCCGGGACGCGTCAATTGAGATCTGGATCCGGTACCGAATTCGCG |
| 1 (SEQ ID | GCCGCCTCGACGACTAGCGTTTAATTAA -- 272 |
| NO: 112) | |
| TTPp (liver- | 273 -- |
| specific | ACGCGTGTCTGTCTGCACATTTCGTAGAGCGAGTGTTCCGATACTCTAATCTCCCTA |
| promoter) (SEQ | GGCAAGGTTCATATTTGTGTAGGTTACTTATTCTCCTTTTGTTGACTAAGTCAATAA |
| ID NO: 113) | TCAGAATCAGCAGGTTTGGAGTCAGCTTGGCAGGGATCAGCAGCCTGGGTTGGAAGG |
| | AGGGGGTATAAAAGCCCCTTCACCAGGAGAAGCCGTCACACAGATCCACAAGCTCCT |
| | G -- 501 |
| PBS-2 (SEQ ID | 502 -- AG -- 503 |
| NO: 114) | |
| Synthetic Intron | 504 -- |
| (SEQ ID NO: 115) | GTAAGTGCCGTGTGTGGTTCCCGCGGGCCTGGCCTCTTTACGGGTTATGGCCCTTGC |
| | GTGCCTTGAATTACTGACACTGACATCCACTTTTTCTTTTTCTCCACAG --609 |
| PBS-3 (SEQ ID | 610 -- CTAGCGCCACC -- 620 |
| NO: 116) | |
| FVIIIco6XTEN | 621 -- |
| (SEQ ID NO: 117) | ATGCAGATTGAGCTGTCCACTTGTTTCTTCCTGTGCCTCCTGCGCTTCTGTTTCTCC |
| (open reading | GCCACTCGCCGGTACTACCTTGGAGCCGTGGAGCTTTCATGGGACTACATGCAGAGC |
| frame for codon- | GACCTGGGCGAACTCCCCGTGGATGCCAGATTCCCCCCCCGCGTGCCAAAGTCCTTC |
| optimized FVIII | CCCTTTAACACCTCCGTGGTGTACAAGAAAACCCTCTTTGTCGAGTTCACTGACCAC |
| version 6 | CTGTTCAACATCGCCAAGCCGCGCCCACCTTGGATGGGCCTCCTGGGACCGACCATT |
| containing | CAAGCTGAAGTGTACGACACCGTGGTGATCACCCTGAAGAACATGGCGTCCCACCCC |
| XTEN144; the | GTGTCCCTGCATGCGGTCGGAGTGTCCTACTGGAAGGCCTCCGAAGGAGCTGAGTAC |
| XTEN sequence | GACGACCAGACTAGCCAGCGGGAAAAGGAGGACGATAAAGTGTTCCCGGGCGGCTCG |
| is marked by | CATACTTACGTGTGGCAAGTCCTGAAGGAAAACGGACCTATGGCATCCGATCCTCTG |
| double | TGCCTGACTTACTCCTACCTTTCCCATGTGGACCTCGTGAAGGACCTGAACAGCGGG |
| underlining (SEQ | CTGATTGGTGCACTTCTCGTGTGCCGCGAAGGTTCGCTCGCTAAGGAAAAGACCCAG |
| ID NO: 118)) | ACCCTCCATAAGTTCATCCTTTTGTTCGCTGTGTTCGATGAAGGAAAGTCATGGCAT |
| | TCCGAAACTAAGAACTCGCTGATGCAGGACCGGGATGCCGCCTCAGCCCGCGCCTGG |
| | CCTAAAATGCATACAGTCAACGGATACGTGAATCGGTCACTGCCCGGGCTCATCGGT |
| | TGTCACAGAAAGTCCGTGTACTGGCACGTCATCGGCATGGGCACTACGCCTGAAGTG |
| | CACTCCATCTTCCTGGAAGGGCACACCTTCCTCGTGCGCAACCACCGCCAGGCCTCT |
| | CTGGAAATCTCCCCGATTACCTTTCTGACCGCCCAGACTCTGCTCATGGACCTGGGG |
| | CAGTTCCTTCTCTTCTGCCACATCTCCAGCCATCAGCACGACGGAATGGAGGCCTAC |
| | GTGAAGGTGGACTCATGCCCGGAAGAACCTCAGTTGCGGATGAAGAACAACGAGGAG |
| | GCCGAGGACTATGACGACGATTTGACTGACTCCGAGATGGACGTCGTGCGGTTCGAT |
| | GACGACAACAGCCCCAGCTTCATCCAGATTCGCAGCGTGGCCAAGAAGCACCCCAAA |
| | ACCTGGGTGCACTACATCGCGGCCGAGGAAGAAGATTGGGACTACGCCCCGTTGGTG |
| | CTGGCACCCGATGACCGGTCGTACAAGTCCCAGTATCTGAACAATGGTCCGCAGCGG |
| | ATTGGCAGAAAGTACAAGAAAGTGCGGTTCATGGCGTACACTGACGAAACGTTTAAG |
| | ACCCGGGAGGCCATTCAACATGAGAGCGGCATTCTGGGACCACTGCTGTACGGAGAG |
| | GTCGGCGATACCCTGCTCATCATCTTCAAAAACCAGGCCTCCCGGCCTTACAACATC |
| | TACCCTCACGGAATCACCGACGTGCGGCCACTCTACTCGCGGCGCCTGCCGAAGGGC |
| | GTCAAGCACCTGAAAGACTTCCCTATCCTGCCGGGCGAAATCTTCAAGTATAAGTGG |
| | ACCGTCACCGTGGAGGACGGGCCCACCAAGAGCGATCCTAGGTGTCTGACTCGGTAC |
| | TACTCCAGCTTCGTGAACATGGAACGGGACCTGGCATCGGGACTCATTGGACCGCTG |
| | CTGATCTGCTACAAAGAGTCGGTGGATCAACGCGGCAACCAGATCATGTCCGACAAG |
| | CGCAACGTGATCCTGTTCTCCGTGTTTGATGAAAACAGATCCTGGTACCTCACTGAA |
| | AACATCCAGAGGTTCCTCCCAAACCCCGCAGGAGTGCAACTGGAGGACCCTGAGTTT |
| | CAGGCCTCGAATATCATGCACTCGATTAACGGTTACGTGTTCGACTCGCTGCAACTG |
| | AGCGTGTGCCTCCATGAAGTCGCTTACTGGTACATTCTGTCCATCGGCGCCCAGACT |
| | GACTTCCTGAGCGTGTTCTTTTCCGGTTACACCTTTAAGCACAAGATGGTGTACGAA |
| | GATACCCTGACCCTGTTCCCTTTCTCCGGCGAAACGGTGTTCATGTCGATGGAGAAC |
| | CCGGGTCTGTGGATTCTGGGATGCCACAACAGCGACTTTCGGAACCGCGGAATGACT |
| | GCCCTGCTGAAGGTGTCCTCATGCGACAAGAACACCGGAGACTACTACGAGGACTCC |
| | TACGAGGATATCTCAGCCTACCTCCTGTCCAAGAACAACGCGATCGAGCCGCGCAGC |
| | TTCAGCCAGAACGGCGCGCCAACATCAGAGAGCGCCACCCCTGAAAGTGGTCCCGGG |
| | AGCGAGCCAGCCACATCTGGGTCGGAAACGCCAGGCACAAGTGAGTCTGCAACTCCC |
| | GAGTCCGGACCTGGCTCCGAGCCTGCCACTAGCGGCTCCGAGACTCCGGGAACTTCC |
| | GAGAGCGCTACACCAGAAAGCGGACCCGGAACCAGTACCGAACCTAGCGAGGGCTCT |
| | GCTCCGGGCAGCCCAGCCGGCTCTCCTACATCCACGGAGGAGGGCACTTCCGAATCC |
| | GCCACCCCGGAGTCAGGGCCAGGATCTGAACCCGCTACCTCAGGCAGTGAGACGCCA |
| | GGAACGAGCGAGTCCGCTACACCGGAGAGTGGGCCAGGGAGCCCTGCTGGATCTCCT |
| | ACGTCCACTGAGGAAGGGTCACCAGCGGGCTCGCCCACCAGCACTGAAGAAGGTGCC |
| | TCGAGCCCGCCTGTGCTGAAGAGGCACCAGCGAGAAATTACCCGGACCACCCTCCAA |
| | TCGGATCAGGAGGAAATCGACTACGACGACACCATCTCGGTGGAAATGAAGAAGGAA |
| | GATTTCGATATCTACGACGAGGACGAAAATCAGTCCCCTCGCTCATTCCAAAAGAAA |
| | ACTAGACACTACTTTATCGCCGCGGTGGAAAGACTGTGGGACTATGGAATGTCATCC |
| | AGCCCTCACGTCCTTCGGAACCGGGCCCAGAGCGGATCGGTGCCTCAGTTCAAGAAA |
| | GTGGTGTTCCAGGAGTTCACCGACGGCAGCTTCACCCAGCCGCTGTACCGGGGAGAA |
| | CTGAACGAACACCTGGGCCTGCTCGGTCCCTACATCCGCGCGGAAGTGGAGGATAAC |
| | ATCATGGTGACCTTCCGTAACCAAGCATCCAGACCTTACTCCTTCTATTCCTCCCTG |
| | ATCTCATACGAGGAGGACCAGCGCCAAGGCGCCGAGCCCCGCAAGAACTTCGTCAAG |
| | CCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGAT |
| | GTCCATTCCGGCCTGATCGGGCCGCTGCTCGTGTGTCACACCAACACCCTGAACCCA |
| | GCGCATGGACGCCAGGTCACCGTCCAGGAGTTTGCTCTGTTCTTCACCATTTTTGAC |
| | GAAACTAAGTCCTGGTACTTCACCGAGAATATGGAGCGAAACTGTAGAGCGCCCTGC |
| | AATATCCAGATGGAAGATCCGACTTTCAAGGAGAACTATAGATTCCACGCCATCAAC |
| | GGGTACATCATGGATACTCTGCCGGGGCTGGTCATGGCCCAGGATCAGAGGATTCGG |
| | TGGTACTTGCTGTCAATGGGATCGAACGAAAACATTCACTCCATTCACTTCTCCGGT |
| | CACGTGTTCACTGTGCGCAAGAAGGAGGAGTACAAGATGGCGCTGTACAATCTGTAC |
| | CCCGGGGTGTTCGAAACTGTGGAGATGCTGCCGTCCAAGGCCGGCATCTGGAGAGTG |
| | GAGTGCCTGATCGGAGAGCACCTCCACGCGGGGATGTCCACCCTCTTCCTGGTGTAC |
| | TCGAATAAGTGCCAGACCCCGCTGGGCATGGCCTCGGGCCACATCAGAGACTTCCAG |
| | ATCACAGCAAGCGGACAATACGGCCAATGGGCGCCGAAGCTGGCCCGCTTGCACTAC |
| | TCCGGATCGATCAACGCATGGTCCACCAAGGAACCGTTCTCGTGGATTAAGGTGGAC |
| | CTCCTGGCCCCTATGATTATCCACGGAATTAAGACCCAGGGCGCCAGGCAGAAGTTC |
| | TCCTCCCTGTACATCTCGCAATTCATCATCATGTACAGCCTGGACGGGAAGAAGTGG |
| | CAGACTTACAGGGGAAACTCCACCGGCACCCTGATGGTCTTTTTCGGCAACGTGGAT |
| | TCCTCCGGCATTAAGCACAACATCTTCAACCCACCGATCATAGCCAGATATATTAGG |
| | CTCCACCCCACTCACTACTCAATCCGCTCAACTCTTCGGATGGAACTCATGGGGTGC |
| | GACCTGAACTCCTGCTCCATGCCGTTGGGGATGGAATCAAAGGCTATTAGCGACGCC |
| | CAGATCACCGCGAGCTCCTACTTCACTAACATGTTCGCCACCTGGAGCCCCTCCAAG |
| | GCCAGGCTGCACTTGCAGGGACGGTCAAATGCCTGGCGGCCGCAAGTGAACAATCCG |
| | AAGGAATGGCTTCAAGTGGATTTCCAAAAGACCATGAAAGTGACCGGAGTCACCACC |
| | CAGGGAGTGAAGTCCCTTCTGACCTCGATGTATGTGAAGGAGTTCCTGATTAGCAGC |
| | AGCCAGGACGGGCACCAGTGGACCCTGTTCTTCCAAAACGGAAAGGTCAAGGTGTTC |
| | CAGGGGAACCAGGACTCGTTCACACCCGTGGTGAACTCCCTGGACCCCCCACTGCTG |
| | ACGCGGTACTTGAGGATTCATCCTCAGTCCTGGGTCCATCAGATTGCATTGCGAATG |
| | GAAGTCCTGGGCTGCGAGGCCCAGGACCTGTACTGA -- 5444 |
| PBS-4 (SEQ ID | 5445 -- ATCAGCCTGAGCTCGCTGA -- 5463 |
| NO: 119) | |
| WPRE (mutated | 5464 -- |
| woodchuck | TCATAATCAACCTCTGGATTACAAAATTTGTGAAAGATTGACTGGTATTCTTAACTA |
| hepatitis virus | TGTTGCTCCTTTTACGCTATGTGGATACGCTGCTTTAATGCCTTTGTATCATGCTAT |
| post- | TGCTTCCCGTATGGCTTTCATTTTCTCCTCCTTGTATAAATCCTGGTTGCTGTCTCT |
| transcriptional | TTATGAGGAGTTGTGGCCCGTTGTCAGGCAACGTGGCGTGGTGTGCACTGTGTTTGC |
| regulatory | TGACGCAACCCCCACTGGTTGGGGCATTGCCACCACCTGTCAGCTCCTTTCCGGGAC |
| element) (SEQ ID | TTTCGCTTTCCCCCTCCCTATTGCCACGGCGGAACTCATCGCCGCCTGCCTTGCCCG |
| NO: 120) | CTGCTGGACAGGGGCTCGGCTGTTGGGCACTGACAATTCCGTGGTGTTGTCGGGGAA |
| | ATCATCGTCCTTTCCTTGGCTGCTCGCCTGTGTTGCCACCTGGATTCTGCGCGGGAC |
| | GTCCTTCTGCTACGTCCCTTCGGCCCTCAATCCAGCGGACCTTCCTTCCCGCGGCCT |
| | GCTGCCGGCTCTGCGGCCTCTTCCGCGTCTTCGCCTTCGCCCTCAGACGAGTCGGAT |
| | CTCCCTTTGGGCCGCCTCCCCGCTG -- 6058 |
| PBS-5 (SEQ ID | 6059 -- ATCAGCCT -- 6066 |
| NO: 121) | |
| bGHpA (bovine | 6067 -- |
| growth hormone | CGACTGTGCCTTCTAGTTGCCAGCCATCTGTTGTTTGCCCCTCCCCCGTGCCTTCCT |
| polyadenylation | TGACCCTGGAAGGTGCCACTCCCACTGTCCTTTCCTAATAAAATGAGGAAATTGCAT |
| signal) (SEQ ID | CGCATTGTCTGAGTAGGTGTCATTCTATTCTGGGGGGTGGGGTGGGGCAGGACAGCA |
| NO: 122) | AGGGGGAGGATTGGGAAGACAATAGCAGGCATGCTGGGGA -- 6277 |
| PBS-6 (SEQ ID | 6278 -- |
| NO: 123) | TGCGGTGGGCTCTATGGCTTCTGAGGCGGAAAGAACGGGCTCGAGAAGCTTCTAGAT |
| | ATCCTCTCTTAAGGTAGCATCGAGATTTAAATTAGGGATAACAGGGTAATGGCGCGG |
| | GCCGC -- 6396 |
| 3′ITR (3′-end | 6397 -- |
| AAV2 inverted | AGGAACCCCTAGTGATGGAGTTGGCCACTCCCTCTCTGCGCGCTCGCTCGCTCACTG |
| terminal repeat) | AGGCCGGGCGACCAAAGGTCGCCCGACGCCCGGGCTTTGCCCGGGCGGCCTCAGTGA |
| (SEQ ID NO: 124) | GCGAGCGAGCGCGCAG -- 6526 |

TABLE 2B
Example B19-FVIII construct bearing B19d135 ITRs (nucleotides 1-6762;
SEQ ID NO: 179)

| Description | Sequence |
|---|---|
| 5′ITR (SEQ ID | 1 - |
| NO: 180) | CTCTGGGCCAGCTTGCTTGGGGTTGCCTTGACACTAAGACAAGCGGCGCGCCGCTTG |
| | ATCTTAGTGGCACGTCAACCCCAAGCGCTGGCCCAGAGCCAACCCTAATTCCGGAAG |
| | TCCCGCCCACCGGAAGTGACGTCACAGGAAATGACGTCACAGGAAATGACGTAATTG |
| | TCCGCCATCTTGTACCGGAAGTCCCGCCTACCGGCGGCGACCGGCGGCATCTGATTT |
| | GGTGTCTTCTTTTAAATTTT -- 248 |
| TTPp (liver - | 391 - |
| specific promoter) | ACGCGTGTCTGTCTGCACATTTCGTAGAGCGAGTGTTCCGATACTCTAATCTCCCTA |
| (SEQ ID NO: 113) | GGCAAGGTTCATATTTGTGTAGGTTACTTATTCTCCTTTTGTTGACTAAGTCAATAA |
| | TCAGAATCAGCAGGTTTGGAGTCAGCTTGGCAGGGATCAGCAGCCTGGGTTGGAAGG |
| | AGGGGGTATAAAAGCCCCTTCACCAGGAGAAGCCGTCACACAGATCCACAAGCTCCT |
| | G -- 619 |
| Synthetic Intron | 622 - |
| (SEQ ID NO: 115) | GTAAGTGCCGTGTGTGGTTCCCGCGGGCCTGGCCTCTTTACGGGTTATGGCCCTTGC |
| | GTGCCTTGAATTACTGACACTGACATCCACTTTTTCTTTTTCTCCACAG --727 |
| FVIIIco6XTEN | 739 - |
| (SEQ ID NO: 117) | ATGCAGATTGAGCTGTCCACTTGTTTCTTCCTGTGCCTCCTGCGCTTCTGTTTCTCC |
| (open reading | GCCACTCGCCGGTACTACCTTGGAGCCGTGGAGCTTTCATGGGACTACATGCAGAGC |
| frame for codon - | GACCTGGGCGAACTCCCCGTGGATGCCAGATTCCCCCCCCGCGTGCCAAAGTCCTTC |
| optimized FVIII | CCCTTTAACACCTCCGTGGTGTACAAGAAAACCCTCTTTGTCGAGTTCACTGACCAC |
| version 6 | CTGTTCAACATCGCCAAGCCGCGCCCACCTTGGATGGGCCTCCTGGGACCGACCATT |
| containing | CAAGCTGAAGTGTACGACACCGTGGTGATCACCCTGAAGAACATGGCGTCCCACCCC |
| XTEN144; the | GTGTCCCTGCATGCGGTCGGAGTGTCCTACTGGAAGGCCTCCGAAGGAGCTGAGTAC |
| XTEN sequence | GACGACCAGACTAGCCAGCGGGAAAAGGAGGACGATAAAGTGTTCCCGGGCGGCTCG |
| is marked by | CATACTTACGTGTGGCAAGTCCTGAAGGAAAACGGACCTATGGCATCCGATCCTCTG |
| double | TGCCTGACTTACTCCTACCTTTCCCATGTGGACCTCGTGAAGGACCTGAACAGCGGG |
| underlining (SEQ | CTGATTGGTGCACTTCTCGTGTGCCGCGAAGGTTCGCTCGCTAAGGAAAAGACCCAG |
| ID NO: 118)) | ACCCTCCATAAGTTCATCCTTTTGTTCGCTGTGTTCGATGAAGGAAAGTCATGGCAT |
| | TCCGAAACTAAGAACTCGCTGATGCAGGACCGGGATGCCGCCTCAGCCCGCGCCTGG |
| | CCTAAAATGCATACAGTCAACGGATACGTGAATCGGTCACTGCCCGGGCTCATCGGT |
| | TGTCACAGAAAGTCCGTGTACTGGCACGTCATCGGCATGGGCACTACGCCTGAAGTG |
| | CACTCCATCTTCCTGGAAGGGCACACCTTCCTCGTGCGCAACCACCGCCAGGCCTCT |
| | CTGGAAATCTCCCCGATTACCTTTCTGACCGCCCAGACTCTGCTCATGGACCTGGGG |
| | CAGTTCCTTCTCTTCTGCCACATCTCCAGCCATCAGCACGACGGAATGGAGGCCTAC |
| | GTGAAGGTGGACTCATGCCCGGAAGAACCTCAGTTGCGGATGAAGAACAACGAGGAG |
| | GCCGAGGACTATGACGACGATTTGACTGACTCCGAGATGGACGTCGTGCGGTTCGAT |
| | GACGACAACAGCCCCAGCTTCATCCAGATTCGCAGCGTGGCCAAGAAGCACCCCAAA |
| | ACCTGGGTGCACTACATCGCGGCCGAGGAAGAAGATTGGGACTACGCCCCGTTGGTG |
| | CTGGCACCCGATGACCGGTCGTACAAGTCCCAGTATCTGAACAATGGTCCGCAGCGG |
| | ATTGGCAGAAAGTACAAGAAAGTGCGGTTCATGGCGTACACTGACGAAACGTTTAAG |
| | ACCCGGGAGGCCATTCAACATGAGAGCGGCATTCTGGGACCACTGCTGTACGGAGAG |
| | GTCGGCGATACCCTGCTCATCATCTTCAAAAACCAGGCCTCCCGGCCTTACAACATC |
| | TACCCTCACGGAATCACCGACGTGCGGCCACTCTACTCGCGGCGCCTGCCGAAGGGC |
| | GTCAAGCACCTGAAAGACTTCCCTATCCTGCCGGGCGAAATCTTCAAGTATAAGTGG |
| | ACCGTCACCGTGGAGGACGGGCCCACCAAGAGCGATCCTAGGTGTCTGACTCGGTAC |
| | TACTCCAGCTTCGTGAACATGGAACGGGACCTGGCATCGGGACTCATTGGACCGCTG |
| | CTGATCTGCTACAAAGAGTCGGTGGATCAACGCGGCAACCAGATCATGTCCGACAAG |
| | CGCAACGTGATCCTGTTCTCCGTGTTTGATGAAAACAGATCCTGGTACCTCACTGAA |
| | AACATCCAGAGGTTCCTCCCAAACCCCGCAGGAGTGCAACTGGAGGACCCTGAGTTT |
| | CAGGCCTCGAATATCATGCACTCGATTAACGGTTACGTGTTCGACTCGCTGCAACTG |
| | AGCGTGTGCCTCCATGAAGTCGCTTACTGGTACATTCTGTCCATCGGCGCCCAGACT |
| | GACTTCCTGAGCGTGTTCTTTTCCGGTTACACCTTTAAGCACAAGATGGTGTACGAA |
| | GATACCCTGACCCTGTTCCCTTTCTCCGGCGAAACGGTGTTCATGTCGATGGAGAAC |
| | CCGGGTCTGTGGATTCTGGGATGCCACAACAGCGACTTTCGGAACCGCGGAATGACT |
| | GCCCTGCTGAAGGTGTCCTCATGCGACAAGAACACCGGAGACTACTACGAGGACTCC |
| | TACGAGGATATCTCAGCCTACCTCCTGTCCAAGAACAACGCGATCGAGCCGCGCAGC |
| | TTCAGCCAGAACGGCGCGCCAACATCAGAGAGCGCCACCCCTGAAAGTGGTCCCGGG |
| | AGCGAGCCAGCCACATCTGGGTCGGAAACGCCAGGCACAAGTGAGTCTGCAACTCCC |
| | GAGTCCGGACCTGGCTCCGAGCCTGCCACTAGCGGCTCCGAGACTCCGGGAACTTCC |
| | GAGAGCGCTACACCAGAAAGCGGACCCGGAACCAGTACCGAACCTAGCGAGGGCTCT |
| | GCTCCGGGCAGCCCAGCCGGCTCTCCTACATCCACGGAGGAGGGCACTTCCGAATCC |
| | GCCACCCCGGAGTCAGGGCCAGGATCTGAACCCGCTACCTCAGGCAGTGAGACGCCA |
| | GGAACGAGCGAGTCCGCTACACCGGAGAGTGGGCCAGGGAGCCCTGCTGGATCTCCT |
| | ACGTCCACTGAGGAAGGGTCACCAGCGGGCTCGCCCACCAGCACTGAAGAAGGTGCC |
| | TCGAGCCCGCCTGTGCTGAAGAGGCACCAGCGAGAAATTACCCGGACCACCCTCCAA |
| | TCGGATCAGGAGGAAATCGACTACGACGACACCATCTCGGTGGAAATGAAGAAGGAA |
| | GATTTCGATATCTACGACGAGGACGAAAATCAGTCCCCTCGCTCATTCCAAAAGAAA |
| | ACTAGACACTACTTTATCGCCGCGGTGGAAAGACTGTGGGACTATGGAATGTCATCC |
| | AGCCCTCACGTCCTTCGGAACCGGGCCCAGAGCGGATCGGTGCCTCAGTTCAAGAAA |
| | GTGGTGTTCCAGGAGTTCACCGACGGCAGCTTCACCCAGCCGCTGTACCGGGGAGAA |
| | CTGAACGAACACCTGGGCCTGCTCGGTCCCTACATCCGCGCGGAAGTGGAGGATAAC |
| | ATCATGGTGACCTTCCGTAACCAAGCATCCAGACCTTACTCCTTCTATTCCTCCCTG |
| | ATCTCATACGAGGAGGACCAGCGCCAAGGCGCCGAGCCCCGCAAGAACTTCGTCAAG |
| | CCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGAT |
| | GTCCATTCCGGCCTGATCGGGCCGCTGCTCGTGTGTCACACCAACACCCTGAACCCA |
| | GCGCATGGACGCCAGGTCACCGTCCAGGAGTTTGCTCTGTTCTTCACCATTTTTGAC |
| | GAAACTAAGTCCTGGTACTTCACCGAGAATATGGAGCGAAACTGTAGAGCGCCCTGC |
| | AATATCCAGATGGAAGATCCGACTTTCAAGGAGAACTATAGATTCCACGCCATCAAC |
| | GGGTACATCATGGATACTCTGCCGGGGCTGGTCATGGCCCAGGATCAGAGGATTCGG |
| | TGGTACTTGCTGTCAATGGGATCGAACGAAAACATTCACTCCATTCACTTCTCCGGT |
| | CACGTGTTCACTGTGCGCAAGAAGGAGGAGTACAAGATGGCGCTGTACAATCTGTAC |
| | CCCGGGGTGTTCGAAACTGTGGAGATGCTGCCGTCCAAGGCCGGCATCTGGAGAGTG |
| | GAGTGCCTGATCGGAGAGCACCTCCACGCGGGGATGTCCACCCTCTTCCTGGTGTAC |
| | TCGAATAAGTGCCAGACCCCGCTGGGCATGGCCTCGGGCCACATCAGAGACTTCCAG |
| | ATCACAGCAAGCGGACAATACGGCCAATGGGCGCCGAAGCTGGCCCGCTTGCACTAC |
| | TCCGGATCGATCAACGCATGGTCCACCAAGGAACCGTTCTCGTGGATTAAGGTGGAC |
| | CTCCTGGCCCCTATGATTATCCACGGAATTAAGACCCAGGGCGCCAGGCAGAAGTTC |
| | TCCTCCCTGTACATCTCGCAATTCATCATCATGTACAGCCTGGACGGGAAGAAGTGG |
| | CAGACTTACAGGGGAAACTCCACCGGCACCCTGATGGTCTTTTTCGGCAACGTGGAT |
| | TCCTCCGGCATTAAGCACAACATCTTCAACCCACCGATCATAGCCAGATATATTAGG |
| | CTCCACCCCACTCACTACTCAATCCGCTCAACTCTTCGGATGGAACTCATGGGGTGC |
| | GACCTGAACTCCTGCTCCATGCCGTTGGGGATGGAATCAAAGGCTATTAGCGACGCC |
| | CAGATCACCGCGAGCTCCTACTTCACTAACATGTTCGCCACCTGGAGCCCCTCCAAG |
| | GCCAGGCTGCACTTGCAGGGACGGTCAAATGCCTGGCGGCCGCAAGTGAACAATCCG |
| | AAGGAATGGCTTCAAGTGGATTTCCAAAAGACCATGAAAGTGACCGGAGTCACCACC |
| | CAGGGAGTGAAGTCCCTTCTGACCTCGATGTATGTGAAGGAGTTCCTGATTAGCAGC |
| | AGCCAGGACGGGCACCAGTGGACCCTGTTCTTCCAAAACGGAAAGGTCAAGGTGTTC |
| | CAGGGGAACCAGGACTCGTTCACACCCGTGGTGAACTCCCTGGACCCCCCACTGCTG |
| | ACGCGGTACTTGAGGATTCATCCTCAGTCCTGGGTCCATCAGATTGCATTGCGAATG |
| | GAAGTCCTGGGCTGCGAGGCCCAGGACCTGTACTGA -- 5562 |
| WPRE (mutated | 5582 - |
| woodchuck | TCATAATCAACCTCTGGATTACAAAATTTGTGAAAGATTGACTGGTATTCTTAACTA |
| hepatitis virus | TGTTGCTCCTTTTACGCTATGTGGATACGCTGCTTTAATGCCTTTGTATCATGCTAT |
| post - | TGCTTCCCGTATGGCTTTCATTTTCTCCTCCTTGTATAAATCCTGGTTGCTGTCTCT |
| transcriptional | TTATGAGGAGTTGTGGCCCGTTGTCAGGCAACGTGGCGTGGTGTGCACTGTGTTTGC |
| regulatory | TGACGCAACCCCCACTGGTTGGGGCATTGCCACCACCTGTCAGCTCCTTTCCGGGAC |
| element) (SEQ ID | TTTCGCTTTCCCCCTCCCTATTGCCACGGCGGAACTCATCGCCGCCTGCCTTGCCCG |
| NO: 120) | CTGCTGGACAGGGGCTCGGCTGTTGGGCACTGACAATTCCGTGGTGTTGTCGGGGAA |
| | ATCATCGTCCTTTCCTTGGCTGCTCGCCTGTGTTGCCACCTGGATTCTGCGCGGGAC |
| | GTCCTTCTGCTACGTCCCTTCGGCCCTCAATCCAGCGGACCTTCCTTCCCGCGGCCT |
| | GCTGCCGGCTCTGCGGCCTCTTCCGCGTCTTCGCCTTCGCCCTCAGACGAGTCGGAT |
| | CTCCCTTTGGGCCGCCTCCCCGCTG -- 6176 |
| bGHpA (bovine | 6185 - |
| growth hormone | CGACTGTGCCTTCTAGTTGCCAGCCATCTGTTGTTTGCCCCTCCCCCGTGCCTTCCT |
| polyadenylation | TGACCCTGGAAGGTGCCACTCCCACTGTCCTTTCCTAATAAAATGAGGAAATTGCAT |
| signal) (SEQ ID | CGCATTGTCTGAGTAGGTGTCATTCTATTCTGGGGGGTGGGGTGGGGCAGGACAGCA |
| NO: 122) | AGGGGGAGGATTGGGAAGACAATAGCAGGCATGCTGGGGA -- 6395 |
| 3′ ITR inverted | 6515 - |
| terminal repeat | AAAATTTAAAAGAAGACACCAAATCAGATGCCGCCGGTCGCCGCCGGTAGGCGGGAC |
| (SEQ ID NO: 181) | TTCCGGTACAAGATGGCGGACAATTACGTCATTTCCTGTGACGTCATTTCCTGTGAC |
| | GTCACTTCCGGTGGGCGGGACTTCCGGAATTAGGGTTGGCTCTGGGCCAGCGCTTGG |
| | GGTTGACGTGCCACTAAGATCAAGCGGCGCGCCGCTTGTCTTAGTGTCAAGGCAACC |
| | CCAAGCAAGCTGGCCCAGAG -- 6762 |

Full-length Sequence (SEQ ID NO: 179)
CTCTGGGCCAGCTTGCTTGGGGTTGCCTTGACACTAAGACAAGCGGCGCGCCGCTTGATCTTAGTGGCACGTC
AACCCCAAGCGCTGGCCCAGAGCCAACCCTAATTCCGGAAGTCCCGCCCACCGGAAGTGACGTCACAGGAAAT
GACGTCACAGGAAATGACGTAATTGTCCGCCATCTTGTACCGGAAGTCCCGCCTACCGGCGGCGACCGGCGGC
ATCTGATTTGGTGTCTTCTTTTAAATTTTGCGGCAATTCAGTCGATAACTATAACGGTCCTAAGGTAGCGATT
TAAATACGCGCTCTCTTAAGGTAGCCCCGGGACGCGTCAATTGAGATCTGGATCCGGTACCGAATTCGCGGCC
GCCTCGACGACTAGCGTTTAATTAAACGCGTGTCTGTCTGCACATTTCGTAGAGCGAGTGTTCCGATACTCTA
ATCTCCCTAGGCAAGGTTCATATTTGTGTAGGTTACTTATTCTCCTTTTGTTGACTAAGTCAATAATCAGAAT
CAGCAGGTTTGGAGTCAGCTTGGCAGGGATCAGCAGCCTGGGTTGGAAGGAGGGGGTATAAAAGCCCCTTCAC
CAGGAGAAGCCGTCACACAGATCCACAAGCTCCTGAGGTAAGTGCCGTGTGTGGTTCCCGCGGGCCTGGCCTC
TTTACGGGTTATGGCCCTTGCGTGCCTTGAATTACTGACACTGACATCCACTTTTTCTTTTTCTCCACAGCTA
GCGCCACCATGCAGATTGAGCTGTCCACTTGTTTCTTCCTGTGCCTCCTGCGCTTCTGTTTCTCCGCCACTCG
CCGGTACTACCTTGGAGCCGTGGAGCTTTCATGGGACTACATGCAGAGCGACCTGGGCGAACTCCCCGTGGAT
GCCAGATTCCCCCCCCGCGTGCCAAAGTCCTTCCCCTTTAACACCTCCGTGGTGTACAAGAAAACCCTCTTTG
TCGAGTTCACTGACCACCTGTTCAACATCGCCAAGCCGCGCCCACCTTGGATGGGCCTCCTGGGACCGACCAT
TCAAGCTGAAGTGTACGACACCGTGGTGATCACCCTGAAGAACATGGCGTCCCACCCCGTGTCCCTGCATGCG
GTCGGAGTGTCCTACTGGAAGGCCTCCGAAGGAGCTGAGTACGACGACCAGACTAGCCAGCGGGAAAAGGAGG
ACGATAAAGTGTTCCCGGGCGGCTCGCATACTTACGTGTGGCAAGTCCTGAAGGAAAACGGACCTATGGCATC
CGATCCTCTGTGCCTGACTTACTCCTACCTTTCCCATGTGGACCTCGTGAAGGACCTGAACAGCGGGCTGATT
GGTGCACTTCTCGTGTGCCGCGAAGGTTCGCTCGCTAAGGAAAAGACCCAGACCCTCCATAAGTTCATCCTTT
TGTTCGCTGTGTTCGATGAAGGAAAGTCATGGCATTCCGAAACTAAGAACTCGCTGATGCAGGACCGGGATGC
CGCCTCAGCCCGCGCCTGGCCTAAAATGCATACAGTCAACGGATACGTGAATCGGTCACTGCCCGGGCTCATC
GGTTGTCACAGAAAGTCCGTGTACTGGCACGTCATCGGCATGGGCACTACGCCTGAAGTGCACTCCATCTTCC
TGGAAGGGCACACCTTCCTCGTGCGCAACCACCGCCAGGCCTCTCTGGAAATCTCCCCGATTACCTTTCTGAC
CGCCCAGACTCTGCTCATGGACCTGGGGCAGTTCCTTCTCTTCTGCCACATCTCCAGCCATCAGCACGACGGA
ATGGAGGCCTACGTGAAGGTGGACTCATGCCCGGAAGAACCTCAGTTGCGGATGAAGAACAACGAGGAGGCCG
AGGACTATGACGACGATTTGACTGACTCCGAGATGGACGTCGTGCGGTTCGATGACGACAACAGCCCCAGCTT
CATCCAGATTCGCAGCGTGGCCAAGAAGCACCCCAAAACCTGGGTGCACTACATCGCGGCCGAGGAAGAAGAT
TGGGACTACGCCCCGTTGGTGCTGGCACCCGATGACCGGTCGTACAAGTCCCAGTATCTGAACAATGGTCCGC
AGCGGATTGGCAGAAAGTACAAGAAAGTGCGGTTCATGGCGTACACTGACGAAACGTTTAAGACCCGGGAGGC
CATTCAACATGAGAGCGGCATTCTGGGACCACTGCTGTACGGAGAGGTCGGCGATACCCTGCTCATCATCTTC
AAAAACCAGGCCTCCCGGCCTTACAACATCTACCCTCACGGAATCACCGACGTGCGGCCACTCTACTCGCGGC
GCCTGCCGAAGGGCGTCAAGCACCTGAAAGACTTCCCTATCCTGCCGGGCGAAATCTTCAAGTATAAGTGGAC
CGTCACCGTGGAGGACGGGCCCACCAAGAGCGATCCTAGGTGTCTGACTCGGTACTACTCCAGCTTCGTGAAC
ATGGAACGGGACCTGGCATCGGGACTCATTGGACCGCTGCTGATCTGCTACAAAGAGTCGGTGGATCAACGCG
GCAACCAGATCATGTCCGACAAGCGCAACGTGATCCTGTTCTCCGTGTTTGATGAAAACAGATCCTGGTACCT
CACTGAAAACATCCAGAGGTTCCTCCCAAACCCCGCAGGAGTGCAACTGGAGGACCCTGAGTTTCAGGCCTCG
AATATCATGCACTCGATTAACGGTTACGTGTTCGACTCGCTGCAACTGAGCGTGTGCCTCCATGAAGTCGCTT
ACTGGTACATTCTGTCCATCGGCGCCCAGACTGACTTCCTGAGCGTGTTCTTTTCCGGTTACACCTTTAAGCA
CAAGATGGTGTACGAAGATACCCTGACCCTGTTCCCTTTCTCCGGCGAAACGGTGTTCATGTCGATGGAGAAC
CCGGGTCTGTGGATTCTGGGATGCCACAACAGCGACTTTCGGAACCGCGGAATGACTGCCCTGCTGAAGGTGT
CCTCATGCGACAAGAACACCGGAGACTACTACGAGGACTCCTACGAGGATATCTCAGCCTACCTCCTGTCCAA
GAACAACGCGATCGAGCCGCGCAGCTTCAGCCAGAACGGCGCGCCAACATCAGAGAGCGCCACCCCTGAAAGT
GGTCCCGGGAGCGAGCCAGCCACATCTGGGTCGGAAACGCCAGGCACAAGTGAGTCTGCAACTCCCGAGTCCG
GACCTGGCTCCGAGCCTGCCACTAGCGGCTCCGAGACTCCGGGAACTTCCGAGAGCGCTACACCAGAAAGCGG
ACCCGGAACCAGTACCGAACCTAGCGAGGGCTCTGCTCCGGGCAGCCCAGCCGGCTCTCCTACATCCACGGAG
GAGGGCACTTCCGAATCCGCCACCCCGGAGTCAGGGCCAGGATCTGAACCCGCTACCTCAGGCAGTGAGACGC
CAGGAACGAGCGAGTCCGCTACACCGGAGAGTGGGCCAGGGAGCCCTGCTGGATCTCCTACGTCCACTGAGGA
AGGGTCACCAGCGGGCTCGCCCACCAGCACTGAAGAAGGTGCCTCGAGCCCGCCTGTGCTGAAGAGGCACCAG
CGAGAAATTACCCGGACCACCCTCCAATCGGATCAGGAGGAAATCGACTACGACGACACCATCTCGGTGGAAA
TGAAGAAGGAAGATTTCGATATCTACGACGAGGACGAAAATCAGTCCCCTCGCTCATTCCAAAAGAAAACTAG
ACACTACTTTATCGCCGCGGTGGAAAGACTGTGGGACTATGGAATGTCATCCAGCCCTCACGTCCTTCGGAAC
CGGGCCCAGAGCGGATCGGTGCCTCAGTTCAAGAAAGTGGTGTTCCAGGAGTTCACCGACGGCAGCTTCACCC
AGCCGCTGTACCGGGGAGAACTGAACGAACACCTGGGCCTGCTCGGTCCCTACATCCGCGCGGAAGTGGAGGA
TAACATCATGGTGACCTTCCGTAACCAAGCATCCAGACCTTACTCCTTCTATTCCTCCCTGATCTCATACGAG
GAGGACCAGCGCCAAGGCGCCGAGCCCCGCAAGAACTTCGTCAAGCCCAACGAGACTAAGACCTACTTCTGGA
AGGTCCAACACCATATGGCCCCGACCAAGGATGAGTTTGACTGCAAGGCCTGGGCCTACTTCTCCGACGTGGA
CCTTGAGAAGGATGTCCATTCCGGCCTGATCGGGCCGCTGCTCGTGTGTCACACCAACACCCTGAACCCAGCG
CATGGACGCCAGGTCACCGTCCAGGAGTTTGCTCTGTTCTTCACCATTTTTGACGAAACTAAGTCCTGGTACT
TCACCGAGAATATGGAGCGAAACTGTAGAGCGCCCTGCAATATCCAGATGGAAGATCCGACTTTCAAGGAGAA
CTATAGATTCCACGCCATCAACGGGTACATCATGGATACTCTGCCGGGGCTGGTCATGGCCCAGGATCAGAGG
ATTCGGTGGTACTTGCTGTCAATGGGATCGAACGAAAACATTCACTCCATTCACTTCTCCGGTCACGTGTTCA
CTGTGCGCAAGAAGGAGGAGTACAAGATGGCGCTGTACAATCTGTACCCCGGGGTGTTCGAAACTGTGGAGAT
GCTGCCGTCCAAGGCCGGCATCTGGAGAGTGGAGTGCCTGATCGGAGAGCACCTCCACGCGGGGATGTCCACC
CTCTTCCTGGTGTACTCGAATAAGTGCCAGACCCCGCTGGGCATGGCCTCGGGCCACATCAGAGACTTCCAGA
TCACAGCAAGCGGACAATACGGCCAATGGGCGCCGAAGCTGGCCCGCTTGCACTACTCCGGATCGATCAACGC
ATGGTCCACCAAGGAACCGTTCTCGTGGATTAAGGTGGACCTCCTGGCCCCTATGATTATCCACGGAATTAAG
ACCCAGGGCGCCAGGCAGAAGTTCTCCTCCCTGTACATCTCGCAATTCATCATCATGTACAGCCTGGACGGGA
AGAAGTGGCAGACTTACAGGGGAAACTCCACCGGCACCCTGATGGTCTTTTTCGGCAACGTGGATTCCTCCGG
CATTAAGCACAACATCTTCAACCCACCGATCATAGCCAGATATATTAGGCTCCACCCCACTCACTACTCAATC
CGCTCAACTCTTCGGATGGAACTCATGGGGTGCGACCTGAACTCCTGCTCCATGCCGTTGGGGATGGAATCAA
AGGCTATTAGCGACGCCCAGATCACCGCGAGCTCCTACTTCACTAACATGTTCGCCACCTGGAGCCCCTCCAA
GGCCAGGCTGCACTTGCAGGGACGGTCAAATGCCTGGCGGCCGCAAGTGAACAATCCGAAGGAATGGCTTCAA
GTGGATTTCCAAAAGACCATGAAAGTGACCGGAGTCACCACCCAGGGAGTGAAGTCCCTTCTGACCTCGATGT
ATGTGAAGGAGTTCCTGATTAGCAGCAGCCAGGACGGGCACCAGTGGACCCTGTTCTTCCAAAACGGAAAGGT
CAAGGTGTTCCAGGGGAACCAGGACTCGTTCACACCCGTGGTGAACTCCCTGGACCCCCCACTGCTGACGCGG
TACTTGAGGATTCATCCTCAGTCCTGGGTCCATCAGATTGCATTGCGAATGGAAGTCCTGGGCTGCGAGGCCC
AGGACCTGTACTGAATCAGCCTGAGCTCGCTGATCATAATCAACCTCTGGATTACAAAATTTGTGAAAGATTG
ACTGGTATTCTTAACTATGTTGCTCCTTTTACGCTATGTGGATACGCTGCTTTAATGCCTTTGTATCATGCTA
TTGCTTCCCGTATGGCTTTCATTTTCTCCTCCTTGTATAAATCCTGGTTGCTGTCTCTTTATGAGGAGTTGTG
GCCCGTTGTCAGGCAACGTGGCGTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCCACGGCGGAACTCATCGCCGCCT
GCCTTGCCCGCTGCTGGACAGGGGCTCGGCTGTTGGGCACTGACAATTCCGTGGTGTTGTCGGGGAAATCATC
GTCCTTTCCTTGGCTGCTCGCCTGTGTTGCCACCTGGATTCTGCGCGGGACGTCCTTCTGCTACGTCCCTTCG
GCCCTCAATCCAGCGGACCTTCCTTCCCGCGGCCTGCTGCCGGCTCTGCGGCCTCTTCCGCGTCTTCGCCTTC
GCCCTCAGACGAGTCGGATCTCCCTTTGGGCCGCCTCCCCGCTGATCAGCCTCGACTGTGCCTTCTAGTTGCC
AGCCATCTGTTGTTTGCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTCTATGGCTTCTGAGGCGG
AAAGAACGGGCTCGAGAAGCTTCTAGATATCCTCTCTTAAGGTAGCATCGAGATTTAAATTAGGGATAACAGG
GTAATGGCGCGGGCCGCAAAATTTAAAAGAAGACACCAAATCAGATGCCGCCGGTCGCCGCCGGTAGGCGGGA
CTTCCGGTACAAGATGGCGGACAATTACGTCATTTCCTGTGACGTCATTTCCTGTGACGTCACTTCCGGTGGG
CGGGACTTCCGGAATTAGGGTTGGCTCTGGGCCAGCGCTTGGGGTTGACGTGCCACTAAGATCAAGCGGCGCG
CCGCTTGTCTTAGTGTCAAGGCAACCCCAAGCAAGCTGGCCCAGAG

TABLE 2C
Example GPV-FVIII construct bearing GPVd162 ITRs
(nucleotides 1-6830; SEQ ID NO: 182)

| Description | Sequence |
|---|---|
| 5′ ITR (SEQ ID | 1 - |
| NO: 183) | CGGTGACGTGTTTCCGGCTGTTAGGTTGACCACGCGCATGCCGCGCGGTCAGCCCAAT |
| | AGTTAAGCCGGAAACACGTCACCGGAAGTCACATGACCGGAAGTCACGTGACCGGAAA |
| | CACGTGACAGGAAGCACGTGACCGGAACTACGTCACCGGATGTGCGTCACCGGAAGCA |
| | TGTGACCGGAACTTGCGTCACTTCCCCCTCCCCTGATTGGCTGGTTCGAACGAACGAA |
| | CCCTCCAATGAGACTCAAGGACAAGAGGATATTTTGCGCGCCAGGAAGTG -- 282 |
| TTPp (liver - | 425 - |
| specific | ACGCGTGTCTGTCTGCACATTTCGTAGAGCGAGTGTTCCGATACTCTAATCTCCCTAG |
| promoter) (SEQ | GCAAGGTTCATATTTGTGTAGGTTACTTATTCTCCTTTTGTTGACTAAGTCAATAATC |
| ID NO: 113) | AGAATCAGCAGGTTTGGAGTCAGCTTGGCAGGGATCAGCAGCCTGGGTTGGAAGGAGG |
| | GGGTATAAAAGCCCCTTCACCAGGAGAAGCCGTCACACAGATCCACAAGCTCCTG- - |
| | 653 |
| Synthetic Intron | 656 - |
| (SEQ ID NO: 115) | GTAAGTGCCGTGTGTGGTTCCCGCGGGCCTGGCCTCTTTACGGGTTATGGCCCTTGCG |
| | TGCCTTGAATTACTGACACTGACATCCACTTTTTCTTTTTCTCCACAG -- 761 |
| FVIIIco6XTEN | 773 - |
| (SEQ ID NO: 117) | ATGCAGATTGAGCTGTCCACTTGTTTCTTCCTGTGCCTCCTGCGCTTCTGTTTCTCCG |
| (open reading | CCACTCGCCGGTACTACCTTGGAGCCGTGGAGCTTTCATGGGACTACATGCAGAGCGA |
| frame for codon - | CCTGGGCGAACTCCCCGTGGATGCCAGATTCCCCCCCCGCGTGCCAAAGTCCTTCCCC |
| optimized FVIII | TTTAACACCTCCGTGGTGTACAAGAAAACCCTCTTTGTCGAGTTCACTGACCACCTGT |
| version 6 | TCAACATCGCCAAGCCGCGCCCACCTTGGATGGGCCTCCTGGGACCGACCATTCAAGC |
| containing | TGAAGTGTACGACACCGTGGTGATCACCCTGAAGAACATGGCGTCCCACCCCGTGTCC |
| XTEN144; the | CTGCATGCGGTCGGAGTGTCCTACTGGAAGGCCTCCGAAGGAGCTGAGTACGACGACC |
| XTEN sequence | AGACTAGCCAGCGGGAAAAGGAGGACGATAAAGTGTTCCCGGGCGGCTCGCATACTTA |
| is marked by | CGTGTGGCAAGTCCTGAAGGAAAACGGACCTATGGCATCCGATCCTCTGTGCCTGACT |
| double | TACTCCTACCTTTCCCATGTGGACCTCGTGAAGGACCTGAACAGCGGGCTGATTGGTG |
| underlining (SEQ | CACTTCTCGTGTGCCGCGAAGGTTCGCTCGCTAAGGAAAAGACCCAGACCCTCCATAA |
| ID NO: 118)) | GTTCATCCTTTTGTTCGCTGTGTTCGATGAAGGAAAGTCATGGCATTCCGAAACTAAG |
| | AACTCGCTGATGCAGGACCGGGATGCCGCCTCAGCCCGCGCCTGGCCTAAAATGCATA |
| | CAGTCAACGGATACGTGAATCGGTCACTGCCCGGGCTCATCGGTTGTCACAGAAAGTC |
| | CGTGTACTGGCACGTCATCGGCATGGGCACTACGCCTGAAGTGCACTCCATCTTCCTG |
| | GAAGGGCACACCTTCCTCGTGCGCAACCACCGCCAGGCCTCTCTGGAAATCTCCCCGA |
| | TTACCTTTCTGACCGCCCAGACTCTGCTCATGGACCTGGGGCAGTTCCTTCTCTTCTG |
| | CCACATCTCCAGCCATCAGCACGACGGAATGGAGGCCTACGTGAAGGTGGACTCATGC |
| | CCGGAAGAACCTCAGTTGCGGATGAAGAACAACGAGGAGGCCGAGGACTATGACGACG |
| | ATTTGACTGACTCCGAGATGGACGTCGTGCGGTTCGATGACGACAACAGCCCCAGCTT |
| | CATCCAGATTCGCAGCGTGGCCAAGAAGCACCCCAAAACCTGGGTGCACTACATCGCG |
| | GCCGAGGAAGAAGATTGGGACTACGCCCCGTTGGTGCTGGCACCCGATGACCGGTCGT |
| | ACAAGTCCCAGTATCTGAACAATGGTCCGCAGCGGATTGGCAGAAAGTACAAGAAAGT |
| | GCGGTTCATGGCGTACACTGACGAAACGTTTAAGACCCGGGAGGCCATTCAACATGAG |
| | AGCGGCATTCTGGGACCACTGCTGTACGGAGAGGTCGGCGATACCCTGCTCATCATCT |
| | TCAAAAACCAGGCCTCCCGGCCTTACAACATCTACCCTCACGGAATCACCGACGTGCG |
| | GCCACTCTACTCGCGGCGCCTGCCGAAGGGCGTCAAGCACCTGAAAGACTTCCCTATC |
| | CTGCCGGGCGAAATCTTCAAGTATAAGTGGACCGTCACCGTGGAGGACGGGCCCACCA |
| | AGAGCGATCCTAGGTGTCTGACTCGGTACTACTCCAGCTTCGTGAACATGGAACGGGA |
| | CCTGGCATCGGGACTCATTGGACCGCTGCTGATCTGCTACAAAGAGTCGGTGGATCAA |
| | CGCGGCAACCAGATCATGTCCGACAAGCGCAACGTGATCCTGTTCTCCGTGTTTGATG |
| | AAAACAGATCCTGGTACCTCACTGAAAACATCCAGAGGTTCCTCCCAAACCCCGCAGG |
| | AGTGCAACTGGAGGACCCTGAGTTTCAGGCCTCGAATATCATGCACTCGATTAACGGT |
| | TACGTGTTCGACTCGCTGCAACTGAGCGTGTGCCTCCATGAAGTCGCTTACTGGTACA |
| | TTCTGTCCATCGGCGCCCAGACTGACTTCCTGAGCGTGTTCTTTTCCGGTTACACCTT |
| | TAAGCACAAGATGGTGTACGAAGATACCCTGACCCTGTTCCCTTTCTCCGGCGAAACG |
| | GTGTTCATGTCGATGGAGAACCCGGGTCTGTGGATTCTGGGATGCCACAACAGCGACT |
| | TTCGGAACCGCGGAATGACTGCCCTGCTGAAGGTGTCCTCATGCGACAAGAACACCGG |
| | AGACTACTACGAGGACTCCTACGAGGATATCTCAGCCTACCTCCTGTCCAAGAACAAC |
| | GCGATCGAGCCGCGCAGCTTCAGCCAGAACGGCGCGCCAACATCAGAGAGCGCCACCC |
| | CTGAAAGTGGTCCCGGGAGCGAGCCAGCCACATCTGGGTCGGAAACGCCAGGCACAAG |
| | TGAGTCTGCAACTCCCGAGTCCGGACCTGGCTCCGAGCCTGCCACTAGCGGCTCCGAG |
| | ACTCCGGGAACTTCCGAGAGCGCTACACCAGAAAGCGGACCCGGAACCAGTACCGAAC |
| | CTAGCGAGGGCTCTGCTCCGGGCAGCCCAGCCGGCTCTCCTACATCCACGGAGGAGGG |
| | CACTTCCGAATCCGCCACCCCGGAGTCAGGGCCAGGATCTGAACCCGCTACCTCAGGC |
| | AGTGAGACGCCAGGAACGAGCGAGTCCGCTACACCGGAGAGTGGGCCAGGGAGCCCTG |
| | CTGGATCTCCTACGTCCACTGAGGAAGGGTCACCAGCGGGCTCGCCCACCAGCACTGA |
| | AGAAGGTGCCTCGAGCCCGCCTGTGCTGAAGAGGCACCAGCGAGAAATTACCCGGACC |
| | ACCCTCCAATCGGATCAGGAGGAAATCGACTACGACGACACCATCTCGGTGGAAATGA |
| | AGAAGGAAGATTTCGATATCTACGACGAGGACGAAAATCAGTCCCCTCGCTCATTCCA |
| | AAAGAAAACTAGACACTACTTTATCGCCGCGGTGGAAAGACTGTGGGACTATGGAATG |
| | TCATCCAGCCCTCACGTCCTTCGGAACCGGGCCCAGAGCGGATCGGTGCCTCAGTTCA |
| | AGAAAGTGGTGTTCCAGGAGTTCACCGACGGCAGCTTCACCCAGCCGCTGTACCGGGG |
| | AGAACTGAACGAACACCTGGGCCTGCTCGGTCCCTACATCCGCGCGGAAGTGGAGGAT |
| | AACATCATGGTGACCTTCCGTAACCAAGCATCCAGACCTTACTCCTTCTATTCCTCCC |
| | TGATCTCATACGAGGAGGACCAGCGCCAAGGCGCCGAGCCCCGCAAGAACTTCGTCAA |
| | GCCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGATG |
| | TCCATTCCGGCCTGATCGGGCCGCTGCTCGTGTGTCACACCAACACCCTGAACCCAGC |
| | GCATGGACGCCAGGTCACCGTCCAGGAGTTTGCTCTGTTCTTCACCATTTTTGACGAA |
| | ACTAAGTCCTGGTACTTCACCGAGAATATGGAGCGAAACTGTAGAGCGCCCTGCAATA |
| | TCCAGATGGAAGATCCGACTTTCAAGGAGAACTATAGATTCCACGCCATCAACGGGTA |
| | CATCATGGATACTCTGCCGGGGCTGGTCATGGCCCAGGATCAGAGGATTCGGTGGTAC |
| | TTGCTGTCAATGGGATCGAACGAAAACATTCACTCCATTCACTTCTCCGGTCACGTGT |
| | TCACTGTGCGCAAGAAGGAGGAGTACAAGATGGCGCTGTACAATCTGTACCCCGGGGT |
| | GTTCGAAACTGTGGAGATGCTGCCGTCCAAGGCCGGCATCTGGAGAGTGGAGTGCCTG |
| | ATCGGAGAGCACCTCCACGCGGGGATGTCCACCCTCTTCCTGGTGTACTCGAATAAGT |
| | GCCAGACCCCGCTGGGCATGGCCTCGGGCCACATCAGAGACTTCCAGATCACAGCAAG |
| | CGGACAATACGGCCAATGGGCGCCGAAGCTGGCCCGCTTGCACTACTCCGGATCGATC |
| | AACGCATGGTCCACCAAGGAACCGTTCTCGTGGATTAAGGTGGACCTCCTGGCCCCTA |
| | TGATTATCCACGGAATTAAGACCCAGGGCGCCAGGCAGAAGTTCTCCTCCCTGTACAT |
| | CTCGCAATTCATCATCATGTACAGCCTGGACGGGAAGAAGTGGCAGACTTACAGGGGA |
| | AACTCCACCGGCACCCTGATGGTCTTTTTCGGCAACGTGGATTCCTCCGGCATTAAGC |
| | ACAACATCTTCAACCCACCGATCATAGCCAGATATATTAGGCTCCACCCCACTCACTA |
| | CTCAATCCGCTCAACTCTTCGGATGGAACTCATGGGGTGCGACCTGAACTCCTGCTCC |
| | ATGCCGTTGGGGATGGAATCAAAGGCTATTAGCGACGCCCAGATCACCGCGAGCTCCT |
| | ACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGAT |
| | TTCCAAAAGACCATGAAAGTGACCGGAGTCACCACCCAGGGAGTGAAGTCCCTTCTGA |
| | CCTCGATGTATGTGAAGGAGTTCCTGATTAGCAGCAGCCAGGACGGGCACCAGTGGAC |
| | CCTGTTCTTCCAAAACGGAAAGGTCAAGGTGTTCCAGGGGAACCAGGACTCGTTCACA |
| | CCCGTGGTGAACTCCCTGGACCCCCCACTGCTGACGCGGTACTTGAGGATTCATCCTC |
| | AGTCCTGGGTCCATCAGATTGCATTGCGAATGGAAGTCCTGGGCTGCGAGGCCCAGGA |
| | CCTGTACTGA -- 5596 |
| WPRE (mutated | 5616 - |
| woodchuck | TCATAATCAACCTCTGGATTACAAAATTTGTGAAAGATTGACTGGTATTCTTAACTAT |
| hepatitis virus | GTTGCTCCTTTTACGCTATGTGGATACGCTGCTTTAATGCCTTTGTATCATGCTATTG |
| post - | CTTCCCGTATGGCTTTCATTTTCTCCTCCTTGTATAAATCCTGGTTGCTGTCTCTTTA |
| transcriptional | TGAGGAGTTGTGGCCCGTTGTCAGGCAACGTGGCGTGGTGTGCACTGTGTTTGCTGAC |
| regulatory | GCAACCCCCACTGGTTGGGGCATTGCCACCACCTGTCAGCTCCTTTCCGGGACTTTCG |
| element) (SEQ | CTTTCCCCCTCCCTATTGCCACGGCGGAACTCATCGCCGCCTGCCTTGCCCGCTGCTG |
| ID NO: 120) | GACAGGGGCTCGGCTGTTGGGCACTGACAATTCCGTGGTGTTGTCGGGGAAATCATCG |
| | TCCTTTCCTTGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTTTGG |
| | GCCGCCTCCCCGCTG -- 6210 |
| bGHpA (bovine | 6219 - |
| growth hormone | CGACTGTGCCTTCTAGTTGCCAGCCATCTGTTGTTTGCCCCTCCCCCGTGCCTTCCTT |
| polyadenylation | GACCCTGGAAGGTGCCACTCCCACTGTCCTTTCCTAATAAAATGAGGAAATTGCATCG |
| signal) (SEQ ID | CATTGTCTGAGTAGGTGTCATTCTATTCTGGGGGGTGGGGTGGGGCAGGACAGCAAGG |
| NO: 122) | GGGAGGATTGGGAAGACAATAGCAGGCATGCTGGGGA -- 6429 |
| 3′ ITR inverted | 6549 -- |
| terminal repeat | CACTTCCTGGCGCGCAAAATATCCTCTTGTCCTTGAGTCTCATTGGAGGGTTCGTTCG |
| (SEQ ID NO: | TTCGAACCAGCCAATCAGGGGAGGGGGAAGTGACGCAAGTTCCGGTCACATGCTTCCG |
| 184) | GTGACGCACATCCGGTGACGTAGTTCCGGTCACGTGCTTCCTGTCACGTGTTTCCGGT |
| | CACGTGACTTCCGGTCATGTGACTTCCGGTGACGTGTTTCCGGCTTAACTATTGGGCT |
| | GACCGCGCGGCATGCGCGTGGTCAACCTAACAGCCGGAAACACGTCACCG -- 6830 |

Full-length Sequence (SEQ ID NO: 182)
CGGTGACGTGTTTCCGGCTGTTAGGTTGACCACGCGCATGCCGCGCGGTCAGCCCAATAGTTAAGCCGGAAAC
ACGTCACCGGAAGTCACATGACCGGAAGTCACGTGACCGGAAACACGTGACAGGAAGCACGTGACCGGAACTA
CGTCACCGGATGTGCGTCACCGGAAGCATGTGACCGGAACTTGCGTCACTTCCCCCTCCCCTGATTGGCTGGT
TCGAACGAACGAACCCTCCAATGAGACTCAAGGACAAGAGGATATTTTGCGCGCCAGGAAGTGGCGGCAATTC
AGTCGATAACTATAACGGTCCTAAGGTAGCGATTTAAATACGCGCTCTCTTAAGGTAGCCCCGGGACGCGTCA
ATTGAGATCTGGATCCGGTACCGAATTCGCGGCCGCCTCGACGACTAGCGTTTAATTAAACGCGTGTCTGTCT
GCACATTTCGTAGAGCGAGTGTTCCGATACTCTAATCTCCCTAGGCAAGGTTCATATTTGTGTAGGTTACTTA
TTCTCCTTTTGTTGACTAAGTCAATAATCAGAATCAGCAGGTTTGGAGTCAGCTTGGCAGGGATCAGCAGCCT
GGGTTGGAAGGAGGGGGTATAAAAGCCCCTTCACCAGGAGAAGCCGTCACACAGATCCACAAGCTCCTGAGGT
AAGTGCCGTGTGTGGTTCCCGCGGGCCTGGCCTCTTTACGGGTTATGGCCCTTGCGTGCCTTGAATTACTGAC
ACTGACATCCACTTTTTCTTTTTCTCCACAGCTAGCGCCACCATGCAGATTGAGCTGTCCACTTGTTTCTTCC
TGTGCCTCCTGCGCTTCTGTTTCTCCGCCACTCGCCGGTACTACCTTGGAGCCGTGGAGCTTTCATGGGACTA
CATGCAGAGCGACCTGGGCGAACTCCCCGTGGATGCCAGATTCCCCCCCCGCGTGCCAAAGTCCTTCCCCTTT
AACACCTCCGTGGTGTACAAGAAAACCCTCTTTGTCGAGTTCACTGACCACCTGTTCAACATCGCCAAGCCGC
GCCCACCTTGGATGGGCCTCCTGGGACCGACCATTCAAGCTGAAGTGTACGACACCGTGGTGATCACCCTGAA
GAACATGGCGTCCCACCCCGTGTCCCTGCATGCGGTCGGAGTGTCCTACTGGAAGGCCTCCGAAGGAGCTGAG
TACGACGACCAGACTAGCCAGCGGGAAAAGGAGGACGATAAAGTGTTCCCGGGCGGCTCGCATACTTACGTGT
GGCAAGTCCTGAAGGAAAACGGACCTATGGCATCCGATCCTCTGTGCCTGACTTACTCCTACCTTTCCCATGT
GGACCTCGTGAAGGACCTGAACAGCGGGCTGATTGGTGCACTTCTCGTGTGCCGCGAAGGTTCGCTCGCTAAG
GAAAAGACCCAGACCCTCCATAAGTTCATCCTTTTGTTCGCTGTGTTCGATGAAGGAAAGTCATGGCATTCCG
AAACTAAGAACTCGCTGATGCAGGACCGGGATGCCGCCTCAGCCCGCGCCTGGCCTAAAATGCATACAGTCAA
CGGATACGTGAATCGGTCACTGCCCGGGCTCATCGGTTGTCACAGAAAGTCCGTGTACTGGCACGTCATCGGC
ATGGGCACTACGCCTGAAGTGCACTCCATCTTCCTGGAAGGGCACACCTTCCTCGTGCGCAACCACCGCCAGG
CCTCTCTGGAAATCTCCCCGATTACCTTTCTGACCGCCCAGACTCTGCTCATGGACCTGGGGCAGTTCCTTCT
CTTCTGCCACATCTCCAGCCATCAGCACGACGGAATGGAGGCCTACGTGAAGGTGGACTCATGCCCGGAAGAA
CCTCAGTTGCGGATGAAGAACAACGAGGAGGCCGAGGACTATGACGACGATTTGACTGACTCCGAGATGGACG
TCGTGCGGTTCGATGACGACAACAGCCCCAGCTTCATCCAGATTCGCAGCGTGGCCAAGAAGCACCCCAAAAC
CTGGGTGCACTACATCGCGGCCGAGGAAGAAGATTGGGACTACGCCCCGTTGGTGCTGGCACCCGATGACCGG
TCGTACAAGTCCCAGTATCTGAACAATGGTCCGCAGCGGATTGGCAGAAAGTACAAGAAAGTGCGGTTCATGG
CGTACACTGACGAAACGTTTAAGACCCGGGAGGCCATTCAACATGAGAGCGGCATTCTGGGACCACTGCTGTA
CGGAGAGGTCGGCGATACCCTGCTCATCATCTTCAAAAACCAGGCCTCCCGGCCTTACAACATCTACCCTCAC
GGAATCACCGACGTGCGGCCACTCTACTCGCGGCGCCTGCCGAAGGGCGTCAAGCACCTGAAAGACTTCCCTA
TCCTGCCGGGCGAAATCTTCAAGTATAAGTGGACCGTCACCGTGGAGGACGGGCCCACCAAGAGCGATCCTAG
GTGTCTGACTCGGTACTACTCCAGCTTCGTGAACATGGAACGGGACCTGGCATCGGGACTCATTGGACCGCTG
CTGATCTGCTACAAAGAGTCGGTGGATCAACGCGGCAACCAGATCATGTCCGACAAGCGCAACGTGATCCTGT
TCTCCGTGTTTGATGAAAACAGATCCTGGTACCTCACTGAAAACATCCAGAGGTTCCTCCCAAACCCCGCAGG
AGTGCAACTGGAGGACCCTGAGTTTCAGGCCTCGAATATCATGCACTCGATTAACGGTTACGTGTTCGACTCG
CTGCAACTGAGCGTGTGCCTCCATGAAGTCGCTTACTGGTACATTCTGTCCATCGGCGCCCAGACTGACTTCC
TGAGCGTGTTCTTTTCCGGTTACACCTTTAAGCACAAGATGGTGTACGAAGATACCCTGACCCTGTTCCCTTT
CTCCGGCGAAACGGTGTTCATGTCGATGGAGAACCCGGGTCTGTGGATTCTGGGATGCCACAACAGCGACTTT
CGGAACCGCGGAATGACTGCCCTGCTGAAGGTGTCCTCATGCGACAAGAACACCGGAGACTACTACGAGGACT
CCTACGAGGATATCTCAGCCTACCTCCTGTCCAAGAACAACGCGATCGAGCCGCGCAGCTTCAGCCAGAACGG
CGCGCCAACATCAGAGAGCGCCACCCCTGAAAGTGGTCCCGGGAGCGAGCCAGCCACATCTGGGTCGGAAACG
CCAGGCACAAGTGAGTCTGCAACTCCCGAGTCCGGACCTGGCTCCGAGCCTGCCACTAGCGGCTCCGAGACTC
CGGGAACTTCCGAGAGCGCTACACCAGAAAGCGGACCCGGAACCAGTACCGAACCTAGCGAGGGCTCTGCTCC
GGGCAGCCCAGCCGGCTCTCCTACATCCACGGAGGAGGGCACTTCCGAATCCGCCACCCCGGAGTCAGGGCCA
GGATCTGAACCCGCTACCTCAGGCAGTGAGACGCCAGGAACGAGCGAGTCCGCTACACCGGAGAGTGGGCCAG
GGAGCCCTGCTGGATCTCCTACGTCCACTGAGGAAGGGTCACCAGCGGGCTCGCCCACCAGCACTGAAGAAGG
TGCCTCGAGCCCGCCTGTGCTGAAGAGGCACCAGCGAGAAATTACCCGGACCACCCTCCAATCGGATCAGGAG
GAAATCGACTACGACGACACCATCTCGGTGGAAATGAAGAAGGAAGATTTCGATATCTACGACGAGGACGAAA
ATCAGTCCCCTCGCTCATTCCAAAAGAAAACTAGACACTACTTTATCGCCGCGGTGGAAAGACTGTGGGACTA
TGGAATGTCATCCAGCCCTCACGTCCTTCGGAACCGGGCCCAGAGCGGATCGGTGCCTCAGTTCAAGAAAGTG
GTGTTCCAGGAGTTCACCGACGGCAGCTTCACCCAGCCGCTGTACCGGGGAGAACTGAACGAACACCTGGGCC
TGCTCGGTCCCTACATCCGCGCGGAAGTGGAGGATAACATCATGGTGACCTTCCGTAACCAAGCATCCAGACC
TTACTCCTTCTATTCCTCCCTGATCTCATACGAGGAGGACCAGCGCCAAGGCGCCGAGCCCCGCAAGAACTTC
GTCAAGCCCAACGAGACTAAGACCTACTTCTGGAAGGTCCAACACCATATGGCCCCGACCAAGGATGAGTTTG
ACTGCAAGGCCTGGGCCTACTTCTCCGACGTGGACCTTGAGAAGGATGTCCATTCCGGCCTGATCGGGCCGCT
GCTCGTGTGTCACACCAACACCCTGAACCCAGCGCATGGACGCCAGGTCACCGTCCAGGAGTTTGCTCTGTTC
TTCACCATTTTTGACGAAACTAAGTCCTGGTACTTCACCGAGAATATGGAGCGAAACTGTAGAGCGCCCTGCA
ATATCCAGATGGAAGATCCGACTTTCAAGGAGAACTATAGATTCCACGCCATCAACGGGTACATCATGGATAC
TCTGCCGGGGCTGGTCATGGCCCAGGATCAGAGGATTCGGTGGTACTTGCTGTCAATGGGATCGAACGAAAAC
ATTCACTCCATTCACTTCTCCGGTCACGTGTTCACTGTGCGCAAGAAGGAGGAGTACAAGATGGCGCTGTACA
ATCTGTACCCCGGGGTGTTCGAAACTGTGGAGATGCTGCCGTCCAAGGCCGGCATCTGGAGAGTGGAGTGCCT
GATCGGAGAGCACCTCCACGCGGGGATGTCCACCCTCTTCCTGGTGTACTCGAATAAGTGCCAGACCCCGCTG
GGCATGGCCTCGGGCCACATCAGAGACTTCCAGATCACAGCAAGCGGACAATACGGCCAATGGGCGCCGAAGC
TGGCCCGCTTGCACTACTCCGGATCGATCAACGCATGGTCCACCAAGGAACCGTTCTCGTGGATTAAGGTGGA
CCTCCTGGCCCCTATGATTATCCACGGAATTAAGACCCAGGGCGCCAGGCAGAAGTTCTCCTCCCTGTACATC
TCGCAATTCATCATCATGTACAGCCTGGACGGGAAGAAGTGGCAGACTTACAGGGGAAACTCCACCGGCACCC
TGATGGTCTTTTTCGGCAACGTGGATTCCTCCGGCATTAAGCACAACATCTTCAACCCACCGATCATAGCCAG
ATATATTAGGCTCCACCCCACTCACTACTCAATCCGCTCAACTCTTCGGATGGAACTCATGGGGTGCGACCTG
AACTCCTGCTCCATGCCGTTGGGGATGGAATCAAAGGCTATTAGCGACGCCCAGATCACCGCGAGCTCCTACT
TCACTAACATGTTCGCCACCTGGAGCCCCTCCAAGGCCAGGCTGCACTTGCAGGGACGGTCAAATGCCTGGCG
GCCGCAAGTGAACAATCCGAAGGAATGGCTTCAAGTGGATTTCCAAAAGACCATGAAAGTGACCGGAGTCACC
ACCCAGGGAGTGAAGTCCCTTCTGACCTCGATGTATGTGAAGGAGTTCCTGATTAGCAGCAGCCAGGACGGGC
ACCAGTGGACCCTGTTCTTCCAAAACGGAAAGGTCAAGGTGTTCCAGGGGAACCAGGACTCGTTCACACCCGT
GGTGAACTCCCTGGACCCCCCACTGCTGACGCGGTACTTGAGGATTCATCCTCAGTCCTGGGTCCATCAGATT
GCATTGCGAATGGAAGTCCTGGGCTGCGAGGCCCAGGACCTGTACTGAATCAGCCTGAGCTCGCTGATCATAA
TCAACCTCTGGATTACAAAATTTGTGAAAGATTGACTGGTATTCTTAACTATGTTGCTCCTTTTACGCTATGT
GGATACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACTGTGTT
TGCTGACGCAACCCCCACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTGCTCGCCTGTGTTGCCACCTGGAT
TCTGCGCGGGACGTCCTTCTGCTACGTCCCTTCGGCCCTCAATCCAGCGGACCTTCCTTCCCGCGGCCTGCTG
CCGGCTCTGCGGCCTCTTCCGCGTCTTCGCCTTCGCCCTCAGACGAGTCGGATCTCCCTTTGGGCCGCCTCCC
CGCTGATCAGCCTCGACTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGCAGGCATGCT
GGGGATGCGGTGGGCTCTATGGCTTCTGAGGCGGAAAGAACGGGCTCGAGAAGCTTCTAGATATCCTCTCTTA
AGGTAGCATCGAGATTTAAATTAGGGATAACAGGGTAATGGCGCGGGCCGCCACTTCCTGGCGCGCAAAATAT
CCTCTTGTCCTTGAGTCTCATTGGAGGGTTCGTTCGTTCGAACCAGCCAATCAGGGGAGGGGGAAGTGACGCA
AGTTCCGGTCACATGCTTCCGGTGACGCACATCCGGTGACGTAGTTCCGGTCACGTGCTTCCTGTCACGTGTT
TCCGGTCACGTGACTTCCGGTCATGTGACTTCCGGTGACGTGTTTCCGGCTTAACTATTGGGCTGACCGCGCG
GCATGCGCGTGGTCAACCTAACAGCCGGAAACACGTCACCG

TABLE 2D
Example B19-FVIII construct bearing full length B19 ITRs
(nucleotides 1-7032; SEQ ID NO: 189)

| Description | Sequence |
|---|---|
| 5′ ITR (SEQ ID | CCAAATCAGATGCCGCCGGTCGCCGCCGGTAGGCGGGACTTCCGGTACAAGATGGCGG |
| NO: 185) | ACAATTACGTCATTTCCTGTGACGTCATTTCCTGTGACGTCACTTCCGGTGGGCGGGA |
| | CTTCCGGAATTAGGGTTGGCTCTGGGCCAGCTTGCTTGGGGTTGCCTTGACACTAAGA |
| | CAAGCGGCGCGCCGCTTGATCTTAGTGGCACGTCAACCCCAAGCGCTGGCCCAGAGCC |
| | AACCCTAATTCCGGAAGTCCCGCCCACCGGAAGTGACGTCACAGGAAATGACGTCACA |
| | GGAAATGACGTAATTGTCCGCCATCTTGTACCGGAAGTCCCGCCTACCGGCGGCGACC |
| | GGCGGCATCTGATTTGGTGTCTTCTTTTAAATTTT |
| TTPp (liver- | ACGCGTGTCTGTCTGCACATTTCGTAGAGCGAGTGTTCCGATACTCTAATCTCCCTAG |
| specific | GCAAGGTTCATATTTGTGTAGGTTACTTATTCTCCTTTTGTTGACTAAGTCAATAATC |
| promoter) (SEQ | AGAATCAGCAGGTTTGGAGTCAGCTTGGCAGGGATCAGCAGCCTGGGTTGGAAGGAGG |
| ID NO: 113) | GGGTATAAAAGCCCCTTCACCAGGAGAAGCCGTCACACAGATCCACAAGCTCCTG |
| Synthetic Intron | GTAAGTGCCGTGTGTGGTTCCCGCGGGCCTGGCCTCTTTACGGGTTATGGCCCTTGCG |
| (SEQ ID NO: 115) | TGCCTTGAATTACTGACACTGACATCCACTTTTTCTTTTTCTCCACAG |
| FVIIIco6XTEN | ATGCAGATTGAGCTGTCCACTTGTTTCTTCCTGTGCCTCCTGCGCTTCTGTTTCTCCG |
| (SEQ ID NO: 117) | CCACTCGCCGGTACTACCTTGGAGCCGTGGAGCTTTCATGGGACTACATGCAGAGCGA |
| (open reading | CCTGGGCGAACTCCCCGTGGATGCCAGATTCCCCCCCCGCGTGCCAAAGTCCTTCCCC |
| frame for codon- | TTTAACACCTCCGTGGTGTACAAGAAAACCCTCTTTGTCGAGTTCACTGACCACCTGT |
| optimized FVIII | TCAACATCGCCAAGCCGCGCCCACCTTGGATGGGCCTCCTGGGACCGACCATTCAAGC |
| version 6 | TGAAGTGTACGACACCGTGGTGATCACCCTGAAGAACATGGCGTCCCACCCCGTGTCC |
| containing | CTGCATGCGGTCGGAGTGTCCTACTGGAAGGCCTCCGAAGGAGCTGAGTACGACGACC |
| XTEN144; the | AGACTAGCCAGCGGGAAAAGGAGGACGATAAAGTGTTCCCGGGCGGCTCGCATACTTA |
| XTEN sequence | CGTGTGGCAAGTCCTGAAGGAAAACGGACCTATGGCATCCGATCCTCTGTGCCTGACT |
| is marked by | TACTCCTACCTTTCCCATGTGGACCTCGTGAAGGACCTGAACAGCGGGCTGATTGGTG |
| double | CACTTCTCGTGTGCCGCGAAGGTTCGCTCGCTAAGGAAAAGACCCAGACCCTCCATAA |
| underlining (SEQ | GTTCATCCTTTTGTTCGCTGTGTTCGATGAAGGAAAGTCATGGCATTCCGAAACTAAG |
| ID NO: 118)) | AACTCGCTGATGCAGGACCGGGATGCCGCCTCAGCCCGCGCCTGGCCTAAAATGCATA |
| | CAGTCAACGGATACGTGAATCGGTCACTGCCCGGGCTCATCGGTTGTCACAGAAAGTC |
| | CGTGTACTGGCACGTCATCGGCATGGGCACTACGCCTGAAGTGCACTCCATCTTCCTG |
| | GAAGGGCACACCTTCCTCGTGCGCAACCACCGCCAGGCCTCTCTGGAAATCTCCCCGA |
| | TTACCTTTCTGACCGCCCAGACTCTGCTCATGGACCTGGGGCAGTTCCTTCTCTTCTG |
| | CCACATCTCCAGCCATCAGCACGACGGAATGGAGGCCTACGTGAAGGTGGACTCATGC |
| | CCGGAAGAACCTCAGTTGCGGATGAAGAACAACGAGGAGGCCGAGGACTATGACGACG |
| | ATTTGACTGACTCCGAGATGGACGTCGTGCGGTTCGATGACGACAACAGCCCCAGCTT |
| | CATCCAGATTCGCAGCGTGGCCAAGAAGCACCCCAAAACCTGGGTGCACTACATCGCG |
| | GCCGAGGAAGAAGATTGGGACTACGCCCCGTTGGTGCTGGCACCCGATGACCGGTCGT |
| | ACAAGTCCCAGTATCTGAACAATGGTCCGCAGCGGATTGGCAGAAAGTACAAGAAAGT |
| | GCGGTTCATGGCGTACACTGACGAAACGTTTAAGACCCGGGAGGCCATTCAACATGAG |
| | AGCGGCATTCTGGGACCACTGCTGTACGGAGAGGTCGGCGATACCCTGCTCATCATCT |
| | TCAAAAACCAGGCCTCCCGGCCTTACAACATCTACCCTCACGGAATCACCGACGTGCG |
| | GCCACTCTACTCGCGGCGCCTGCCGAAGGGCGTCAAGCACCTGAAAGACTTCCCTATC |
| | CTGCCGGGCGAAATCTTCAAGTATAAGTGGACCGTCACCGTGGAGGACGGGCCCACCA |
| | AGAGCGATCCTAGGTGTCTGACTCGGTACTACTCCAGCTTCGTGAACATGGAACGGGA |
| | CCTGGCATCGGGACTCATTGGACCGCTGCTGATCTGCTACAAAGAGTCGGTGGATCAA |
| | CGCGGCAACCAGATCATGTCCGACAAGCGCAACGTGATCCTGTTCTCCGTGTTTGATG |
| | AAAACAGATCCTGGTACCTCACTGAAAACATCCAGAGGTTCCTCCCAAACCCCGCAGG |
| | AGTGCAACTGGAGGACCCTGAGTTTCAGGCCTCGAATATCATGCACTCGATTAACGGT |
| | TACGTGTTCGACTCGCTGCAACTGAGCGTGTGCCTCCATGAAGTCGCTTACTGGTACA |
| | TTCTGTCCATCGGCGCCCAGACTGACTTCCTGAGCGTGTTCTTTTCCGGTTACACCTT |
| | TAAGCACAAGATGGTGTACGAAGATACCCTGACCCTGTTCCCTTTCTCCGGCGAAACG |
| | GTGTTCATGTCGATGGAGAACCCGGGTCTGTGGATTCTGGGATGCCACAACAGCGACT |
| | TTCGGAACCGCGGAATGACTGCCCTGCTGAAGGTGTCCTCATGCGACAAGAACACCGG |
| | AGACTACTACGAGGACTCCTACGAGGATATCTCAGCCTACCTCCTGTCCAAGAACAAC |
| | GCGATCGAGCCGCGCAGCTTCAGCCAGAACGGCGCGCCAACATCAGAGAGCGCCACCC |
| | CTGAAAGTGGTCCCGGGAGCGAGCCAGCCACATCTGGGTCGGAAACGCCAGGCACAAG |
| | TGAGTCTGCAACTCCCGAGTCCGGACCTGGCTCCGAGCCTGCCACTAGCGGCTCCGAG |
| | ACTCCGGGAACTTCCGAGAGCGCTACACCAGAAAGCGGACCCGGAACCAGTACCGAAC |
| | CTAGCGAGGGCTCTGCTCCGGGCAGCCCAGCCGGCTCTCCTACATCCACGGAGGAGGG |
| | CACTTCCGAATCCGCCACCCCGGAGTCAGGGCCAGGATCTGAACCCGCTACCTCAGGC |
| | AGTGAGACGCCAGGAACGAGCGAGTCCGCTACACCGGAGAGTGGGCCAGGGAGCCCTG |
| | CTGGATCTCCTACGTCCACTGAGGAAGGGTCACCAGCGGGCTCGCCCACCAGCACTGA |
| | AGAAGGTGCCTCGAGCCCGCCTGTGCTGAAGAGGCACCAGCGAGAAATTACCCGGACC |
| | ACCCTCCAATCGGATCAGGAGGAAATCGACTACGACGACACCATCTCGGTGGAAATGA |
| | AGAAGGAAGATTTCGATATCTACGACGAGGACGAAAATCAGTCCCCTCGCTCATTCCA |
| | AAAGAAAACTAGACACTACTTTATCGCCGCGGTGGAAAGACTGTGGGACTATGGAATG |
| | TCATCCAGCCCTCACGTCCTTCGGAACCGGGCCCAGAGCGGATCGGTGCCTCAGTTCA |
| | AGAAAGTGGTGTTCCAGGAGTTCACCGACGGCAGCTTCACCCAGCCGCTGTACCGGGG |
| | AGAACTGAACGAACACCTGGGCCTGCTCGGTCCCTACATCCGCGCGGAAGTGGAGGAT |
| | AACATCATGGTGACCTTCCGTAACCAAGCATCCAGACCTTACTCCTTCTATTCCTCCC |
| | TGATCTCATACGAGGAGGACCAGCGCCAAGGCGCCGAGCCCCGCAAGAACTTCGTCAA |
| | GCCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGATG |
| | TCCATTCCGGCCTGATCGGGCCGCTGCTCGTGTGTCACACCAACACCCTGAACCCAGC |
| | GCATGGACGCCAGGTCACCGTCCAGGAGTTTGCTCTGTTCTTCACCATTTTTGACGAA |
| | ACTAAGTCCTGGTACTTCACCGAGAATATGGAGCGAAACTGTAGAGCGCCCTGCAATA |
| | TCCAGATGGAAGATCCGACTTTCAAGGAGAACTATAGATTCCACGCCATCAACGGGTA |
| | CATCATGGATACTCTGCCGGGGCTGGTCATGGCCCAGGATCAGAGGATTCGGTGGTAC |
| | TTGCTGTCAATGGGATCGAACGAAAACATTCACTCCATTCACTTCTCCGGTCACGTGT |
| | TCACTGTGCGCAAGAAGGAGGAGTACAAGATGGCGCTGTACAATCTGTACCCCGGGGT |
| | GTTCGAAACTGTGGAGATGCTGCCGTCCAAGGCCGGCATCTGGAGAGTGGAGTGCCTG |
| | ATCGGAGAGCACCTCCACGCGGGGATGTCCACCCTCTTCCTGGTGTACTCGAATAAGT |
| | GCCAGACCCCGCTGGGCATGGCCTCGGGCCACATCAGAGACTTCCAGATCACAGCAAG |
| | CGGACAATACGGCCAATGGGCGCCGAAGCTGGCCCGCTTGCACTACTCCGGATCGATC |
| | AACGCATGGTCCACCAAGGAACCGTTCTCGTGGATTAAGGTGGACCTCCTGGCCCCTA |
| | TGATTATCCACGGAATTAAGACCCAGGGCGCCAGGCAGAAGTTCTCCTCCCTGTACAT |
| | CTCGCAATTCATCATCATGTACAGCCTGGACGGGAAGAAGTGGCAGACTTACAGGGGA |
| | AACTCCACCGGCACCCTGATGGTCTTTTTCGGCAACGTGGATTCCTCCGGCATTAAGC |
| | ACAACATCTTCAACCCACCGATCATAGCCAGATATATTAGGCTCCACCCCACTCACTA |
| | CTCAATCCGCTCAACTCTTCGGATGGAACTCATGGGGTGCGACCTGAACTCCTGCTCC |
| | ATGCCGTTGGGGATGGAATCAAAGGCTATTAGCGACGCCCAGATCACCGCGAGCTCCT |
| | ACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGAT |
| | TTCCAAAAGACCATGAAAGTGACCGGAGTCACCACCCAGGGAGTGAAGTCCCTTCTGA |
| | CCTCGATGTATGTGAAGGAGTTCCTGATTAGCAGCAGCCAGGACGGGCACCAGTGGAC |
| | CCTGTTCTTCCAAAACGGAAAGGTCAAGGTGTTCCAGGGGAACCAGGACTCGTTCACA |
| | CCCGTGGTGAACTCCCTGGACCCCCCACTGCTGACGCGGTACTTGAGGATTCATCCTC |
| | AGTCCTGGGTCCATCAGATTGCATTGCGAATGGAAGTCCTGGGCTGCGAGGCCCAGGA |
| | CCTGTACTGA |
| WPRE (mutated | TCATAATCAACCTCTGGATTACAAAATTTGTGAAAGATTGACTGGTATTCTTAACTAT |
| woodchuck | GTTGCTCCTTTTACGCTATGTGGATACGCTGCTTTAATGCCTTTGTATCATGCTATTG |
| hepatitis virus | CTTCCCGTATGGCTTTCATTTTCTCCTCCTTGTATAAATCCTGGTTGCTGTCTCTTTA |
| post- | TGAGGAGTTGTGGCCCGTTGTCAGGCAACGTGGCGTGGTGTGCACTGTGTTTGCTGAC |
| transcriptional | GCAACCCCCACTGGTTGGGGCATTGCCACCACCTGTCAGCTCCTTTCCGGGACTTTCG |
| regulatory | CTTTCCCCCTCCCTATTGCCACGGCGGAACTCATCGCCGCCTGCCTTGCCCGCTGCTG |
| element) (SEQ | GACAGGGGCTCGGCTGTTGGGCACTGACAATTCCGTGGTGTTGTCGGGGAAATCATCG |
| ID NO: 120) | TCCTTTCCTTGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTTTGG |
| | GCCGCCTCCCCGCTG |
| bGHpA (bovine | CGACTGTGCCTTCTAGTTGCCAGCCATCTGTTGTTTGCCCCTCCCCCGTGCCTTCCTT |
| growth hormone | GACCCTGGAAGGTGCCACTCCCACTGTCCTTTCCTAATAAAATGAGGAAATTGCATCG |
| polyadenylation | CATTGTCTGAGTAGGTGTCATTCTATTCTGGGGGGTGGGGTGGGGCAGGACAGCAAGG |
| signal) (SEQ ID | GGGAGGATTGGGAAGACAATAGCAGGCATGCTGGGGA |
| NO: 122) | |
| 3′ ITR inverted | AAAATTTAAAAGAAGACACCAAATCAGATGCCGCCGGTCGCCGCCGGTAGGCGGGACT |
| terminal repeat | TCCGGTACAAGATGGCGGACAATTACGTCATTTCCTGTGACGTCATTTCCTGTGACGT |
| (SEQ ID NO: | CACTTCCGGTGGGCGGGACTTCCGGAATTAGGGTTGGCTCTGGGCCAGCGCTTGGGGT |
| 186) | TGACGTGCCACTAAGATCAAGCGGCGCGCCGCTTGTCTTAGTGTCAAGGCAACCCCAA |
| | GCAAGCTGGCCCAGAGCCAACCCTAATTCCGGAAGTCCCGCCCACCGGAAGTGACGTC |
| | ACAGGAAATGACGTCACAGGAAATGACGTAATTGTCCGCCATCTTGTACCGGAAGTCC |
| | CGCCTACCGGCGGCGACCGGCGGCATCTGATTTGG |

Full-length Sequence (SEQ ID NO: 189)
CCAAATCAGATGCCGCCGGTCGCCGCCGGTAGGCGGGACTTCCGGTACAAGATGGCGGACAATTACGTCATTT
CCTGTGACGTCATTTCCTGTGACGTCACTTCCGGTGGGCGGGACTTCCGGAATTAGGGTTGGCTCTGGGCCAG
CTTGCTTGGGGTTGCCTTGACACTAAGACAAGCGGCGCGCCGCTTGATCTTAGTGGCACGTCAACCCCAAGCG
CTGGCCCAGAGCCAACCCTAATTCCGGAAGTCCCGCCCACCGGAAGTGACGTCACAGGAAATGACGTCACAGG
AAATGACGTAATTGTCCGCCATCTTGTACCGGAAGTCCCGCCTACCGGCGGCGACCGGCGGCATCTGATTTGG
TGTCTTCTTTTAAATTTTGCGGCAATTCAGTCGATAACTATAACGGTCCTAAGGTAGCGATTTAAATACGCGC
TCTCTTAAGGTAGCCCCGGGACGCGTCAATTGAGATCTGGATCCGGTACCGAATTCGCGGCCGCCTCGACGAC
TAGCGTTTAATTAAACGCGTGTCTGTCTGCACATTTCGTAGAGCGAGTGTTCCGATACTCTAATCTCCCTAGG
CAAGGTTCATATTTGTGTAGGTTACTTATTCTCCTTTTGTTGACTAAGTCAATAATCAGAATCAGCAGGTTTG
GAGTCAGCTTGGCAGGGATCAGCAGCCTGGGTTGGAAGGAGGGGGTATAAAAGCCCCTTCACCAGGAGAAGCC
GTCACACAGATCCACAAGCTCCTGAGGTAAGTGCCGTGTGTGGTTCCCGCGGGCCTGGCCTCTTTACGGGTTA
TGGCCCTTGCGTGCCTTGAATTACTGACACTGACATCCACTTTTTCTTTTTCTCCACAGCTAGCGCCACCATG
CAGATTGAGCTGTCCACTTGTTTCTTCCTGTGCCTCCTGCGCTTCTGTTTCTCCGCCACTCGCCGGTACTACC
TTGGAGCCGTGGAGCTTTCATGGGACTACATGCAGAGCGACCTGGGCGAACTCCCCGTGGATGCCAGATTCCC
CCCCCGCGTGCCAAAGTCCTTCCCCTTTAACACCTCCGTGGTGTACAAGAAAACCCTCTTTGTCGAGTTCACT
GACCACCTGTTCAACATCGCCAAGCCGCGCCCACCTTGGATGGGCCTCCTGGGACCGACCATTCAAGCTGAAG
TGTACGACACCGTGGTGATCACCCTGAAGAACATGGCGTCCCACCCCGTGTCCCTGCATGCGGTCGGAGTGTC
CTACTGGAAGGCCTCCGAAGGAGCTGAGTACGACGACCAGACTAGCCAGCGGGAAAAGGAGGACGATAAAGTG
TTCCCGGGCGGCTCGCATACTTACGTGTGGCAAGTCCTGAAGGAAAACGGACCTATGGCATCCGATCCTCTGT
GCCTGACTTACTCCTACCTTTCCCATGTGGACCTCGTGAAGGACCTGAACAGCGGGCTGATTGGTGCACTTCT
CGTGTGCCGCGAAGGTTCGCTCGCTAAGGAAAAGACCCAGACCCTCCATAAGTTCATCCTTTTGTTCGCTGTG
TTCGATGAAGGAAAGTCATGGCATTCCGAAACTAAGAACTCGCTGATGCAGGACCGGGATGCCGCCTCAGCCC
GCGCCTGGCCTAAAATGCATACAGTCAACGGATACGTGAATCGGTCACTGCCCGGGCTCATCGGTTGTCACAG
AAAGTCCGTGTACTGGCACGTCATCGGCATGGGCACTACGCCTGAAGTGCACTCCATCTTCCTGGAAGGGCAC
ACCTTCCTCGTGCGCAACCACCGCCAGGCCTCTCTGGAAATCTCCCCGATTACCTTTCTGACCGCCCAGACTC
TGCTCATGGACCTGGGGCAGTTCCTTCTCTTCTGCCACATCTCCAGCCATCAGCACGACGGAATGGAGGCCTA
CGTGAAGGTGGACTCATGCCCGGAAGAACCTCAGTTGCGGATGAAGAACAACGAGGAGGCCGAGGACTATGAC
GACGATTTGACTGACTCCGAGATGGACGTCGTGCGGTTCGATGACGACAACAGCCCCAGCTTCATCCAGATTC
GCAGCGTGGCCAAGAAGCACCCCAAAACCTGGGTGCACTACATCGCGGCCGAGGAAGAAGATTGGGACTACGC
CCCGTTGGTGCTGGCACCCGATGACCGGTCGTACAAGTCCCAGTATCTGAACAATGGTCCGCAGCGGATTGGC
AGAAAGTACAAGAAAGTGCGGTTCATGGCGTACACTGACGAAACGTTTAAGACCCGGGAGGCCATTCAACATG
AGAGCGGCATTCTGGGACCACTGCTGTACGGAGAGGTCGGCGATACCCTGCTCATCATCTTCAAAAACCAGGC
CTCCCGGCCTTACAACATCTACCCTCACGGAATCACCGACGTGCGGCCACTCTACTCGCGGCGCCTGCCGAAG
GGCGTCAAGCACCTGAAAGACTTCCCTATCCTGCCGGGCGAAATCTTCAAGTATAAGTGGACCGTCACCGTGG
AGGACGGGCCCACCAAGAGCGATCCTAGGTGTCTGACTCGGTACTACTCCAGCTTCGTGAACATGGAACGGGA
CCTGGCATCGGGACTCATTGGACCGCTGCTGATCTGCTACAAAGAGTCGGTGGATCAACGCGGCAACCAGATC
ATGTCCGACAAGCGCAACGTGATCCTGTTCTCCGTGTTTGATGAAAACAGATCCTGGTACCTCACTGAAAACA
TCCAGAGGTTCCTCCCAAACCCCGCAGGAGTGCAACTGGAGGACCCTGAGTTTCAGGCCTCGAATATCATGCA
CTCGATTAACGGTTACGTGTTCGACTCGCTGCAACTGAGCGTGTGCCTCCATGAAGTCGCTTACTGGTACATT
CTGTCCATCGGCGCCCAGACTGACTTCCTGAGCGTGTTCTTTTCCGGTTACACCTTTAAGCACAAGATGGTGT
ACGAAGATACCCTGACCCTGTTCCCTTTCTCCGGCGAAACGGTGTTCATGTCGATGGAGAACCCGGGTCTGTG
GATTCTGGGATGCCACAACAGCGACTTTCGGAACCGCGGAATGACTGCCCTGCTGAAGGTGTCCTCATGCGAC
AAGAACACCGGAGACTACTACGAGGACTCCTACGAGGATATCTCAGCCTACCTCCTGTCCAAGAACAACGCGA
TCGAGCCGCGCAGCTTCAGCCAGAACGGCGCGCCAACATCAGAGAGCGCCACCCCTGAAAGTGGTCCCGGGAG
CGAGCCAGCCACATCTGGGTCGGAAACGCCAGGCACAAGTGAGTCTGCAACTCCCGAGTCCGGACCTGGCTCC
GAGCCTGCCACTAGCGGCTCCGAGACTCCGGGAACTTCCGAGAGCGCTACACCAGAAAGCGGACCCGGAACCA
GTACCGAACCTAGCGAGGGCTCTGCTCCGGGCAGCCCAGCCGGCTCTCCTACATCCACGGAGGAGGGCACTTC
CGAATCCGCCACCCCGGAGTCAGGGCCAGGATCTGAACCCGCTACCTCAGGCAGTGAGACGCCAGGAACGAGC
GAGTCCGCTACACCGGAGAGTGGGCCAGGGAGCCCTGCTGGATCTCCTACGTCCACTGAGGAAGGGTCACCAG
CGGGCTCGCCCACCAGCACTGAAGAAGGTGCCTCGAGCCCGCCTGTGCTGAAGAGGCACCAGCGAGAAATTAC
CCGGACCACCCTCCAATCGGATCAGGAGGAAATCGACTACGACGACACCATCTCGGTGGAAATGAAGAAGGAA
GATTTCGATATCTACGACGAGGACGAAAATCAGTCCCCTCGCTCATTCCAAAAGAAAACTAGACACTACTTTA
TCGCCGCGGTGGAAAGACTGTGGGACTATGGAATGTCATCCAGCCCTCACGTCCTTCGGAACCGGGCCCAGAG
CGGATCGGTGCCTCAGTTCAAGAAAGTGGTGTTCCAGGAGTTCACCGACGGCAGCTTCACCCAGCCGCTGTAC
CGGGGAGAACTGAACGAACACCTGGGCCTGCTCGGTCCCTACATCCGCGCGGAAGTGGAGGATAACATCATGG
TGACCTTCCGTAACCAAGCATCCAGACCTTACTCCTTCTATTCCTCCCTGATCTCATACGAGGAGGACCAGCG
CCAAGGCGCCGAGCCCCGCAAGAACTTCGTCAAGCCCAACGAGACTAAGACCTACTTCTGGAAGGTCCAACAC
CATATGGCCCCGACCAAGGATGAGTTTGACTGCAAGGCCTGGGCCTACTTCTCCGACGTGGACCTTGAGAAGG
ATGTCCATTCCGGCCTGATCGGGCCGCTGCTCGTGTGTCACACCAACACCCTGAACCCAGCGCATGGACGCCA
GGTCACCGTCCAGGAGTTTGCTCTGTTCTTCACCATTTTTGACGAAACTAAGTCCTGGTACTTCACCGAGAAT
ATGGAGCGAAACTGTAGAGCGCCCTGCAATATCCAGATGGAAGATCCGACTTTCAAGGAGAACTATAGATTCC
ACGCCATCAACGGGTACATCATGGATACTCTGCCGGGGCTGGTCATGGCCCAGGATCAGAGGATTCGGTGGTA
CTTGCTGTCAATGGGATCGAACGAAAACATTCACTCCATTCACTTCTCCGGTCACGTGTTCACTGTGCGCAAG
AAGGAGGAGTACAAGATGGCGCTGTACAATCTGTACCCCGGGGTGTTCGAAACTGTGGAGATGCTGCCGTCCA
AGGCCGGCATCTGGAGAGTGGAGTGCCTGATCGGAGAGCACCTCCACGCGGGGATGTCCACCCTCTTCCTGGT
GTACTCGAATAAGTGCCAGACCCCGCTGGGCATGGCCTCGGGCCACATCAGAGACTTCCAGATCACAGCAAGC
GGACAATACGGCCAATGGGCGCCGAAGCTGGCCCGCTTGCACTACTCCGGATCGATCAACGCATGGTCCACCA
AGGAACCGTTCTCGTGGATTAAGGTGGACCTCCTGGCCCCTATGATTATCCACGGAATTAAGACCCAGGGCGC
CAGGCAGAAGTTCTCCTCCCTGTACATCTCGCAATTCATCATCATGTACAGCCTGGACGGGAAGAAGTGGCAG
ACTTACAGGGGAAACTCCACCGGCACCCTGATGGTCTTTTTCGGCAACGTGGATTCCTCCGGCATTAAGCACA
ACATCTTCAACCCACCGATCATAGCCAGATATATTAGGCTCCACCCCACTCACTACTCAATCCGCTCAACTCT
TCGGATGGAACTCATGGGGTGCGACCTGAACTCCTGCTCCATGCCGTTGGGGATGGAATCAAAGGCTATTAGC
GACGCCCAGATCACCGCGAGCTCCTACTTCACTAACATGTTCGCCACCTGGAGCCCCTCCAAGGCCAGGCTGC
ACTTGCAGGGACGGTCAAATGCCTGGCGGCCGCAAGTGAACAATCCGAAGGAATGGCTTCAAGTGGATTTCCA
AAAGACCATGAAAGTGACCGGAGTCACCACCCAGGGAGTGAAGTCCCTTCTGACCTCGATGTATGTGAAGGAG
TTCCTGATTAGCAGCAGCCAGGACGGGCACCAGTGGACCCTGTTCTTCCAAAACGGAAAGGTCAAGGTGTTCC
AGGGGAACCAGGACTCGTTCACACCCGTGGTGAACTCCCTGGACCCCCCACTGCTGACGCGGTACTTGAGGAT
TCATCCTCAGTCCTGGGTCCATCAGATTGCATTGCGAATGGAAGTCCTGGGCTGCGAGGCCCAGGACCTGTAC
TGAATCAGCCTGAGCTCGCTGATCATAATCAACCTCTGGATTACAAAATTTGTGAAAGATTGACTGGTATTCT
TAACTATGTTGCTCCTTTTACGCTATGTGGATACGCTGCTTTAATGCCTTTGTATCATGCTATTGCTTCCCGT
ATGGCTTTCATTTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGGGAAATCATCGTCCTTTCCTT
GGCTGCTCGCCTGTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTCGCCCTCAGACG
AGTCGGATCTCCCTTTGGGCCGCCTCCCCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTTCTGAGGCGGAAAGAACGGGC
TCGAGAAGCTTCTAGATATCCTCTCTTAAGGTAGCATCGAGATTTAAATTAGGGATAACAGGGTAATGGCGCG
GGCCGCAAAATTTAAAAGAAGACACCAAATCAGATGCCGCCGGTCGCCGCCGGTAGGCGGGACTTCCGGTACA
AGATGGCGGACAATTACGTCATTTCCTGTGACGTCATTTCCTGTGACGTCACTTCCGGTGGGCGGGACTTCCG
GAATTAGGGTTGGCTCTGGGCCAGCGCTTGGGGTTGACGTGCCACTAAGATCAAGCGGCGCGCCGCTTGTCTT
AGTGTCAAGGCAACCCCAAGCAAGCTGGCCCAGAGCCAACCCTAATTCCGGAAGTCCCGCCCACCGGAAGTGA
CGTCACAGGAAATGACGTCACAGGAAATGACGTAATTGTCCGCCATCTTGTACCGGAAGTCCCGCCTACCGGC
GGCGACCGGCGGCATCTGATTTGG

TABLE 2E
Example AAV-FVIII construct (nucleotides 1-6824; SEQ ID NO: 190)

| Description | Sequence |
|---|---|
| 5′ ITR (SEQ ID | CTGCGCGCTCGCTCGCTCACTGAGGCCGCCCGGGCAAAGCCCGGGCGTCGGGCGACCT |
| NO: 111) | TTGGTCGCCCGGCCTCAGTGAGCGAGCGAGCGCGCAGAGAGGGAGTGGCCAACTCCAT |
| | CACTAGGGGTTCCT |
| CAGp | TCGAGGTGAGCCCCACGTTCTGCTTCACTCTCCCCATCTCCCCCCCCTCCCCACCCCC |
| (ubiquitous | AATTTTGTATTTATTTATTTTTTAATTATTTTGTGCAGCGATGGGGGCGGGGGGGGGG |
| promoter) (SEQ | GGGGGGCGCGCGCCAGGCGGGGCGGGGCGGGGCGAGGGGCGGGGCGGGGCGAGGCGGA |
| ID NO: 191) | GAGGTGCGGCGGCAGCCAATCAGAGCGGCGCGCTCCGAAAGTTTCCTTTTATGGCGAG |
| | GCGGCGGCGGCGGCGGCCCTATAAAAAGCGAAGCGCGCGGCGGGCG |
| Synthetic Intron | GTGAGCGGGCGGGACGGCCCTTCTCCTTCGGGCTGTAATTAGCGCTTGGTTTAATGAC |
| (SEQ ID NO: 192) | GGCTTGTTTCTTTTCTGTGGCTGCGTGAAAGCCTTGAGGGGCTCCGGGAGGGCCCTTT |
| | GTGCGGGGGGAGCGGCTCGGGGCTGTCCGCGGGGGGACGGCTGCCTTCGGGGGGGACG |
| | GGGCAGGGCGGGGTTCGGCTTCTGGCGTGTGACCGGCGGCTCTAGAGCCTCTGCTAAC |
| | CATGTTCATGCCTTCTTCTTTTTCCTACAG |
| FVIIIco6XTEN | ATGCAGATTGAGCTGTCCACTTGTTTCTTCCTGTGCCTCCTGCGCTTCTGTTTCTCCG |
| (SEQ ID NO: 117) | CCACTCGCCGGTACTACCTTGGAGCCGTGGAGCTTTCATGGGACTACATGCAGAGCGA |
| (open reading | CCTGGGCGAACTCCCCGTGGATGCCAGATTCCCCCCCCGCGTGCCAAAGTCCTTCCCC |
| frame for codon- | TTTAACACCTCCGTGGTGTACAAGAAAACCCTCTTTGTCGAGTTCACTGACCACCTGT |
| optimized FVIII | TCAACATCGCCAAGCCGCGCCCACCTTGGATGGGCCTCCTGGGACCGACCATTCAAGC |
| version 6 | TGAAGTGTACGACACCGTGGTGATCACCCTGAAGAACATGGCGTCCCACCCCGTGTCC |
| containing | CTGCATGCGGTCGGAGTGTCCTACTGGAAGGCCTCCGAAGGAGCTGAGTACGACGACC |
| XTEN144; the | AGACTAGCCAGCGGGAAAAGGAGGACGATAAAGTGTTCCCGGGCGGCTCGCATACTTA |
| XTEN sequence | CGTGTGGCAAGTCCTGAAGGAAAACGGACCTATGGCATCCGATCCTCTGTGCCTGACT |
| is marked by | TACTCCTACCTTTCCCATGTGGACCTCGTGAAGGACCTGAACAGCGGGCTGATTGGTG |
| double | CACTTCTCGTGTGCCGCGAAGGTTCGCTCGCTAAGGAAAAGACCCAGACCCTCCATAA |
| underlining (SEQ | GTTCATCCTTTTGTTCGCTGTGTTCGATGAAGGAAAGTCATGGCATTCCGAAACTAAG |
| ID NO: 118)) | AACTCGCTGATGCAGGACCGGGATGCCGCCTCAGCCCGCGCCTGGCCTAAAATGCATA |
| | CAGTCAACGGATACGTGAATCGGTCACTGCCCGGGCTCATCGGTTGTCACAGAAAGTC |
| | CGTGTACTGGCACGTCATCGGCATGGGCACTACGCCTGAAGTGCACTCCATCTTCCTG |
| | GAAGGGCACACCTTCCTCGTGCGCAACCACCGCCAGGCCTCTCTGGAAATCTCCCCGA |
| | TTACCTTTCTGACCGCCCAGACTCTGCTCATGGACCTGGGGCAGTTCCTTCTCTTCTG |
| | CCACATCTCCAGCCATCAGCACGACGGAATGGAGGCCTACGTGAAGGTGGACTCATGC |
| | CCGGAAGAACCTCAGTTGCGGATGAAGAACAACGAGGAGGCCGAGGACTATGACGACG |
| | ATTTGACTGACTCCGAGATGGACGTCGTGCGGTTCGATGACGACAACAGCCCCAGCTT |
| | CATCCAGATTCGCAGCGTGGCCAAGAAGCACCCCAAAACCTGGGTGCACTACATCGCG |
| | GCCGAGGAAGAAGATTGGGACTACGCCCCGTTGGTGCTGGCACCCGATGACCGGTCGT |
| | ACAAGTCCCAGTATCTGAACAATGGTCCGCAGCGGATTGGCAGAAAGTACAAGAAAGT |
| | GCGGTTCATGGCGTACACTGACGAAACGTTTAAGACCCGGGAGGCCATTCAACATGAG |
| | AGCGGCATTCTGGGACCACTGCTGTACGGAGAGGTCGGCGATACCCTGCTCATCATCT |
| | TCAAAAACCAGGCCTCCCGGCCTTACAACATCTACCCTCACGGAATCACCGACGTGCG |
| | GCCACTCTACTCGCGGCGCCTGCCGAAGGGCGTCAAGCACCTGAAAGACTTCCCTATC |
| | CTGCCGGGCGAAATCTTCAAGTATAAGTGGACCGTCACCGTGGAGGACGGGCCCACCA |
| | AGAGCGATCCTAGGTGTCTGACTCGGTACTACTCCAGCTTCGTGAACATGGAACGGGA |
| | CCTGGCATCGGGACTCATTGGACCGCTGCTGATCTGCTACAAAGAGTCGGTGGATCAA |
| | CGCGGCAACCAGATCATGTCCGACAAGCGCAACGTGATCCTGTTCTCCGTGTTTGATG |
| | AAAACAGATCCTGGTACCTCACTGAAAACATCCAGAGGTTCCTCCCAAACCCCGCAGG |
| | AGTGCAACTGGAGGACCCTGAGTTTCAGGCCTCGAATATCATGCACTCGATTAACGGT |
| | TACGTGTTCGACTCGCTGCAACTGAGCGTGTGCCTCCATGAAGTCGCTTACTGGTACA |
| | TTCTGTCCATCGGCGCCCAGACTGACTTCCTGAGCGTGTTCTTTTCCGGTTACACCTT |
| | TAAGCACAAGATGGTGTACGAAGATACCCTGACCCTGTTCCCTTTCTCCGGCGAAACG |
| | GTGTTCATGTCGATGGAGAACCCGGGTCTGTGGATTCTGGGATGCCACAACAGCGACT |
| | TTCGGAACCGCGGAATGACTGCCCTGCTGAAGGTGTCCTCATGCGACAAGAACACCGG |
| | AGACTACTACGAGGACTCCTACGAGGATATCTCAGCCTACCTCCTGTCCAAGAACAAC |
| | GCGATCGAGCCGCGCAGCTTCAGCCAGAACGGCGCGCCAACATCAGAGAGCGCCACCC |
| | CTGAAAGTGGTCCCGGGAGCGAGCCAGCCACATCTGGGTCGGAAACGCCAGGCACAAG |
| | TGAGTCTGCAACTCCCGAGTCCGGACCTGGCTCCGAGCCTGCCACTAGCGGCTCCGAG |
| | ACTCCGGGAACTTCCGAGAGCGCTACACCAGAAAGCGGACCCGGAACCAGTACCGAAC |
| | CTAGCGAGGGCTCTGCTCCGGGCAGCCCAGCCGGCTCTCCTACATCCACGGAGGAGGG |
| | CACTTCCGAATCCGCCACCCCGGAGTCAGGGCCAGGATCTGAACCCGCTACCTCAGGC |
| | AGTGAGACGCCAGGAACGAGCGAGTCCGCTACACCGGAGAGTGGGCCAGGGAGCCCTG |
| | CTGGATCTCCTACGTCCACTGAGGAAGGGTCACCAGCGGGCTCGCCCACCAGCACTGA |
| | AGAAGGTGCCTCGAGCCCGCCTGTGCTGAAGAGGCACCAGCGAGAAATTACCCGGACC |
| | ACCCTCCAATCGGATCAGGAGGAAATCGACTACGACGACACCATCTCGGTGGAAATGA |
| | AGAAGGAAGATTTCGATATCTACGACGAGGACGAAAATCAGTCCCCTCGCTCATTCCA |
| | AAAGAAAACTAGACACTACTTTATCGCCGCGGTGGAAAGACTGTGGGACTATGGAATG |
| | TCATCCAGCCCTCACGTCCTTCGGAACCGGGCCCAGAGCGGATCGGTGCCTCAGTTCA |
| | AGAAAGTGGTGTTCCAGGAGTTCACCGACGGCAGCTTCACCCAGCCGCTGTACCGGGG |
| | AGAACTGAACGAACACCTGGGCCTGCTCGGTCCCTACATCCGCGCGGAAGTGGAGGAT |
| | AACATCATGGTGACCTTCCGTAACCAAGCATCCAGACCTTACTCCTTCTATTCCTCCC |
| | TGATCTCATACGAGGAGGACCAGCGCCAAGGCGCCGAGCCCCGCAAGAACTTCGTCAA |
| | GCCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGATG |
| | TCCATTCCGGCCTGATCGGGCCGCTGCTCGTGTGTCACACCAACACCCTGAACCCAGC |
| | GCATGGACGCCAGGTCACCGTCCAGGAGTTTGCTCTGTTCTTCACCATTTTTGACGAA |
| | ACTAAGTCCTGGTACTTCACCGAGAATATGGAGCGAAACTGTAGAGCGCCCTGCAATA |
| | TCCAGATGGAAGATCCGACTTTCAAGGAGAACTATAGATTCCACGCCATCAACGGGTA |
| | CATCATGGATACTCTGCCGGGGCTGGTCATGGCCCAGGATCAGAGGATTCGGTGGTAC |
| | TTGCTGTCAATGGGATCGAACGAAAACATTCACTCCATTCACTTCTCCGGTCACGTGT |
| | TCACTGTGCGCAAGAAGGAGGAGTACAAGATGGCGCTGTACAATCTGTACCCCGGGGT |
| | GTTCGAAACTGTGGAGATGCTGCCGTCCAAGGCCGGCATCTGGAGAGTGGAGTGCCTG |
| | ATCGGAGAGCACCTCCACGCGGGGATGTCCACCCTCTTCCTGGTGTACTCGAATAAGT |
| | GCCAGACCCCGCTGGGCATGGCCTCGGGCCACATCAGAGACTTCCAGATCACAGCAAG |
| | CGGACAATACGGCCAATGGGCGCCGAAGCTGGCCCGCTTGCACTACTCCGGATCGATC |
| | AACGCATGGTCCACCAAGGAACCGTTCTCGTGGATTAAGGTGGACCTCCTGGCCCCTA |
| | TGATTATCCACGGAATTAAGACCCAGGGCGCCAGGCAGAAGTTCTCCTCCCTGTACAT |
| | CTCGCAATTCATCATCATGTACAGCCTGGACGGGAAGAAGTGGCAGACTTACAGGGGA |
| | AACTCCACCGGCACCCTGATGGTCTTTTTCGGCAACGTGGATTCCTCCGGCATTAAGC |
| | ACAACATCTTCAACCCACCGATCATAGCCAGATATATTAGGCTCCACCCCACTCACTA |
| | CTCAATCCGCTCAACTCTTCGGATGGAACTCATGGGGTGCGACCTGAACTCCTGCTCC |
| | ATGCCGTTGGGGATGGAATCAAAGGCTATTAGCGACGCCCAGATCACCGCGAGCTCCT |
| | ACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGAT |
| | TTCCAAAAGACCATGAAAGTGACCGGAGTCACCACCCAGGGAGTGAAGTCCCTTCTGA |
| | CCTCGATGTATGTGAAGGAGTTCCTGATTAGCAGCAGCCAGGACGGGCACCAGTGGAC |
| | CCTGTTCTTCCAAAACGGAAAGGTCAAGGTGTTCCAGGGGAACCAGGACTCGTTCACA |
| | CCCGTGGTGAACTCCCTGGACCCCCCACTGCTGACGCGGTACTTGAGGATTCATCCTC |
| | AGTCCTGGGTCCATCAGATTGCATTGCGAATGGAAGTCCTGGGCTGCGAGGCCCAGGA |
| | CCTGTACTGA |
| WPRE (mutated | TCATAATCAACCTCTGGATTACAAAATTTGTGAAAGATTGACTGGTATTCTTAACTAT |
| woodchuck | GTTGCTCCTTTTACGCTATGTGGATACGCTGCTTTAATGCCTTTGTATCATGCTATTG |
| hepatitis virus | CTTCCCGTATGGCTTTCATTTTCTCCTCCTTGTATAAATCCTGGTTGCTGTCTCTTTA |
| post- | TGAGGAGTTGTGGCCCGTTGTCAGGCAACGTGGCGTGGTGTGCACTGTGTTTGCTGAC |
| transcriptional | GCAACCCCCACTGGTTGGGGCATTGCCACCACCTGTCAGCTCCTTTCCGGGACTTTCG |
| regulatory | CTTTCCCCCTCCCTATTGCCACGGCGGAACTCATCGCCGCCTGCCTTGCCCGCTGCTG |
| element) (SEQ | GACAGGGGCTCGGCTGTTGGGCACTGACAATTCCGTGGTGTTGTCGGGGAAATCATCG |
| ID NO: 120) | TCCTTTCCTTGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTTTGG |
| | GCCGCCTCCCCGCTG |
| bGHpA (bovine | CGACTGTGCCTTCTAGTTGCCAGCCATCTGTTGTTTGCCCCTCCCCCGTGCCTTCCTT |
| growth hormone | GACCCTGGAAGGTGCCACTCCCACTGTCCTTTCCTAATAAAATGAGGAAATTGCATCG |
| polyadenylation | CATTGTCTGAGTAGGTGTCATTCTATTCTGGGGGGTGGGGTGGGGCAGGACAGCAAGG |
| signal) (SEQ ID | GGGAGGATTGGGAAGACAATAGCAGGCATGCTGGGGA |
| NO: 122) | |
| 3′ ITR inverted | AGGAACCCCTAGTGATGGAGTTGGCCACTCCCTCTCTGCGCGCTCGCTCGCTCACTGA |
| terminal repeat | GGCCGGGCGACCAAAGGTCGCCCGACGCCCGGGCTTTGCCCGGGCGGCCTCAGTGAGC |
| (SEQ ID NO: | GAGCGAGCGCGCAG |
| 193) | |

Full-length Sequence (SEQ ID NO: 190)
CTGCGCGCTCGCTCGCTCACTGAGGCCGCCCGGGCAAAGCCCGGGCGTCGGGCGACCTTTGGTCGCCCGGCCT
CAGTGAGCGAGCGAGCGCGCAGAGAGGGAGTGGCCAACTCCATCACTAGGGGTTCCTGCGGCAATTCAGTCGA
TAACTATAACGGTCCTAAGGTAGCGATTTAAATACGCGCTCTCTTAAGGTAGCCCCGGGACGCGTCAATTGAG
ATCTGGATCCGGTACCGAATTCGCGGCCGCCTCGACGACTAGCGTTTAATTAAATCGAGGTGAGCCCCACGTT
CTGCTTCACTCTCCCCATCTCCCCCCCCTCCCCACCCCCAATTTTGTATTTATTTATTTTTTAATTATTTTGT
GCAGCGATGGGGGCGGGGGGGGGGGGGGGGCGCGCGCCAGGCGGGGCGGGGCGGGGCGAGGGGCGGGGCGGGG
CGAGGCGGAGAGGTGCGGCGGCAGCCAATCAGAGCGGCGCGCTCCGAAAGTTTCCTTTTATGGCGAGGCGGCG
GCGGCGGCGGCCCTATAAAAAGCGAAGCGCGCGGCGGGCGGGAGTCGCTGCGCGCTGCCTTCGCCCCGTGCCC
CGCTCCGCCGCCGCCTCGCGCCGCCCGCCCCGGCTCTGACTGACCGCGTTACTCCCACAGGTGAGCGGGCGGG
ACGGCCCTTCTCCTTCGGGCTGTAATTAGCGCTTGGTTTAATGACGGCTTGTTTCTTTTCTGTGGCTGCGTGA
AAGCCTTGAGGGGCTCCGGGAGGGCCCTTTGTGCGGGGGGAGCGGCTCGGGGCTGTCCGCGGGGGGACGGCTG
CCTTCGGGGGGGACGGGGCAGGGCGGGGTTCGGCTTCTGGCGTGTGACCGGCGGCTCTAGAGCCTCTGCTAAC
CATGTTCATGCCTTCTTCTTTTTCCTACAGGCTAGCGCCACCATGCAGATTGAGCTGTCCACTTGTTTCTTCC
TGTGCCTCCTGCGCTTCTGTTTCTCCGCCACTCGCCGGTACTACCTTGGAGCCGTGGAGCTTTCATGGGACTA
CATGCAGAGCGACCTGGGCGAACTCCCCGTGGATGCCAGATTCCCCCCCCGCGTGCCAAAGTCCTTCCCCTTT
AACACCTCCGTGGTGTACAAGAAAACCCTCTTTGTCGAGTTCACTGACCACCTGTTCAACATCGCCAAGCCGC
GCCCACCTTGGATGGGCCTCCTGGGACCGACCATTCAAGCTGAAGTGTACGACACCGTGGTGATCACCCTGAA
GAACATGGCGTCCCACCCCGTGTCCCTGCATGCGGTCGGAGTGTCCTACTGGAAGGCCTCCGAAGGAGCTGAG
TACGACGACCAGACTAGCCAGCGGGAAAAGGAGGACGATAAAGTGTTCCCGGGCGGCTCGCATACTTACGTGT
GGCAAGTCCTGAAGGAAAACGGACCTATGGCATCCGATCCTCTGTGCCTGACTTACTCCTACCTTTCCCATGT
GGACCTCGTGAAGGACCTGAACAGCGGGCTGATTGGTGCACTTCTCGTGTGCCGCGAAGGTTCGCTCGCTAAG
GAAAAGACCCAGACCCTCCATAAGTTCATCCTTTTGTTCGCTGTGTTCGATGAAGGAAAGTCATGGCATTCCG
AAACTAAGAACTCGCTGATGCAGGACCGGGATGCCGCCTCAGCCCGCGCCTGGCCTAAAATGCATACAGTCAA
CGGATACGTGAATCGGTCACTGCCCGGGCTCATCGGTTGTCACAGAAAGTCCGTGTACTGGCACGTCATCGGC
ATGGGCACTACGCCTGAAGTGCACTCCATCTTCCTGGAAGGGCACACCTTCCTCGTGCGCAACCACCGCCAGG
CCTCTCTGGAAATCTCCCCGATTACCTTTCTGACCGCCCAGACTCTGCTCATGGACCTGGGGCAGTTCCTTCT
CTTCTGCCACATCTCCAGCCATCAGCACGACGGAATGGAGGCCTACGTGAAGGTGGACTCATGCCCGGAAGAA
CCTCAGTTGCGGATGAAGAACAACGAGGAGGCCGAGGACTATGACGACGATTTGACTGACTCCGAGATGGACG
TCGTGCGGTTCGATGACGACAACAGCCCCAGCTTCATCCAGATTCGCAGCGTGGCCAAGAAGCACCCCAAAAC
CTGGGTGCACTACATCGCGGCCGAGGAAGAAGATTGGGACTACGCCCCGTTGGTGCTGGCACCCGATGACCGG
TCGTACAAGTCCCAGTATCTGAACAATGGTCCGCAGCGGATTGGCAGAAAGTACAAGAAAGTGCGGTTCATGG
CGTACACTGACGAAACGTTTAAGACCCGGGAGGCCATTCAACATGAGAGCGGCATTCTGGGACCACTGCTGTA
CGGAGAGGTCGGCGATACCCTGCTCATCATCTTCAAAAACCAGGCCTCCCGGCCTTACAACATCTACCCTCAC
GGAATCACCGACGTGCGGCCACTCTACTCGCGGCGCCTGCCGAAGGGCGTCAAGCACCTGAAAGACTTCCCTA
TCCTGCCGGGCGAAATCTTCAAGTATAAGTGGACCGTCACCGTGGAGGACGGGCCCACCAAGAGCGATCCTAG
GTGTCTGACTCGGTACTACTCCAGCTTCGTGAACATGGAACGGGACCTGGCATCGGGACTCATTGGACCGCTG
CTGATCTGCTACAAAGAGTCGGTGGATCAACGCGGCAACCAGATCATGTCCGACAAGCGCAACGTGATCCTGT
TCTCCGTGTTTGATGAAAACAGATCCTGGTACCTCACTGAAAACATCCAGAGGTTCCTCCCAAACCCCGCAGG
AGTGCAACTGGAGGACCCTGAGTTTCAGGCCTCGAATATCATGCACTCGATTAACGGTTACGTGTTCGACTCG
CTGCAACTGAGCGTGTGCCTCCATGAAGTCGCTTACTGGTACATTCTGTCCATCGGCGCCCAGACTGACTTCC
TGAGCGTGTTCTTTTCCGGTTACACCTTTAAGCACAAGATGGTGTACGAAGATACCCTGACCCTGTTCCCTTT
CTCCGGCGAAACGGTGTTCATGTCGATGGAGAACCCGGGTCTGTGGATTCTGGGATGCCACAACAGCGACTTT
CGGAACCGCGGAATGACTGCCCTGCTGAAGGTGTCCTCATGCGACAAGAACACCGGAGACTACTACGAGGACT
CCTACGAGGATATCTCAGCCTACCTCCTGTCCAAGAACAACGCGATCGAGCCGCGCAGCTTCAGCCAGAACGG
CGCGCCAACATCAGAGAGCGCCACCCCTGAAAGTGGTCCCGGGAGCGAGCCAGCCACATCTGGGTCGGAAACG
CCAGGCACAAGTGAGTCTGCAACTCCCGAGTCCGGACCTGGCTCCGAGCCTGCCACTAGCGGCTCCGAGACTC
CGGGAACTTCCGAGAGCGCTACACCAGAAAGCGGACCCGGAACCAGTACCGAACCTAGCGAGGGCTCTGCTCC
GGGCAGCCCAGCCGGCTCTCCTACATCCACGGAGGAGGGCACTTCCGAATCCGCCACCCCGGAGTCAGGGCCA
GGATCTGAACCCGCTACCTCAGGCAGTGAGACGCCAGGAACGAGCGAGTCCGCTACACCGGAGAGTGGGCCAG
GGAGCCCTGCTGGATCTCCTACGTCCACTGAGGAAGGGTCACCAGCGGGCTCGCCCACCAGCACTGAAGAAGG
TGCCTCGAGCCCGCCTGTGCTGAAGAGGCACCAGCGAGAAATTACCCGGACCACCCTCCAATCGGATCAGGAG
GAAATCGACTACGACGACACCATCTCGGTGGAAATGAAGAAGGAAGATTTCGATATCTACGACGAGGACGAAA
ATCAGTCCCCTCGCTCATTCCAAAAGAAAACTAGACACTACTTTATCGCCGCGGTGGAAAGACTGTGGGACTA
TGGAATGTCATCCAGCCCTCACGTCCTTCGGAACCGGGCCCAGAGCGGATCGGTGCCTCAGTTCAAGAAAGTG
GTGTTCCAGGAGTTCACCGACGGCAGCTTCACCCAGCCGCTGTACCGGGGAGAACTGAACGAACACCTGGGCC
TGCTCGGTCCCTACATCCGCGCGGAAGTGGAGGATAACATCATGGTGACCTTCCGTAACCAAGCATCCAGACC
TTACTCCTTCTATTCCTCCCTGATCTCATACGAGGAGGACCAGCGCCAAGGCGCCGAGCCCCGCAAGAACTTC
GTCAAGCCCAACGAGACTAAGACCTACTTCTGGAAGGTCCAACACCATATGGCCCCGACCAAGGATGAGTTTG
ACTGCAAGGCCTGGGCCTACTTCTCCGACGTGGACCTTGAGAAGGATGTCCATTCCGGCCTGATCGGGCCGCT
GCTCGTGTGTCACACCAACACCCTGAACCCAGCGCATGGACGCCAGGTCACCGTCCAGGAGTTTGCTCTGTTC
TTCACCATTTTTGACGAAACTAAGTCCTGGTACTTCACCGAGAATATGGAGCGAAACTGTAGAGCGCCCTGCA
ATATCCAGATGGAAGATCCGACTTTCAAGGAGAACTATAGATTCCACGCCATCAACGGGTACATCATGGATAC
TCTGCCGGGGCTGGTCATGGCCCAGGATCAGAGGATTCGGTGGTACTTGCTGTCAATGGGATCGAACGAAAAC
ATTCACTCCATTCACTTCTCCGGTCACGTGTTCACTGTGCGCAAGAAGGAGGAGTACAAGATGGCGCTGTACA
ATCTGTACCCCGGGGTGTTCGAAACTGTGGAGATGCTGCCGTCCAAGGCCGGCATCTGGAGAGTGGAGTGCCT
GATCGGAGAGCACCTCCACGCGGGGATGTCCACCCTCTTCCTGGTGTACTCGAATAAGTGCCAGACCCCGCTG
GGCATGGCCTCGGGCCACATCAGAGACTTCCAGATCACAGCAAGCGGACAATACGGCCAATGGGCGCCGAAGC
TGGCCCGCTTGCACTACTCCGGATCGATCAACGCATGGTCCACCAAGGAACCGTTCTCGTGGATTAAGGTGGA
CCTCCTGGCCCCTATGATTATCCACGGAATTAAGACCCAGGGCGCCAGGCAGAAGTTCTCCTCCCTGTACATC
TCGCAATTCATCATCATGTACAGCCTGGACGGGAAGAAGTGGCAGACTTACAGGGGAAACTCCACCGGCACCC
TGATGGTCTTTTTCGGCAACGTGGATTCCTCCGGCATTAAGCACAACATCTTCAACCCACCGATCATAGCCAG
ATATATTAGGCTCCACCCCACTCACTACTCAATCCGCTCAACTCTTCGGATGGAACTCATGGGGTGCGACCTG
AACTCCTGCTCCATGCCGTTGGGGATGGAATCAAAGGCTATTAGCGACGCCCAGATCACCGCGAGCTCCTACT
TCACTAACATGTTCGCCACCTGGAGCCCCTCCAAGGCCAGGCTGCACTTGCAGGGACGGTCAAATGCCTGGCG
GCCGCAAGTGAACAATCCGAAGGAATGGCTTCAAGTGGATTTCCAAAAGACCATGAAAGTGACCGGAGTCACC
ACCCAGGGAGTGAAGTCCCTTCTGACCTCGATGTATGTGAAGGAGTTCCTGATTAGCAGCAGCCAGGACGGGC
ACCAGTGGACCCTGTTCTTCCAAAACGGAAAGGTCAAGGTGTTCCAGGGGAACCAGGACTCGTTCACACCCGT
GGTGAACTCCCTGGACCCCCCACTGCTGACGCGGTACTTGAGGATTCATCCTCAGTCCTGGGTCCATCAGATT
GCATTGCGAATGGAAGTCCTGGGCTGCGAGGCCCAGGACCTGTACTGAATCAGCCTGAGCTCGCTGATCATAA
TCAACCTCTGGATTACAAAATTTGTGAAAGATTGACTGGTATTCTTAACTATGTTGCTCCTTTTACGCTATGT
GGATACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACTGTGTT
TGCTGACGCAACCCCCACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGGGCTCGGCTGTTGGGCA
CTGACAATTCCGTGGTGTTGTCGGGGAAATCATCGTCCTTTCCTTGGCTGCTCGCCTGTGTTGCCACCTGGAT
TCTGCGCGGGACGTCCTTCTGCTACGTCCCTTCGGCCCTCAATCCAGCGGACCTTCCTTCCCGCGGCCTGCTG
CCGGCTCTGCGGCCTCTTCCGCGTCTTCGCCTTCGCCCTCAGACGAGTCGGATCTCCCTTTGGGCCGCCTCCC
CGCTGATCAGCCTCGACTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGCAGGCATGCT
GGGGATGCGGTGGGCTCTATGGCTTCTGAGGCGGAAAGAACGGGCTCGAGAAGCTTCTAGATATCCTCTCTTA
AGGTAGCATCGAGATTTAAATTAGGGATAACAGGGTAATGGCGCGGGCCGCAGGAACCCCTAGTGATGGAGTT
GGCCACTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAG

TABLE 2F
Example GPV-FVIII construct bearing full length GPV ITRs (nucleotides
1-7154; SEQ ID NO: 194)

| Description | Sequence |
|---|---|
| 5′ ITR (SEQ ID | CTCATTGGAGGGTTCGTTCGTTCGAACCAGCCAATCAGGGGAGGGGGAAGTGACGCAA |
| NO: 187) | GTTCCGGTCACATGCTTCCGGTGACGCACATCCGGTGACGTAGTTCCGGTCACGTGCT |
| | TCCTGTCACGTGTTTCCGGTCACGTGACTTCCGGTCATGTGACTTCCGGTGACGTGTT |
| | TCCGGCTGTTAGGTTGACCACGCGCATGCCGCGCGGTCAGCCCAATAGTTAAGCCGGA |
| | AACACGTCACCGGAAGTCACATGACCGGAAGTCACGTGACCGGAAACACGTGACAGGA |
| | AGCACGTGACCGGAACTACGTCACCGGATGTGCGTCACCGGAAGCATGTGACCGGAAC |
| | TTGCGTCACTTCCCCCTCCCCTGATTGGCTGGTTCGAACGAACGAACCCTCCAATGAG |
| | ACTCAAGGACAAGAGGATATTTTGCGCGCCAGGAAGTG |
| TTPp (liver- | ACGCGTGTCTGTCTGCACATTTCGTAGAGCGAGTGTTCCGATACTCTAATCTCCCTAG |
| specific | GCAAGGTTCATATTTGTGTAGGTTACTTATTCTCCTTTTGTTGACTAAGTCAATAATC |
| promoter) (SEQ | AGAATCAGCAGGTTTGGAGTCAGCTTGGCAGGGATCAGCAGCCTGGGTTGGAAGGAGG |
| ID NO: 113) | GGGTATAAAAGCCCCTTCACCAGGAGAAGCCGTCACACAGATCCACAAGCTCCTG |
| Synthetic Intron | GTAAGTGCCGTGTGTGGTTCCCGCGGGCCTGGCCTCTTTACGGGTTATGGCCCTTGCG |
| (SEQ ID NO: 115) | TGCCTTGAATTACTGACACTGACATCCACTTTTTCTTTTTCTCCACAG |
| FVIIIco6XTEN | ATGCAGATTGAGCTGTCCACTTGTTTCTTCCTGTGCCTCCTGCGCTTCTGTTTCTCCG |
| (SEQ ID NO: 117) | CCACTCGCCGGTACTACCTTGGAGCCGTGGAGCTTTCATGGGACTACATGCAGAGCGA |
| (open reading | CCTGGGCGAACTCCCCGTGGATGCCAGATTCCCCCCCCGCGTGCCAAAGTCCTTCCCC |
| frame for codon- | TTTAACACCTCCGTGGTGTACAAGAAAACCCTCTTTGTCGAGTTCACTGACCACCTGT |
| optimized FVIII | TCAACATCGCCAAGCCGCGCCCACCTTGGATGGGCCTCCTGGGACCGACCATTCAAGC |
| version 6 | TGAAGTGTACGACACCGTGGTGATCACCCTGAAGAACATGGCGTCCCACCCCGTGTCC |
| containing | CTGCATGCGGTCGGAGTGTCCTACTGGAAGGCCTCCGAAGGAGCTGAGTACGACGACC |
| XTEN144; the | AGACTAGCCAGCGGGAAAAGGAGGACGATAAAGTGTTCCCGGGCGGCTCGCATACTTA |
| XTEN sequence | CGTGTGGCAAGTCCTGAAGGAAAACGGACCTATGGCATCCGATCCTCTGTGCCTGACT |
| is marked by | TACTCCTACCTTTCCCATGTGGACCTCGTGAAGGACCTGAACAGCGGGCTGATTGGTG |
| double | CACTTCTCGTGTGCCGCGAAGGTTCGCTCGCTAAGGAAAAGACCCAGACCCTCCATAA |
| underlining (SEQ | GTTCATCCTTTTGTTCGCTGTGTTCGATGAAGGAAAGTCATGGCATTCCGAAACTAAG |
| ID NO: 118)) | AACTCGCTGATGCAGGACCGGGATGCCGCCTCAGCCCGCGCCTGGCCTAAAATGCATA |
| | CAGTCAACGGATACGTGAATCGGTCACTGCCCGGGCTCATCGGTTGTCACAGAAAGTC |
| | CGTGTACTGGCACGTCATCGGCATGGGCACTACGCCTGAAGTGCACTCCATCTTCCTG |
| | GAAGGGCACACCTTCCTCGTGCGCAACCACCGCCAGGCCTCTCTGGAAATCTCCCCGA |
| | TTACCTTTCTGACCGCCCAGACTCTGCTCATGGACCTGGGGCAGTTCCTTCTCTTCTG |
| | CCACATCTCCAGCCATCAGCACGACGGAATGGAGGCCTACGTGAAGGTGGACTCATGC |
| | CCGGAAGAACCTCAGTTGCGGATGAAGAACAACGAGGAGGCCGAGGACTATGACGACG |
| | ATTTGACTGACTCCGAGATGGACGTCGTGCGGTTCGATGACGACAACAGCCCCAGCTT |
| | CATCCAGATTCGCAGCGTGGCCAAGAAGCACCCCAAAACCTGGGTGCACTACATCGCG |
| | GCCGAGGAAGAAGATTGGGACTACGCCCCGTTGGTGCTGGCACCCGATGACCGGTCGT |
| | ACAAGTCCCAGTATCTGAACAATGGTCCGCAGCGGATTGGCAGAAAGTACAAGAAAGT |
| | GCGGTTCATGGCGTACACTGACGAAACGTTTAAGACCCGGGAGGCCATTCAACATGAG |
| | AGCGGCATTCTGGGACCACTGCTGTACGGAGAGGTCGGCGATACCCTGCTCATCATCT |
| | TCAAAAACCAGGCCTCCCGGCCTTACAACATCTACCCTCACGGAATCACCGACGTGCG |
| | GCCACTCTACTCGCGGCGCCTGCCGAAGGGCGTCAAGCACCTGAAAGACTTCCCTATC |
| | CTGCCGGGCGAAATCTTCAAGTATAAGTGGACCGTCACCGTGGAGGACGGGCCCACCA |
| | AGAGCGATCCTAGGTGTCTGACTCGGTACTACTCCAGCTTCGTGAACATGGAACGGGA |
| | CCTGGCATCGGGACTCATTGGACCGCTGCTGATCTGCTACAAAGAGTCGGTGGATCAA |
| | CGCGGCAACCAGATCATGTCCGACAAGCGCAACGTGATCCTGTTCTCCGTGTTTGATG |
| | AAAACAGATCCTGGTACCTCACTGAAAACATCCAGAGGTTCCTCCCAAACCCCGCAGG |
| | AGTGCAACTGGAGGACCCTGAGTTTCAGGCCTCGAATATCATGCACTCGATTAACGGT |
| | TACGTGTTCGACTCGCTGCAACTGAGCGTGTGCCTCCATGAAGTCGCTTACTGGTACA |
| | TTCTGTCCATCGGCGCCCAGACTGACTTCCTGAGCGTGTTCTTTTCCGGTTACACCTT |
| | TAAGCACAAGATGGTGTACGAAGATACCCTGACCCTGTTCCCTTTCTCCGGCGAAACG |
| | GTGTTCATGTCGATGGAGAACCCGGGTCTGTGGATTCTGGGATGCCACAACAGCGACT |
| | TTCGGAACCGCGGAATGACTGCCCTGCTGAAGGTGTCCTCATGCGACAAGAACACCGG |
| | AGACTACTACGAGGACTCCTACGAGGATATCTCAGCCTACCTCCTGTCCAAGAACAAC |
| | GCGATCGAGCCGCGCAGCTTCAGCCAGAACGGCGCGCCAACATCAGAGAGCGCCACCC |
| | CTGAAAGTGGTCCCGGGAGCGAGCCAGCCACATCTGGGTCGGAAACGCCAGGCACAAG |
| | TGAGTCTGCAACTCCCGAGTCCGGACCTGGCTCCGAGCCTGCCACTAGCGGCTCCGAG |
| | ACTCCGGGAACTTCCGAGAGCGCTACACCAGAAAGCGGACCCGGAACCAGTACCGAAC |
| | CTAGCGAGGGCTCTGCTCCGGGCAGCCCAGCCGGCTCTCCTACATCCACGGAGGAGGG |
| | CACTTCCGAATCCGCCACCCCGGAGTCAGGGCCAGGATCTGAACCCGCTACCTCAGGC |
| | AGTGAGACGCCAGGAACGAGCGAGTCCGCTACACCGGAGAGTGGGCCAGGGAGCCCTG |
| | CTGGATCTCCTACGTCCACTGAGGAAGGGTCACCAGCGGGCTCGCCCACCAGCACTGA |
| | AGAAGGTGCCTCGAGCCCGCCTGTGCTGAAGAGGCACCAGCGAGAAATTACCCGGACC |
| | ACCCTCCAATCGGATCAGGAGGAAATCGACTACGACGACACCATCTCGGTGGAAATGA |
| | AGAAGGAAGATTTCGATATCTACGACGAGGACGAAAATCAGTCCCCTCGCTCATTCCA |
| | AAAGAAAACTAGACACTACTTTATCGCCGCGGTGGAAAGACTGTGGGACTATGGAATG |
| | TCATCCAGCCCTCACGTCCTTCGGAACCGGGCCCAGAGCGGATCGGTGCCTCAGTTCA |
| | AGAAAGTGGTGTTCCAGGAGTTCACCGACGGCAGCTTCACCCAGCCGCTGTACCGGGG |
| | AGAACTGAACGAACACCTGGGCCTGCTCGGTCCCTACATCCGCGCGGAAGTGGAGGAT |
| | AACATCATGGTGACCTTCCGTAACCAAGCATCCAGACCTTACTCCTTCTATTCCTCCC |
| | TGATCTCATACGAGGAGGACCAGCGCCAAGGCGCCGAGCCCCGCAAGAACTTCGTCAA |
| | GCCCAACGAGACTAAGACCTACTTCTGGAAGGTCCAACACCATATGGCCCCGACCAAG |
| | GATGAGTTTGACTGCAAGGCCTGGGCCTACTTCTCCGACGTGGACCTTGAGAAGGATG |
| | TCCATTCCGGCCTGATCGGGCCGCTGCTCGTGTGTCACACCAACACCCTGAACCCAGC |
| | GCATGGACGCCAGGTCACCGTCCAGGAGTTTGCTCTGTTCTTCACCATTTTTGACGAA |
| | ACTAAGTCCTGGTACTTCACCGAGAATATGGAGCGAAACTGTAGAGCGCCCTGCAATA |
| | TCCAGATGGAAGATCCGACTTTCAAGGAGAACTATAGATTCCACGCCATCAACGGGTA |
| | CATCATGGATACTCTGCCGGGGCTGGTCATGGCCCAGGATCAGAGGATTCGGTGGTAC |
| | TTGCTGTCAATGGGATCGAACGAAAACATTCACTCCATTCACTTCTCCGGTCACGTGT |
| | TCACTGTGCGCAAGAAGGAGGAGTACAAGATGGCGCTGTACAATCTGTACCCCGGGGT |
| | GTTCGAAACTGTGGAGATGCTGCCGTCCAAGGCCGGCATCTGGAGAGTGGAGTGCCTG |
| | ATCGGAGAGCACCTCCACGCGGGGATGTCCACCCTCTTCCTGGTGTACTCGAATAAGT |
| | GCCAGACCCCGCTGGGCATGGCCTCGGGCCACATCAGAGACTTCCAGATCACAGCAAG |
| | CGGACAATACGGCCAATGGGCGCCGAAGCTGGCCCGCTTGCACTACTCCGGATCGATC |
| | AACGCATGGTCCACCAAGGAACCGTTCTCGTGGATTAAGGTGGACCTCCTGGCCCCTA |
| | TGATTATCCACGGAATTAAGACCCAGGGCGCCAGGCAGAAGTTCTCCTCCCTGTACAT |
| | CTCGCAATTCATCATCATGTACAGCCTGGACGGGAAGAAGTGGCAGACTTACAGGGGA |
| | AACTCCACCGGCACCCTGATGGTCTTTTTCGGCAACGTGGATTCCTCCGGCATTAAGC |
| | ACAACATCTTCAACCCACCGATCATAGCCAGATATATTAGGCTCCACCCCACTCACTA |
| | CTCAATCCGCTCAACTCTTCGGATGGAACTCATGGGGTGCGACCTGAACTCCTGCTCC |
| | ATGCCGTTGGGGATGGAATCAAAGGCTATTAGCGACGCCCAGATCACCGCGAGCTCCT |
| | ACTTCACTAACATGTTCGCCACCTGGAGCCCCTCCAAGGCCAGGCTGCACTTGCAGGG |
| | ACGGTCAAATGCCTGGCGGCCGCAAGTGAACAATCCGAAGGAATGGCTTCAAGTGGAT |
| | TTCCAAAAGACCATGAAAGTGACCGGAGTCACCACCCAGGGAGTGAAGTCCCTTCTGA |
| | CCTCGATGTATGTGAAGGAGTTCCTGATTAGCAGCAGCCAGGACGGGCACCAGTGGAC |
| | CCTGTTCTTCCAAAACGGAAAGGTCAAGGTGTTCCAGGGGAACCAGGACTCGTTCACA |
| | CCCGTGGTGAACTCCCTGGACCCCCCACTGCTGACGCGGTACTTGAGGATTCATCCTC |
| | AGTCCTGGGTCCATCAGATTGCATTGCGAATGGAAGTCCTGGGCTGCGAGGCCCAGGA |
| | CCTGTACTGA |
| WPRE (mutated | TCATAATCAACCTCTGGATTACAAAATTTGTGAAAGATTGACTGGTATTCTTAACTAT |
| woodchuck | GTTGCTCCTTTTACGCTATGTGGATACGCTGCTTTAATGCCTTTGTATCATGCTATTG |
| hepatitis virus | CTTCCCGTATGGCTTTCATTTTCTCCTCCTTGTATAAATCCTGGTTGCTGTCTCTTTA |
| post- | TGAGGAGTTGTGGCCCGTTGTCAGGCAACGTGGCGTGGTGTGCACTGTGTTTGCTGAC |
| transcriptional | GCAACCCCCACTGGTTGGGGCATTGCCACCACCTGTCAGCTCCTTTCCGGGACTTTCG |
| regulatory | CTTTCCCCCTCCCTATTGCCACGGCGGAACTCATCGCCGCCTGCCTTGCCCGCTGCTG |
| element) (SEQ | GACAGGGGCTCGGCTGTTGGGCACTGACAATTCCGTGGTGTTGTCGGGGAAATCATCG |
| ID NO: 120) | TCCTTTCCTTGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTTTGG |
| | GCCGCCTCCCCGCTG |
| bGHpA (bovine | CGACTGTGCCTTCTAGTTGCCAGCCATCTGTTGTTTGCCCCTCCCCCGTGCCTTCCTT |
| growth hormone | GACCCTGGAAGGTGCCACTCCCACTGTCCTTTCCTAATAAAATGAGGAAATTGCATCG |
| polyadenylation | CATTGTCTGAGTAGGTGTCATTCTATTCTGGGGGGTGGGGTGGGGCAGGACAGCAAGG |
| signal) (SEQ ID | GGGAGGATTGGGAAGACAATAGCAGGCATGCTGGGGA |
| NO: 122) | |
| 3′ ITR inverted | CACTTCCTGGCGCGCAAAATATCCTCTTGTCCTTGAGTCTCATTGGAGGGTTCGTTCG |
| terminal repeat | TTCGAACCAGCCAATCAGGGGAGGGGGAAGTGACGCAAGTTCCGGTCACATGCTTCCG |
| (SEQ ID NO: | GTGACGCACATCCGGTGACGTAGTTCCGGTCACGTGCTTCCTGTCACGTGTTTCCGGT |
| 188) | CACGTGACTTCCGGTCATGTGACTTCCGGTGACGTGTTTCCGGCTTAACTATTGGGCT |
| | GACCGCGCGGCATGCGCGTGGTCAACCTAACAGCCGGAAACACGTCACCGGAAGTCAC |
| | ATGACCGGAAGTCACGTGACCGGAAACACGTGACAGGAAGCACGTGACCGGAACTACG |
| | TCACCGGATGTGCGTCACCGGAAGCATGTGACCGGAACTTGCGTCACTTCCCCCTCCC |
| | CTGATTGGCTGGTTCGAACGAACGAACCCTCCAATGAG |

Full-length Sequence (SEQ ID NO: 194)
CTCATTGGAGGGTTCGTTCGTTCGAACCAGCCAATCAGGGGAGGGGGAAGTGACGCAAGTTCCGGTCACATGC
TTCCGGTGACGCACATCCGGTGACGTAGTTCCGGTCACGTGCTTCCTGTCACGTGTTTCCGGTCACGTGACTT
CCGGTCATGTGACTTCCGGTGACGTGTTTCCGGCTGTTAGGTTGACCACGCGCATGCCGCGCGGTCAGCCCAA
TAGTTAAGCCGGAAACACGTCACCGGAAGTCACATGACCGGAAGTCACGTGACCGGAAACACGTGACAGGAAG
CACGTGACCGGAACTACGTCACCGGATGTGCGTCACCGGAAGCATGTGACCGGAACTTGCGTCACTTCCCCCT
CCCCTGATTGGCTGGTTCGAACGAACGAACCCTCCAATGAGACTCAAGGACAAGAGGATATTTTGCGCGCCAG
GAAGTGGCGGCAATTCAGTCGATAACTATAACGGTCCTAAGGTAGCGATTTAAATACGCGCTCTCTTAAGGTA
GCCCCGGGACGCGTCAATTGAGATCTGGATCCGGTACCGAATTCGCGGCCGCCTCGACGACTAGCGTTTAATT
AAACGCGTGTCTGTCTGCACATTTCGTAGAGCGAGTGTTCCGATACTCTAATCTCCCTAGGCAAGGTTCATAT
TTGTGTAGGTTACTTATTCTCCTTTTGTTGACTAAGTCAATAATCAGAATCAGCAGGTTTGGAGTCAGCTTGG
CAGGGATCAGCAGCCTGGGTTGGAAGGAGGGGGTATAAAAGCCCCTTCACCAGGAGAAGCCGTCACACAGATC
CACAAGCTCCTGAGGTAAGTGCCGTGTGTGGTTCCCGCGGGCCTGGCCTCTTTACGGGTTATGGCCCTTGCGT
GCCTTGAATTACTGACACTGACATCCACTTTTTCTTTTTCTCCACAGCTAGCGCCACCATGCAGATTGAGCTG
TCCACTTGTTTCTTCCTGTGCCTCCTGCGCTTCTGTTTCTCCGCCACTCGCCGGTACTACCTTGGAGCCGTGG
AGCTTTCATGGGACTACATGCAGAGCGACCTGGGCGAACTCCCCGTGGATGCCAGATTCCCCCCCCGCGTGCC
AAAGTCCTTCCCCTTTAACACCTCCGTGGTGTACAAGAAAACCCTCTTTGTCGAGTTCACTGACCACCTGTTC
AACATCGCCAAGCCGCGCCCACCTTGGATGGGCCTCCTGGGACCGACCATTCAAGCTGAAGTGTACGACACCG
TGGTGATCACCCTGAAGAACATGGCGTCCCACCCCGTGTCCCTGCATGCGGTCGGAGTGTCCTACTGGAAGGC
CTCCGAAGGAGCTGAGTACGACGACCAGACTAGCCAGCGGGAAAAGGAGGACGATAAAGTGTTCCCGGGCGGC
TCGCATACTTACGTGTGGCAAGTCCTGAAGGAAAACGGACCTATGGCATCCGATCCTCTGTGCCTGACTTACT
CCTACCTTTCCCATGTGGACCTCGTGAAGGACCTGAACAGCGGGCTGATTGGTGCACTTCTCGTGTGCCGCGA
AGGTTCGCTCGCTAAGGAAAAGACCCAGACCCTCCATAAGTTCATCCTTTTGTTCGCTGTGTTCGATGAAGGA
AAGTCATGGCATTCCGAAACTAAGAACTCGCTGATGCAGGACCGGGATGCCGCCTCAGCCCGCGCCTGGCCTA
AAATGCATACAGTCAACGGATACGTGAATCGGTCACTGCCCGGGCTCATCGGTTGTCACAGAAAGTCCGTGTA
CTGGCACGTCATCGGCATGGGCACTACGCCTGAAGTGCACTCCATCTTCCTGGAAGGGCACACCTTCCTCGTG
CGCAACCACCGCCAGGCCTCTCTGGAAATCTCCCCGATTACCTTTCTGACCGCCCAGACTCTGCTCATGGACC
TGGGGCAGTTCCTTCTCTTCTGCCACATCTCCAGCCATCAGCACGACGGAATGGAGGCCTACGTGAAGGTGGA
CTCATGCCCGGAAGAACCTCAGTTGCGGATGAAGAACAACGAGGAGGCCGAGGACTATGACGACGATTTGACT
GACTCCGAGATGGACGTCGTGCGGTTCGATGACGACAACAGCCCCAGCTTCATCCAGATTCGCAGCGTGGCCA
AGAAGCACCCCAAAACCTGGGTGCACTACATCGCGGCCGAGGAAGAAGATTGGGACTACGCCCCGTTGGTGCT
GGCACCCGATGACCGGTCGTACAAGTCCCAGTATCTGAACAATGGTCCGCAGCGGATTGGCAGAAAGTACAAG
AAAGTGCGGTTCATGGCGTACACTGACGAAACGTTTAAGACCCGGGAGGCCATTCAACATGAGAGCGGCATTC
TGGGACCACTGCTGTACGGAGAGGTCGGCGATACCCTGCTCATCATCTTCAAAAACCAGGCCTCCCGGCCTTA
CAACATCTACCCTCACGGAATCACCGACGTGCGGCCACTCTACTCGCGGCGCCTGCCGAAGGGCGTCAAGCAC
CTGAAAGACTTCCCTATCCTGCCGGGCGAAATCTTCAAGTATAAGTGGACCGTCACCGTGGAGGACGGGCCCA
CCAAGAGCGATCCTAGGTGTCTGACTCGGTACTACTCCAGCTTCGTGAACATGGAACGGGACCTGGCATCGGG
ACTCATTGGACCGCTGCTGATCTGCTACAAAGAGTCGGTGGATCAACGCGGCAACCAGATCATGTCCGACAAG
CGCAACGTGATCCTGTTCTCCGTGTTTGATGAAAACAGATCCTGGTACCTCACTGAAAACATCCAGAGGTTCC
TCCCAAACCCCGCAGGAGTGCAACTGGAGGACCCTGAGTTTCAGGCCTCGAATATCATGCACTCGATTAACGG
TTACGTGTTCGACTCGCTGCAACTGAGCGTGTGCCTCCATGAAGTCGCTTACTGGTACATTCTGTCCATCGGC
GCCCAGACTGACTTCCTGAGCGTGTTCTTTTCCGGTTACACCTTTAAGCACAAGATGGTGTACGAAGATACCC
TGACCCTGTTCCCTTTCTCCGGCGAAACGGTGTTCATGTCGATGGAGAACCCGGGTCTGTGGATTCTGGGATG
CCACAACAGCGACTTTCGGAACCGCGGAATGACTGCCCTGCTGAAGGTGTCCTCATGCGACAAGAACACCGGA
GACTACTACGAGGACTCCTACGAGGATATCTCAGCCTACCTCCTGTCCAAGAACAACGCGATCGAGCCGCGCA
GCTTCAGCCAGAACGGCGCGCCAACATCAGAGAGCGCCACCCCTGAAAGTGGTCCCGGGAGCGAGCCAGCCAC
ATCTGGGTCGGAAACGCCAGGCACAAGTGAGTCTGCAACTCCCGAGTCCGGACCTGGCTCCGAGCCTGCCACT
AGCGGCTCCGAGACTCCGGGAACTTCCGAGAGCGCTACACCAGAAAGCGGACCCGGAACCAGTACCGAACCTA
GCGAGGGCTCTGCTCCGGGCAGCCCAGCCGGCTCTCCTACATCCACGGAGGAGGGCACTTCCGAATCCGCCAC
CCCGGAGTCAGGGCCAGGATCTGAACCCGCTACCTCAGGCAGTGAGACGCCAGGAACGAGCGAGTCCGCTACA
CCGGAGAGTGGGCCAGGGAGCCCTGCTGGATCTCCTACGTCCACTGAGGAAGGGTCACCAGCGGGCTCGCCCA
CCAGCACTGAAGAAGGTGCCTCGAGCCCGCCTGTGCTGAAGAGGCACCAGCGAGAAATTACCCGGACCACCCT
CCAATCGGATCAGGAGGAAATCGACTACGACGACACCATCTCGGTGGAAATGAAGAAGGAAGATTTCGATATC
TACGACGAGGACGAAAATCAGTCCCCTCGCTCATTCCAAAAGAAAACTAGACACTACTTTATCGCCGCGGTGG
AAAGACTGTGGGACTATGGAATGTCATCCAGCCCTCACGTCCTTCGGAACCGGGCCCAGAGCGGATCGGTGCC
TCAGTTCAAGAAAGTGGTGTTCCAGGAGTTCACCGACGGCAGCTTCACCCAGCCGCTGTACCGGGGAGAACTG
AACGAACACCTGGGCCTGCTCGGTCCCTACATCCGCGCGGAAGTGGAGGATAACATCATGGTGACCTTCCGTA
ACCAAGCATCCAGACCTTACTCCTTCTATTCCTCCCTGATCTCATACGAGGAGGACCAGCGCCAAGGCGCCGA
GCCCCGCAAGAACTTCGTCAAGCCCAACGAGACTAAGACCTACTTCTGGAAGGTCCAACACCATATGGCCCCG
ACCAAGGATGAGTTTGACTGCAAGGCCTGGGCCTACTTCTCCGACGTGGACCTTGAGAAGGATGTCCATTCCG
GCCTGATCGGGCCGCTGCTCGTGTGTCACACCAACACCCTGAACCCAGCGCATGGACGCCAGGTCACCGTCCA
GGAGTTTGCTCTGTTCTTCACCATTTTTGACGAAACTAAGTCCTGGTACTTCACCGAGAATATGGAGCGAAAC
TGTAGAGCGCCCTGCAATATCCAGATGGAAGATCCGACTTTCAAGGAGAACTATAGATTCCACGCCATCAACG
GGTACATCATGGATACTCTGCCGGGGCTGGTCATGGCCCAGGATCAGAGGATTCGGTGGTACTTGCTGTCAAT
GGGATCGAACGAAAACATTCACTCCATTCACTTCTCCGGTCACGTGTTCACTGTGCGCAAGAAGGAGGAGTAC
AAGATGGCGCTGTACAATCTGTACCCCGGGGTGTTCGAAACTGTGGAGATGCTGCCGTCCAAGGCCGGCATCT
GGAGAGTGGAGTGCCTGATCGGAGAGCACCTCCACGCGGGGATGTCCACCCTCTTCCTGGTGTACTCGAATAA
GTGCCAGACCCCGCTGGGCATGGCCTCGGGCCACATCAGAGACTTCCAGATCACAGCAAGCGGACAATACGGC
CAATGGGCGCCGAAGCTGGCCCGCTTGCACTACTCCGGATCGATCAACGCATGGTCCACCAAGGAACCGTTCT
CGTGGATTAAGGTGGACCTCCTGGCCCCTATGATTATCCACGGAATTAAGACCCAGGGCGCCAGGCAGAAGTT
CTCCTCCCTGTACATCTCGCAATTCATCATCATGTACAGCCTGGACGGGAAGAAGTGGCAGACTTACAGGGGA
AACTCCACCGGCACCCTGATGGTCTTTTTCGGCAACGTGGATTCCTCCGGCATTAAGCACAACATCTTCAACC
CACCGATCATAGCCAGATATATTAGGCTCCACCCCACTCACTACTCAATCCGCTCAACTCTTCGGATGGAACT
CATGGGGTGCGACCTGAACTCCTGCTCCATGCCGTTGGGGATGGAATCAAAGGCTATTAGCGACGCCCAGATC
ACCGCGAGCTCCTACTTCACTAACATGTTCGCCACCTGGAGCCCCTCCAAGGCCAGGCTGCACTTGCAGGGAC
GGTCAAATGCCTGGCGGCCGCAAGTGAACAATCCGAAGGAATGGCTTCAAGTGGATTTCCAAAAGACCATGAA
AGTGACCGGAGTCACCACCCAGGGAGTGAAGTCCCTTCTGACCTCGATGTATGTGAAGGAGTTCCTGATTAGC
AGCAGCCAGGACGGGCACCAGTGGACCCTGTTCTTCCAAAACGGAAAGGTCAAGGTGTTCCAGGGGAACCAGG
ACTCGTTCACACCCGTGGTGAACTCCCTGGACCCCCCACTGCTGACGCGGTACTTGAGGATTCATCCTCAGTC
CTGGGTCCATCAGATTGCATTGCGAATGGAAGTCCTGGGCTGCGAGGCCCAGGACCTGTACTGAATCAGCCTG
AGCTCGCTGATCATAATCAACCTCTGGATTACAAAATTTGTGAAAGATTGACTGGTATTCTTAACTATGTTGC
TCCTTTTACGCTATGTGGATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTTCCTTGGCTGCTCGCCT
GTGTTGCCACCTGGATTCTGCGCGGGACGTCCTTCTGCTACGTCCCTTCGGCCCTCAATCCAGCGGACCTTCC
TTCCCGCGGCCTGCTGCCGGCTCTGCGGCCTCTTCCGCGTCTTCGCCTTCGCCCTCAGACGAGTCGGATCTCC
CTTTGGGCCGCCTCCCCGCTGATCAGCCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGATTGGGAAGA
CAATAGCAGGCATGCTGGGGATGCGGTGGGCTCTATGGCTTCTGAGGCGGAAAGAACGGGCTCGAGAAGCTTC
TAGATATCCTCTCTTAAGGTAGCATCGAGATTTAAATTAGGGATAACAGGGTAATGGCGCGGGCCGCCACTTC
CTGGCGCGCAAAATATCCTCTTGTCCTTGAGTCTCATTGGAGGGTTCGTTCGTTCGAACCAGCCAATCAGGGG
AGGGGGAAGTGACGCAAGTTCCGGTCACATGCTTCCGGTGACGCACATCCGGTGACGTAGTTCCGGTCACGTG
CTTCCTGTCACGTGTTTCCGGTCACGTGACTTCCGGTCATGTGACTTCCGGTGACGTGTTTCCGGCTTAACTA
TTGGGCTGACCGCGCGGCATGCGCGTGGTCAACCTAACAGCCGGAAACACGTCACCGGAAGTCACATGACCGG
AAGTCACGTGACCGGAAACACGTGACAGGAAGCACGTGACCGGAACTACGTCACCGGATGTGCGTCACCGGAA
GCATGTGACCGGAACTTGCGTCACTTCCCCCTCCCCTGATTGGCTGGTTCGAACGAACGAACCCTCCAATGAG

In one embodiment, the genetic cassette comprises a phenylalanine hydroxylase (PAH) construct, which includes a polynucleotide sequence as listed in Tables 10A and 10B. In one embodiment, the genetic cassette comprises a PAH construct, which includes a polynucleotide sequence set forth in Table 10A. In one embodiment, the genetic cassette comprises a PAH construct, which includes a polynucleotide sequence set forth in Table 10B.

In certain embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 197 or 198. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 197. In some embodiments, the isolated nucleic acid molecule comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence of SEQ ID NO: 198. In some embodiments, the isolated nucleic acid molecule retains the ability to express a functional phenylalanine hydroxylase.

### A. Codon Adaptation Index

In one embodiment, the genetic cassette comprises a codon optimized nucleotide sequence encoding a FVIII polypeptide, wherein the human codon adaptation index of the codon optimized nucleotide sequence is increased relative to SEQ ID NO: 16. For example, the codon optimized nucleotide sequence can have a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), at least about 0.88 (88%), at least about 0.89 (89%), at least about 0.90 (90%), at least about 0.91 (91%), at least about 0.92 (92%), at least about 0.93 (93%), at least about 0.94 (94%), at least about 0.95 (95%), at least about 0.96 (96%), at least about 0.97 (97%), at least about 0.98 (98%), or at least about 0.99 (99%). In some embodiments, the codon optimized nucleotide sequence has a human codon adaptation index that is at least about 0.88 (88%). In other embodiments, the codon optimized nucleotide sequence has a human codon adaptation index that is at least about 0.91 (91%). In other embodiments, the codon optimized nucleotide sequence has a human codon adaptation index that is at least about 0.91 (97%).

In one particular embodiment, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), at least about 0.88 (88%), at least about 0.89 (89%), at least about 0.90 (90%), or at least about 0.91 (91%). In one particular embodiment, the nucleotide sequence has a human codon adaptation index that is at least about 0.88 (88%). In another embodiment, the nucleotide sequence has a human codon adaptation index that is at least about 0.91 (91%).

In another embodiment, the codon optimized nucleotide sequence encoding a FVIII polypeptide comprises a nucleotide sequence which comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-2277 and 2320-4374 of SEQ ID NO: 5 or (ii) 1792-2277 and 2320-4374 of SEQ ID NO: 6; wherein the N-terminal portion and the C-terminal portion together have a FVIII polypeptide activity; and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), or at least about 0.88 (88%). In one particular embodiment, the nucleotide sequence has a human codon adaptation index that is at least about 0.83 (83%). In another embodiment, the nucleotide sequence has a human codon adaptation index that is at least about 0.88 (88%).

In other embodiments, the codon optimized nucleotide sequence encoding a FVIII polypeptide comprises a nucleotide sequence encoding a polypeptide with FVIII activity, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the human codon adaptation index of the nucleotide sequence is increased relative to SEQ ID NO: 16. In some embodiments, the nucleotide sequence has a human codon adaptation index that is at least about 0.75 (75%), at least about 0.76 (76%), at least about 0.77 (77%), at least about 0.78 (78%), at least about 0.79 (79%), at least about 0.80 (80%), at least about 0.81 (81%), at least about 0.82 (82%), at least about 0.83 (83%), at least about 0.84 (84%), at least about 0.85 (85%), at least about 0.86 (86%), at least about 0.87 (87%), or at least about 0.88 (88%). In one particular embodiment, the nucleotide sequence has a human codon adaptation index that is at least about 0.75 (75%). In another embodiment, the nucleotide sequence has a human codon adaptation index that is at least about 0.83 (83%). In another embodiment, the nucleotide sequence has a human codon adaptation index that is at least about 0.88 (88%). In another embodiment, the nucleotide sequence has a human codon adaptation index that is at least about 0.91 (91%). In another embodiment, the nucleotide sequence has a human codon adaptation index that is at least about 0.97 (97%).

In some embodiments, the codon optimized nucleotide sequence encoding a FVIII polypeptide of the present disclosure has an increased frequency of optimal codons (FOP) relative to SEQ ID NO: 16. In certain embodiments, the FOP of the codon optimized nucleotide sequence encoding a FVIII polypeptide is at least about 40, at least about 45, at least about 50, at least about 55, at least about 60, at least about 64, at least about 65, at least about 70, at least about 75, at least about 79, at least about 80, at least about 85, or at least about 90.

In other embodiments, the codon optimized nucleotide sequence encoding a FVIII polypeptide of the present disclosure has an increased relative synonymous codon usage (RCSU) relative to SEQ ID NO: 16. In some embodiments, the RCSU of the isolated nucleic acid molecule is greater than 1.5. In other embodiments, the RCSU of the isolated nucleic acid molecule is greater than 2.0. In certain embodiments, the RCSU of the isolated nucleic acid molecule is at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, or at least about 2.7.

In still other embodiments, the codon optimized nucleotide sequence encoding a FVIII polypeptide of the present disclosure has a decreased effective number of codons relative to SEQ ID NO: 16. In some embodiments, the codon optimized nucleotide sequence encoding a FVIII polypeptide has an effective number of codons of less than about 50, less than about 45, less than about 40, less than about 35, less than about 30, or less than about 25. In one particular embodiment, the isolated nucleic acid molecule has an effective number of codons of about 40, about 35, about 30, about 25, or about 20.

### B. G/C Content Optimization

In some embodiments, the genetic cassette comprises a codon optimized nucleotide sequence encoding a FVIII polypeptide, wherein the codon optimized nucleotide sequence contains a higher percentage of G/C nucleotides compared to the percentage of G/C nucleotides in SEQ ID NO: 16. In other embodiments, the codon optimized nucleotide sequence encoding a FVIII polypeptide has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, or at least about 60%.

In one particular embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the nucleotide sequence contains a higher percentage of G/C nucleotides compared to the percentage of G/C nucleotides in SEQ ID NO: 16. In some embodiments, the codon optimized nucleotide sequence encoding a FVIII polypeptide comprises has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, or at least about 58%. In one particular embodiment, the nucleotide sequence that encodes a polypeptide with FVIII activity has a G/C content that is at least about 58%.

In another embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment), or (iv) 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the codon optimized nucleotide sequence contains a higher percentage of G/C nucleotides compared to the percentage of G/C nucleotides in SEQ ID NO: 16. In other embodiments, the codon optimized nucleotide sequence encoding a FVIII polypeptide has a G/C content that is at least about 45%, at least about 46%, at least about 47%, at least about 48%, at least about 49%, at least about 50%, at least about 51%, at least about 52%, at least about 53%, at least about 54%, at least about 55%, at least about 56%, or at least about 57%. In one particular embodiment, the codon optimized nucleotide sequence encoding a FVIII polypeptide has a G/C content that is at least about 52%. In another embodiment, the codon optimized nucleotide sequence encoding a FVIII polypeptide has a G/C content that is at least about 55%. In another embodiment, the codon optimized nucleotide sequence encoding a FVIII polypeptide has a G/C content that is at least about 57%.

In other embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the nucleotide sequence contains a higher percentage of G/C nucleotides compared to the percentage of G/C nucleotides in SEQ ID NO: 16. In other embodiments, the codon optimized nucleotide sequence encoding a FVIII polypeptide has a G/C content that is at least about 45%. In one particular embodiment, the codon optimized nucleotide sequence encoding a FVIII polypeptide has a G/C content that is at least about 52%. In another embodiment, the codon optimized nucleotide sequence encoding a FVIII polypeptide has a G/C content that is at least about 55%. In another embodiment, the codon optimized nucleotide sequence encoding a FVIII polypeptide has a G/C content that is at least about 57%. In another embodiment, the codon optimized nucleotide sequence encoding a FVIII polypeptide has a G/C content that is at least about 58%. In still another embodiment, the n codon optimized nucleotide sequence encoding a FVIII polypeptide has a G/C content that is at least about 60%.

“G/C content” (or guanine-cytosine content), or “percentage of G/C nucleotides,” refers to the percentage of nitrogenous bases in a DNA molecule that are either guanine or cytosine. G/C content can be calculated using the following formula:

$$\frac{G + C}{A + T + G + C} \times 100 \qquad ( III )$$

Human genes are highly heterogeneous in their G/C content, with some genes having a G/C content as low as 20%, and other genes having a G/C content as high as 95%. In general, G/C rich genes are more highly expressed. In fact, it has been demonstrated that increasing the G/C content of a gene can lead to increased expression of the gene, due mostly to an increase in transcription and higher steady state mRNA levels. See Kudla et al., PLoS Biol., 4(6): e180 (2006).

### C. Matrix Attachment Region-Like Sequences

In some embodiments, the genetic cassette comprises a codon optimized nucleotide sequence encoding a FVIII polypeptide, wherein the codon optimized nucleotide sequence contains fewer MARS/ARS sequences relative to SEQ ID NO: 16. In other embodiments, the codon optimized nucleotide sequence encoding a FVIII polypeptide contains at most 6, at most 5, at most 4, at most 3, or at most 2 MARS/ARS sequences. In other embodiments, the codon optimized nucleotide sequence encoding a FVIII polypeptide contains at most 1 MARS/ARS sequence. In yet other embodiments, the codon optimized nucleotide sequence encoding a FVIII polypeptide does not contain a MARS/ARS sequence.

In one particular embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence contains fewer MARS/ARS sequences relative to SEQ ID NO: 16. In other embodiments, the nucleotide sequence that encodes a polypeptide with FVIII activity contains at most 6, at most 5, at most 4, at most 3, or at most 2 MARS/ARS sequences. In other embodiments, the codon optimized nucleotide sequence encoding a FVIII polypeptide contains at most 1 MARS/ARS sequence. In yet other embodiments, the codon optimized nucleotide sequence encoding a FVIII polypeptide does not contain a MARS/ARS sequence.

In another embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the nucleotide sequence contains fewer MARS/ARS sequences relative to SEQ ID NO: 16. In other embodiments, the codon optimized nucleotide sequence encoding a FVIII polypeptide contains at most 6, at most 5, at most 4, at most 3, or at most 2 MARS/ARS sequences. In other embodiments, the codon optimized nucleotide sequence encoding a FVIII polypeptide contains at most 1 MARS/ARS sequence. In yet other embodiments, the codon optimized nucleotide sequence encoding a FVIII polypeptide does not contain a MARS/ARS sequence.

In other embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of SEQ ID NOs: 1, 2, 3, 4, 5, 6, 70, or 71 or (ii) nucleotides 58-2277 and 2320-4374 of SEQ ID NOs: 1, 2, 3, 4, 5, 6, 70, or 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence contains fewer MARS/ARS sequences relative to SEQ ID NO: 16. In other embodiments, the codon optimized nucleotide sequence encoding a FVIII polypeptide contains at most 6, at most 5, at most 4, at most 3, or at most 2 MARS/ARS sequences. In other embodiments, the codon optimized nucleotide sequence encoding a FVIII polypeptide contains at most 1 MARS/ARS sequence. In yet other embodiments, the codon optimized nucleotide sequence encoding a FVIII polypeptide does not contain a MARS/ARS sequence.

AT-rich elements in the human FVIII nucleotide sequence that share sequence similarity with Saccharomyces cerevisiae autonomously replicating sequences (ARSs) and nuclear-matrix attachment regions (MARs) have been identified. (Fallux et al., Mol. Cell. Biol. 16:4264-4272 (1996). One of these elements has been demonstrated to bind nuclear factors in vitro and to repress the expression of a chloramphenicol acetyltransferase (CAT) reporter gene. Id. It has been hypothesized that these sequences can contribute to the transcriptional repression of the human FVIII gene. Thus, in one embodiment, all MAR/ARS sequences are abolished in the codon optimized nucleotide sequence encoding a FVIII polypeptide of the present disclosure. There are four MAR/ARS ATATTT sequences (SEQ ID NO: 21) and three MAR/ARS AAATAT sequences (SEQ ID NO: 22) in the parental FVIII sequence (SEQ ID NO: 16). All of these sites were mutated to destroy the MAR/ARS sequences in the optimized FVIII sequences (SEQ ID NOs: 1-6). The location of each of these elements, and the sequence of the corresponding nucleotides in the optimized sequences are shown in Table 3, below.

TABLE 3
Summary of Changes to Repressive Elements

| | Starting | | | | | | | | |
|---|---|---|---|---|---|---|---|---|---|
| | BDD | | | | | | | | |
| | FVIII | | | | | | | | |
| Location | Sequence | Optimized BDD FVIII Sequence | | | | | | | |
| of | (SEQ ID | SEQ ID | SEQ ID | SEQ ID | SEQ ID | SEQ ID | SEQ ID | SEQ ID | SEQ ID |
| Element | NO: 16) | NO: 1 | NO: 2 | NO: 3 | NO: 4 | NO: 5 | NO: 6 | NO: 70 | NO: 71 |

Destabilizing Sequences

| | | | | | | | | | |
|---|---|---|---|---|---|---|---|---|---|
| 639 | ATTTA | GTTTA | GTTCA | GTTCA | GTTCA | GTTCA | GTTCA | GTTCA | GTTCA |
| 1338 | ATTTA | GTTTA | GTTCA | CTTCA | GTTCA | GTTCA | GTTCA | CTTCA | GTTCA |
| 1449 | ATTTA | CTTTA | CTTCA | CTTCA | CTTCA | CTTCA | CTTCA | CTTCA | CTTCA |
| 1590 | TAAAT | TAAAT | CAAGT | CAAGT | TAAGT | CAAGT | CAAGT | CAAGT | TAAGT |
| 1623 | TAAAT | CAAAA | GAAGA | CTAAG | CAAGA | CAAGA | CAAGA | TAAGT | CAAGA |
| 2410 | ATTTA | ATCTA | ATCTA | ATCTA | ATCTA | ATCTA | ATCTA | ATCTA | ATCTA |
| 2586 | ATTTA | GTTTA | GTTCA | GTTCA | GTTCA | GTTCA | GTTCA | GTTCA | GTTCA |
| 2630 | TAAAT | TGAAT | TGAAC | TGAAC | TGAAC | TCAAT | TGAAC | TCAAT | TGAAC |
| 3884 | ATTTA | ATCTG | ACCTG | ACCTG | ACCTG | ATCTG | ACCTG | ATCTG | ACCTG |
| 3887 | TAAAT | TGAAC | TGAAC | TGAAC | TGAAC | TGAAC | TGAAC | TGAAC | TGAAC |

Potential Promoter Binding Sites

| | | | | | | | | | |
|---|---|---|---|---|---|---|---|---|---|
| 641 | TTATA | TTATC | TCATC | TCATT | TCATC | TCATC | TCATC | TCATT | TCATC |
| 1275 | TATAA | CTATA | TTACA | CTACA | GTACA | CTACA | CTACA | CTACA | GTACA |
| 1276 | TTATA | TATAA | TACAA | TACAA | TACAA | TACAA | TACAA | TACAA | TACAA |
| 1445 | TTATA | TCATC | TCATC | TTATC | TCATC | TCATC | TCATC | TTATC | TCATC |
| 1474 | TATAA | TATAA | TACAA | TACAA | TACAA | TACAA | TACAA | TACAA | TACAA |
| 1588 | TATAA | TATAA | TACAA | TACAA | TATAA | TACAA | TACAA | TACAA | TATAA |
| 2614 | TTATA | CTGTA | CTGTA | CTGTA | CTGTA | TTGTA | CTGTA | TTGTA | CTGTA |
| 2661 | TATAA | CATCA | CATCA | CATCA | CATCA | CATCA | CATCC | CATCA | CATCC |
| 3286 | TATAA | TATAA | TACAA | TACAA | TACAA | TACAA | TACAA | TACAA | TACAA |
| 3840 | TTATA | TTATA | TTACT | CTACA | CTACA | CTACA | CTACT | CTACA | CTACT |

Matrix Attachment-Like Sequences (MARS/ARS)

| | | | | | | | | | |
|---|---|---|---|---|---|---|---|---|---|
| 1287 | ATATTT | GTATCT | GTACCT | GTACCT | GTATCT | GTACCT | GTACCT | GTACCT | GTATCT |
| 1447 | ATATTT | ATCTTT | ATCTTC | ATCTTC | ATCTTC | ATCTTC | ATCTTC | ATCTTC | ATCTTC |
| 1577 | AAATAT | AAATCT | AGATCT | AAATCT | AAATCT | AGATCT | AGATCT | AAATCT | AAATCT |
| 1585 | AAATAT | AAGTAT | AAGTAC | AAGTAC | AAGTAT | AAGTAC | AAGTAC | AAGTAC | AAGTAT |
| 2231 | ATATTT | ACATCA | ATATCA | ACATCA | ACATCA | ACATCT | ATATCT | ACATCT | ATATCT |
| 3054 | AAATAT | AAACAT | GAATAT | GAACAT | GAACAT | GAACAT | GAATAT | GAACAT | GAATAT |
| 3788 | ATATTT | ATATCT | ATATCT | ACATCT | ACATCT | ACATCT | ACATCT | ACATCT | ACATCT |

AU Rich Sequence Elements (AREs)

| | | | | | | | | | |
|---|---|---|---|---|---|---|---|---|---|
| 2468 | ATTTTA | ACTTCATC | ACTTCATC | ACTTCATT | ACTTCATT | ACTTTATT | ACTTTATC | ACTTTATT | ACTTTATC |
| | TT | | | | | | | | |
| 3790 | ATTTTT | ATCTTTAA | ATCTTCAA | ATCTTCAA | ATCTTCAA | ATCTTCAA | ATCTTCAA | ATCTTCAA | ATCTTCAA |
| | AA | | | | | | | | |

Poly A/Poly T Sequences

| | | | | | | | | | |
|---|---|---|---|---|---|---|---|---|---|
| 3273 | AAAAAAA | GAAAAAA | GAAGAAG | GAAGAAG | GAAGAAG | GAAGAAG | CAAGAAG | GAAGAAG | CAAGAAG |
| 4195 | TTTTTT | TTCTTT | TTCTTC | TTCTTC | TTCTTC | TTCTTC | TTCTTC | TTCTTCC | TTCTTCC |

Splice Sites

| | | | | | | | | | |
|---|---|---|---|---|---|---|---|---|---|
| 2203 | GGTGAT | GGGGAC | GGCGAC | GGGGAC | GGGGAC | GGAGAC | GGAGAC | GGAGAC | GGAGAC |

### D. Destabilizing Sequences

In some embodiments, the genetic cassette comprises a codon optimized nucleotide sequence encoding a FVIII polypeptide, wherein the codon optimized nucleotide sequence contains fewer destabilizing elements relative to SEQ ID NO: 16. In other embodiments, the codon optimized nucleotide sequence encoding a FVIII polypeptide contains at most 9, at most 8, at most 7, at most 6, or at most 5 destabilizing elements. In other embodiments, the codon optimized nucleotide sequence encoding a FVIII polypeptide contains at most 4, at most 3, at most 2, or at most 1 destabilizing elements. In yet other embodiments, the codon optimized nucleotide sequence encoding a FVIII polypeptide does not contain a destabilizing element.

In one particular embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the codon optimized nucleotide sequence encoding a FVIII polypeptide contains at most 4, at most 3, at most 2, or at most 1 destabilizing elements. In yet other embodiments, the codon optimized nucleotide sequence encoding a FVIII polypeptide does not contain a destabilizing element.

In another embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the codon optimized nucleotide sequence contains fewer destabilizing elements relative to SEQ ID NO: 16. In other embodiments, the nucleotide sequence that encodes a polypeptide with FVIII activity contains at most 9, at most 8, at most 7, at most 6, or at most 5 destabilizing elements. In other embodiments, the codon optimized nucleotide sequence encoding a FVIII polypeptide contains at most 4, at most 3, at most 2, or at most 1 destabilizing elements. In yet other embodiments, the codon optimized nucleotide sequence encoding a FVIII polypeptide does not contain a destabilizing element.

In other embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence contains fewer destabilizing elements relative to SEQ ID NO: 16. In other embodiments, the codon optimized nucleotide sequence encoding a FVIII polypeptide contains at most 9, at most 8, at most 7, at most 6, or at most 5 destabilizing elements. In other embodiments, the codon optimized nucleotide sequence encoding a FVIII polypeptide contains at most 4, at most 3, at most 2, or at most 1 destabilizing elements. In yet other embodiments, the codon optimized nucleotide sequence encoding a FVIII polypeptide does not contain a destabilizing element.

There are ten destabilizing elements in the parental FVIII sequence (SEQ ID NO: 16): six ATTTA sequences (SEQ ID NO: 23) and four TAAAT sequences (SEQ ID NO: 24). In one embodiment, sequences of these sites were mutated to destroy the destabilizing elements in optimized FVIII SEQ ID NOs: 1-6, 70, and 71. The location of each of these elements, and the sequence of the corresponding nucleotides in the optimized sequences are shown in Table 3.

### E. Potential Promoter Binding Sites

In some embodiments, the genetic cassette comprises a codon optimized nucleotide sequence encoding a FVIII polypeptide, wherein the nucleotide sequence contains fewer potential promoter binding sites relative to SEQ ID NO: 16. In other embodiments, the codon optimized nucleotide sequence encoding a FVIII polypeptide contains at most 9, at most 8, at most 7, at most 6, or at most 5 potential promoter binding sites. In other embodiments, the codon optimized nucleotide sequence encoding a FVIII polypeptide contains at most 4, at most 3, at most 2, or at most 1 potential promoter binding sites. In yet other embodiments, the codon optimized nucleotide sequence encoding a FVIII polypeptide does not contain a potential promoter binding site.

In one particular embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence contains fewer potential promoter binding sites relative to SEQ ID NO: 16. In other embodiments, the codon optimized nucleotide sequence encoding a FVIII polypeptide contains at most 9, at most 8, at most 7, at most 6, or at most 5 potential promoter binding sites. In other embodiments, the codon optimized nucleotide sequence encoding a FVIII polypeptide contains at most 4, at most 3, at most 2, or at most 1 potential promoter binding sites. In yet other embodiments, the codon optimized nucleotide sequence encoding a FVIII polypeptide does not contain a potential promoter binding site.

In another embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6 (i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the codon optimized nucleotide sequence contains fewer potential promoter binding sites relative to SEQ ID NO: 16. In other embodiments, the codon optimized nucleotide sequence encoding a FVIII polypeptide contains at most 9, at most 8, at most 7, at most 6, or at most 5 potential promoter binding sites. In other embodiments, the codon optimized nucleotide sequence encoding a FVIII polypeptide contains at most 4, at most 3, at most 2, or at most 1 potential promoter binding sites. In yet other embodiments, the codon optimized nucleotide sequence encoding a FVIII polypeptide does not contain a potential promoter binding site.

In other embodiments, the genetic cassette comprises a codon optimized nucleotide sequence encoding a FVIII polypeptide, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence contains fewer potential promoter binding sites relative to SEQ ID NO: 16. In other embodiments, the codon optimized nucleotide sequence encoding a FVIII polypeptide contains at most 9, at most 8, at most 7, at most 6, or at most 5 potential promoter binding sites. In other embodiments, the codon optimized nucleotide sequence encoding a FVIII polypeptide contains at most 4, at most 3, at most 2, or at most 1 potential promoter binding sites. In yet other embodiments, the codon optimized nucleotide sequence encoding a FVIII polypeptide does not contain a potential promoter binding site.

TATA boxes are regulatory sequences often found in the promoter regions of eukaryotes. They serve as the binding site of TATA binding protein (TBP), a general transcription factor. TATA boxes usually comprise the sequence TATAA (SEQ ID NO: 28) or a close variant. TATA boxes within a coding sequence, however, can inhibit the translation of full-length protein. There are ten potential promoter binding sequences in the wild type BDD FVIII sequence (SEQ ID NO: 16): five TATAA sequences (SEQ ID NO: 28) and five TTATA sequences (SEQ ID NO: 29). In some embodiments, at least 1, at least 2, at least 3, or at least 4 of the promoter binding sites are abolished in the FVIII genes of the present disclosure. In some embodiments, at least 5 of the promoter binding sites are abolished in the FVIII genes of the present disclosure. In other embodiments, at least 6, at least 7, or at least 8 of the promoter binding sites are abolished in the FVIII genes of the present disclosure. In one embodiment, at least 9 of the promoter binging sites are abolished in the FVIII genes of the present disclosure. In one particular embodiment, all promoter binding sites are abolished in the FVIII genes of the present disclosure. The location of each potential promoter binding site and the sequence of the corresponding nucleotides in the optimized sequences are shown in Table 3.

### F. Other Cis Acting Negative Regulatory Elements

In addition to the MAR/ARS sequences, destabilizing elements, and potential promoter sites described above, several additional potentially inhibitory sequences can be identified in the wild type BDD FVIII sequence (SEQ ID NO: 16). Two AU rich sequence elements (AREs) can be identified (ATTTTATT (SEQ ID NOs: 30); and ATTTTTAA (SEQ ID NO: 31), along with a poly-A site (AAAAAAA; SEQ ID NO: 26), a poly-T site (TTTTTT; SEQ ID NO: 25), and a splice site (GGTGAT; SEQ ID NO: 27) in the non-optimized BDD FVIII sequence. One or more of these elements can be removed from the optimized FVIII sequences. The location of each of these sites and the sequence of the corresponding nucleotides in the optimized sequences are shown in Table 3.

In certain embodiments, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence does not contain one or more cis-acting negative regulatory elements, for example, a splice site, a poly-T sequence, a poly-A sequence, an ARE sequence, or any combinations thereof.

In another embodiment, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the second nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 1792-4374 of SEQ ID NO: 5; (ii) nucleotides 1792-4374 of SEQ ID NO: 6; (iii) nucleotides 1792-2277 and 2320-4374 of SEQ ID NO: 5 (i.e., nucleotides 1792-4374 of SEQ ID NO: 5 without the nucleotides encoding the B domain or B domain fragment); or (iv) nucleotides 1792-2277 and 2320-4374 of SEQ ID NO: 6(i.e., nucleotides 1792-4374 of SEQ ID NO: 6 without the nucleotides encoding the B domain or B domain fragment); wherein the N-terminal portion and the C-terminal portion together have a FVIII polypeptide activity; and wherein the codon optimized nucleotide sequence does not contain one or more cis-acting negative regulatory elements, for example, a splice site, a poly-T sequence, a poly-A sequence, an ARE sequence, or any combinations thereof.

In other embodiments, the genetic cassette comprises a codon optimized nucleotide sequence encoding a FVIII polypeptide, wherein the nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence does not contain one or more cis-acting negative regulatory elements, for example, a splice site, a poly-T sequence, a poly-A sequence, an ARE sequence, or any combinations thereof.

In some embodiments, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence does not contain the splice site GGTGAT (SEQ ID NO: 27). In some embodiments, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence does not contain a poly-T sequence (SEQ ID NO: 25). In some embodiments, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence does not contain a poly-A sequence (SEQ ID NO: 26). In some embodiments, the codon optimized nucleotide sequence encoding a FVIII polypeptide comprises a first nucleic acid sequence encoding an N-terminal portion of a FVIII polypeptide and a second nucleic acid sequence encoding a C-terminal portion of a FVIII polypeptide; wherein the first nucleic acid sequence has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-1791 of SEQ ID NO: 3; (ii) nucleotides 1-1791 of SEQ ID NO: 3; (iii) nucleotides 58-1791 of SEQ ID NO: 4; or (iv) nucleotides 1-1791 of SEQ ID NO: 4; wherein the N-terminal portion and the C-terminal portion together have a FVIII polypeptide activity; and wherein the codon optimized nucleotide sequence does not contain an ARE element (SEQ ID NO: 30 or SEQ ID NO: 31).

In some embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence does not contain the splice site GGTGAT (SEQ ID NO: 27). In some embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence does not contain a poly-T sequence (SEQ ID NO: 25). In some embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence does not contain a poly-A sequence (SEQ ID NO: 26). In some embodiments, the genetic cassette comprises a codon optimized nucleotide sequence encoding a FVIII polypeptide, wherein the codon optimized nucleotide sequence comprises a nucleic acid sequence having at least about 80%, at least about 85%, at least about 89%, at least about 90%, at least about 91%, at least about 92%, at least about 93%, at least about 94%, at least about 95%, at least about 96%, at least about 97%, at least about 98%, or at least about 99% sequence identity to (i) nucleotides 58-4374 of an amino acid sequence selected from SEQ ID NOs: 1, 2, 3, 4, 5, 6, 70, and 71 or (ii) nucleotides 58-2277 and 2320-4374 of an amino acid sequence selected from SEQ ID NOs: 1, 2, 3, 4, 5, 6, 70, and 71 (i.e., nucleotides 58-4374 of SEQ ID NO: 1, 2, 3, 4, 5, 6, 70, or 71 without the nucleotides encoding the B domain or B domain fragment); and wherein the codon optimized nucleotide sequence does not contain an ARE element (SEQ ID NO: 30 or SEQ ID NO: 31).

In other embodiments, an optimized FVIII sequence of the disclosure does not comprise one or more of antiviral motifs, stem-loop structures, and repeat sequences.

In still other embodiments, the nucleotides surrounding the transcription start site are changed to a kozak consensus sequence (GCCGCCACCATGC (SEQ ID NO: 32), wherein the underlined nucleotides are the start codon). In other embodiments, restriction sites can be added or removed to facilitate the cloning process.

b. FIX and Polynucleotide Sequences Encoding the FIX Protein

In some embodiments, the nucleic acid molecule comprises a first ITR, a second ITR, and a genetic cassette encoding a target sequence, wherein the target sequence encodes a therapeutic protein, wherein the therapeutic protein comprises a FIX polypeptide. In some embodiments, the FIX polypeptide comprises FIX or a variant or fragment thereof, wherein the FIX or the variant or fragment thereof has a FIX activity.

Human FIX is a serine protease that is an important component of the intrinsic pathway of the blood coagulation cascade. “Factor IX” or “FIX,” as used herein, refers to a coagulation factor protein and species and sequence variants thereof, and includes, but is not limited to, the 461 single-chain amino acid sequence of human FIX precursor polypeptide (“prepro”), the 415 single-chain amino acid sequence of mature human FIX (SEQ ID NO: 125), and the R338L FIX (Padua) variant (SEQ ID NO: 126). FIX includes any form of FIX molecule with the typical characteristics of blood coagulation FIX. As used herein “Factor IX” and “FIX” are intended to encompass polypeptides that comprise the domains Gla (region containing γ-carboxyglutamic acid residues), EGF1 and EGF2 (regions containing sequences homologous to human epidermal growth factor), activation peptide (“AP,” formed by residues R136-R180 of the mature FIX), and the C-terminal protease domain (“Pro”), or synonyms of these domains known in the art, or can be a truncated fragment or a sequence variant that retains at least a portion of the biological activity of the native protein. FIX or sequence variants have been cloned, as described in U.S. Pat. Nos. 4,770,999 and 7,700,734, and cDNA coding for human FIX has been isolated, characterized, and cloned into expression vectors (see, for example, Choo et al., Nature 299:178-180 (1982); Fair et al., Blood 64:194-204 (1984); and Kurachi et al., Proc. Natl. Acad. Sci., U.S.A. 79:6461-6464 (1982)). One particular variant of FIX, the R338L FIX (Padua) variant (SEQ ID NO: 2), characterized by Simioni et al, 2009, comprises a gain-of-function mutation, which correlates with a nearly 8-fold increase in the activity of the Padua variant relative to native FIX (Table 4). FIX variants can also include any FIX polypeptide having one or more conservative amino acid substitutions, which do not affect the FIX activity of the FIX polypeptide. In some embodiments, the FIX variant comprises rFIX-albumin fused by a cleavable linker, e.g., IDELVION®. See U.S. Pat. No. 7,939,632, incorporated herein by reference in its entirety.

TABLE 4
Example FIX Sequences

```
SEQ ID NO: 125 (mature FIX polypeptide)
    1: YNSGKLEEFV QGNLERECME EKCSFEEARE VFENTERTTE FWKQYVDGDQ CESNPCLNGG
  61: SCKDDINSYE CWCPFGFEGK NCELDVTCNI KNGRCEQFCK NSADNKVVCS CTEGYRLAEN
121: QKSCEPAVPF PCGRVSVSQT SKLTRAETVF PDVDYVNSTE AETILDNITQ STQSFNDFTR
181: VVGGEDAKPG QFPWQVVLNG KVDAFCGGSI VNEKWIVTAA HCVETGVKIT VVAGEHNIEE
241: TEHTEQKRNV IRIIPHHNYN AAINKYNHDI ALLELDEPLV LNSYVTPICI ADKEYTNIFL
301: KFGSGYVSGW GRVFHKGRSA LVLQYLRVPL VDRATCLRST KFTIYNNMFC AGFHEGGRDS
361: CQGDSGGPHV TEVEGTSFLT GIISWGEECA MKGKYGIYTK VSRYVNWIKE KTKLT
SEQ ID NO: 126 (mature Padua(R338L)FIX Polypeptide)
    1: YNSGKLEEFV QGNLERECME EKCSFEEARE VFENTERTTE FWKQYVDGDQ CESNPCLNGG
  61: SCKDDINSYE CWCPFGFEGK NCELDVTCNI KNGRCEQFCK NSADNKVVCS CTEGYRLAEN
121: QKSCEPAVPF PCGRVSVSQT SKLTRAETVF PDVDYVNSTE AETILDNITQ STQSFNDFTR
181: VVGGEDAKPG QFPWQVVLNG KVDAFCGGSI VNEKWIVTAA HCVETGVKIT VVAGEHNIEE
241: TEHTEQKRNV IRIIPHHNYN AAINKYNHDI ALLELDEPLV LNSYVTPICI ADKEYTNIFL
301: KFGSGYVSGW GRVFHKGRSA LVLQYLRVPL VDRATCLLST KFTIYNNMFC AGFHEGGRDS
361: CQGDSGGPHV TEVEGTSFLT GIISWGEECA MKGKYGIYTK VSRYVNWIKE KTKLT
SEQ ID NO: 127 (FIX Signal Polypeptide and Propeptide)
    1: MQRVNMIMAE SPGLITICLL GYLLSAECTV FLDHENANKI LNRPKR
SEQ ID NO: 160 (FIX-Linker-Albumin)
YNSGKLEEFV QGNLERECME EKCSFEEARE VFENTERTTE FWKQYVDGDQ 50
CESNPCLNGG SCKDDINSYE CWCPFGFEGK NCELDVTCNI KNGRCEQFCK 100
NSADNKVVCS CTEGYRLAEN QKSCEPAVPF PCGRVSVSQT SKLTRAETVF 150
PDVDYVNSTE AETILDNITQ STQSFNDFTR VVGGEDAKPG QFPWQVVLNG 200
KVDAFCGGSI VNEKWIVTAA HCVETGVKIT VVAGEHNIEE TEHTEQKRNV 250
IRIIPHHNYN AAINKYNHDI ALLELDEPLV LNSYVTPICI ADKEYTNIFL 300
KFGSGYVSGW GRVFHKGRSA LVLQYLRVPL VDRATCLRST KFTIYNNMFC 350
AGFHEGGRDS CQGDSGGPHV TEVEGTSFLT GIISWGEECA MKGKYGIYTK 400
VSRYVNWIKE KTKLTPVSQT SKLTRAETVF PDVDAHKSEV AHRFKDLGEE 450
NFKALVLIAF AQYLQQCPFE DHVKLVNEVT EFAKTCVADE SAENCDKSLH 500
TLFGDKLCTV ATLRETYGEM ADCCAKQEPE RNECFLQHKD DNPNLPRLVR 550
PEVDVMCTAF HDNEETFLKK YLYEIARRHP YFYAPELLFF AKRYKAAFTE 600
CCQAADKAAC LLPKLDELRD EGKASSAKQR LKCASLQKFG ERAFKAWAVA 650
RLSQRFPKAE FAEVSKLVTD LTKVHTECCH GDLLECADDR ADLAKYICEN 700
QDSISSKLKE CCEKPLLEKS HCIAEVENDE MPADLPSLAA DFVESKDVCK 750
NYAEAKDVFL GMFLYEYARR HPDYSVVLLL RLAKTYETTL EKCCAAADPH 800
ECYAKVFDEF KPLVEEPQNL IKQNCELFEQ LGEYKFQNAL LVRYTKKVPQ 850
VSTPTLVEVS RNLGKVGSKC CKHPEAKRMP CAEDYLSVVL NQLCVLHEKT 900
PVSDRVTKCC TESLVNRRPC FSALEVDETY VPKEFNAETF TFHADICTLS 950
EKERQIKKQT ALVELVKHKP KATKEQLKAV MDDFAAFVEK CCKADDKETC 1000
FAEEGKKLVA ASQAALGL 1018
SEQ ID NO: 161 (FIX)
YNSGKLEEFVQGNLERECMEEKCSFEEAREVFENTERTTEFWKQYVDGDQCESNPCLNGGSCKDDINSYE
CWCPFGFEGKNCELDVTCNIKNGRCEQFCKNSADNKVVCSCTEGYRLAENQKSCEPAVPFPCGRVSVSQT
SKLTRAETVFPDVDYVNSTEAETILDNITQSTQSFNDFTRVVGGEDAKPGQFPWQVVLNGKVDAFCGGSI
VNEKWIVTAAHCVETGVKITVVAGEHNIEETEHTEQKRNVIRIIPHHNYNAAINKYNHDIALLELDEPLV
LNSYVTPICIADKEYTNIFLKFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTKVSRYVNWIKEKTKLTPVSQT
SKLT
SEQ ID NO: 162 (Linker)
RAETVFPDV
SEQ ID NO: 163 (Albumin)
DAHKSEVAHRFKDLGEENFKALVLIAFAQYLQQCPFEDHVKLVNEVTEFAKTCVADESAENCDKSLHTLF
GDKLCTVATLRETYGEMADCCAKQEPERNECFLQHKDDNPNLPRLVRPEVDVMCTAFHDNEETFLKKYLY
EIARRHPYFYAPELLFFAKRYKAAFTECCQAADKAACLLPKLDELRDEGKASSAKQRLKCASLQKFGERA
FKAWAVARLSQRFPKAEFAEVSKLVTDLTKVHTECCHGDLLECADDRADLAKYICENQDSISSKLKECCE
KPLLEKSHCIAEVENDEMPADLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGEYKFQNALLVRYTKKVPQVST
PTLVEVSRNLGKVGSKCCKHPEAKRMPCAEDYLSVVLNQLCVLHEKTPVSDRVTKCCTESLVNRRPCFSA
LEVDETYVPKEFNAETFTFHADICTLSEKERQIKKQTALVELVKHKPKATKEQLKAVMDDFAAFVEKCCK
ADDKETCFAEEGKKLVAASQAALGL
SEQ ID NO: 164 (FIX(XTEN)-Fc)*

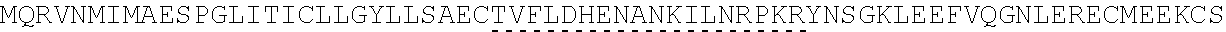

FEEAREVFENTERTTEFWKQYVDGDQCESNPCLNGGSCKDDINSYECWCPFGFEGKNCELDVTCNIKNGR
CEQFCKNSADNKVVCSCTEGYRLAENQKSCEPAVPFPCGRVSVSQTSKLTRAETVFPDVDYVNSTEAETI
LDGPSPGSPTSTEEGTSESATPESGPGSEPATSGSETPGTSESATPESGPGTSTEPSEGSAPGTSTEPSE
GSAPGASSNITQSTQSFNDFTRVVGGEDAKPGQFPWQVVLNGKVDAFCGGSIVNEKWIVTAAHCVETGVK
ITVVAGEHNIEETEHTEQKRNVIRIIPHHNYNAAINKYNHDIALLELDEPLVLNSYVTPICIADKEYTNI
FLKFGSGYVSGWGRVFHKGRSALVLQYLRVPLVDRATCLLSTKFTIYNNMFCAGFHEGGRDSCQGDSGGP
HVTEVEGTSFLTGIISWGEECAMKGKYGIYTKVSRYVNWIKEKTKLTDKTHTCPPCPAPELLGGPSVFLF
PPKPKDTLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPG
SEQ ID NO: 165 (FIX-FXIa-AE288)*
YNSGKLEEFVQGNLERECMEEKCSFEEAREVFENTERTTEFWKQYVDGDQCESNPCLNGGSCKDDINSYE
CWCPFGFEGKNCELDVTCNIKNGRCEQFCKNSADNKVVCSCTEGYRLAENQKSCEPAVPFPCGRVSVSQT
SKLTRAETVFPDVDYVNSTEAETILDNITQSTQSFNDFTRVVGGEDAKPGQFPWQVVLNGKVDAFCGGSI
VNEKWIVTAAHCVETGVKITVVAGEHNIEETEHTEQKRNVIRIIPHHNYNAAINKYNHDIALLELDEPLV
LNSYVTPICIADKEYTNIFLKFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTKVSRYVNWIKEKTKLTGKLTR
AETGGTSESATPESGPGSEPATSGSETPGTSESATPESGPGSEPATSGSETPGTSESATPESGPGTSTEP
SEGSAPGSPAGSPTSTEEGTSESATPESGPGSEPATSGSETPGTSESATPESGPGSPAGSPTSTEEGSPA
GSPTSTEEGTSTEPSEGSAPGTSESATPESGPGTSESATPESGPGTSESATPESGPGSEPATSGSETPGS
EPATSGSETPGSPAGSPTSTEEGTSTEPSEGSAPGTSTEPSEGSAPGSEPATSGSETPGTSESATPESGP
GTSTEPSEGSAP
SEQ ID NO: 166 (FIX-Fc-Fc)**
MQRVNMIMAESPGLITICLLGYLLSAECTVELDHENANKILNRPKRYNSGKLEEFVQGNLERECMEEKCS
FEEAREVFENTERTTEFWKQYVDGDQCESNPCLNGGSCKDDINSYECWCPEGFEGKNCELDVTCNIKNGR
CEQFCKNSADNKVVCSCTEGYRLAENQKSCEPAVPFPCGRVSVSQTSKLTRAETVFPDVDYVNSTEAETI
LDNITQSTQSENDFTRVVGGEDAKPGQFPWQVVLNGKVDAFCGGSIVNEKWIVTAAHCVETGVKITVVAG
EHNIEETEHTEQKRNVIRIIPHHNYNAAINKYNHDIALLELDEPLVLNSYVTPICIADKEYTNIFLKEGS
GYVSGWGRVEHKGRSALVLQYLRVPLVDRATCLRSTKFTIYNNMECAGEHEGGRDSCQGDSGGPHVTEVE
GTSFLTGIISWGEECAMKGKYGIYTKVSRYVNWIKEKTKLTDKTHTCPPCPAPELLGGPSVFLEPPKPKD
TLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESNGQPEN
NYKTTPPVLDSDGSFELYSKLTVDKSRWQQGNVESCSVMHEALHNHYTQKSLSLSPGKRRRRSGGGGSGG
GGSGGGGSGGGGSRRRRDKTHTCPPCPAPELLGGPSVFLEPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
*Grey shading = signal peptide; underline = XTEN sequence; bold = Fc.
**SEQ ID NO: 67 of U.S. Pat. No. 9,856,468, which is incorporated by reference herein in its entirety.
```

The FIX polypeptide is 55 kDa, synthesized as a prepropolypetide chain (SEQ ID NO: 125) composed of three regions: a signal peptide of 28 amino acids (amino acids 1 to 28 of SEQ ID NO: 127), a propeptide of 18 amino acids (amino acids 29 to 46), which is required for gamma-carboxylation of glutamic acid residues, and a mature Factor IX of 415 amino acids (SEQ ID NO: 125 or 126). The propeptide is an 18-amino acid residue sequence N-terminal to the gamma-carboxyglutamate domain. The propeptide binds vitamin K-dependent gamma carboxylase and then is cleaved from the precursor polypeptide of FIX by an endogenous protease, most likely PACE (paired basic amino acid cleaving enzyme), also known as furin or PCSK3. Without the gamma carboxylation, the Gla domain is unable to bind calcium to assume the correct conformation necessary to anchor the protein to negatively charged phospholipid surfaces, thereby rendering Factor IX nonfunctional. Even if it is carboxylated, the Gla domain also depends on cleavage of the propeptide for proper function, since retained propeptide interferes with conformational changes of the Gla domain necessary for optimal binding to calcium and phospholipid. In humans, the resulting mature Factor IX is secreted by liver cells into the blood stream as an inactive zymogen, a single chain protein of 415 amino acid residues that contains approximately 17% carbohydrate by weight (Schmidt, A. E., et al. (2003) Trends Cardiovasc Med, 13: 39).

The mature FIX is composed of several domains that in an N- to C-terminus configuration are: a GLA domain, an EGF1 domain, an EGF2 domain, an activation peptide (AP) domain, and a protease (or catalytic) domain. A short linker connects the EGF2 domain with the AP domain. FIX contains two activation peptides formed by R145-A146 and R180-V181, respectively. Following activation, the single-chain FIX becomes a 2-chain molecule, in which the two chains are linked by a disulfide bond. Clotting factors can be engineered by replacing their activation peptides resulting in altered activation specificity. In mammals, mature FIX must be activated by activated Factor XI to yield Factor IXa. The protease domain provides, upon activation of FIX to FIXa, the catalytic activity of FIX. Activated Factor VIII (FVIIIa) is the specific cofactor for the full expression of FIXa activity.

In certain embodiments, a FIX polypeptide comprises an Thr148 allelic form of plasma derived FIX and has structural and functional characteristics similar to endogenous FIX.

Many functional FIX variants are known in the art. International publication number WO 02/040544 A3 discloses mutants that exhibit increased resistance to inhibition by heparin at page 4, lines 9-30 and page 15, lines 6-31. International publication number WO 03/020764 A2 discloses FIX mutants with reduced T cell immunogenicity in Tables 2 and 3 (on pages 14-24), and at page 12, lines 1-27. International publication number WO 2007/149406 A2 discloses functional mutant FIX molecules that exhibit increased protein stability, increased in vivo and in vitro half-life, and increased resistance to proteases at page 4, line 1 to page 19, line 11. WO 2007/149406 A2 also discloses chimeric and other variant FIX molecules at page 19, line 12 to page 20, line 9. International publication number WO 08/118507 A2 discloses FIX mutants that exhibit increased clotting activity at page 5, line 14 to page 6, line 5. International publication number WO 09/051717 A2 discloses FIX mutants having an increased number of N-linked and/or O-linked glycosylation sites, which results in an increased half-life and/or recovery at page 9, line 11 to page 20, line 2. International publication number WO 09/137254 A2 also discloses Factor IX mutants with increased numbers of glycosylation sites at page 2, paragraph [006] to page 5, paragraph [011] and page 16, paragraph [044] to page 24, paragraph [057]. International publication number WO 09/130198 A2 discloses functional mutant FIX molecules that have an increased number of glycosylation sites, which result in an increased half-life, at page 4, line 26 to page 12, line 6. International publication number WO 09/140015 A2 discloses functional FIX mutants that an increased number of Cys residues, which can be used for polymer (e.g., PEG) conjugation, at page 11, paragraph [0043] to page 13, paragraph [0053]. The FIX polypeptides described in International Application No. PCT/US2011/043569 filed Jul. 11, 2011 and published as WO 2012/006624 on Jan. 12, 2012 are also incorporated herein by reference in its entirety. In some embodiments, the FIX polypeptide comprises a FIX polypeptide fused to an albumin, e.g., FIX-albumin. In certain embodiments, the FIX polypeptide is IDELVION® or rIX-FP.

In addition, hundreds of non-functional mutations in FIX have been identified in hemophilia subjects, many of which are disclosed in Table 6, at pages 11-14 of International publication number WO 09/137254 A2. Such non-functional mutations are not included in the invention, but provide additional guidance for which mutations are more or less likely to result in a functional FIX polypeptide.

In one embodiment, the FIX polypeptide (or Factor IX portion of a fusion polypeptide) comprises an amino acid sequence at least 70%, at least 80%, at least 85%, at least 90%, at least 95%, at least 96%, at least 97%, at least 98%, at least 99%, or 100% identical to the sequence set forth in SEQ ID NO: 1 or 2 (amino acids 1 to 415 of SEQ ID NO: 125 or 126), or alternatively, with a propeptide sequence, or with a propeptide and signal sequence (full length FIX). In another embodiment, the FIX polypeptide comprises an amino acid sequence at least 70%, at least 80%, at least 85%, at least 90%, at least 95%, at least 96%, at least 97%, at least 98%, at least 99%, or 100% identical to the sequence set forth in SEQ ID NO: 2.

FIX coagulant activity is expressed as International Unit(s) (IU). One IU of FIX activity corresponds approximately to the quantity of FIX in one milliliter of normal human plasma. Several assays are available for measuring FIX activity, including the one stage clotting assay (activated partial thromboplastin time; aPTT), thrombin generation time (TGA) and rotational thromboelastometry (ROTEM®). The invention contemplates sequences that have homology to FIX sequences, sequence fragments that are natural, such as from humans, non-human primates, mammals (including domestic animals), and non-natural sequence variants which retain at least a portion of the biologic activity or biological function of FIX and/or that are useful for preventing, treating, mediating, or ameliorating a coagulation factor-related disease, deficiency, disorder or condition (e.g., bleeding episodes related to trauma, surgery, of deficiency of a coagulation factor). Sequences with homology to human FIX can be found by standard homology searching techniques, such as NCBI BLAST.

In certain embodiments, the FIX sequence is codon-optimized. Examples of codon-optimized FIX sequences include, but are not limited to, SEQ ID NOs: 1 and 54-58 of International Publication No. WO 2016/004113 A1, which is incorporated by reference herein in its entirety.

c. FVII and Polynucleotide Sequences Encoding the FVII Protein

In some embodiments, the nucleic acid molecule comprises a first ITR, a second ITR, and a genetic cassette encoding a target sequence, wherein the target sequence encodes a therapeutic protein, wherein the therapeutic protein comprises a Factor VII polypeptide. In some embodiments, the FVII polypeptide comprises FVII or a variant or fragment thereof, wherein the variant or fragment thereof has a FVII activity.

“Factor VII” (“FVII,” or “F7;” also referred to as Factor 7, coagulation factor VII, serum factor VII, serum prothrombin conversion accelerator, SPCA, proconvertin and eptacog alpha) is a serine protease that is part of the coagulation cascade. In one embodiment, the clotting factor in the nucleic acid described herein is FVII. Recombinant activated Factor VII (“FVII”) has become widely used for the treatment of major bleeding, such as that which occurs in patients having hemophilia A or B, deficiency of coagulation Factor XI, FVII, defective platelet function, thrombocytopenia, or von Willebrand's disease.

Recombinant activated FVII (rFVIIa; NOVOSEVEN®) is used to treat bleeding episodes in (i) hemophilia patients with neutralizing antibodies against FVIII or FIX (inhibitors), (ii) patients with FVII deficiency, or (iii) patients with hemophilia A or B with inhibitors undergoing surgical procedures. However, NOVOSEVEN® displays poor efficacy. Repeated doses of FVIIa at high concentration are often required to control a bleed, due to its low affinity for activated platelets, short half-life, and poor enzymatic activity in the absence of tissue factor. Accordingly, there is an unmet medical need for better treatment and prevention options for hemophilia patients with FVIII and FIX inhibitors and/or with FVII deficiency.

In one embodiment, the genetic cassette encodes a mature form of FVII or a variant thereof. FVII includes a Gla domain, two EGF domains (EGF-1 and EGF-2), and a serine protease domain (or peptidase S1 domain) that is highly conserved among all members of the peptidase S1 family of serine proteases, such as for example with chymotrypsin. FVII occurs as a single chain zymogen (i.e., activatable FVII) and a fully activated two-chain form.

### C. Growth Factors

In some embodiments, the nucleic acid molecule comprises a first ITR, a second ITR, and a genetic cassette encoding a target sequence, wherein the target sequence encodes a therapeutic protein, and wherein the therapeutic protein comprises a growth factor. The growth factor can be selected from any growth factor known in the art. In some embodiments, the growth factor is a hormone. In other embodiments, the growth factor is a cytokine. In some embodiments, the growth factor is a chemokine.

In some embodiments, the growth factor is adrenomedullin (AM). In some embodiments, the growth factor is angiopoietin (Ang). In some embodiments, the growth factor is autocrine motility factor. In some embodiments, the growth factor is a Bone morphogenetic protein (BMP). In some embodiments, the BMP is selects from BMP2, BMP4, BMP5, and BMP7. In some embodiments, the growth factor is a ciliary neurotrophic factor family member. In some embodiments, the ciliary neurotrophic factor family member is selected from ciliary neurotrophic factor (CNTF), leukemia inhibitory factor (LIF), interleukin-6 (IL-6). In some embodiments, the growth factor is a colony-stimulating factor. In some embodiments, the colony-stimulating factor is selected from macrophage colony-stimulating factor (m-CSF), granulocyte colony-stimulating factor (G-CSF), and granulocyte macrophage colony-stimulating factor (GM-CSF). In some embodiments, the growth factor is an epidermal growth factor (EGF). In some embodiments, the growth factor is an ephrin. In some embodiments, the ephrin is selected from ephrin A1, ephrin A2, ephrin A3, ephrin A4, ephrin A5, ephrin 1, ephrin B2, and ephrin B3. In some embodiments, the growth factor is erythropoietin (EPO). In some embodiments, the growth factor is a fibroblast growth factor (FGF). In some embodiments, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some embodiments, the growth factor is foetal bovine somatotrophin (FBS). In some embodiments, the growth factor is a GDNF family member. In some embodiments, the GDNF family member is selected from glial cell line-derived neurotrophic factor (GDNF), neurturin, persephin, and artemin. In some embodiments, the growth factor is growth differentiation factor-9 (GDF9). In some embodiments, the growth factor is hepatocyte growth factor (HGF). In some embodiments, the growth factor is hepatoma-derived growth factor (HDGF). In some embodiments, the growth factor is insulin. In some embodiments, the growth factor is an insulin-like growth factor. In some embodiments, the insulin-like growth factor is insulin-like growth factor-1 (IGF-1) or IGF-2. In some embodiments, the growth factor is an interleukin (IL). In some embodiments, the IL is selected from IL-1, IL-2, IL-3, IL-4, IL-5, IL-6, and IL-7. In some embodiments, the growth factor is keratinocyte growth factor (KGF). In some embodiments, the growth factor is migration-stimulating factor (MSF). In some embodiments, the growth factor is macrophage-stimulating protein (MSP or hepatocyte growth factor-like protein (HGFLP)). In some embodiments, the growth factor is myostatin (GDF-8). In some embodiments, the growth factor is a neuregulin. In some embodiments, the neuregulin is selected from neuregulin 1 (NRG1), NRG2, NRG3, and NRG4. In some embodiments, the growth factor is a neurotrophin. In some embodiments, the growth factor is brain-derived neurotrophic factor (BDNF). In some embodiments, the growth factor is nerve growth factor (NGF). In some embodiments, the NGF is neurotrophin-3 (NT-3) or NT-4. In some embodiments, the growth factor is placental growth factor (PGF). In some embodiments, the growth factor is platelet-derived growth factor (PDGF). In some embodiments, the growth factor is renalase (RNLS). In some embodiments, the growth factor is T-cell growth factor (TCGF). In some embodiments, the growth factor is thrombopoietin (TPO). In some embodiments, the growth factor is a transforming growth factor. In some embodiments, the transforming growth factor is transforming growth factor alpha (TGF-α) or TGF-β. In some embodiments, the growth factor is tumor necrosis factor-alpha (TNF-α). In some embodiments, the growth factor is vascular endothelial growth factor (VEGF).

D. Micro RNAs (miRNAs)

MicroRNAs (miRNAs) are small non-coding RNA molecules (about 18-22 nucleotides) that negatively regulate gene expression by inhibiting translation or inducing messenger RNA (mRNA) degradation. Since their discovery, miRNAs have been implicated in various cellular processes including apoptosis, differentiation and cell proliferation and they have shown to play a key role in carcinogenesis. The ability of miRNAs to regulate gene expression makes expression of miRNAs in vivo a valuable tool in gene therapy.

Certain aspects of the present disclosure are directed to plasmid-like nucleic acid molecules comprising a first ITR, a second ITR, and a genetic cassette encoding a target sequence, wherein the target sequence encodes a miRNA, and wherein the first ITR and/or the second ITR are an ITR of a non-adeno-associated virus (e.g., the first ITR and/or the second ITR are from a non-AAV). The miRNA can be any miRNA known in the art. In some embodiments, the miRNA down regulates the expression of a target gene. In certain embodiments, the target gene is selected from SOD1, HTT, RHO, or any combination thereof.

In some embodiments, the genetic cassette encodes one miRNA. In some embodiments, the genetic cassette encodes more than one miRNA. In some embodiments, the genetic cassette encodes two or more different miRNAs. In some embodiments, the genetic cassette encodes two or more copies of the same miRNA. In some embodiments, the genetic cassette encodes two or more variants of the same therapeutic protein. In certain embodiments, the genetic cassette encodes one or more miRNA and one or more therapeutic protein.

In some embodiments, the miRNA is a naturally occurring miRNA. In some embodiments, the miRNA is an engineered miRNA. In some embodiments, the miRNA is an artificial miRNA. In certain embodiments, the miRNA comprises the miHTT engineered miRNA disclosed by Evers et al., Molecular Therapy 26(9):1-15 (epub ahead of print June 2018). In certain embodiments, the miRNA comprises the miR SOD1 artificial miRNA disclosed by Dirren et al., Annals of Clinical and Translational Neurology 2(2):167-84 (February 2015). In certain embodiments, the miRNA comprises miR-708, which targets RHO (see Behrman et al., JCB 192(6):919-27 (2011).

In some embodiments, the miRNA upregulates expression of a gene by down regulating the expression of an inhibitor of the gene. In some embodiments, the inhibitor is a natural, e.g., wild-type, inhibitor. In some embodiments, the inhibitor results from a mutated, heterologous, and/or misexpressed gene.

### E. Heterologous Moieties

In some embodiments, the nucleic acid molecule comprises a first ITR, a second ITR, and a genetic cassette encoding a target sequence, wherein the target sequence encodes a therapeutic protein, and wherein the therapeutic protein comprises at least one heterologous moiety. In some embodiments, the heterologous moiety is fused to the N-terminus or C-terminus of the therapeutic protein. In other embodiments, the heterologous moiety is inserted between two amino acids within the therapeutic protein.

In some embodiments, the therapeutic protein comprises a FVIII polypeptide and a heterologous moiety, which is inserted between two amino acids within the FVIII polypeptide. In some embodiments, the heterologous moiety is inserted within the FVIII polypeptide at one or more insertion site selected from Table 5. In some embodiments, the heterologous amino acid sequence can be inserted within the clotting factor polypeptide encoded by the nucleic acid molecule of the disclosure at any site disclosed in International Publication No. WO 2013/123457 A1, WO 2015/106052 A1 or U.S. Publication No. 2015/0158929 A1, which are herein incorporated by reference in their entirety. In one particular embodiment, the therapeutic protein comprises a FVIII and a heterologous moiety, wherein the heterologous moietyis inserted within the FVIII immediately downstream of amino acid 745 relative to mature FVIII. In one particular embodiment, the therapeutic protein comprises a FVIII and an XTEN wherein the XTEN is inserted within the FVIII immediately downstream of amino acid 745 relative to mature FVIII. In one particular embodiment, the FVIII comprises a deletion of amino acids 746-1646, corresponding to mature human FVIII (SEQ ID NO:15), and the heterologous moiety is inserted immediately downstream of amino acid 745, corresponding to mature human FVIII (SEQ ID NO:15).

TABLE 5
FVIII Heterologous Moiety Insertion Sites

| Insertion | |
|---|---|
| Site | Domain |
| 3 | A1 |
| 18 | A1 |
| 22 | A1 |
| 26 | A1 |
| 40 | A1 |
| 60 | A1 |
| 65 | A1 |
| 81 | A1 |
| 116 | A1 |
| 119 | A1 |
| 130 | A1 |
| 188 | A1 |
| 211 | A1 |
| 216 | A1 |
| 220 | A1 |
| 224 | A1 |
| 230 | A1 |
| 333 | A1 |
| 336 | A1 |
| 339 | A1 |
| 375 | A2 |
| 378 | A2 |
| 399 | A2 |
| 403 | A2 |
| 409 | A2 |
| 416 | A2 |
| 442 | A2 |
| 487 | A2 |
| 490 | A2 |
| 494 | A2 |
| 500 | A2 |
| 518 | A2 |
| 599 | A2 |
| 603 | A2 |
| 713 | A2 |
| 745 | B |
| 1656 | a3 region |
| 1711 | A3 |
| 1720 | A3 |
| 1725 | A3 |
| 1749 | A3 |
| 1796 | A3 |
| 1802 | A3 |
| 1827 | A3 |
| 1861 | A3 |
| 1896 | A3 |
| 1900 | A3 |
| 1904 | A3 |
| 1905 | A3 |
| 1910 | A3 |
| 1937 | A3 |
| 2019 | A3 |
| 2068 | C1 |
| 2111 | C1 |
| 2120 | C1 |
| 2171 | C2 |
| 2188 | C2 |
| 2227 | C2 |
| 2332 | CT |

In some embodiments, the therapeutic protein comprises a FIX polypeptide and a heterologous moiety, which is inserted between two amino acids within the FIX polypeptide. In some embodiments, the heterologous moiety is inserted within the FIX polypeptide at one or more insertion site selected from Table 5. In some embodiments, the heterologous amino acid sequence can be inserted within the clotting factor polypeptide encoded by the nucleic acid molecule of the disclosure at any site disclosed in International Application No. PCT/US2017/015879, which is herein incorporated by reference in their entirety. In one particular embodiment, the therapeutic protein comprises a FIX polypeptide and a heterologous moiety, wherein the heterologous moiety is inserted within the FIX polypeptide immediately downstream of amino acid 166 relative to mature FIX. In one particular embodiment, the therapeutic protein comprises a FIX polypeptide and an XTEN, wherein the XTEN is inserted within the FIX immediately downstream of amino acid 166 relative to mature FVIII.

TABLE 6
FIX Heterologous Moiety Insertion Sites

| Insertion | |
|---|---|
| Site | Domain |
| 52 | EGF1 |
| 59 | EGF1 |
| 66 | EGF1 |
| 80 | EGF1 |
| 85 | EGF2 |
| 89 | EGF2 |
| 103 | EGF2 |
| 105 | EGF2 |
| 113 | EGF2 |
| 129 | Linker |
| 142 | Linker |
| 149 | AP |
| 162 | AP |
| 166 | AP |
| 174 | AP |
| 188 | Catalytic |
| 202 | Catalytic |
| 224 | Catalytic |
| 226 | Catalytic |
| 228 | Catalytic |
| 230 | Catalytic |
| 240 | Catalytic |
| 257 | Catalytic |
| 265 | Catalytic |
| 277 | Catalytic |
| 283 | Catalytic |
| 292 | Catalytic |
| 316 | Catalytic |
| 341 | Catalytic |
| 354 | Catalytic |
| 392 | Catalytic |
| 403 | Catalytic |
| 413 | Catalytic |

In other embodiments, the therapeutic proteins of the disclosure further comprise two, three, four, five, six, seven, or eight heterologous nucleotide sequences. In some embodiments, all the heterologous moieties are identical. In some embodiments, at least one heterologous moiety is different from the other heterologous moieties. In some embodiments, the disclosure can comprise two, three, four, five, six, or more than seven heterologous moieties in tandem.

In some embodiments, the heterologous moiety increases the half-life (is a “half-life extender”) of the therapeutic protein.

In some embodiments, the heterologous moiety is a peptide or a polypeptide with either unstructured or structured characteristics that are associated with the prolongation of in vivo half-life when incorporated in a protein of the disclosure. Non-limiting examples include albumin, albumin fragments, Fc fragments of immunoglobulins, the C-terminal peptide (CTP) of the β subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides. In one particular embodiment, the heterologous amino acid sequence is an immunoglobulin constant region or a portion thereof, transferrin, albumin, or a PAS sequence. In some aspects, a heterologous moiety includes von Willebrand factor or a fragment thereof. In other related aspects a heterologous moiety can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety comprises a cysteine amino acid that functions as an attachment site for a non-polypeptide moiety such as polyethylene glycol (PEG), hydroxyethyl starch (HES), polysialic acid, or any derivatives, variants, or combinations of these elements.

In one specific embodiment, a first heterologous moiety is a half-life extending molecule which is known in the art, and a second heterologous moiety is a half-life extending molecule which is known in the art. In certain embodiments, the first heterologous moiety (e.g., a first Fc moiety) and the second heterologous moiety (e.g., a second Fc moiety) are associated with each other to form a dimer. In one embodiment, the second heterologous moiety is a second Fc moiety, wherein the second Fc moiety is linked to or associated with the first heterologous moiety, e.g., the first Fc moiety. For example, the second heterologous moiety (e.g., the second Fc moiety) can be linked to the first heterologous moiety (e.g., the first Fc moiety) by a linker or associated with the first heterologous moiety by a covalent or non-covalent bond.

In some embodiments, the heterologous moiety is a polypeptide comprising, consisting essentially of, or consisting of at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, or at least about 4000 amino acids. In other embodiments, the heterologous moiety is a polypeptide comprising, consisting essentially of, or consisting of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous moiety improves one or more pharmacokinetic properties of the therapeutic protein without significantly affecting its biological activity or function.

In certain embodiments, a heterologous moiety increases the in vivo and/or in vitro half-life of the therapeutic protein of the disclosure. In other embodiments, a heterologous moiety facilitates visualization or localization of the therapeutic protein of the disclosure or a fragment thereof (e.g., a fragment comprising a heterologous moiety after proteolytic cleavage of the FVIII protein). Visualization and/or location of the therapeutic protein of the disclosure or a fragment thereof can be in vivo, in vitro, ex vivo, or combinations thereof.

In other embodiments, a heterologous moiety increases stability of the therapeutic protein of the disclosure or a fragment thereof (e.g., a fragment comprising a heterologous moiety after proteolytic cleavage of the therapeutic protein, e.g., a clotting factor). As used herein, the term “stability” refers to an art-recognized measure of the maintenance of one or more physical properties of the therapeutic protein in response to an environmental condition (e.g., an elevated or lowered temperature). In certain aspects, the physical property can be the maintenance of the covalent structure of the therapeutic protein (e.g., the absence of proteolytic cleavage, unwanted oxidation or deamidation). In other aspects, the physical property can also be the presence of the therapeutic protein in a properly folded state (e.g., the absence of soluble or insoluble aggregates or precipitates). In one aspect, the stability of the therapeutic protein is measured by assaying a biophysical property of the therapeutic protein, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In certain aspects, a therapeutic protein encoded by the nucleic acid molecule of the disclosure comprises at least one half-life extender, i.e., a heterologous moiety which increases the in vivo half-life of the therapeutic protein with respect to the in vivo half-life of the corresponding therapeutic protein lacking such heterologous moiety. In vivo half-life of a therapeutic protein can be determined by any methods known to those of skill in the art, e.g., activity assays (e.g., chromogenic assay or one stage clotting aPTT assay wherein the therapeutic protein comprises a FVIII polypeptide), ELISA, ROTEM®, etc.

In some embodiments, the presence of one or more half-life extenders results in the half-life of the therapeutic protein to be increased compared to the half-life of the corresponding protein lacking such one or more half-life extenders. The half-life of the therapeutic protein comprising a half-life extender is at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than the in vivo half-life of the corresponding therapeutic protein lacking such half-life extender.

In one embodiment, the half-life of the therapeutic protein comprising a half-life extender is about 1.5-fold to about 20-fold, about 1.5-fold to about 15-fold, or about 1.5-fold to about 10-fold longer than the in vivo half-life of the corresponding protein lacking such half-life extender. In another embodiment, the half-life of therapeutic protein comprising a half-life extender is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to the in vivo half-life of the corresponding protein lacking such half-life extender.

In other embodiments, the half-life of the therapeutic protein comprising a half-life extender is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours.

In still other embodiments, the half-life of the therapeutic protein comprising a half-life extender is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life per subject of the therapeutic protein comprising a half-life extender is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days.

One or more half-life extenders can be fused to C-terminus or N-terminus of therapeutic protein or inserted within therapeutic protein.

1. An immunoglobulin Constant Region or a Portion Thereof

In another aspect, a heterologous moiety comprises one or more immunoglobulin constant regions or portions thereof (e.g., an Fc region). In one embodiment, an isolated nucleic acid molecule of the disclosure further comprises a heterologous nucleic acid sequence that encodes an immunoglobulin constant region or a portion thereof. In some embodiments, the immunoglobulin constant region or portion thereof is an Fc region.

An immunoglobulin constant region is comprised of domains denoted CH (constant heavy) domains (CH1, CH2, etc.). Depending on the isotype, (i.e. IgG, IgM, IgA IgD, or IgE), the constant region can be comprised of three or four CH domains. Some isotypes (e.g. IgG) constant regions also contain a hinge region. See Janeway et al. 2001, Immunobiology, Garland Publishing, N.Y., N.Y.

An immunoglobulin constant region or a portion thereof of the present disclosure can be obtained from a number of different sources. In one embodiment, an immunoglobulin constant region or a portion thereof is derived from a human immunoglobulin. It is understood, however, that the immunoglobulin constant region or a portion thereof can be derived from an immunoglobulin of another mammalian species, including for example, a rodent (e.g., a mouse, rat, rabbit, guinea pig) or non-human primate (e.g., chimpanzee, macaque) species. Moreover, the immunoglobulin constant region or a portion thereof can be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

A variety of the immunoglobulin constant region gene sequences (e.g., human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g., hinge, CH2, and/or CH3 sequences, or portions thereof) can be derived from these sequences using art recognized techniques. The genetic material obtained using any of the foregoing methods can then be altered or synthesized to obtain polypeptides of the present disclosure. It will further be appreciated that the scope of this disclosure encompasses alleles, variants and mutations of constant region DNA sequences.

The sequences of the immunoglobulin constant region or a portion thereof can be cloned, e.g., using the polymerase chain reaction and primers which are selected to amplify the domain of interest. To clone a sequence of the immunoglobulin constant region or a portion thereof from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, CA (1990); Ho et al. 1989. Gene 77:51; Horton et al. 1993. Methods Enzymol. 217:270). PCR can be initiated by consensus constant region primers or by more specific primers based on the published heavy and light chain DNA and amino acid sequences. PCR also can be used to isolate DNA clones encoding the antibody light and heavy chains. In this case the libraries can be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

An immunoglobulin constant region used herein can include all domains and the hinge region or portions thereof. In one embodiment, the immunoglobulin constant region or a portion thereof comprises CH2 domain, CH3 domain, and a hinge region, i.e., an Fc region or an FcRn binding partner.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence). See International Publication No. WO 2012/006635, incorporated herein by reference in its entirety.

In one embodiment, the “Fc region” refers to the portion of a single Ig heavy chain beginning in the hinge region just upstream of the papain cleavage site (i.e., residue 216 in IgG, taking the first residue of heavy chain constant region to be 114) and ending at the C-terminus of the antibody. Accordingly, a complete Fc region comprises at least a hinge domain, a CH2 domain, and a CH3 domain.

An immunoglobulin constant region or a portion thereof can be an FcRn binding partner. FcRn is active in adult epithelial tissues and expressed in the lumen of the intestines, pulmonary airways, nasal surfaces, vaginal surfaces, colon and rectal surfaces (U.S. Pat. No. 6,485,726). An FcRn binding partner is a portion of an immunoglobulin that binds to FcRn.

The FcRn receptor has been isolated from several mammalian species including humans. The sequences of the human FcRn, monkey FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,485,726, 6,030,613, 6,086,875; WO 03/077834; US2003-0235536A1) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners useful in the present disclosure encompass molecules that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

Fc regions or FcRn binding partners bound to FcRn can be effectively shuttled across epithelial barriers by FcRn, thus providing a non-invasive means to systemically administer a desired therapeutic molecule. Additionally, fusion proteins comprising an Fc region or an FcRn binding partner are endocytosed by cells expressing the FcRn. But instead of being marked for degradation, these fusion proteins are recycled out into circulation again, thus increasing the in vivo half-life of these proteins. In certain embodiments, the portions of immunoglobulin constant regions are an Fc region or an FcRn binding partner that typically associates, via disulfide bonds and other non-specific interactions, with another Fc region or another FcRn binding partner to form dimers and higher order multimers.

Two FcRn receptors can bind a single Fc molecule. Crystallographic data suggest that each FcRn molecule binds a single polypeptide of the Fc homodimer. In one embodiment, linking the FcRn binding partner, e.g., an Fc fragment of an IgG, to a biologically active molecule provides a means of delivering the biologically active molecule orally, buccally, sublingually, rectally, vaginally, as an aerosol administered nasally or via a pulmonary route, or via an ocular route. In another embodiment, the clotting factor protein can be administered invasively, e.g., subcutaneously, intravenously.

An FcRn binding partner region is a molecule or portion thereof that can be specifically bound by the FcRn receptor with consequent active transport by the FcRn receptor of the Fc region. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 10⁸ M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g., serum albumin, milk casein), etc., can be optimized by a skilled artisan using routine techniques.

In certain embodiments, a therapeutic protein encoded by the nucleic acid molecule of the disclosure comprises one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgG1, EU numbering (with the primary contact sites being amino acids 248, 250-257, 272, 285, 288, 290-291, 308-311, and 314 of the CH2 domain and amino acid residues 385-387, 428, and 433-436 of the CH3 domain). Thus, an Fc region of the disclosure can comprise or consist of an FcRn binding portion. FcRn binding portions can be derived from heavy chains of any isotype, including IgGI, IgG2, IgG3 and IgG4. In one embodiment, an FcRn binding portion from an antibody of the human isotype IgG1 is used. In another embodiment, an FcRn binding portion from an antibody of the human isotype IgG4 is used.

The Fc region can be obtained from a number of different sources. In one embodiment, an Fc region of the polypeptide is derived from a human immunoglobulin. It is understood, however, that an Fc moiety can be derived from an immunoglobulin of another mammalian species, including for example, a rodent (e.g., a mouse, rat, rabbit, guinea pig) or non-human primate (e.g., chimpanzee, macaque) species. Moreover, the polypeptide of the Fc domains or portions thereof can be derived from any immunoglobulin class, including IgM, IgG, IgD, IgA and IgE, and any immunoglobulin isotype, including IgG1, IgG2, IgG3 and IgG4. In another embodiment, the human isotype IgG1 is used.

In certain embodiments, the Fc variant confers a change in at least one effector function imparted by an Fc moiety comprising said wild-type Fc domain (e.g., an improvement or reduction in the ability of the Fc region to bind to Fc receptors (e.g. FcγRI, FcγRII, or FcγRIII) or complement proteins (e.g., C1q), or to trigger antibody-dependent cytotoxicity (ADCC), phagocytosis, or complement-dependent cytotoxicity (CDCC)). In other embodiments, the Fc variant provides an engineered cysteine residue.

The Fc region of the disclosure can employ art-recognized Fc variants which are known to impart a change (e.g., an enhancement or reduction) in effector function and/or FcR or FcRn binding. Specifically, an Fc region of the disclosure can include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, each of which is incorporated by reference herein. In one embodiment, the specific change (e.g., the specific substitution of one or more amino acids disclosed in the art) can be made at one or more of the disclosed amino acid positions. In another embodiment, a different change at one or more of the disclosed amino acid positions (e.g., the different substitution of one or more amino acid position disclosed in the art) can be made.

The Fc region or FcRn binding partner of IgG can be modified according to well recognized procedures such as site directed mutagenesis and the like to yield modified IgG or Fc fragments or portions thereof that will be bound by FcRn. Such modifications include modifications remote from the FcRn contact sites as well as modifications within the contact sites that preserve or even enhance binding to the FcRn. For example, the following single amino acid residues in human IgG1 Fc (Fc γ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wild type proline substituted by alanine at position number 238. As an example, a specific embodiment incorporates the N297A mutation, removing a highly conserved N-glycosylation site. In addition to alanine other amino acids can be substituted for the wild type amino acids at the positions specified above. Mutations can be introduced singly into Fc giving rise to more than one hundred Fc regions distinct from the native Fc. Additionally, combinations of two, three, or more of these individual mutations can be introduced together, giving rise to hundreds more Fc regions.

Certain of the above mutations can confer new functionality upon the Fc region or FcRn binding partner. For example, one embodiment incorporates N297A, removing a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity for FcRn (Routledge et al. 1995, Transplantation 60:847; Friend et al. 1999, Transplantation 68:1632; Shields et al. 1995, J. Biol. Chem. 276:6591). As a further example of new functionality arising from mutations described above affinity for FcRn can be increased beyond that of wild type in some instances. This increased affinity can reflect an increased “on” rate, a decreased “off” rate or both an increased “on” rate and a decreased “off” rate. Examples of mutations believed to impart an increased affinity for FcRn include, but not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity can arise from mutations of this region, as for example by replacing amino acids 233-236 of human IgG1 “ELLG” (SEQ ID NO: 45) to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In another embodiment, the immunoglobulin constant region or a portion thereof comprises an amino acid sequence in the hinge region or a portion thereof that forms one or more disulfide bonds with a second immunoglobulin constant region or a portion thereof. The second immunoglobulin constant region or a portion thereof can be linked to a second polypeptide, bringing the therapeutic protein and the second polypeptide together. In some embodiments, the second polypeptide is an enhancer moiety. As used herein, the term “enhancer moiety” refers to a molecule, fragment thereof or a component of a polypeptide which is capable of enhancing the activity of the therapeutic protein. The enhancer moiety can be a cofactor, such as, wherein the therapeutic protein is a clotting factor, a soluble tissue factor (sTF), or a procoagulant peptide. Thus, upon activation of the clotting factor, the enhancer moiety is available to enhance clotting factor activity.

In certain embodiments, a therapeutic protein encoded by a nucleic acid molecule of the disclosure comprises an amino acid substitution to an immunoglobulin constant region or a portion thereof (e.g., Fc variants), which alters the antigen-independent effector functions of the Ig constant region, in particular the circulating half-life of the protein.

2. scFc Regions

In another aspect, a heterologous moiety comprises a scFc (single chain Fc) region. In one embodiment, an isolated nucleic acid molecule of the disclosure further comprises a heterologous nucleic acid sequence that encodes a scFc region. The scFc region comprises at least two immunoglobulin constant regions or portions thereof (e.g., Fc moieties or domains (e.g., 2, 3, 4, 5, 6, or more Fc moieties or domains)) within the same linear polypeptide chain that are capable of folding (e.g., intramolecularly or intermolecularly folding) to form one functional scFc region which is linked by an Fc peptide linker. For example, in one embodiment, a polypeptide of the disclosure is capable of binding, via its scFc region, to at least one Fc receptor (e.g., an FcRn, an FcγR receptor (e.g., FcγRIII), or a complement protein (e.g., C1q)) in order to improve half-life or trigger an immune effector function (e.g., antibody-dependent cytotoxicity (ADCC), phagocytosis, or complement-dependent cytotoxicity (CDCC) and/or to improve manufacturability).

### 3. CTP

In another aspect, a heterologous moiety comprises one C-terminal peptide (CTP) of the β subunit of human chorionic gonadotropin or fragment, variant, or derivative thereof. One or more CTP peptides inserted into a recombinant protein is known to increase the in vivo half-life of that protein. See, e.g., U.S. Pat. No. 5,712,122, incorporated by reference herein in its entirety.

Exemplary CTP peptides include DPRFQDSSSSKAPPPSLPSPSRLPGPSDTPIL (SEQ ID NO: 33) or SSSSKAPPPSLPSPSRLPGPSDTPILPQ (SEQ ID NO: 34). See, e.g., U.S. Patent Application Publication No. US 2009/0087411 A1, incorporated by reference.

### 4. XTEN Sequence

In some embodiments, a heterologous moiety comprises one or more XTEN sequences, fragments, variants, or derivatives thereof. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

The incorporation of a heterologous moiety comprising an XTEN sequence into a protein of the disclosure can confer to the protein one or more of the following advantageous properties: conformational flexibility, enhanced aqueous solubility, high degree of protease resistance, low immunogenicity, low binding to mammalian receptors, or increased hydrodynamic (or Stokes) radii.

In certain aspects, an XTEN sequence can increase pharmacokinetic properties such as longer in vivo half-life or increased area under the curve (AUC), so that a protein of the disclosure stays in vivo and has procoagulant activity for an increased period of time compared to a protein with the same but without the XTEN heterologous moiety.

In some embodiments, the XTEN sequence useful for the disclosure is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence of the disclosure can comprise one or more sequence motif of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine (S), threonine (T), glutamate (E) and proline (P). See US 2010-0239554 A1.

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from Table 7, resulting in a family sequence. As used herein, “family” means that the XTEN has motifs selected only from a single motif category from Table 7; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to the therapeutic protein. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of Table 7. These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues.

TABLE 7
XTEN Sequence Motifs of 12 Amino Acids and Motif
Families

| Motif | | |
|---|---|---|
| Family* | MOTIF SEQUENCE | SEQ ID NO: |
| AD | GESPGGSSGSES | 73 |
| AD | GSEGSSGPGESS | 74 |
| AD | GSSESGSSEGGP | 75 |
| AD | GSGGEPSESGSS | 76 |
| AE, AM | GSPAGSPTSTEE | 77 |
| AE, AM, AQ | GSEPATSGSETP | 78 |
| AE, AM, AQ | GTSESATPESGP | 79 |
| AE, AM, AQ | GTSTEPSEGSAP | 80 |
| AF, AM | GSTSESPSGTAP | 81 |
| AF, AM | GTSTPESGSASP | 82 |
| AF, AM | GTSPSGESSTAP | 83 |
| AF, AM | GSTSSTAESPGP | 84 |
| AG, AM | GTPGSGTASSSP | 85 |
| AG, AM | GSSTPSGATGSP | 86 |
| AG, AM | GSSPSASTGTGP | 87 |
| AG, AM | GASPGTSSTGSP | 88 |
| AQ | GEPAGSPTSTSE | 89 |
| AQ | GTGEPSSTPASE | 90 |
| AQ | GSGPSTESAPTE | 91 |
| AQ | GSETPSGPSETA | 92 |
| AQ | GPSETSTSEPGA | 93 |
| AQ | GSPSEPTEGTSA | 94 |
| BC | GSGASEPTSTEP | 95 |
| BC | GSEPATSGTEPS | 96 |
| BC | GTSEPSTSEPGA | 97 |
| BC | GTSTEPSEPGSA | 98 |
| BD | GSTAGSETSTEA | 99 |
| BD | GSETATSGSETA | 100 |
| BD | GTSESATSESGA | 101 |
| BD | GTSTEASEGSAS | 102 |

*Denotes individual motif sequences that, when used together in various permutations, results in a “family sequence”

Examples of XTEN sequences that can be used as heterologous moieties in the therapeutic proteins of the disclosure are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122A1, WO 2010144502 A2, WO 2010144508 A1, WO 2011028228 A1, WO 2011028229 A1, or WO 2011028344 A2, each of which is incorporated by reference herein in its entirety.

XTEN can have varying lengths for insertion into or linkage to a therapeutic protein. In one embodiment, the length of the XTEN sequence(s) is chosen based on the property or function to be achieved in the fusion protein. Depending on the intended property or function, XTEN can be short or intermediate length sequence or longer sequence that can serve as carriers. In certain embodiments, the XTEN includes short segments of about 6 to about 99 amino acid residues, intermediate lengths of about 100 to about 399 amino acid residues, and longer lengths of about 400 to about 1000 and up to about 3000 amino acid residues. Thus, the XTEN inserted into or linked to a therapeutic protein can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN inserted into or linked to a therapeutic protein can vary without adversely affecting the activity of the therapeutic protein. In one embodiment, one or more of the XTENs used herein have 42 amino acids, 72 amino acids, 144 amino acids, 288 amino acids, 576 amino acids, or 864 amino acids in length and can be selected from one or more of the XTEN family sequences; i.e., AD, AE, AF, AG, AM, AQ, BC or BD.

In some embodiments, the therapeutic protein comprises a FVIII polypeptide and an XTEN, wherein the XTEN comprises 288 amino acids. In one embodiment, the therapeutic protein comprises a FVIII polypeptide and an XTEN, wherein the XTEN comprises 288 amino acids, and the XTEN is inserted within the B domain of the FVIII polypeptide. In one particular embodiment, the therapeutic protein comprises a FVIII polypeptide and an XTEN comprising SEQ ID NO:109, and the XTEN is inserted within the B domain of the FVIII polypeptide. In one particular embodiment, the therapeutic protein comprises a FVIII polypeptide and an XTEN comprising SEQ ID NO:109, and the XTEN is inserted within the FVIII polypeptide immediately downstream of amino acid 745 of mature FVIII.

In some embodiments, the therapeutic protein comprises a FIX polypeptide and an XTEN, wherein the XTEN comprises 72 amino acids. In one embodiment, the therapeutic protein comprises a FIX polypeptide and an XTEN, wherein the XTEN comprises 72 amino acids, and the XTEN is inserted XTEN is inserted within the FIX polypeptide immediately downstream of amino acid 166 of mature FIX.

In some embodiments, the XTEN sequence used in the disclosure is at least 60%, 70%, 80%, 85%, 90%, 91%, 92%, 93%, 94%, 95%, 96%, 97%, 98%, 99%, or 100% identical to a sequence selected from the group consisting of AE42, AG42, AE48, AM48, AE72, AG72, AE108, AG108, AE144, AF144, AG144, AE180, AG180, AE216, AG216, AE252, AG252, AE288, AG288, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, AG2004, and any combination thereof. See US 2010-0239554 A1. In one particular embodiment, the XTEN comprises AE42, AE72, AE144, AE288, AE576, AE864, AG 42, AG72, AG144, AG288, AG576, AG864, or any combination thereof.

Exemplary XTEN sequences that can be used as heterologous moieties in the therapeutic protein of the disclosure include XTEN AE42-4 (SEQ ID NO: 46, encoded by SEQ ID NO: 47), XTEN AE144-2A (SEQ ID NO: 48, encoded by SEQ ID NO: 49), XTEN AE144-3B (SEQ ID NO: 50, encoded by SEQ ID NO: 51), XTEN AE144-4A (SEQ ID NO: 52, encoded by SEQ ID NO: 53), XTEN AE144-5A (SEQ ID NO: 54, encoded by SEQ ID NO: 55), XTEN AE144-6B (SEQ ID NO: 56, encoded by SEQ ID NO: 57), XTEN AG144-1 (SEQ ID NO: 58, encoded by SEQ ID NO: 59), XTEN AG144-A (SEQ ID NO: 60, encoded by SEQ ID NO: 61), XTEN AG144-B (SEQ ID NO: 62, encoded by SEQ ID NO: 63), XTEN AG144-C(SEQ ID NO: 64, encoded by SEQ ID NO: 65), and XTEN AG144-F (SEQ ID NO: 66, encoded by SEQ ID NO: 67). In one particular embodiment, the XTEN is encoded by SEQ ID NO:18.

In another embodiment, the XTEN sequence is selected from the group consisting of AE36 (SEQ ID NO: 130), AE42 (SEQ ID NO: 131), AE72 (SEQ ID NO: 132), AE78 (SEQ ID NO: 133), AE144 (SEQ ID NO: 134), AE144_2A (SEQ ID NO: 48), AE144_3B (SEQ ID NO: 50), AE144_4A (SEQ ID NO: 52), AE144_5A (SEQ ID NO: 54), AE144_6B (SEQ ID NO: 135), AG144 (SEQ ID NO: 136), AG144_A (SEQ ID NO: 137), AG144_B (SEQ ID NO: 62), AG144_C (SEQ ID NO: 64), AG144_F (SEQ ID NO: 66), AE288 (SEQ ID NO: 138), AE288_2 (SEQ ID NO: 139), AG288 (SEQ ID NO: 140), AE576 (SEQ ID NO: 141), AG576 (SEQ ID NO: 142), AE864 (SEQ ID NO: 143), AG864 (SEQ ID NO: 144), XTEN_AE72_2A_1 (SEQ ID NO:145), XTEN_AE72_2A_2 (SEQ ID NO: 146), XTEN_AE72_3B_1 (SEQ ID NO: 147), XTEN_AE72_3B_2 (SEQ ID NO: 148), XTEN_AE72_4A_2 (SEQ ID NO: 149), XTEN_AE72_5A2 (SEQ ID NO: 150), XTEN_AE72_6B_1 (SEQ ID NO: 151), XTEN_AE72_6S2 (SEQ ID NO: 152), XTEN_AE72_1A_1 (SEQ ID NO: 153), XTEN_AE72_1A_2 (SEQ ID NO: 154), XTEN_AE144_1A (SEQ ID NO: 155), AE150 (SEQ ID NO: 156), AG150 (SEQ ID NO: 157), AE294 (SEQ ID NO: 158), AG294 (SEQ ID NO: 159), and any combinations thereof. In a specific embodiment, the XTEN sequence is selected from the group consisting of AE72, AE144, and AE288. The amino acid sequences for certain XTEN sequences of the invention are shown in Table 8.

TABLE 8
XTEN Sequences

| XTEN | Amino Acid Sequence |
|---|---|
| AE42-4 (SEQ ID | GAPGSPAGSPTSTEEGTSESATPESGPGSEPATSGSETPASS |
| NO: 46) | |
| AE144-2A (SEQ ID | TSTEPSEGSAPGSPAGSPTSTEEGTSTEPSEGSAPGTSTEPSEGSAPGTSESATPESG |
| NO: 48) | PGTSTEPSEGSAPGTSESATPESGPGSEPATSGSETPGTSTEPSEGSAPGTSTEPSEG |
| | SAPGTSESATPESGPGTSESATPESGPG |
| A144-3B (SEQ ID | SPAGSPTSTEEGTSESATPESGPGSEPATSGSETPGTSESATPESGPGTSTEPSEGSA |
| NO: 50) | PGTSTEPSEGSAPGTSTEPSEGSAPGTSTEPSEGSAPGTSTEPSEGSAPGTSTEPSEG |
| | SAPGSPAGSPTSTEEGTSTEPSEGSAPG |
| AE144-4A (SEQ ID | TSESATPESGPGSEPATSGSETPGTSESATPESGPGSEPATSGSETPGTSESATPESG |
| NO: 52) | PGTSTEPSEGSAPGTSESATPESGPGSPAGSPTSTEEGSPAGSPTSTEEGSPAGSPTS |
| | TEEGTSESATPESGPGTSTEPSEGSAPG |
| AE144-5A (SEQ ID | TSESATPESGPGSEPATSGSETPGTSESATPESGPGSEPATSGSETPGTSESATPESG |
| NO: 54) | PGTSTEPSEGSAPGSPAGSPTSTEEGTSESATPESGPGSEPATSGSETPGTSESATPE |
| | SGPGSPAGSPTSTEEGSPAGSPTSTEEG |
| AE144-6B (SEQ ID | TSTEPSEGSAPGTSESATPESGPGTSESATPESGPGTSESATPESGPGSEPATSGSET |
| NO: 56) | PGSEPATSGSETPGSPAGSPTSTEEGTSTEPSEGSAPGTSTEPSEGSAPGSEPATSGS |
| | ETPGTSESATPESGPGTSTEPSEGSAPG |
| AG144-1 (SEQ ID | PGSSPSASTGTGPGSSPSASTGTGPGTPGSGTASSSPGSSTPSGATGSPGSSPSASTG |
| NO: 58) | TGPGASPGTSSTGSPGTPGSGTASSSPGSSTPSGATGSPGTPGSGTASSSPGASPGTS |
| | STGSPGASPGTSSTGSPGTPGSGTASSS |
| AG144-A (SEQ ID | GASPGTSSTGSPGSSPSASTGTGPGSSPSASTGTGPGTPGSGTASSSPGSSTPSGATG |
| NO: 60) | SPGSSPSASTGTGPGASPGTSSTGSPGTPGSGTASSSPGSSTPSGATGSPGTPGSGTA |
| | SSSPGASPGTSSTGSPGASPGTSSTGSP |
| AG144-B (SEQ ID | GTPGSGTASSSPGSSTPSGATGSPGASPGTSSTGSPGTPGSGTASSSPGSSTPSGATG |
| NO: 62) | SPGSSPSASTGTGPGSSPSASTGTGPGSSTPSGATGSPGSSTPSGATGSPGASPGTSS |
| | TGSPGASPGTSSTGSPGASPGTSSTGSP |
| AG144-C (SEQ ID | GTPGSGTASSSPGASPGTSSTGSPGASPGTSSTGSPGASPGTSSTGSPGSSPSASTGT |
| NO: 64) | GPGTPGSGTASSSPGASPGTSSTGSPGASPGTSSTGSPGASPGTSSTGSPGSSTPSGA |
| | TGSPGSSTPSGATGSPGASPGTSSTGSP |
| XTEN AG144-F | GSSPSASTGTGPGSSPSASTGTGPGASPGTSSTGSPGASPGTSSTGSPGSSTPSGATG |
| (SEQ ID NO: 66) | SPGSSPSASTGTGPGASPGTSSTGSPGSSPSASTGTGPGTPGSGTASSSPGSSTPSGA |
| | TGSPGSSTPSGATGSPGASPGTSSTGSP |
| AE36 | GSPAGSPTSTEEGTSESATPESGPGSEPATSGSETP |
| (SEQ ID NO: 130) | |
| AE42 | GAPGSPAGSPTSTEEGTSESATPESGPGSEPATSGSETPASS |
| (SEQ ID NO: 131) | |
| AE72 | GAPTSESATPESGPGSEPATSGSETPGTSESATPESGPGSEPATSGSETPGTSESATP |
| (SEQ ID NO: 132) | ESGPGTSTEPSEGSAPGASS |
| AE78 | GAPTSESATPESGPGSEPATSGSETPGTSESATPESGPGSEPATSGSETPGTSESATP |
| (SEQ ID NO: 133) | ESGPGTSTEPSEGSAPGASS |
| AE144 | GSEPATSGSETPGTSESATPESGPGSEPATSGSETPGSPAGSPTSTEEGTSTEPSEGS |
| (SEQ ID NO: 134) | APGSEPATSGSETPGSEPATSGSETPGSEPATSGSETPGTSTEPSEGSAPGTSESAPE |
| | SGPGSEPATSGSETPGTSTEPSEGSAP |
| AE144_6B | TSTEPSEGSAPGTSESATPESGPGTSESATPESGPGTSESATPESGPGSEPATSGSET |
| (SEQ ID NO: 135) | PGSEPATSGSETPGSPAGSPTSTEEGTSTEPSEGSAPGTSTEPSEGSAPGSEPATSGS |
| | ETPGTSESATPESGPGTSTEPSEGSAPG |
| AG144 | GTPGSGTASSSPGSSTPSGATGSPGSSPSASTGTGPGSSPSASTGTGPGASPGTSSTG |
| (SEQ ID NO: 136) | SPGASPGTSSTGSPGSSTPSGATGSPGSSPSASTGTGPGASPGTSSTGSPGSSPSAST |
| | GTGPGTPGSGTASSSPGSSTPSGATGSP |
| AG144_A | GASPGTSSTGSPGSSPSASTGTGPGSSPSASTGTGPGTPGSGTASSSPGSSTPSGATG |
| (SEQ ID NO: 137) | SPGSSPSASTGTGPGASPGTSSTGSPGTPGSGTASSSPGSSTPSGATGSPGTPGSGTA |
| | SSSPGASPGTSSTGSPGASPGTSSTGSP |
| AE288 | GTSESATPESGPGSEPATSGSETPGTSESATPESGPGSEPATSGSETPGTSESATPES |
| (SEQ ID NO: 138) | GPGTSTEPSEGSAPGSPAGSPTSTEEGTSESATPESGPGSEPATSGSETPGTSESATP |
| | ESGPGSPAGSPTSTEEGSPAGSPTSTEEGTSTEPSEGSAPGTSESATPESGPGTSESA |
| | TPESGPGTSESATPESGPGSEPATSGSETPGSEPATSGSETPGSPAGSPTSTEEGTST |
| | EPSEGSAPGTSTEPSEGSAPGSEPATSGSETPGTSESATPESGPGTSTEPSEGSAP |
| AE288_2 | GSPAGSPTSTEEGTSESATPESGPGSEPATSGSETPGTSESATPESGPGTSTEPSEGS |
| (SEQ ID NO: 139) | APGTSTEPSEGSAPGTSTEPSEGSAPGTSTEPSEGSAPGTSTEPSEGSAPGTSTEPSE |
| | GSAPGSPAGSPTSTEEGTSTEPSEGSAPGTSESATPESGPGSEPATSGSETPGTSESA |
| | TPESGPGSEPATSGSETPGTSESATPESGPGTSTEPSEGSAPGTSESATPESGPGSPA |
| | GSPTSTEEGSPAGSPTSTEEGSPAGSPTSTEEGTSESATPESGPGTSTEPSEGSAP |
| AG288 | PGASPGTSSTGSPGASPGTSSTGSPGTPGSGTASSSPGSSTPSGATGSPGTPGSGTAS |
| (SEQ ID NO: 140) | SSPGSSTPSGATGSPGTPGSGTASSSPGSSTPSGATGSPGSSTPSGATGSPGSSPSAS |
| | TGTGPGSSPSASTGTGPGASPGTSSTGSPGTPGSGTASSSPGSSTPSGATGSPGSSPS |
| | ASTGTGPGSSPSASTGTGPGASPGTSSTGSPGASPGTSSTGSPGSSTPSGATGSPGSS |
| | PSASTGTGPGASPGTSSTGSPGSSPSASTGTGPGTPGSGTASSSPGSSTPSGATGS |
| AE576 | GSPAGSPTSTEEGTSESATPESGPGTSTEPSEGSAPGSPAGSPTSTEEGTSTEPSEGS |
| (SEQ ID NO: 141) | APGTSTEPSEGSAPGTSESATPESGPGSEPATSGSETPGSEPATSGSETPGSPAGSPT |
| | STEEGTSESATPESGPGTSTEPSEGSAPGTSTEPSEGSAPGSPAGSPTSTEEGTSTEP |
| | SEGSAPGTSTEPSEGSAPGTSESATPESGPGTSTEPSEGSAPGTSESATPESGPGSEP |
| | ATSGSETPGTSTEPSEGSAPGTSTEPSEGSAPGTSESATPESGPGTSESATPESGPGS |
| | PAGSPTSTEEGTSESATPESGPGSEPATSGSETPGTSESATPESGPGTSTEPSEGSAP |
| | GTSTEPSEGSAPGTSTEPSEGSAPGTSTEPSEGSAPGTSTEPSEGSAPGTSTEPSEGS |
| | APGSPAGSPTSTEEGTSTEPSEGSAPGTSESATPESGPGSEPATSGSETPGTSESATP |
| | ESGPGSEPATSGSETPGTSESATPESGPGTSTEPSEGSAPGTSESATPESGPGSPAGS |
| | PTSTEEGSPAGSPTSTEEGSPAGSPTSTEEGTSESATPESGPGTSTEPSEGSAP |
| AG576 | PGTPGSGTASSSPGSSTPSGATGSPGSSPSASTGTGPGSSPSASTGTGPGSSTPSGAT |
| (SEQ ID NO: 142) | GSPGSSTPSGATGSPGASPGTSSTGSPGASPGTSSTGSPGASPGTSSTGSPGTPGSGT |
| | ASSSPGASPGTSSTGSPGASPGTSSTGSPGASPGTSSTGSPGSSPSASTGTGPGTPGS |
| | GTASSSPGASPGTSSTGSPGASPGTSSTGSPGASPGTSSTGSPGSSTPSGATGSPGSS |
| | TPSGATGSPGASPGTSSTGSPGTPGSGTASSSPGSSTPSGATGSPGSSTPSGATGSPG |
| | SSTPSGATGSPGSSPSASTGTGPGASPGTSSTGSPGASPGTSSTGSPGTPGSGTASSS |
| | PGASPGTSSTGSPGASPGTSSTGSPGASPGTSSTGSPGASPGTSSTGSPGTPGSGTAS |
| | SSPGSSTPSGATGSPGTPGSGTASSSPGSSTPSGATGSPGTPGSGTASSSPGSSTPSG |
| | ATGSPGSSTPSGATGSPGSSPSASTGTGPGSSPSASTGTGPGASPGTSSTGSPGTPGS |
| | GTASSSPGSSTPSGATGSPGSSPSASTGTGPGSSPSASTGTGPGASPGTSSTGS |
| AE864 | GSPAGSPTSTEEGTSESATPESGPGTSTEPSEGSAPGSPAGSPTSTEEGTSTEPSEGS |
| (SEQ ID NO: 143) | APGTSTEPSEGSAPGTSESATPESGPGSEPATSGSETPGSEPATSGSETPGSPAGSPT |
| | STEEGTSESATPESGPGTSTEPSEGSAPGTSTEPSEGSAPGSPAGSPTSTEEGTSTEP |
| | SEGSAPGTSTEPSEGSAPGTSESATPESGPGTSTEPSEGSAPGTSESATPESGPGSEP |
| | ATSGSETPGTSTEPSEGSAPGTSTEPSEGSAPGTSESATPESGPGTSESATPESGPGS |
| | PAGSPTSTEEGTSESATPESGPGSEPATSGSETPGTSESATPESGPGTSTEPSEGSAP |
| | GTSTEPSEGSAPGTSTEPSEGSAPGTSTEPSEGSAPGTSTEPSEGSAPGTSTEPSEGS |
| | APGSPAGSPTSTEEGTSTEPSEGSAPGTSESATPESGPGSEPATSGSETPGTSESATP |
| | ESGPGSEPATSGSETPGTSESATPESGPGTSTEPSEGSAPGTSESATPESGPGSPAGS |
| | PTSTEEGSPAGSPTSTEEGSPAGSPTSTEEGTSESATPESGPGTSTEPSEGSAPGTSE |
| | SATPESGPGSEPATSGSETPGTSESATPESGPGSEPATSGSETPGTSESATPESGPGT |
| | STEPSEGSAPGSPAGSPTSTEEGTSESATPESGPGSEPATSGSETPGTSESATPESGP |
| | GSPAGSPTSTEEGSPAGSPTSTEEGTSTEPSEGSAPGTSESATPESGPGTSESATPES |
| | GPGTSESATPESGPGSEPATSGSETPGSEPATSGSETPGSPAGSPTSTEEGTSTEPSE |
| | GSAPGTSTEPSEGSAPGSEPATSGSETPGTSESATPESGPGTSTEPSEGSAP |
| AG864 | GASPGTSSTGSPGSSPSASTGTGPGSSPSASTGTGPGTPGSGTASSSPGSSTPSGATG |
| (SEQ ID NO: 144) | SPGSSPSASTGTGPGASPGTSSTGSPGTPGSGTASSSPGSSTPSGATGSPGTPGSGTA |
| | SSSPGASPGTSSTGSPGASPGTSSTGSPGTPGSGTASSSPGSSTPSGATGSPGASPGT |
| | SSTGSPGTPGSGTASSSPGSSTPSGATGSPGSSPSASTGTGPGSSPSASTGTGPGSST |
| | PSGATGSPGSSTPSGATGSPGASPGTSSTGSPGASPGTSSTGSPGASPGTSSTGSPGT |
| | PGSGTASSSPGASPGTSSTGSPGASPGTSSTGSPGASPGTSSTGSPGSSPSASTGTGP |
| | GTPGSGTASSSPGASPGTSSTGSPGASPGTSSTGSPGASPGTSSTGSPGSSTPSGATG |
| | SPGSSTPSGATGSPGASPGTSSTGSPGTPGSGTASSSPGSSTPSGATGSPGSSTPSGA |
| | TGSPGSSTPSGATGSPGSSPSASTGTGPGASPGTSSTGSPGASPGTSSTGSPGTPGSG |
| | TASSSPGASPGTSSTGSPGASPGTSSTGSPGASPGTSSTGSPGASPGTSSTGSPGTPG |
| | SGTASSSPGSSTPSGATGSPGTPGSGTASSSPGSSTPSGATGSPGTPGSGTASSSPGS |
| | STPSGATGSPGSSTPSGATGSPGSSPSASTGTGPGSSPSASTGTGPGASPGTSSTGSP |
| | GTPGSGTASSSPGSSTPSGATGSPGSSPSASTGTGPGSSPSASTGTGPGASPGTSSTG |
| | SPGASPGTSSTGSPGSSTPSGATGSPGSSPSASTGTGPGASPGTSSTGSPGSSPSAST |
| | GTGPGTPGSGTASSSPGSSTPSGATGSPGSSTPSGATGSPGASPGTSSTGSP |
| XTEN_AE72_2A_1 | TSTEPSEGSAPGSPAGSPTSTEEGTSTEPSEGSAPGTSTEPSEGSAPGTSESATPESG |
| (SEQ ID NO: 145) | PGTSTEPSEGSAPG |
| XTEN_AE72_2A_2 | TSESATPESGPGSEPATSGSETPGTSTEPSEGSAPGTSTEPSEGSAPGTSESATPESG |
| (SEQ ID NO: 146) | PGTSESATPESGPG |
| XTEN_AE72_3B_1 | SPAGSPTSTEEGTSESATPESGPGSEPATSGSETPGTSESATPESGPGTSTEPSEGSA |
| (SEQ ID NO: 147) | PGTSTEPSEGSAPG |
| XTEN_AE72_3B_2 | TSTEPSEGSAPGTSTEPSEGSAPGTSTEPSEGSAPGTSTEPSEGSAPGSPAGSPTSTE |
| (SEQ ID NO: 148) | EGTSTEPSEGSAPG |
| XTEN_AE72_4A_2 | TSESATPESGPGSPAGSPTSTEEGSPAGSPTSTEEGSPAGSPTSTEEGTSESATPESG |
| (SEQ ID NO: 149) | PGTSTEPSEGSAPG |
| XTEN_AE72_5A_2 | SPAGSPTSTEEGTSESATPESGPGSEPATSGSETPGTSESATPESGPGSPAGSPTSTE |
| (SEQ ID NO: 150) | EGSPAGSPTSTEEG |
| XTEN_AE72_6B_1 | TSTEPSEGSAPGTSESATPESGPGTSESATPESGPGTSESATPESGPGSEPATSGSET |
| (SEQ ID NO: 151) | PGSEPATSGSETPG |
| XTEN_AE72_6B_2 | SPAGSPTSTEEGTSTEPSEGSAPGTSTEPSEGSAPGSEPATSGSETPGTSESATPESG |
| (SEQ ID NO: 152) | PGTSTEPSEGSAPG |
| XTEN_AE72_1A_1 | SPAGSPTSTEEGTSESATPESGPGTSTEPSEGSAPGSPAGSPTSTEEGTSTEPSEGSA |
| (SEQ ID NO: 153) | PGTSTEPSEGSAPG |
| XTEN_AE72_1A 2 | TSESATPESGPGSEPATSGSETPGSEPATSGSETPGSPAGSPTSTEEGTSESATPESG |
| (SEQ ID NO: 154) | PGTSTEPSEGSAPG |
| XTEN_AE144_1A | SPAGSPTSTEEGTSESATPESGPGTSTEPSEGSAPGSPAGSPTSTEEGTSTEPSEGSA |
| (SEQ ID NO: 155) | PGTSTEPSEGSAPGTSESATPESGPGSEPATSGSETPGSEPATSGSETPGSPAGSPTS |
| | TEEGTSESATPESGPGTSTEPSEGSAPG |
| AE150 | GAPGSEPATSGSETPGTSESATPESGPGSEPATSGSETPGSPAGSPTSTEEGTSTEPS |
| (SEQ ID NO: 156) | EGSAPGSEPATSGSETPGSEPATSGSETPGSEPATSGSETPGTSTEPSEGSAPGTSES |
| | ATPESGPGSEPATSGSETPGTSTEPSEGSAPASS |
| G150 | GAPGTPGSGTASSSPGSSTPSGATGSPGSSPSASTGTGPGSSPSASTGTGPGASPGTS |
| (SEQ ID NO: 157) | STGSPGASPGTSSTGSPGSSTPSGATGSPGSSPSASTGTGPGASPGTSSTGSPGSSPS |
| | ASTGTGPGTPGSGTASSSPGSSTPSGATGSPASS |
| AE294 | GAPGTSESATPESGPGSEPATSGSETPGTSESATPESGPGSEPATSGSETPGTSESAT |
| (SEQ ID NO: 158) | PESGPGTSTEPSEGSAPGSPAGSPTSTEEGTSESATPESGPGSEPATSGSETPGTSES |
| | ATPESGPGSPAGSPTSTEEGSPAGSPTSTEEGTSTEPSEGSAPGTSESATPESGPGTS |
| | ESATPESGPGTSESATPESGPGSEPATSGSETPGSEPATSGSETPGSPAGSPTSTEEG |
| | TSTEPSEGSAPGTSTEPSEGSAPGSEPATSGSETPGTSESATPESGPGTSTEPSEGSA |
| | PASS |
| AG294 | GAPPGASPGTSSTGSPGASPGTSSTGSPGTPGSGTASSSPGSSTPSGATGSPGTPGSG |
| (SEQ ID NO: 159) | TASSSPGSSTPSGATGSPGTPGSGTASSSPGSSTPSGATGSPGSSTPSGATGSPGSSP |
| | SASTGTGPGSSPSASTGTGPGASPGTSSTGSPGTPGSGTASSSPGSSTPSGATGSPGS |
| | SPSASTGTGPGSSPSASTGTGPGASPGTSSTGSPGASPGTSSTGSPGSSTPSGATGSP |
| | GSSPSASTGTGPGASPGTSSTGSPGSSPSASTGTGPGTPGSGTASSSPGSSTPSGATG |
| | SASS |

In some embodiments, less than 100% of amino acids of an XTEN are selected from glycine (G), alanine (A), serine (S), threonine (T), glutamate (E) and proline (P), or less than 100% of the sequence consists of the sequence motifs from Table 7 or an XTEN sequence provided herein. In such embodiments, the remaining amino acid residues of the XTEN are selected from any of the other 14 natural L-amino acids, but can be preferentially selected from hydrophilic amino acids such that the XTEN sequence contains at least about 90%, 91%, 92%, 93%, 94%, 95%, 96%, 97%, 98%, or at least about 99% hydrophilic amino acids. The content of hydrophobic amino acids in the XTEN utilized in the conjugation constructs can be less than 5%, or less than 2%, or less than 1% hydrophobic amino acid content. Hydrophobic residues that are less favored in construction of XTEN include tryptophan, phenylalanine, tyrosine, leucine, isoleucine, valine, and methionine. Additionally, XTEN sequences can contain less than 5% or less than 4% or less than 3% or less than 2% or less than 1% or none of the following amino acids: methionine (for example, to avoid oxidation), or asparagine and glutamine (to avoid desamidation).

The one or more XTEN sequences can be inserted at the C-terminus or at the N-terminus of the therapeutic protein or inserted between two amino acids in the amino acid sequence of the therapeutic protein. For example, where the therapeutic protein comprises a FVIII polypeptide, the XTEN can be inserted between two amino acids at one or more insertion site selected from Table 5. Where the therapeutic protein comprises a FIX polypeptide, the XTEN can be inserted between two amino acids at one or more insertion site selected from Table 5.

Additional examples of XTEN sequences that can be used according to the present invention and are disclosed in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, WO 2011028344 A2, WO 2014/011819 A2, or WO 2015/023891.

### 5. Albumin or Fragment, Derivative, or Variant Thereof

In some embodiments, a heterologous moiety comprises albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, which are incorporated herein by reference in their entireties.

In one embodiment, the therapeutic protein of the disclosure comprises albumin, a fragment, or a variant thereof which is further linked to a second heterologous moiety selected from the group consisting of an immunoglobulin constant region or portion thereof (e.g., an Fc region), a PAS sequence, HES, PEG and any combination thereof.

### 6. Albumin-binding Moiety

In certain embodiments, the heterologous moiety is an albumin-binding moiety, which comprises an albumin-binding peptide, a bacterial albumin-binding domain, an albumin-binding antibody fragment, or any combinations thereof.

For example, the albumin-binding protein can be a bacterial albumin-binding protein, an antibody or an antibody fragment including domain antibodies (see U.S. Pat. No. 6,696,245). An albumin-binding protein, for example, can be a bacterial albumin-binding domain, such as the one of streptococcal protein G (Konig, T. and Skerra, A. (1998) J. Immunol. Methods 218, 73-83). Other examples of albumin-binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, lie, Leu, or Lys; Xaa₃ is Ala, Asp, Phe, Trp, or Tyr; and Xaa₄ is Asp, Gly, Leu, Phe, Ser, or Thr as described in US patent application 2003/0069395 or Dennis et al. (Dennis et al. (2002) J. Biol. Chem. 277, 35035-35043).

Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO: 35). See, e,g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties. An example of such albumin-binding moiety is 2-(3-maleimidopropanamido)-6-(4-(4-iodophenyl)butanamido) hexanoate (“Albu” tag) as disclosed by Trussel et al., Bioconjugate Chem. 20:2286-2292 (2009).

Fatty acids, in particular long chain fatty acids (LCFA) and long chain fatty acid-like albumin-binding compounds can be used to extend the in vivo half-life of clotting factor proteins of the disclosure. An example of a LCFA-like albumin-binding compound is 16-(I-(3-(9-(((2,5-dioxopyrrolidin-I-yloxy) carbonyloxy)-methyi)-7-sulfo-9H-fluoren-2-ylamino)-3-oxopropyl)-2,5-dioxopyrrolidin-3-ylthio) hexadecanoic acid (see, e.g., WO 2010/140148).

### 7. PAS Sequence

In other embodiments, the heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. Yet, the skilled person is aware that an amino acid polymer also can form random coil conformation when residues other than alanine, serine, and proline are added as a minor constituent in the PAS sequence. The term “minor constituent” as used herein means that amino acids other than alanine, serine, and proline can be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline can be selected from the group consisting of Arg, Asn, Asp, Cys, Gln, Glu, Gly, His, lie, Leu, Lys, Met, Phe, Thr, Trp, Tyr, and Val.

Under physiological conditions, the PAS sequence stretch forms a random coil conformation and thereby can mediate an increased in vivo and/or in vitro stability to the clotting factor protein. Since the random coil domain does not adopt a stable structure or function by itself, the biological activity mediated by the clotting factor protein is essentially preserved. In other embodiments, the PAS sequences that form random coil domain are biologically inert, especially with respect to proteolysis in blood plasma, immunogenicity, isoelectric point/electrostatic behaviour, binding to cell surface receptors or internalisation, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO: 36), AAPASPAPAAPSAPAPAAPS (SEQ ID NO: 37), APSSPSPSAPSSPSPASPSS (SEQ ID NO: 38), APSSPSPSAPSSPSPASPS (SEQ ID NO: 39), SSPSAPSPSSPASPSPSSPA (SEQ ID NO: 40), AASPAAPSAPPAAASPAAPSAPPA (SEQ ID NO: 41), ASAAAPAAASAAASAPSAAA (SEQ ID NO: 42) and any combinations thereof. Additional examples of PAS sequences are known from, e.g., US Pat. Publ. No. 2010/0292130 A1 and PCT Appl. Publ. No. WO 2008/155134 A1.

### 8. HAP Sequence

In certain embodiments, the heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

### 9. Transferrin or Fragment Thereof

In certain embodiments, the heterologous moiety is transferrin or a fragment thereof. Any transferrin can be used to make the clotting factor proteins of the disclosure. As an example, wild-type human TF (TF) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin heterologous moiety includes a transferrin splice variant. In one example, a transferrin splice variant can be a splice variant of human transferrin, e.g., Genbank Accession AAA61140. In another embodiment, the transferrin portion of the chimeric protein includes one or more domains of the transferrin sequence, e.g., N domain, C domain, N1 domain, N2 domain, C1 domain, C2 domain or any combinations thereof.

### 10. Clearance Receptors

In certain embodiments, the heterologous moiety is a clearance receptor, fragment, variant, or derivative thereof. LRP1 is a 600 kDa integral membrane protein that is implicated in the receptor-mediate clearance of a variety of proteins, such as Factor X. See, e.g., Narita et al., Blood 91:555-560 (1998).

### 11. von Willebrand Factor or Fragments Thereof

In certain embodiments, the heterologous moiety is von Willebrand Factor (VWF) or one or more fragments thereof.

VWF (also known as F8VWF) is a large multimeric glycoprotein present in blood plasma and produced constitutively in endothelium (in the Weibel-Palade bodies), megakaryocytes (α-granules of platelets), and subendothelian connective tissue. The basic VWF monomer is a 2813 amino acid protein. Every monomer contains a number of specific domains with a specific function, the D′ and D3 domains (which together bind to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (βHCG)).

The 2813 monomer amino acid sequence for human VWF is reported as Accession Number NP000543.2 in Genbank. The nucleotide sequence encoding the human VWF is reported as Accession Number NM000552.3 in Genbank. SEQ ID NO: 129 is the amino acid sequence encoded by SEQ ID NO: 128. The D′ domain includes amino acids 764 to 866 of SEQ ID NO: 129. The D3 domain includes amino acids 867 to 1240 of SEQ ID NO: 44.

In plasma, 95-98% of FVIII circulates in a tight non-covalent complex with full-length VWF. The formation of this complex is important for the maintenance of appropriate plasma levels of FVIIII in vivo. Lenting et al., Blood. 92(11): 3983-96 (1998); Lenting et al., J. Thromb. Haemost. 5(7): 1353-60 (2007). When FVIII is activated due to proteolysis at positions 372 and 740 in the heavy chain and at position 1689 in the light chain, the VWF bound to FVIII is removed from the activated FVIII.

In certain embodiments, the heterologous moiety is full length von Willebrand Factor. In other embodiments, the heterologous moiety is a von Willebrand Factor fragment. As used herein, the term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. In a specific embodiment, the heterologous moiety is a (VWF) fragment comprising a D′ domain and a D3 domain of VWF. The VWF fragment comprising the D′ domain and the D3 domain can further comprise a VWF domain selected from the group consisting of an A1 domain, an A2 domain, an A3 domain, a D1 domain, a D2 domain, a D4 domain, a B1 domain, a B2 domain, a B3 domain, a C1 domain, a C2 domain, a CK domain, one or more fragments thereof, and any combinations thereof. Additional examples of the polypeptide having FVIII activity fused to the VWF fragment are disclosed in U.S. provisional patent application No. 61/667,901, filed Jul. 3, 2012, and U.S. Publication No. 2015/0023959 A1, which are both incorporated herein by reference in its entirety.

### 12. Linker Moieties

In certain embodiments, the heterologous moiety is a peptide linker.

As used herein, the terms “peptide linkers” or “linker moieties” refer to a peptide or polypeptide sequence (e.g., a synthetic peptide or polypeptide sequence) which connects two domains in a linear amino acid sequence of a polypeptide chain.

In some embodiments, peptide linkers can be inserted between the therapeutic protein of the disclosure and a heterologous moiety described above, such as albumin. Peptide linkers can provide flexibility to the chimeric polypeptide molecule. Linkers are not typically cleaved, however such cleavage can be desirable. In one embodiment, these linkers are not removed during processing.

A type of linker which can be present in a chimeric protein of the disclosure is a protease cleavable linker which comprises a cleavage site (i.e., a protease cleavage site substrate, e.g., a factor XIa, Xa, or thrombin cleavage site) and which can include additional linkers on either the N-terminal of C-terminal or both sides of the cleavage site. These cleavable linkers when incorporated into a construct of the disclosure result in a chimeric molecule having a heterologous cleavage site.

In one embodiment, a therapeutic protein encoded by a nucleic acid molecule of the instant disclosure comprises two or more Fc domains or moieties linked via a cscFc linker to form an Fc region comprised in a single polypeptide chain. The cscFc linker is flanked by at least one intracellular processing site, i.e., a site cleaved by an intracellular enzyme. Cleavage of the polypeptide at the at least one intracellular processing site results in a polypeptide which comprises at least two polypeptide chains.

Other peptide linkers can optionally be used in a construct of the disclosure, e.g., to connect a clotting factor protein to an Fc region. Some exemplary linkers that can be used in connection with the disclosure include, e.g., polypeptides comprising GlySer amino acids described in more detail below.

In one embodiment, the peptide linker is synthetic, i.e., non-naturally occurring. In one embodiment, a peptide linker includes peptides (or polypeptides) (which can or cannot be naturally occurring) which comprise an amino acid sequence that links or genetically fuses a first linear sequence of amino acids to a second linear sequence of amino acids to which it is not naturally linked or genetically fused in nature. For example, in one embodiment the peptide linker can comprise non-naturally occurring polypeptides which are modified forms of naturally occurring polypeptides (e.g., comprising a mutation such as an addition, substitution or deletion). In another embodiment, the peptide linker can comprise non-naturally occurring amino acids. In another embodiment, the peptide linker can comprise naturally occurring amino acids occurring in a linear sequence that does not occur in nature. In still another embodiment, the peptide linker can comprise a naturally occurring polypeptide sequence.

For example, in certain embodiments, a peptide linker can be used to fuse identical Fc moieties, thereby forming a homodimeric scFc region. In other embodiments, a peptide linker can be used to fuse different Fc moieties (e.g. a wild-type Fc moiety and an Fc moiety variant), thereby forming a heterodimeric scFc region.

In another embodiment, a peptide linker comprises or consists of a gly-ser linker. In one embodiment, a scFc or cscFc linker comprises at least a portion of an immunoglobulin hinge and a gly-ser linker. As used herein, the term “gly-ser linker” refers to a peptide that consists of glycine and serine residues. In certain embodiments, said gly-ser linker can be inserted between two other sequences of the peptide linker. In other embodiments, a gly-ser linker is attached at one or both ends of another sequence of the peptide linker. In yet other embodiments, two or more gly-ser linker are incorporated in series in a peptide linker. In one embodiment, a peptide linker of the disclosure comprises at least a portion of an upper hinge region (e.g., derived from an IgG1, IgG2, IgG3, or IgG4 molecule), at least a portion of a middle hinge region (e.g., derived from an IgG1, IgG2, IgG3, or IgG4 molecule) and a series of gly/ser amino acid residues.

Peptide linkers of the disclosure are at least one amino acid in length and can be of varying lengths. In one embodiment, a peptide linker of the disclosure is from about 1 to about 50 amino acids in length. As used in this context, the term “about” indicates +/− two amino acid residues. Since linker length must be a positive integer, the length of from about 1 to about 50 amino acids in length, means a length of from 1-3 to 48-52 amino acids in length. In another embodiment, a peptide linker of the disclosure is from about 10 to about 20 amino acids in length. In another embodiment, a peptide linker of the disclosure is from about 15 to about 50 amino acids in length. In another embodiment, a peptide linker of the disclosure is from about 20 to about 45 amino acids in length. In another embodiment, a peptide linker of the disclosure is from about 15 to about 35 or about 20 to about 30 amino acids in length. In another embodiment, a peptide linker of the disclosure is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 500, 1000, or 2000 amino acids in length. In one embodiment, a peptide linker of the disclosure is 20 or 30 amino acids in length.

In some embodiments, the peptide linker can comprise at least two, at least three, at least four, at least five, at least 10, at least 20, at least 30, at least 40, at least 50, at least 60, at least 70, at least 80, at least 90, or at least 100 amino acids. In other embodiments, the peptide linker can comprise at least 200, at least 300, at least 400, at least 500, at least 600, at least 700, at least 800, at least 900, or at least 1,000 amino acids. In some embodiments, the peptide linker can comprise at least about 10, 20, 30, 40, 50, 60, 70, 80, 90, 100, 150, 200, 300, 400, 500, 600, 700, 800, 900, 1000, 1100, 1200, 1300, 1400, 1500, 1600, 1700, 1800, 1900, or 2000 amino acids. The peptide linker can comprise 1-5 amino acids, 1-10 amino acids, 1-20 amino acids, 10-50 amino acids, 50-100 amino acids, 100-200 amino acids, 200-300 amino acids, 300-400 amino acids, 400-500 amino acids, 500-600 amino acids, 600-700 amino acids, 700-800 amino acids, 800-900 amino acids, or 900-1000 amino acids.

Peptide linkers can be introduced into polypeptide sequences using techniques known in the art. Modifications can be confirmed by DNA sequence analysis. Plasmid DNA can be used to transform host cells for stable production of the polypeptides produced.

### 13. Monomer-Dimer Hybrids

In some embodiments, the therapeutic protein of the disclosure comprises a monomer-dimer hybrid molecule comprising a clotting factor.

The term “monomer-dimer hybrid” used herein refers to a chimeric protein comprising a first polypeptide chain and a second polypeptide chain, which are associated with each other by a disulfide bond, wherein the first chain comprises a clotting factor, e.g., FVIII, and a first Fc region and the second chain comprises, consists essentially of, or consists of a second Fc region without the clotting factor. The monomer-dimer hybrid construct thus is a hybrid comprising a monomer aspect having only one clotting factor and a dimer aspect having two Fc regions.

### 14. Expression Control Sequences

In some embodiments, the nucleic acid molecule of the disclosure further comprises at least one expression control sequence. An expression control sequence, as used herein, is any regulatory nucleotide sequence, such as a promoter sequence or promoter-enhancer combination, which facilitates the efficient transcription and translation of the coding nucleic acid to which it is operably linked. For example, the nucleic acid molecule of the disclosure can be operably linked to at least one transcription control sequence. The gene expression control sequence can, for example, be a mammalian or viral promoter, such as a constitutive or inducible promoter.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one embodiment, the disclosure includes expression of a transgene under the control of a tissue specific promoter and/or enhancer. In another embodiment, the promoter or other expression control sequence selectively enhances expression of the transgene in liver cells. In certain embodiments, the promoter or other expression control sequence selective enhances expression of the transgene in hepatocytes, sinusoidal cells, and/or endothelial cells. In one particular embodiment, the promoter or other expression control sequence selective enhances expression of the transgene in endothelial cells. In certain embodiments, the promoter or other expression control sequence selective enhances expression of the transgene in muscle cells, the central nervous system, the eye, the liver, the heart, or any combination thereof. Examples of liver specific promoters include, but are not limited to, a mouse thyretin promoter (mTTR), an endogenous human factor VIII promoter (F8), human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The F8 promoter is described in Figueiredo and Brownlee, 1995, J. Biol. Chem. 270:11828-11838. In some embodiments, the promoter is selected from a liver specific promoter (e.g., α1-antitrypsin (AAT)), a muscle specific promoter (e.g., muscle creatine kinase (MCK), myosin heavy chain alpha (αMHC), myoglobin (MB), and desmin (DES)), a synthetic promoter (e.g., SPc5-12, 2R5Sc5-12, dMCK, and tMCK), and any combination thereof.

In one embodiment, the promoter is selected from the group consisting of a mouse thyretin promoter (mTTR), an endogenous human factor VIII promoter (F8), human alpha-1-antitrypsin promoter (hAAT), human albumin minimal promoter, mouse albumin promoter, TTPp, a CASI promoter, a CAG promoter, a cytomegalovirus (CMV) promoter, α1-antitrypsin (AAT), muscle creatine kinase (MCK), myosin heavy chain alpha (αMHC), myoglobin (MB), desmin (DES), SPc5-12, 2R5Sc5-12, dMCK, and tMCK, a phosphoglycerate kinase (PGK) promoter and any combination thereof.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

In a particular example, a promoter useful for the disclosure comprises SEQ ID NO: 69 (i.e., ET promoter), which is also known as GenBank No. AY661265. See also Vigna et al., Molecular Therapy 11(5):763 (2005). Examples of other suitable vectors and gene regulatory elements are described in WO 02/092134, EP1395293, or U.S. Pat. Nos. 6,808,905, 7,745,179, or 7,179,903, which are incorporated by reference herein in their entireties.

In one embodiment, the nucleic acid molecules of the present disclosure further comprises an intronic sequence. In some embodiments, the intronic sequence is positioned 5′ to the nucleic acid sequence encoding the FVIII polypeptide. In some embodiments, the intronic sequence is a naturally occurring intronic sequence. In some embodiments, the intronic sequence is a synthetic sequence. In some embodiments, the intronic sequence is derived from a naturally occurring intronic sequence. In certain embodiments, the intronic sequence comprises the SV40 small T intron. In one embodiment, the intronic sequence comprises SEQ ID NO: 115.

In some embodiments, the nucleic acid molecule further comprises a post-transcriptional regulatory element. In certain embodiments, the post-transcriptional regulatory element comprises a mutated woodchuck hepatitis virus post-transcriptional regulatory element (WPRE). In one particular embodiment, the post-transcriptional regulatory element comprises SEQ ID NO: 120.

In some embodiments, the nucleic acid molecule comprises a microRNA (miRNA) binding site. In one embodiment, the miRNA binding site is a miRNA binding site for miR-142-3p. In other embodiments, the miRNA binding site is selected from a miRNA binding site disclosed by Rennie et al., RNA Biol. 13(6):554-560 (2016), and STarMirDB, available at http://sfold.wadsworth.org/starmirDB.php, which are incorporated by reference herein in their entirety.

In some embodiments, the nucleic acid molecule comprises one or more DNA nuclear targeting sequences (DTSs). A DTS promotes translocation of DNA molecules containing such sequences into the nucleus. In certain embodiments, the DTS comprises an SV40 enhancer sequence. In certain embodiments, the DTS comprises a c-Myc enhancer sequence. In some embodiments, DTSs are between the first ITR and the second ITR. In some embodiments, the DTS is 3′ to the first ITR and 5′ to the therapeutic protein. In other embodiments, the DTS is 3′ to the therapeutic protein and 5′ to the second ITR.

In some embodiments, the nucleic acid molecule further comprises a 3′UTR poly(A) tail sequence. In one embodiment, the 3′UTR poly(A) tail sequence comprises bGH poly(A). In one embodiment, the 3′UTR poly(A) tail comprises an actin poly(A) site. In one embodiment, the 3′UTR poly(A) tail comprises a hemoglobin poly(A) site.

In one particular embodiment, the 3′UTR poly(A) tail sequence comprises SEQ ID NO: 122.

### III. Tissue Specific Expression

In certain embodiments, it will be useful to include within the vector one or more miRNA target sequences which, for example, are operably linked to the clotting factor transgene. Thus, the disclosure also provides at least one miRNA sequence target operably linked to the clotting factor nucleotide sequence or otherwise inserted within a vector. More than one copy of a miRNA target sequence included in the vector can increase the effectiveness of the system. Also included are different miRNA target sequences. For example, vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the vector in antisense orientation. Antisense orientation can be useful in the production of viral particles to avoid expression of gene products which can otherwise be toxic to the producer cells. In other embodiments, the vector comprises 1, 2, 3, 4, 5, 6, 7 or 8 copies of the same or different miRNA target sequence. However, in certain other embodiments, the vector will not include any miRNA target sequence. Choice of whether or not to include a miRNA target sequence (and how many) will be guided by known parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (tccataaagt aggaaacact aca (SEQ ID NO: 43)). In one embodiment, the vector comprises 4 copies of miR-142 target sequences. In certain embodiments, the complementary sequence of hematopoietic-specific microRNAs, such as miR-142 (142T), is incorporated into the 3′ untranslated region of a vector, e.g., lentiviral vectors (LV), making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can imposes a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

In some embodiments, the transgene expression is targeted to the liver. In certain embodiments, the transgene expression is targeted to hepatocytes. In other embodiment, the transgene expression is targeted to endothelial cells. In one particular embodiment, the transgene expression is targeted to any tissue that naturally expressed endogenous FVIII.

In some embodiments, the transgene expression is targeted to the central nervous system. In certain embodiments, the transgene expression is targeted to neurons. In some embodiments, the transgene expression is targeted to afferent neurons. In some embodiments, the transgene expression is targeted to efferent neurons. In some embodiments, the transgene expression is targeted to interneurons. In some embodiments, the transgene expression is targeted to glial cells. In some embodiments, the transgene expression is targeted to astrocytes. In some embodiments, the transgene expression is targeted to oligodendrocytes. In some embodiments, the transgene expression is targeted to microglia. In some embodiments, the transgene expression is targeted to ependymal cells. In some embodiments, the transgene expression is targeted to Schwann cells. In some embodiments, the transgene expression is targeted to satellite cells.

In some embodiments, the transgene expression is targeted to muscle tissue. In some embodiments, the transgene expression is targeted to smooth muscle. In some embodiments, the transgene expression is targeted to cardiac muscle. In some embodiments, the transgene expression is targeted to skeletal muscle.

In some embodiments, the transgene expression is targeted to the eye. In some embodiments, the transgene expression is targeted to a photoreceptor cell. In some embodiments, the transgene expression is targeted to retinal ganglion cell.

### IV. Host Cells

The disclosure also provides a host cell comprising a nucleic acid molecule or vector of the disclosure. As used herein, the term “transformation” shall be used in a broad sense to refer to the introduction of DNA into a recipient host cell that changes the genotype and consequently results in a change in the recipient cell.

“Host cells” refers to cells that have been transformed with vectors constructed using recombinant DNA techniques and encoding at least one heterologous gene. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte), PER.C6®, NS0, CAP, BHK21, and HEK293 (human kidney). In one particular embodiment, the host cell is selected from the group consisting of: a CHO cell, a HEK293 cell, a BHK21 cell, a PER.C6® cell, a NS0 cell, a CAP cell and any combination thereof. In some embodiments, the host cells of the present disclosure are of insect origin. In one particular embodiment, the host cells are SF9 cells. Host cell lines are typically available from commercial services, the American Tissue Culture Collection, or from published literature.

Introduction of the nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

Host cells comprising the isolated nucleic acid molecules or vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, CA.). In another embodiment, the medium is CD17 (Invitrogen, Carlsbad, CA.). Selection of a medium appropriate for the particular cell line used is within the level of those ordinary skilled in the art.

### V. Preparation of Polypeptides

The disclosure also provides a polypeptide encoded by a nucleic acid molecule of the disclosure. In other embodiments, the polypeptide of the disclosure is encoded by a vector comprising the nucleic molecules of the disclosure. In yet other embodiments, the polypeptide of the disclosure is produced by a host cell comprising the nucleic molecules of the disclosure.

In other embodiments, the disclosure also provides a method of producing a polypeptide with clotting factor, e.g., FVIII, activity, comprising culturing a host cell of the disclosure under conditions whereby a polypeptide with clotting factor, e.g., FVIII, activity is produced, and recovering the polypeptide with clotting factor, e.g., FVIII, activity. In some embodiments, the expression of the polypeptide with clotting factor, e.g., FVIII, activity is increased relative to a host cell cultured under the same conditions but comprising a reference nucleotide sequence (e.g., SEQ ID NO: 16, the parental FVIII gene sequence).

In other embodiments, the disclosure provides a method of increasing the expression of a polypeptide with clotting factor, e.g., FVIII, activity comprising culturing a host cell of the disclosure under conditions whereby a polypeptide with clotting factor, e.g., FVIII, activity is expressed by the nucleic acid molecule, wherein the expression of the polypeptide with clotting factor, e.g., FVIII, activity is increased relative to a host cell cultured under the same conditions comprising a reference nucleic acid molecule (e.g., SEQ ID NO: 16, the parental FVIII gene sequence).

In other embodiments, the disclosure provides a method of improving yield of a polypeptide with clotting factor, e.g., FVIII, activity comprising culturing a host cell under conditions whereby a polypeptide with clotting factor, e.g., FVIII, activity is produced by the nucleic acid molecule disclosed herein, wherein the yield of polypeptide with clotting factor, e.g., FVIII, activity is increased relative to a host cell cultured under the same conditions comprising a reference nucleic acid sequence (e.g., SEQ ID NO: 16, the parental FVIII gene sequence).

The therapeutic protein, e.g. the clotting factor, of the disclosure can be synthesized in a transgenic animal, such as a rodent, goat, sheep, pig, or cow. The term “transgenic animals” refers to non-human animals that have incorporated a foreign gene into their genome. Because this gene is present in germline tissues, it is passed from parent to offspring. Exogenous genes are introduced into single-celled embryos (Brinster et al. 1985, Proc. Natl. Acad. Sci. USA 82:4438). Methods of producing transgenic animals are known in the art including transgenics that produce immunoglobulin molecules (Wagner et al. 1981, Proc. Natl. Acad. Sci. USA 78: 6376; McKnight et al. 1983, Cell 34: 335; Brinster et al. 1983, Nature 306: 332; Ritchie et al. 1984, Nature 312: 517; Baldassarre et al. 2003, Theriogenology 59: 831; Robl et al. 2003, Theriogenology 59: 107; Malassagne et al. 2003, Xenotransplantation 10 (3): 267).

### VII. Pharmaceutical Composition

Compositions containing a nucleic acid molecule, a polypeptide encoded by the nucleic acid molecule, a vector, or a host cell of the present disclosure can contain a suitable pharmaceutically acceptable carrier. For example, they can contain excipients and/or auxiliaries that facilitate processing of the active compounds into preparations designed for delivery to the site of action.

In one embodiment, the present disclosure is directed to a pharmaceutical composition comprising (a) a nucleic acid molecule, a vector, a polypeptide, or a host cell disclosed herein; and (b) a pharmaceutically acceptable excipient.

In some embodiments, the pharmaceutical composition further comprises a delivery agent. In certain embodiments, the delivery agent comprises a lipid nanoparticle (LNP). In other embodiments, the pharmaceutical composition further comprises liposomes, other polymeric molecules, and exosomes.

As used herein a “lipid nanoparticle” refers to a nanoparticle that comprises a plurality of lipid molecules physically associated with each other by intermolecular forces. The lipid nanoparticles may be, e.g., microspheres (including unilamellar and multilamellar vesicles, e.g. liposomes), a dispersed phase in an emulsion, micelles or an internal phase in a suspension.

In some embodiments, the present disclosure provides an encapsulated nucleic acid molecule composition which may include a lipid nanoparticle host encapsulating a nucleic acid molecule of the invention. The lipid nanoparticle may comprise one or more lipids (e.g., cationic lipids, non-cationic lipids, and PEG-modified lipids). In certain embodiments, lipid nanoparticles of the present disclosure are formulated to deliver one or more nucleic acid molecules of the invention to one or more target cells. Examples of suitable lipids include, without limitation, phosphatidyl compounds (e.g., phosphatidylethanolamine, sphingolipids, phosphatidylcholine, phosphatidylserine, phosphatidylglycerol, gangliosides, and cerebrosides). A “cationic lipid” refers to any lipid species that carry a net positive charge at a certain pH (e.g., physiological pH).

In certain embodiments, the lipid nanoparticles of the present disclosure have a certain N/P ratio. As used herein “N/P ratio” or “NP ratio” refers to the ratio of positively-chargeable polymer amine groups to negatively-charged nucleic acid phosphate groups. The N/P character of a lipid nanoparticle/nucleic acid molecule complex can influence properties such as net surface charge, stability, and size. The NP ratio of the lipid nanoparticles as described herein may be about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, and any ratio in between. For example, the NP ratio of the lipid nanoparticles as described herein may be about 18, about 36, or about 72.

Accordingly, in certain embodiments, a pharmaceutical composition comprises a nucleic acid molecule of the present disclosure encapsulated in a lipid nanoparticle, and a pharmaceutically acceptable excipient.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides. Aqueous injection suspensions can contain substances, which increase the viscosity of the suspension, including, for example, sodium carboxymethyl cellulose, sorbitol and dextran. Optionally, the suspension can also contain stabilizers. Liposomes also can be used to encapsulate the molecules of the disclosure for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the disclosure can be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See, e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, the nucleic acid molecule of the disclosure is formulated with a clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition can take the form of tablets or lozenges according to conventional protocols.

For administration by inhalation, the compounds for use according to the present disclosure are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

In some embodiments, the composition is administered by a route selected from the group consisting of topical administration, intraocular administration, parenteral administration, intrathecal administration, subdural administration and oral administration. The parenteral administration can be intravenous or subcutaneous administration.

### VIII. Methods of Treatment

In some aspects, the present disclosure is directed to methods of treating a disease or condition in a subject in need thereof, comprising administering a nucleic acid molecule, a vector, a polypeptide, or a pharmaceutical composition disclosed herein.

In some embodiments, the nucleic acid molecule comprises a first ITR, a second ITR, and a genetic cassette, wherein the genetic cassette encodes a target sequence, wherein the target sequence encodes a therapeutic protein, and wherein the nucleic acid molecule is used to treat a disease or condition in a subject in need thereof. In some embodiments, the disease or condition affects an organ selected from the muscle, central nervous system (CNS), ocular, liver, heart, kidney, pancreas, lungs, skin, bladder, urinary tract, and any combination thereof. In some embodiments, the subject has a disease or condition selected from the group consisting of DMD (Duchenne muscular dystrophy), XLMTM (X-linked myotubular myopathy), Parkinson, SMA (spinal muscular atrophy), Friedreich's Ataxia, GUCY2D-LCA (Leber Congenital Amaurosis), XLRS (X-Linked Retinoschisis), AMD (Age-related Macular Degeneration), ACHM (Achromatopsia), RPF65 mediated IRD, and any combination thereof.

In some embodiments, the nucleic acid molecule comprises a first ITR, a second ITR, and a genetic cassette, wherein the genetic cassette encodes a target sequence, wherein the target sequence encodes a miRNA, and wherein the nucleic acid molecule is used to treat a disease or condition in a subject in need thereof. In some embodiments, the disease or condition comprises Amyotrophic lateral sclerosis (ALS), Huntington's disease, and/or autosomal dominant retinitis pigmentosa.

In some embodiments, the nucleic acid molecule comprises a first ITR, a second ITR, and a genetic cassette, wherein the genetic cassette encodes a target sequence, wherein the target sequence encodes a clotting factor, and wherein the nucleic acid molecule is used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In yet other embodiments, the treatment is prophylactic or on-demand.

The disclosure provides a method of treating a bleeding disorder comprising administering to a subject in need thereof a nucleic acid molecule, vector, or polypeptide of the disclosure. In some embodiments, the bleeding disorder is characterized by a deficiency in a clotting factor, e.g., FVIII. In some embodiments, the bleeding disorder is hemophilia. In some embodiments, the bleeding disorder is hemophilia A. In some embodiments of the method of treating a bleeding disorder, plasma activity of a clotting factor, e.g., FVIII, at 24 hours post administration is increased relative to a subject administered a reference nucleic acid molecule (e.g., SEQ ID NO: 16, the parental FVIII gene sequence), a vector comprising a reference nucleic acid molecule, or a polypeptide encoded by a reference nucleic acid molecule.

The disclosure also relates to a method of treating, ameliorating, or preventing a hemostatic disorder in a subject comprising administering a therapeutically effective amount of an isolated nucleic acid molecule of the disclosure or a polypeptide having clotting factor, e.g., FVIII, activity encoded by the nucleic acid molecule of the disclosure. The treatment, amelioration, and prevention by the isolated nucleic acid molecule or the encoded polypeptide can be a bypass therapy. The subject receiving bypass therapy can have already developed an inhibitor to a clotting factor, e.g., FVIII, or is subject to developing a clotting factor inhibitor.

The nucleic acid molecules, vectors, or polypeptides of the disclosure treat or prevent a hemostatic disorder by promoting the formation of a fibrin clot. The polypeptide having clotting factor, e.g., FVIII, activity encoded by the nucleic acid molecule of the disclosure can activate a member of a coagulation cascade. The clotting factor can be a participant in the extrinsic pathway, the intrinsic pathway or both.

The nucleic acid molecules, vectors, or polypeptides of the disclosure can be used to treat hemostatic disorders known to be treatable with a clotting factor. The hemostatic disorders that can be treated using methods of the disclosure include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

In some embodiments, the hemostatic disorder is an inherited disorder. In one embodiment, the subject has hemophilia A. In other embodiments, the hemostatic disorder is the result of a deficiency in a clotting factor. In other embodiments, the hemostatic disorder is the result of a deficiency in FVIII. In other embodiments, the hemostatic disorder can be the result of a defective FVIII clotting factor.

In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g., cancer chemotherapy).

The disclosure also relates to methods of treating a subject that does not have a hemostatic disorder or a secondary disease or condition resulting in acquisition of a hemostatic disorder. The disclosure thus relates to a method of treating a subject in need of a general hemostatic agent comprising administering a therapeutically effective amount of the isolated nucleic acid molecule, vector, or polypeptide of the disclosure. For example, in one embodiment, the subject in need of a general hemostatic agent is undergoing, or is about to undergo, surgery. The isolated nucleic acid molecule, vector, or polypeptide of the disclosure can be administered prior to or after surgery as a prophylactic. The isolated nucleic acid molecule, vector, or polypeptide of the disclosure can be administered during or after surgery to control an acute bleeding episode. The surgery can include, but is not limited to, liver transplantation, liver resection, or stem cell transplantation.

In another embodiment, the isolated nucleic acid molecule, vector, or polypeptide of the disclosure can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The isolated nucleic acid molecule, vector, or protein can be used to prophylactically treat a subject with a hemostatic disorder. The isolated nucleic acid molecule, vector, or protein can be used to treat an acute bleeding episode in a subject with a hemostatic disorder.

In another embodiment, expression of the clotting factor protein by administering the isolated nucleic acid molecule or vector of the disclosure does not induce an immune response in a subject. In some embodiments, the immune response comprises development of antibodies against a clotting factor. In one embodiment, the immune response comprises development of antibodies against FVIII. In some embodiments, the immune response comprises cytokine secretion. In some embodiments, the immune response comprises activation of B cells, T cells, or both B cells and T cells. In some embodiments, the immune response is an inhibitory immune response, wherein the immune response in the subject reduces the activity of a clotting factor protein relative to the activity of the clotting factor in a subject that has not developed an immune response. In certain embodiments, expression of a clotting factor protein by administering the isolated nucleic acid molecule or vector, of the disclosure prevents an inhibitory immune response against the clotting factor protein or the clotting factor protein expressed from the isolated nucleic acid molecule or the vector.

In some embodiments, an isolated nucleic acid molecule, vector, or protein composition of the disclosure is administered in combination with at least one other agent that promotes hemostasis. Said other agent that promotes hemostasis in a therapeutic with demonstrated clotting activity. As an example, but not as a limitation, the hemostatic agent can include FV, FVII, FIX, FX, FXI, FXII, FXIII, prothrombin, or fibrinogen or activated forms of any of the preceding. The clotting factor or hemostatic agent can also include anti-fibrinolytic drugs, e.g., epsilon-amino-caproic acid, tranexamic acid.

In one embodiment of the disclosure, the composition (e.g., the isolated nucleic acid molecule, vector, or polypeptide) is one in which the clotting factor is present in activatable form when administered to a subject. Such an activatable molecule can be activated in vivo at the site of clotting after administration to a subject.

Accordingly, in some embodiments, the present disclosure provides a method of treating a bleeding disorder in a subject in need thereof, comprising administering to the subject a nucleic acid molecule comprising a first inverted terminal repeat (ITR) and a second ITR flanking a genetic cassette comprising a heterologous polynucleotide sequence encoding a clotting factor, wherein the first ITR and/or second ITR are an ITR of a non-adeno-associated virus (non-AAV). In some embodiments, the present disclosure provides a method of treating a bleeding disorder in a subject in need thereof, comprising administering to the subject a nucleic acid molecule comprising a first inverted terminal repeat (ITR) and a second ITR flanking a genetic cassette comprising a heterologous polynucleotide sequence encoding a clotting factor, wherein the first ITR and/or second ITR comprises a nucleotide sequence set forth in SEQ ID NO: 180, 181, 183, 184, 185, 186, 187 or 188. In some embodiments, the present disclosure provides a method of treating a bleeding disorder in a subject in need thereof, comprising administering to the subject a nucleic acid molecule comprising a first inverted terminal repeat (ITR) and a second ITR flanking a genetic cassette comprising a heterologous polynucleotide sequence encoding a clotting factor, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

Accordingly, in some embodiments, the present disclosure provides a method of treating hemophilia A in a subject in need thereof, comprising administering to the subject a nucleic acid molecule comprising a first inverted terminal repeat (ITR) and a second ITR flanking a genetic cassette comprising a heterologous polynucleotide sequence encoding factor VIII, wherein the first ITR and/or second ITR are an ITR of a non-adeno-associated virus (non-AAV). In some embodiments, the present disclosure provides a method of treating hemophilia A in a subject in need thereof, comprising administering to the subject a nucleic acid molecule comprising a first inverted terminal repeat (ITR) and a second ITR flanking a genetic cassette comprising a heterologous polynucleotide sequence encoding factor VIII, wherein the first ITR and/or second ITR comprises a nucleotide sequence set forth in SEQ ID NO: 180, 181, 183, 184, 185, 186, 187 or 188. In some embodiments, the present disclosure provides a method of treating hemophilia A in a subject in need thereof, comprising administering to the subject a nucleic acid molecule comprising a first inverted terminal repeat (ITR) and a second ITR flanking a genetic cassette comprising a heterologous polynucleotide sequence encoding factor VIII, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

The disclosure also provides a method of treating a metabolic disorder of the liver comprising administering to a subject in need thereof a nucleic acid molecule, vector, or polypeptide of the disclosure. In some embodiments, the metabolic disorder of the liver is selected from the group consisting of phenylketonuria (

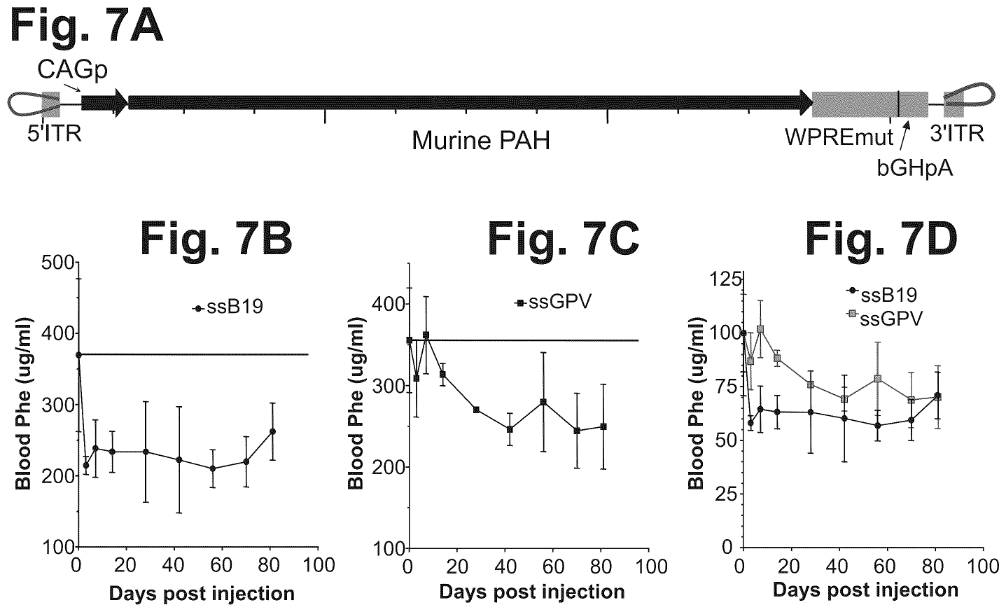

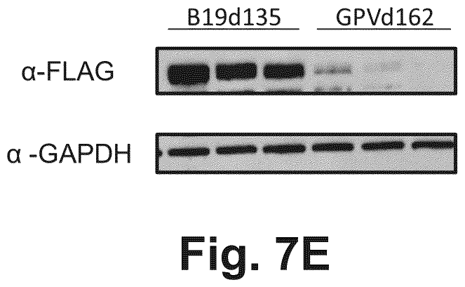

FIG. 7E shows a Western immunoblot of liver lysates from PKU mice treated with ssDNA containing the murine PAH transgene and either B19d135 or GPCd165 ITRs. Livers were collected at day 81 post treatment and protein lysates were extracted. Each well represents a single animal. The FLAG-tagged murine PAH protein was detected using the M2 anti-FLAG antibody and a GAPDH loading control is included for comparison.

), a urea cycle disease (e.g., a deficiency in transcarbamylase (OTC), or argininosuccinate synthetase (ASS)), a lysosomal storage disorder (e.g., mucopolysaccharidoses), and a glycogen storage disease (e.g., Type I, II, III, IV glycogen storage disease). Other metabolic disorders of the liver include, without limitation, Wilson's disease, alpha-1 antitrypsin deficiency, gestational alloimmune liver disease (GALD), fatty acid oxidation defects, galactosemia, lipid storage diseases, tyrosinemia, and peroxisomal disorders.

In some embodiments, the present disclosure provides a method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a liver-associated metabolic enzyme that is deficient in the subject, wherein the first ITR and/or second ITR are an ITR of a non-adeno-associated virus (non-AAV). In some embodiments, the present disclosure provides a method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a therapeutic protein (e.g., a protein required for proper metabolic function of the liver), wherein the first ITR and/or second ITR comprises a nucleotide sequence set forth in SEQ ID NO: 180, 181, 183, 184, 185, 186, 187 or 188. In some embodiments, the present disclosure provides a method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a therapeutic protein (e.g., a protein required for proper metabolic function of the liver), wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

In some embodiments, the present disclosure provides a method of treating a phenylketonuria (PKU) in a subject in need thereof, comprising administering to the subject a nucleic acid molecule comprising a first inverted terminal repeat (ITR) and a second ITR flanking a genetic cassette comprising a heterologous polynucleotide sequence encoding phenylalanine hydroxylase (PAH), wherein the first ITR and/or second ITR are an ITR of a non-adeno-associated virus (non-AAV). In some embodiments, the present disclosure provides a method of treating phenylketonuria (PKU) in a subject in need thereof, comprising administering to the subject a nucleic acid molecule comprising a first inverted terminal repeat (ITR) and a second ITR flanking a genetic cassette comprising a heterologous polynucleotide sequence encoding phenylalanine hydroxylase (PAH), wherein the first ITR and/or second ITR comprises a nucleotide sequence set forth in SEQ ID NO: 180, 181, 183, 184, 185, 186, 187 or 188. In some embodiments, the present disclosure provides a method of treating phenylketonuria (PKU) in a subject in need thereof, comprising administering to the subject a nucleic acid molecule comprising a first inverted terminal repeat (ITR) and a second ITR flanking a genetic cassette comprising a heterologous polynucleotide sequence encoding phenylalanine hydroxylase, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

The isolated nucleic acid molecule, vector, or polypeptide can be administered intravenously, subcutaneously, intramuscularly, or via any mucosal surface, e.g., orally, sublingually, buccally, sublingually, nasally, rectally, vaginally or via pulmonary route. The clotting factor protein can be implanted within or linked to a biopolymer solid support that allows for the slow release of the chimeric protein to the desired site.

For oral administration, the pharmaceutical composition can take the form of tablets or capsules prepared by conventional means. The composition can also be prepared as a liquid for example a syrup or a suspension. The liquid can include suspending agents (e.g. sorbitol syrup, cellulose derivatives or hydrogenated edible fats), emulsifying agents (lecithin or acacia), non-aqueous vehicles (e.g. almond oil, oily esters, ethyl alcohol, or fractionated vegetable oils), and preservatives (e.g. methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration, the composition can take the form of tablets, lozenges or fast dissolving films according to conventional protocols.

For administration by inhalation, the polypeptide having clotting factor activity for use according to the present disclosure are conveniently delivered in the form of an aerosol spray from a pressurized pack or nebulizer (e.g., in PBS), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In one embodiment, the route of administration of the isolated nucleic acid molecule, vector, or polypeptide is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the isolated nucleic acid molecule, vector, or polypeptide can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

The pharmaceutical composition can also be formulated for rectal administration as a suppository or retention enema, e.g., containing conventional suppository bases such as cocoa butter or other glycerides.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The nucleic acid molecule, vector, or polypeptides of the disclosure can optionally be administered in combination with other agents that are effective in treating the disorder or condition in need of treatment (e.g., prophylactic or therapeutic).

As used herein, the administration of isolated nucleic acid molecules, vectors, or polypeptides of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the isolated nucleic acid molecule, vector, or polypeptide of the instant disclosure can be used in conjunction or combination with an agent or agents (e.g., to provide a combined therapeutic regimen). Exemplary agents with which a polypeptide or polynucleotide of the disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the polynucleotides or polypeptides of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996). In another embodiment, an amount of such an agent consistent with the standard of care is administered.

In one embodiment, also disclosed herein is a kit, comprising the nucleic acid molecule disclosed herein and instructions for administering the nucleic acid molecule to a subject in need thereof. In another embodiment, disclosed herein is a baculovirus system for production of the nucleic acid molecule provided herein. The nucleic acid molecule is produced in insect cells. In another embodiment, a nanoparticle delivery system for expression constructs is provided. The expression construct comprises the nucleic acid molecule disclosed herein.

### IX. Gene Therapy

Certain aspects of the present disclosure provide a method of expressing a genetic construct in a subject, comprising administering the isolated nucleic acid molecule of the disclosure to a subject in need thereof. In some aspects, the disclosure provides a method of increasing expression of a polypeptide in a subject comprising administering the isolated nucleic acid molecule of the disclosure to a subject in need thereof. In other aspects, the disclosure provides a method of modulating expression of a polypeptide in a subject in need thereof comprising administering an isolated nucleic acid molecule of the disclosure, e.g., a nucleic acid sequence comprising a miRNA, to the subject. In some aspects, the disclosure provides a method of down regulating the expression of a target gene in a subject in need thereof comprising administering an isolated nucleic acid molecule of the disclosure, e.g., a nucleic acid sequence comprising a miRNA, to the subject.

Somatic gene therapy has been explored as a possible treatment for a variety of conditions, including, but not limited to, hemophilia A. Gene therapy is a particularly appealing treatment for hemophilia because of its potential to cure the disease through continuous endogenous production of a clotting factor, e.g., FVIII, following a single administration of vector. Haemophilia A is well suited for a gene replacement approach because its clinical manifestations are entirely attributable to the lack of a single gene product (e.g., FVIII) that circulates in minute amounts (200 ng/ml) in the plasma.

The use of conventional viral based gene delivery has been shown to induce an immune response in humans. Viral capsid proteins can trigger various components of the human immune system. AAV based gene delivery has been attractive as AAV is a common virus in the human population, most people have been exposed to AAV, and AAV has been shown to be less immunogenic than, e.g., Adenovirus. Accordingly, most people have already developed an immune response against the particular variants to which they had previously been exposed. This pre-existing adaptive response can include NAbs and T cells that could diminish the clinical efficacy of subsequent re-infections with AAV and/or the elimination of cells that have been transduced, which disqualifies patients with pre-existing anti-AAV immunity to AVV based gene therapy treatment. The nucleic acid molecules of the present disclosure find use in non-viral based gene therapy. As viral capsids are not necessary for gene delivery using the nucleic acid molecules of the present disclosure, no immunity will be developed to viral components barring the subsequent re-administration (or re-dosing) of a subject. As such, the nucleic acid molecules of the present disclosure allow for re-dosing for long term gene delivery strategies.

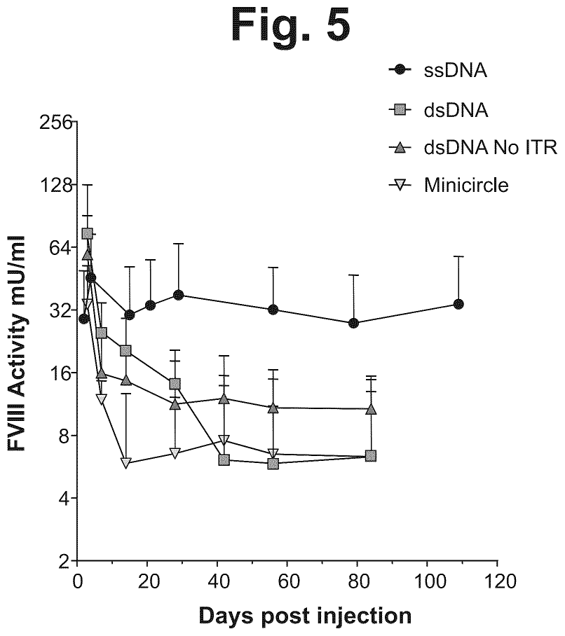

In addition, as described herein, the nucleic acid molecules of the present disclosure comprise non-AAV parvoviral ITRs flanking a genetic cassette to drive stable transgene expression upon administration. The presence of the ITRs are necessary for stable transgene expression, as shown in FIG. 5, where nucleic acids without ITRs were unable to effect stable transgene expression (see, “dsDNA no ITR” and “minicircle”).

A clotting factor protein of the disclosure can be produced in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A.

Other conditions are also suitable for treatment using the nucleic acid molecules disclosed herein. In certain embodiments, the methods described herein are used for treating a disease or condition that affects a target organ selected from the muscle, central nervous system (CNS), ocular, liver, heart, kidney, pancreas, lungs, skin, bladder, urinary tract, or any combination thereof. In certain embodiments, the methods described herein are used for treating a disease or condition selected from DMD (Duchenne muscular dystrophy), XLMTM (X-linked myotubular myopathy), Parkinson, SMA (spinal muscular atrophy), Friedreich's Ataxia, GUCY2D-LCA (Leber Congenital Amaurosis), XLRS (X-Linked Retinoschisis), AMD (Age-related Macular Degeneration), ACHM (Achromatopsia), RPF65 mediated IRD (Table 9).

TABLE 9
Diseases and disorders treatable by the methods disclosed herein.

| | | | Gene |
|---|---|---|---|
| | Target organ | Defective gene | therapy |
| Disease | | | |
| DMD | Muscle | Dystrophin | Gene |
| (Duchenne muscular | | X-linked | introduction |
| dystrophy) | | | |
| XLMTM | Muscle | MTM1 | Gene |
| (X-linked myotubular | | (myotubularin) | introduction |
| myopathy) | | | |
| Parkinson | CNS | Tyrosine | Gene |
| | | hydroxylase, | introduction |
| | | AADC, | |
| | | cyclohydrolase | |
| SMA | CNS | SMN1 | Gene |
| (spinal muscular | | | introduction |
| atrophy) | | | |
| Friedreich's Ataxia | CNS | FXN (Frataxin) | Gene |
| | | | introduction |
| GUCY2D-LCA | Ocular | GUCY2D | Gene |
| Leber Congenital | | | introduction |
| Amaurosis | | | |
| XLRS | Ocular | RS1 | Gene |
| X-Linked Retinoschisis | | | introduction |
| AMD | Ocular | CFH | Gene |
| Age-related Macular | | HTRA | introduction |
| Degeneration | | ARMS | |
| | | CFB/CC2 | |
| ACHM | Ocular | CNGA/CNGB | Gene |
| Achromatopsia | | | introduction |
| RPF65 mediated IRD | Ocular | Prf65 | Gene |
| | | | introduction |
| Lysosomal storage | | | |
| disorders | | | |
| MLD | CNS | ARSA | Gene |
| metachromatic | | PSAP | introduction |
| leukodystrophy | | | |
| (Lysosomal storage | | | |
| disorder) | | | |
| MPS | Liver | IDUA (MPS I) | Gene |
| Mucopolysaccharidoses | | IDS (MPS II) | introduction |
| (Lysosomal storage | | | |
| disorder) | | | |
| PKU | Liver | PAH | Gene |
| Phenylketonuria | | | introduction |
| (Lysosomal storage | | | |
| disorder) | | | |
| Pompe | Heart, liver, | GAA | Gene |
| Glycogen storage | muscle, CNS | (acid alpha- | introduction |
| disease type II | | glucosidase) | |
| Micro RNA therapies | | | |
| ALS | CNS | SOD1¹ | miRNA |
| Amyotrophic lateral | | | |
| sclerosis | | | |
| Huntington's disease | CNS | HTT² | miRNA |
| AdRP | Ocular | RHO³ | miRNA |
| Autosomal Dominant | | (Rhodopsin) | |
| Retinitis Pigmentosa | | | |

¹Mutation of SOD1 gene accounted to 20% of the inherited ALS case. Wildtype SOD1 has demonstrated antiapoptotic properties in neural cultures, while mutant SOD1 has been observed to promote apoptosis in spinal cord mitochondria, but not in liver mitochondria, though it is equally expressed in both. Down regulate mutated SOD1 expression might inhibit motor neuron degeneration in ALS.
²HD is one of several trinucleotide repeat disorders which are caused by the length of a repeated section of a gene exceeding a normal range. HTT contains a sequence of three DNA bases-cytosine-adenine-guanine (CAG)-repeated multiple times (i.e. . . . CAGCAGCAG . . . ), known as a trinucleotide repeat. CAG is the 3-letter genetic code (codon) for the amino acid glutamine, so a series of them results in the production of a chain of glutamine known as a polyglutamine tract (or polyQ tract), and the repeated part of the gene, the PolyQ region. Generally, people have fewer than 36 repeated glutamines in the polyQ region which results in production of the cytoplasmic protein Huntingtin. However, a sequence of 36 or more glutamines results in the production of a protein which has different characteristics. This altered form, called mutant huntingtin (mHTT), increases the decay rate of certain types of neurons. Generally, the number of CAG repeats is related to how much this process is affected, and accounts for about 60% of the variation of the age of the onset of symptoms. The remaining variation is attributed to environment and other genes that modify the mechanism of HD. 36-39 repeats result in a reduced-penetrance form of the disease, with a much later onset and slower progression of symptoms. In some cases the onset may be so late that symptoms are never noticed. With very large repeat counts, HD has full penetrance and can occur under the age of 20, when it is then referred to as juvenile HD, akinetic-rigid, or Westphal variant HD. This accounts for about 7% of HD carriers.
³Most of the RHO gene mutations responsible for retinitis pigmentosa alter the folding or transport of the rhodopsin protein. A few mutations cause rhodopsin to be constitutively activated instead of being activated in response to light. Studies suggest that altered versions of rhodopsin interfere with essential cell functions, causing rods to self-destruct (undergo apoptosis). Because rods are essential for vision under low-light conditions, the loss of these cells leads to progressive night blindness in people with retinitis pigmentosa.

In some embodiments, the methods described herein are used for treating a lysosomal storage disorder. In some embodiments, the lysosomal storage disorder is selected from MLD (metachromatic leukodystrophy), MPS (mucopolysaccharidoses), PKU (phenylketonuria), pompe glycogen storage disease type II, or any combination thereof.

In some embodiments, the methods described herein are used in a microRNA (miRNA) therapy. In some embodiments, the miRNA treats a condition caused by the overexpression of a gene or a protein. In some embodiments, the miRNA treats a condition caused by the accumulation of a protein. In some embodiments, the miRNA treats a condition caused by the misexpression of a gene or protein. In some embodiments, the miRNA treats a condition caused by the expression of a mutant gene. In some embodiments, the miRNA treats a condition caused by the expression of an heterologous gene. In certain embodiments, the miRNA therapy treats a condition selected from ALS (amytrophic lateral sclerosis), Huntington's disease, AdRP (autosomal dominant retinitis pigmentosa), and any combination thereof. In certain embodiments, the methods of the present disclosure comprise targeting treating ALS by administering a nucleic acid molecule disclosed herein, wherein the nucleic acid molecule comprises a genetic cassette encoding a miRNA, wherein the miRNA targets the expression of SOD1. In certain embodiments, the miRNA comprises the miR SOD1 artificial miRNA disclosed by Dirren et al., Annals of Clinical and Translational Neurology 2(2):167-84 (February 2015). Mutation of SOD1 gene accounts for 20% of inherited ALS cases. Wildtype SOD1 has demonstrated antiapoptotic properties in neural cultures, while mutant SOD1 has been observed to promote apoptosis in spinal cord mitochondria, but not in liver mitochondria, though it is equally expressed in both. Down regulation of mutated SOD1 expression might inhibit motor neuron degeneration in ALS.

In certain embodiments, the methods of the present disclosure comprise targeting treating Huntington's disease by administering a nucleic acid molecule disclosed herein, wherein the nucleic acid molecule comprises a genetic cassette encoding a miRNA, wherein the miRNA targets the expression of HTT. In certain embodiments, the miRNA comprises the miHTT engineered miRNA disclosed by Evers et al., Molecular Therapy 26(9):1-15 (epub ahead of print June 2018). Huntington's disease is one of several trinucleotide repeat disorders which are caused by the length of a repeated section of a gene exceeding a normal range. HTT contains a sequence of three DNA bases—cytosine-adenine-guanine (CAG)—repeated multiple times (i.e. . . . CAGCAGCAG . . . ), which is known as a trinucleotide repeat. CAG is the 3-letter genetic code (codon) for the amino acid glutamine, so a series of these repeats results in the production of a chain of glutamine known as a polyglutamine tract (or polyQ tract), and the repeated part of the gene, the PolyQ region. Generally, people have fewer than 36 repeated glutamines in the polyQ region which results in production of the cytoplasmic protein huntingtin. However, a sequence of 36 or more glutamines results in the production of a protein which has different characteristics. This altered form, called mutant huntingtin (mHTT), increases the decay rate of certain types of neurons. Generally, the number of CAG repeats is related to how much this process is affected, and accounts for about 60% of the variation of the age of the onset of symptoms. The remaining variation is attributed to environment and other genes that modify the mechanism of Huntington's disease. 36-39 repeats result in a reduced-penetrance form of the disease, with a much later onset and slower progression of symptoms. In some cases the onset may be so late that symptoms are never noticed. With very large repeat counts, Huntington's disease has full penetrance and can occur under the age of 20, when it is then referred to as juvenile Huntington's disease, akinetic-rigid, or Westphal variant Huntington's disease. This accounts for about 7% of Huntington's disease carriers.

In certain embodiments, the methods of the present disclosure comprise targeting treating Autosomal Dominant Retinitis Pigmentosa (AdRP) by administering a nucleic acid molecule disclosed herein, wherein the nucleic acid molecule comprises a genetic cassette encoding a miRNA, wherein the miRNA targets the expression of RHO (rhodopsin). In certain embodiments, the miRNA comprises miR-708 (see Behrman et al., JCB 192(6):919-27 (2011). Most of the RHO gene mutations responsible for retinitis pigmentosa alter the folding or transport of the rhodopsin protein. A few mutations cause rhodopsin to be constitutively activated instead of being activated in response to light. Studies suggest that altered versions of rhodopsin interfere with essential cell functions, causing rods to self-destruct (undergo apoptosis). Because rods are essential for vision under low-light conditions, the loss of these cells leads to progressive night blindness in people with retinitis pigmentosa.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1. Generation of FVIII Expression Constructs Bearing AAV and Non-AAV Parvoviral ITRs

### Example 1a. Cloning of Codon-Optimized FVIII Gene and Inverted Terminal Repeat (ITR) Regions from AAV into Genetic Cassettes

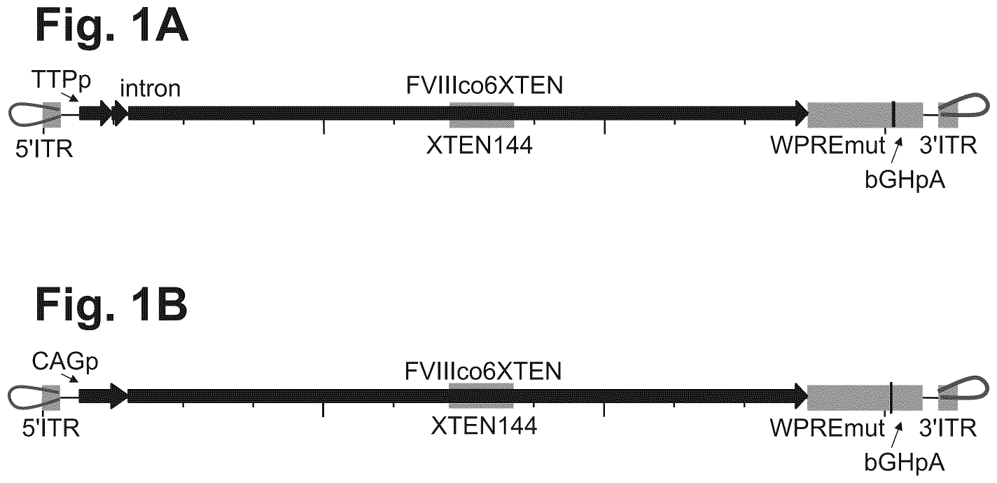

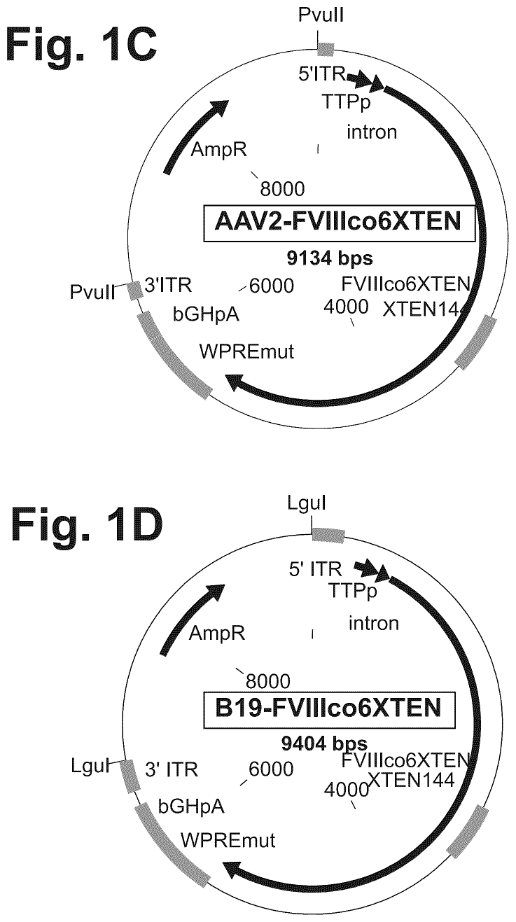

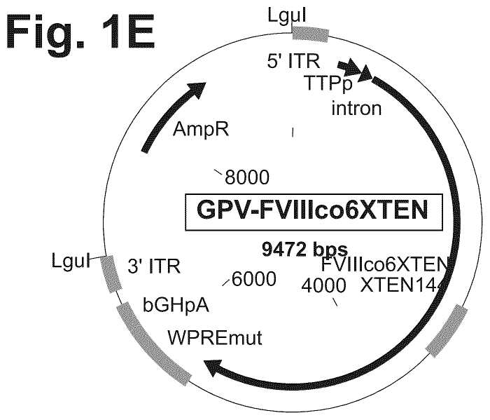

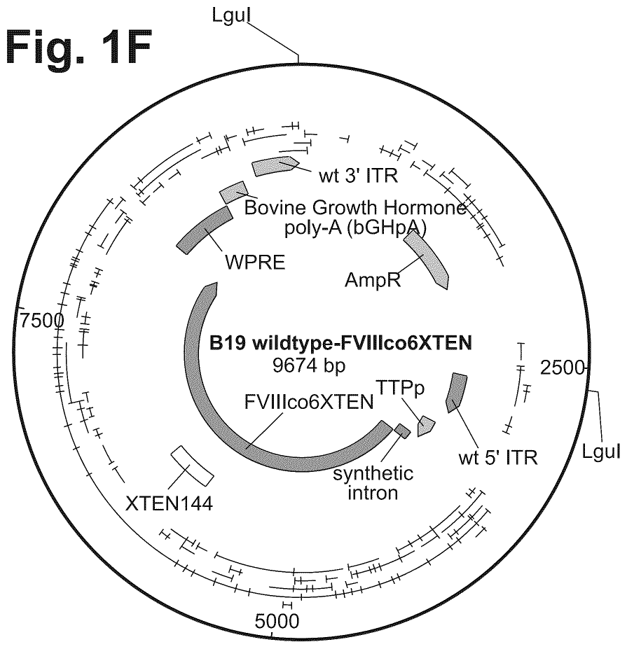

FVIII genetic cassette was generated based on the genome of AAV serotype 2. However, ITR regions originated from any serotype (including synthetic) can be used in this approach (FIG. 1A).

Expression plasmid AAV2-FVIIIco6XTEN encoding a codon-optimized FVIII coding sequence under the regulation of a liver-specific promoter (TTPp) or a ubiquitous promoter (CAGp, FIGS. 1A and 1B) flanked by inverted terminal repeat (ITR) regions from AAV (AAV-FVIII) was designed for in vitro and in vivo expression, as shown in FIG. 1C. The genetic cassette also contains WPRE and bGHpA elements for optimal expression of the transgene (FIGS. 1A-1C). ITR-flanked codon-optimized FVIII sequence was cloned into a plasmid backbone comprising a CoIE1 origin of replication and an expression cassette for beta-lactamase, which confers ampicillin resistance (FIG. 1C). Recognition sites for the restriction endonuclease Pvull flanking the expression cassette were engineered to allow for precise excision of the AAV-FVIII construct upon Pvull digestion (FIG. 1C).

### Example 1b. Cloning of Codon-Optimized FVIII Gene and Inverted Terminal Repeat (ITR) Regions from Non-AAV Parvoviruses into Genetic Cassettes

Based on the phylogenic relationship between the members of the viral family Parvoviridae, which AAV belongs to (FIG. 2A), it was hypothesized that other non-AAV members of the genus Dependovirus and the members of the genus Erythrovirus utilize similar cellular mechanisms for the maintenance of the viral life cycle and establishment of persistent, latent infection. Therefore, the ITR regions originated from the genomes of these viruses could be utilized to develop AAV-like (but not AAV-based) genetic expression cassettes. The following parvoviruses were tested for the suitability of their ITR regions for the development of genetic constructs for gene therapy applications: Dependovirus Goose parvovirus (GPV) strain B and Erythrovirus B19 parvovirus (FIG. 2A).

Instability of parvoviral ITR regions during propagation of plasmid vectors in bacterial cells presents a challenge for the generation and manipulation of genetic constructs. Some genetic constructs containing the full-length AAV2 ITRs (145 nt) have been successfully generated but these constructs are highly unstable and most AAV2 ITR-based plasmids contain a truncated 130 nt version of the ITR region (exemplified in Table 1). Similarly, the plasmid constructs bearing full-length sequences of both B19 and GPV ITRs that were generated exhibited a high degree of instability in bacterial host (data not shown), which significantly limits the utility of these ITRs for the development of genetic vectors for gene therapy applications.

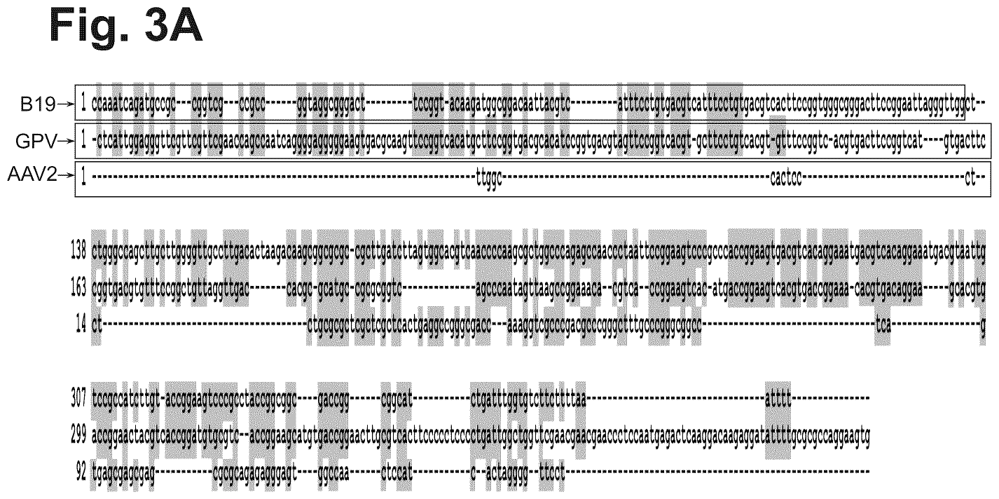

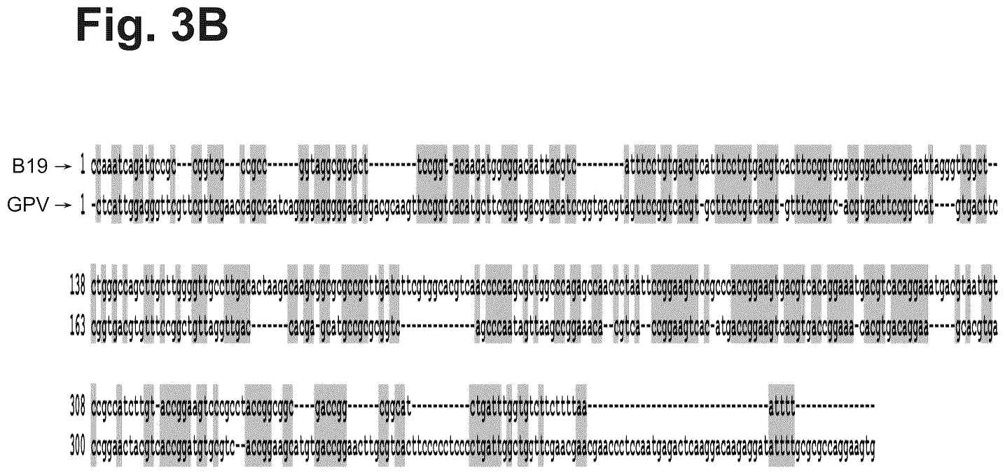

Previously, a reverse genetics system for the rescue of recombinant B19 virus has been developed bearing a truncated version of the ITR (Manaresi, et al. Virology 508 (2017): 54-62) (Table 2B, ITR ID: B19d135). Thus, B19d135 ITR was utilized to generate a genetically stable FVIII expression plasmid B19-FVIIIco6XTEN (FIG. 1D). To further utilize this approach for the synthesis of the GPV ITR-based construct, full-length wild type sequences of B19, GPV, and AAV2 ITRs were compared (FIG. 3A). Based on the homology to the first 135 and 15 nucleotides of B19 and AVV2 ITR sequences, respectively, that are dispensable for ITR function, it was hypothesized that the first 162 nucleotides of the GPV ITR could be removed in order to synthesize stable genetic constructs with fully functional ITRs (FIG. 3A, boxed sequences). Therefore, similarly to the constructs AAV2-FVIIIco6XTEN and B19-FVIIIco6XTEN that bear truncated versions of their corresponding ITRs, GPVd162 was used (Table 2C) to generate a stable FVIII expression plasmid construct GPV-FVIIIco6XTEN (FIG. 1E). Notably, both full-length B19 and GPV ITRs are much longer than full-length AAV2 ITR (Table 1) and do not form the distinctive T-shaped hairpin structure ofAAV ITRs (FIG. 2B).

Plasmids containing full length B19 ITR sequences exhibited a high degree of instability in bacterial host cells as a FVIIIco6XTEN expression construct containing only the 3′ITR could be generated. Using standard molecular cloning techniques, no positive clones could be obtained that contained both the 5′ and 3′ full length B19 ITRs. In order to generate a FVIIIco6XTEN expression construct, B19 wt-FVIIIco6XTEN, flanked by the full length B19 ITRs (FIG. 1F), the specific host E. coli strain PMC103 was used. PMC103 contains a deletion in the gene sbcC, which encodes an exonuclease that recognizes and eliminates cruciform DNA structures. Without being bound by theory, it was thought that use of the strain PMC103, lacking sbcC, may allow for the replication of long palindromes (i.e., sequences that contain complex secondary structure) and successful cloning of B19 wt-FVIIIco6XTEN as well as GPVwt-FVIIIco6XTEN. The resulting plasmid encodes the 383 base pair wildtype B19 5′ and 3′ ITR sequence (Table 2D) and another plasmid encodes the 444 base pair wildtype GPV 5′ and 3′ ITR sequence (Table 2F).

The plasmids B19-FVIIIco6XTEN (FIG. 1D; Table 2B), GPV-FVIIIco6XTEN (FIG. 1E; Table 2C) and B19 wt-FVIIIco6XTEN (FIG. 1F, Table 2D) containing FVIII-expression cassettes flanked by non-AAV parvoviral ITRs (B19d135, GPVd162, and B19 wt) were generated as described in Example 1a. Recognition sites for the restriction endonuclease Lgul were used to flank all FVIII expression cassettes (FIGS. 1D-1F).

### Example 1c. Preparation of Single-Stranded DNA Fragments Containing FVIII Expression Cassettes Flanked with AAV and Non-AAV Parvoviral ITRs

It was hypothesized that formation of hairpin structures within the ITR regions flanking the FVIII expression cassette would drive persistent transduction of target cells. For proof-of-concept studies, AAV ITR-based plasmid AAV2-FVIIIco6XTEN and non-AAV ITR-based plasmids B19-FVIIIco6XTEN and GPV-FVIIIco6XTEN were digested with Pvull and Lgul, respectively. Single-stranded (ss) AAV-FVIII, B19-FVIII, or GPV-FVIII fragment with formed hairpin ITR structures were generated by denaturing the double-stranded DNA fragment products (FVIII expression cassette and plasmid backbone) of Pvull or Lgul digestion at 95° C. and then cooling down at 4° C. to allow the palindromic ITR sequences to fold (FIG. 1A-1B). The resulting ssAAV-FVIII, ssB19-FVIII, or ssGPV-FVIII was tested in the HemA (hemophilia A) mouse model for the ability to establish persistent transduction of hepatocytes.

### Example 1d. Use of a Baculovirus Expression System to Generate FVIII Expression Constructs

A baculovirus expression system described in Li et al., PLoS ONE 8(8): e69879 (2013) for production of AAV-FVIII, B19-FVIII, and GPV-FVIII constructs in a form of closed-end DNA (ceDNA) molecules in insect cells will be utilized. Systemic delivery of ceDNA expression cassettes has been demonstrated to establish persistent transduction of hepatocytes and drive stable long-term transgene expression in the liver.

### Example 2. Systemic Injection of Genetic Constructs Comprising FVIII Expression Cassettes Flanked by AAV and Non-AAV Parvoviral ITRs Results in Long-Term FVIII Expression in HemA Mice

### Example 2a. In Vivo Evaluation of ssAAV-FVIII-Mediated FVIII Expression

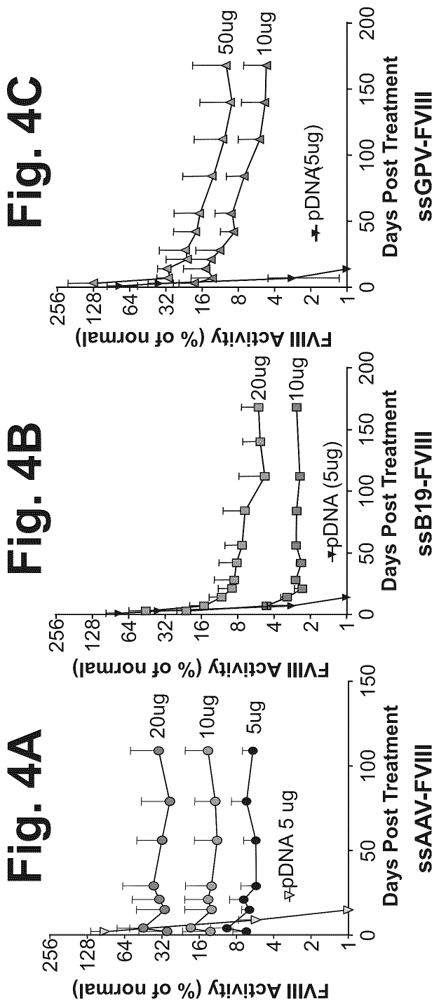

To validate the ability of ssAAV-FVIII bearing AAV ITR regions to mediate persistent transgene expression in vivo, the genetic expression cassette was delivered systemically via hydrodynamic injection (HDI) in 5-12-week old hemophilia A (HemA) mice (4 animals/group) at 5 μg, 10 μg, 20 μg of ssDNA genetic expression cassette (ssAAV-FVIII) (FIG. 4A). HDI results in primary delivery of the injected material into the liver of experimental animals. Plasma samples were collected from experimental animals at 18 hours, 3 days, 2 weeks, 3 weeks, 1 month, 2 months, 3 months and 4 months after a single hydrodynamic injection of ssAAV-FVIII. FVIII plasma activity in blood was analyzed by chromogenic FVIII activity assay. Control animals injected with 5 μg/mouse of the parental expression plasmid showed high levels of FVIII plasma activity shortly after administration. However, the level of circulating FVIII rapidly declined and became undetectable by 15 days post-injection (p.i.). In contrast, the experimental animals injected with 5, 10, and 20 μg/mouse of ssAAV-FVIII developed long-term expression of the transgene with stable levels of circulating FVIII about 8, 16, and 32% of normal FVIII level, respectively (FIG. 4A). A strong dose response was observed suggesting a high degree of correlation between injected dose and treatment outcome.

### Example 2b. In Vivo Evaluation of ssB19-FVIII- and ssGPV-FVIII-Mediated FVIII Expression

To evaluate in vivo expression of FVIII from ssB19-FVIII and ssGPV-FVIII that bear non-AAV parvoviral ITR regions B19d135 and GPVd162, respectively, 10 or 20 μg/mouse of ssB19-FVIII, and 10 or 50 μg/mouse of ssGPV-FVIII genetic expression cassette was delivered systemically via HDI in 5-12-week old hemophilia A (HemA) mice. Blood samples were collected at 1, 3, 7, 14, 21, 28, 42, 56, 84, 112, 140, and 168 days p.i. and FVIII activity in blood was analyzed by the chromogenic FVIII activity assay. As observed with the AAV-FVIII construct, control animals injected with 5 μg/mouse of parental FVIII expression plasmid showed high levels of FVIII plasma activity at 24 hours p.i. that rapidly declined and became undetectable by 14 days p.i. The experimental animals injected with ssB19-FVIII showed peak FVIII plasma activity at 3 days p.i. that then gradually declined over the period of 21 days and stabilized around 28 days p.i. (FIG. 4B) The HemA mice injected with ssGPV-FVIII, on the other hand, developed stable levels of FVIII plasma activity around day 112 that were maintained during the remaining observation period (FIG. 4C). Notably, the animals injected with 10 μg/mouse of either ssAAV-FVIII (FIG. 4A) or ssGPV-FVIII (FIG. 4C) developed highly similar stable levels of FVIII plasma activity suggesting that both AAV2 and GPV ITR regions comprise genetic factors required for efficient establishment of persistent transduction of target cells.

### Example 2c. In Vivo Evaluation of ITR and Hairpin Requirement for Stable Long-Term Expression of FVIII in hemA Mice

To compare the stability and long-term expression of single-stranded DNA cassettes to alternative nucleic acid therapeutics, the FVIIIco6XTEN plasmid construct (FIG. 1A) was digested with Pvull or AfIII to create double-stranded linear DNA with or without the AAV ITR sequences. The linear double stranded DNA without ITRs was purified to generate the ‘dsDNA No ITR’ construct. Finally, ligation of the purified dsDNA without ITRs via overlapping AfIII recognition sites resulted in the formation of minicircle DNA. This small, circular, plasmid-like DNA construct is devoid of any bacterial sequence and/or ITR sequence. HemA mice were injected with equimolar concentrations of DNA construct via hydrodynamic injection and FVIII activity levels were determined from plasma collections over 2-4 months. All DNA constructs generated initial therapeutic levels of FVIII in the 30-60% normal range, however, only single-stranded DNA demonstrated stable persistence of transgene expression at 32% for 4 months post injection (FIG. 5). All double stranded DNA and minicircle DNA reached stable levels of expression at 6-10% normal at days 14-42, however, these plateaus represent only 10% of the initial FVIII activity observed. Because transient and elevated levels of FVIII expression can result in the formation of neutralizing anti-drug antibodies, stable expression is required for immune tolerance in a hemophilia A setting.

### Example 2d. In Vivo Comparison of Wildtype and Derivative B19 ITRs

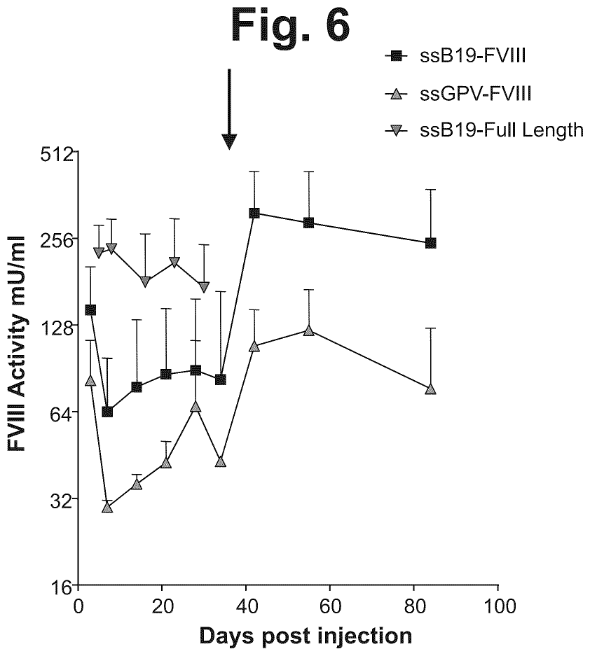

To compare the effect of a B19 derivative ITR (B19d135, FIG. 1D, Table 2C) to the full length B19 ITR (Table 2C), FVIIIco6XTEN expression cassette flanked by the 248 base pair ITRs (FIG. 1F) was generated. Hemophilia A mice were hydrodynamically injected with 30 μg single stranded FVIII-DNA flanked by either B19d135 (FIG. 1D), GPVd165 (FIG. 1E), or wildtype B19 ITRs (FIG. 1F). Plasma was collected at 3, 7, 14, 21, 28, and 35 days post injection for all cohorts, with additional samples taken at days 42, 55, and 84 for B19d135 and GPVd165 constructs and analyzed for FVIII activity by chromogenic assay (FIG. 6). Compared to the derivative B19 ITR, the full length ITR resulted in a roughly 2.5-fold increase in FVIII expression. Furthermore, the expression of FVIII from the wildtype ITR was stable at the onset.

### Example 2e. Evaluation of Re-Administration of Single-Stranded Naked DNA In Vivo

A critical limitation in current gene therapy modalities is the inability to re-administer the therapeutic due to the formation of anti-drug antibodies against the viral capsid of the gene therapy vector. However, gene therapy systems absent in immunogenic proteins could be re-dosed to titrate the patient to a desired therapeutic level. To evaluate if our non-AAV ITR flanked single stranded cassettes could be re-administered, hemA mice were injected with 30 μg ssDNA containing the B19d135 and GPVd165 ITRs at days 0 and 35 (FIG. 6). Mice administered GPVd165-FVIII reached stable FVIII levels of approximately 5% normal during the first month of observation. Following a second dose of ssDNA, the levels of FVIII rose to 10% before decreasing slightly, demonstrating a 2-fold increase in FVIII levels. Mice administered B19d135-FVIII reached stable FVIII levels of 8% during the first week which rose roughly 3.5-fold to 30% before decreasing to 25%. These data demonstrate the re-administration of single-stranded DNA with non-AAV ITRs can increase the stable expression levels of FVIII in hemophilia A mice.

### Example 3. Generation and In Vivo Evaluation of FVIII Expression Constructs Bearing Derivatives of B19d135 and GPVd162 Non-AAV Parvoviral ITRs

### Example 3a. Determination of Minimal Essential Sequences of B19 and GPV ITRs

Based on the comparison between the ITR sequences of dependoviruses AAV2 and GPV, and Erythrovirus B19 (Gene Bank accession numbers NC_001401.2, U25749.1, and KY940273.1, respectively), minimal sequences of GPV and B19 parvovirus ITRs were designed that would be required with or without additional sequences (spacers, insertions, inversions, additions, and/or recombination with wild-type sequences of other parvoviral ITRs) for persistent transduction of eukaryotic cells with genetic constructs bearing such ITRs (FIGS. 3A and 3B). Sequence alignment ofAAV2, GPV, and B19 ITRs revealed conserved regions B19v1 and GPVv1 between all three viral species (presented in Tables 2A-2C) as continuous sequences without spacer regions of variable sequence. Likewise, minimal essential sequence variants B19v3 and GPVv3 were designed based on sequence comparison between B19 and GPV ITRs. Since FVIII expression constructs bearing GPVd162 ITRs performed better in in vivo experiments than genetic constructs bearing B19d135 ITRs, it was hypothesized that B19v3 sequence comprises minimal B19 ITR sequence regions that are conserved between B19 and GPV ITR sequences, and GPVv3 sequence comprises minimal GPV ITR sequence regions that are present in the GPV ITR sequence and are lacking from the B19 ITR sequence (Tables 2B and 2C). The sequences B19v2 and GPVv2 were generated by excluding the first 135 and 162 nucleotides and corresponding complementary 135 and 162 nucleotides in the ITR palindrome regions of the B19 and GPV ITR sequences, respectively (Tables 2B and 2C).

### Example 3b. Orientation of the Palindromic Regions of B19 and GPV ITRs and their Derivatives on Functional Genetic Constructs

Part of parvoviral ITRs consists of a self-complimentary palindromic region. It has previously been demonstrated for recombinant infectious B19 parvoviruses that rescued viruses bearing palindromic regions in direct and reverse orientations exhibit similar growth properties (Manaresi, et al. Virology 508 (2017): 54-62). Therefore, genetic expression constructs bearing B19 and GPV ITRs and their derivatives are proposed to remain functional regardless of whether the palindromic regions of such ITRs are in direct, reverse, or any possible combination of 5′ and 3′ ITR combination with respect to the genetic expression cassette. To validate this hypothesis, B19d135 and GPVd162 ITRs, as well as wildtype B19 and GPV ITRs will be incorporated in the FVIIIco6XTEN expression cassette in forward, reverse, and inverted orientations using identical as well as reverse complimentary sequences for ITRs of the same species. Single-stranded DNA from these plasmids will be generated and tested in hemophilia A mice for liver directed FVIII expression driven by the TTPp promoter as described in Example 2a, 2b, and 2d. In addition to investigating all orientations of ITRs of the same species, combinations of GPV and B19 wildtype ITR and derivatives thereof will also be generated and tested for FVIII expression in hemophilia A mice. These expression cassettes will contain one ITR of 19 origin and one ITR of GPV origin to determine if non-homologous ITR sequences can enhance episomal concatemerization and long-term expression of the desired transgene. Hemophilia A mice will be injected via hydrodynamic injection with 10, 20, or 50 μg ssDNA containing the aforementioned expression cassettes and FVIII will be measured from murine plasma collected at weekly intervals post injection. The affect on FVIII expression and longevity in mice administered these expression cassettes will be directly compared with FVIII expression and longevity in mice administered B19d135, GPVd162, and corresponding wildtype ITR expression cassettes (Tables 2B, 2C, 2D, and 2F).

### Example 3c. Systemic Injection of Genetic Constructs Bearing Derivatives of B19d135 and GPVd162 Non-AAV Parvoviral ITRs in HemA Mice

To evaluate FVIII in vivo expression from ssDNA constructs that bear derivatives of B19d135 and GPVd162 non-AAV parvoviral ITRs, 5, 10, 20, or 50 μg/mouse of each ssDNA genetic expression cassette will be delivered systemically via HDI in 5-12-week old HemA mice. Blood samples will be collected at 1, 3, 7, 14, 21, and 28 days p.i., and then once monthly for a period of 4 months. FVIII activity in blood will be analyzed by the chromogenic FVIII activity assay.

### Example 4. Production and In Vivo Evaluation of ceDNA Expression Constructs Bearing Derivatives of B19d135 and GPVd162 Non-AAV Parvoviral ITRs in Insect Cells

### Example 4a. Use of a Baculovirus Expression System to Generate ceDNA Expression Constructs Bearing Derivatives of B19d135 and GPVd162 ITRs

Similarly to AAV-FVIII, B19-FVIII, and GPV-FVIII constructs described in Example 1d, the baculovirus expression system will be used for production of FVIII expression for genetic constructs bearing derivatives of B19d135 and GPVd162 non-AAV parvoviral ITRs in a form of ceDNA in insect cells.

### Example 4b. Systemic Injection of ceDNA Expression Constructs Bearing Derivatives of B19d135 and GPVd162 Non-AAV Parvoviral ITRs in HemA Mice

To evaluate FVIII in vivo expression from ceDNA constructs that bear derivatives of B19d135 and GPVd162 non-AAV parvoviral ITRs, 5, 10, 20, or 50 μg/mouse of each ceDNA genetic expression cassette will be delivered systemically via HDI in 5-12-week old HemA mice. Blood samples will be collected at 1, 3, 7, 14, 21, and 28 days p.i., and then once monthly for a period of 4 months. FVIII activity in blood will be analyzed by the chromogenic FVIII activity assay.

### Example 5. Generation of Lipid Nanoparticle Formulations of ssDNA and ceDNA FVIII Expression Constructs

After each ssDNA or ceDNA is produced as described in Examples 1 and 4, each genetic construct will be formulated into lipid nanoparticles (LNPs) using appropriate lipid compositions by microfluidic mixing (LNP-ssDNA and LNP-ceDNA). The ratio of lipid to DNA (N/P) will be adjusted to optimize cellular transduction and FVIII expression. The parental plasmids encoding FVIII expression cassettes flanked by either AAV or non-AAV parvoviral ITRs formulated into LNPs will be used as controls for transduction efficiency.

### Example 6. In Vitro and In Vivo Evaluation of LNP-ssDNA and LNP-ceDNA

### Example 6a. In Vitro Evaluation of ssDNA- and ceDNA-Mediated FVIII Expression in Cultured Hepatocytes

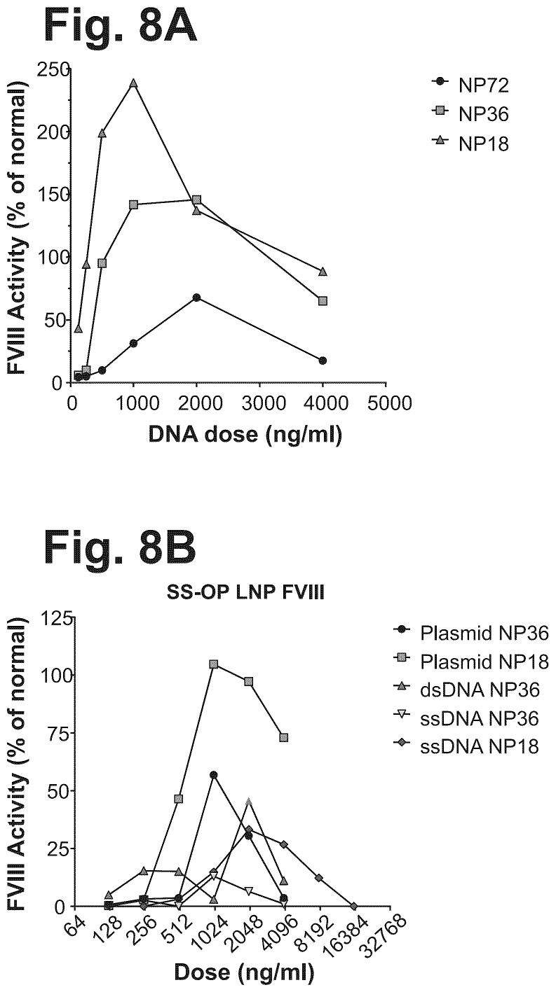

ssDNA or ceDNA FVIII expression genetic constructs and corresponding parental control plasmids were formulated into LNPs, as described in Example 5, for targeted gene delivery. Huh7 cells were seeded into 24-well tissue culture plates at 1×10⁵ cells/well and incubated overnight. On the next day, LNP-ssDNA or formulations were added onto the cells at 1000, 500, 250, 125 and 62.5 ng/well. Culture medium was harvested at 48 hours post-transduction following a media change at 24 hours post-transduction. FVIII activity in culture medium was measured by the chromogenic FVIII activity assay compared to a human plasma FACT standard. Plasmid bearing the FVIIIco6XTEN cassette under the CAGp promoter and flanked by AAV ITRs was encapsulated in lipid nanoparticles at N/P ratios of 72, 36, and 18 (FIG. 8A). Following transduction of Huh7 cells, FVIII was measured in the conditioned media. Transduction of cells with the N/P ratio of 18 generated increase FVIII levels of ratios of 36 and 72, with a peak dose of 1 μg/ml resulting in over 2 IU/ml. This data demonstrates the utility of LNP delivery in liver target cells. To investigate the transduction efficiency of ssDNA under a liver specific promoter via LNP delivery, the FVIIIco6XTEN cassette under the TTPp promoter was encapsulated at 2 N/p ratios and Huh7 cells were transduced (FIG. 8B). Consistent with our previous data (FIG. 8A), the N/P ratio of 18 resulted in increased FVIII activity levels compared to the ratio of 36. Additionally, this data demonstrated proof-of-concept LNP delivery of FVIII ssDNA in liver cells. After 24 hours, roughly 2×10⁵ Huh7 cells transduced with 2 μg/ml single-stranded FVIIIco6XTEN-AAV produced 0.33 IU/ml FVIII.

In addition, it has been shown in the literature that cellular histones are regularly positioned along the rAAV episomes, creating a chromatin-like structure that is similar to the cellular chromosomal DNA nucleosome pattern. Therefore, the ability of these constructs to establish chromatin-like nucleosomal structures required for persistent transduction of target cells will also be assessed by Southern blot.

### Example 6b. Evaluation of LNP-Formulated ssDNA- and ceDNA-Mediated Long-Term FVIII Expression in HemA Mice after Intravenous Administration

5-12-weeks old HemA mice will be administered either LNP-ssDNA, LNP-ceDNA, or LNP-pDNA (plasmid control) at 5, 10, 20, 40, 100 ug/mouse via IV injection, N=4/group. Blood samples will be collected at selected time points starting at 48 hours post-injection for up to 6 month and FVIII activity in blood will be analyzed by the chromogenic FVIII activity assay. FVIII expression profile in mice treated with LNP-ssDNA or LNP-ceDNA will be compared to that of mice treated with LNP-pDNA for each genetic construct described in Examples 1 and 4.

### Example 6c. In Vivo Evaluation of ssDNA- or ceDNA-Mediated FVIII Expression after a Booster Injection

A subset of mice treated with LNP-ssDNA or LNP-ceDNA in Example 6b will be given an additional IV injection boost of the corresponding LNPs at the same dose 2 months after the initial injection. Blood samples will be collected at selected time points starting at 48 hours after the booster injection for up to 6 months. FVIII activity in blood will be analyzed by the chromogenic FVIII activity assay. FVIII expression profile in mice treated with LNP-ssDNA or LNP-ceDNA will be compared to that of mice treated with corresponding LNP-pDNA.

### Example 7. Utility of Genetic Expression Constructs Bearing ITRs of B19 or GPV Origin for General Use in Gene Therapy

### Example 7a. Generation of Reporter Genetic Constructs Bearing ITRs of B19 or GPV Origin

In order to demonstrate the utility of non-AAV ITR-based genetic expression systems as a platform for general use in gene therapy applications, reporter constructs comprising an expression cassette were generated with green fluorescent protein (GFP) or luciferase (luc) flanked with either B19d135 or GPVd162 ITRs based on the constructs described in Example 1b. Thus, the open reading frame (ORF) of FVIII in B19-FVIIIco6XTEN (FIG. 1C) and GPV-FVIIIco6XTEN (FIG. 1D) were replaced with either ORF of GFP or luc by conventional molecular cloning techniques.

Expression cassettes flanked by B19d135 or GPVd162 ITRs were also generated containing the murine phenylalanine hydroxylase (PAH) transgene (FIG. 7A), which were used to evaluate PAH expression and reduction of blood phenylalanine concentrations in a relevant mouse model of phenylketonuria. Using this model, PKU mice (n=3) were administered 200 μg of ssDNA flanked by non-AAV ITRs via hydrodynamic injection for liver expression. Blood samples were collected at days 3, 7, 14, 28, 42, 56, 70, and 81 and plasma was isolated for phenylalanine concentration determination (FIG. 7B-7C). Mice receiving the expression cassette containing the B19d135 ITR exhibited a reduction of phenylalanine levels from 370 μg/ml to 210 μg/ml at day 3 which was stably maintained through day 81 (FIG. 7B). Mice receiving the GPVd162 ITR cassette demonstrated reduction of blood phenylalanine levels from 350 μg/ml to 310 μg/ml at day 14 which continued to decline to a stable level of 250 μg/ml by day 42 (FIG. 7C). These decreases in blood phenylalanine concentrations represent a 45% and 30% reduction compared to concentrations prior to injection (FIG. 7D). To confirm the presence of murine PAH protein in the liver, a Western blot was performed on liver lysates taken from treated mice at day 81 post injection. Using the anti-FLAG tag antibody to detect murine PAH protein, FIG. 7E demonstrates detectable murine PAH protein in 5 of 6 animals treated, with significantly higher protein levels observed in mice treated with ssDNA containing the B19d135 ITRs. These data are consistent with the blood phenyalanine reductions observed in FIGS. 7B-7D. Together, these demonstrate that single stranded DNA delivery can result in long term expression of functional liver enzymes.

Sequences of the various PAH constructs used in the experiment are set forth in Tables 10A and 10B.

TABLE 10A
B19-PAH construct bearing B19d135 ITRs (nucleotides 1-4146; SEQ ID NO: 197)

| Description | Sequence |
|---|---|
| 5′ ITR (SEQ | CTCTGGGCCAGCTTGCTTGGGGTTGCCTTGACACTAAGACAAGCGGCGCGCCGCTTGATC |
| ID NO: 180) | TTAGTGGCACGTCAACCCCAAGCGCTGGCCCAGAGCCAACCCTAATTCCGGAAGTCCCGC |
| | CCACCGGAAGTGACGTCACAGGAAATGACGTCACAGGAAATGACGTAATTGTCCGCCATC |
| | TTGTACCGGAAGTCCCGCCTACCGGCGGCGACCGGCGGCATCTGATTTGGTGTCTTCTTT |
| | TAAATTTT |
| CAGp | CTAGTTATTAATAGTAATCAATTACGGGGTCATTAGTTCATAGCCCATATATGGAGTTCC |
| promoter | GCGTTACATAACTTACGGTAAATGGCCCGCCTGGCTGACCGCCCAACGACCCCCGCCCAT |
| (SEQ ID | TGACGTCAATAATGACGTATGTTCCCATAGTAACGCCAATAGGGACTTTCCATTGACGTC |
| NO: 195) | AATGGGTGGAGTATTTACGGTAAACTGCCCACTTGGCAGTACATCAAGTGTATCATATGC |
| | CAAGTACGCCCCCTATTGACGTCAATGACGGTAAATGGCCCGCCTGGCATTATGCCCAGT |
| | ACATGACCTTATGGGACTTTCCTACTTGGCAGTACATCTACGTATTAGTCATCGCTATTA |
| | CCATGCATGGTCGAGGTGAGCCCCACGTTCTGCTTCACTCTCCCCATCTCCCCCCCCTCC |
| | CCACCCCCAATTTTGTATTTATTTATTTTTTAATTATTTTGTGCAGCGATGGGGGCGGGG |
| | GGGGGGGGGGGGCGCGCGCCAGGCGGGGCGGGGCGGGGCGAGGGGCGGGGCGGGGCGAGG |
| | CGGAGAGGTGCGGCGGCAGCCAATCAGAGCGGCGCGCTCCGAAAGTTTCCTTTTATGGCG |
| | AGGCGGCGGCGGCGGCGGCCCTATAAAAAGCGAAGCGCGCGGCGGGCG |
| Synthetic | GTGAGCGGGCGGGACGGCCCTTCTCCTTCGGGCTGTAATTAGCGCTTGGTTTAATGACGG |
| Intron (SEQ ID | CTTGTTTCTTTTCTGTGGCTGCGTGAAAGCCTTGAGGGGCTCCGGGAGGGCCCTTTGTGC |
| NO: 192) | GGGGGGAGCGGCTCGGGGCTGTCCGCGGGGGGACGGCTGCCTTCGGGGGGGACGGGGCAG |
| | GGCGGGGTTCGGCTTCTGGCGTGTGACCGGCGGCTCTAGAGCCTCTGCTAACCATGTTCA |
| | TGCCTTCTTCTTTTTCCTACAG |
| Murine PAH | ATGGACTACAAAGACCATGACGGTGATTATAAAGATCATGATATCGATTACAAGGATGAC |
| sequence | GATGACAAGGCTGCTGTGGTTCTGGAAAATGGCGTGCTGAGCCGGAAGCTGAGCGACTTC |
| (SEQ ID | GGACAAGAGACAAGCTACATCGAGGACAACAGCAACCAGAATGGCGCCGTGTCTCTGATC |
| NO: 196) | TTCAGCCTGAAAGAAGAAGTGGGCGCCCTGGCCAAGGTGCTGAGACTGTTCGAGGAAAAC |
| | GAGATCAATCTGACCCACATCGAGAGCAGACCCAGCAGACTGAACAAGGACGAGTACGAG |
| | TTCTTCACCTACCTGGACAAGCGGAGCAAGCCTGTGCTGGGCAGCATCATCAAGAGCCTG |
| | AGAAACGACATCGGCGCCACCGTGCACGAGCTGAGCAGAGACAAAGAAAAGAACACCGTG |
| | CCATGGTTCCCCAGGACCATCCAAGAGCTGGACAGATTCGCCAACCAGATCCTGAGCTAT |
| | GGCGCCGAGCTGGACGCTGATCACCCTGGCTTTAAGGACCCCGTGTACCGGGCCAGAAGA |
| | AAGCAGTTTGCCGATATCGCCTACAACTACCGGCACGGCCAGCCTATTCCTCGGGTCGAG |
| | TACACCGAGGAAGAGAGAAAGACCTGGGGCACCGTGTTCAGAACCCTGAAGGCCCTGTAC |
| | AAGACCCACGCCTGCTACGAGCACAACCACATCTTCCCACTGCTGGAAAAGTACTGCGGC |
| | TTCCGCGAGGACAATATCCCTCAGCTCGAAGACGTGTCCCAGTTCCTGCAGACCTGCACC |
| | GGCTTTAGACTGAGGCCTGTTGCCGGACTGCTGAGCAGCAGAGATTTTCTCGGCGGCCTG |
| | GCCTTCAGAGTGTTCCACTGTACCCAGTACATCAGACACGGCAGCAAGCCCATGTACACC |
| | CCTGAGCCTGATATCTGCCACGAGCTGCTGGGACATGTGCCCCTGTTCAGCGATAGAAGC |
| | TTCGCCCAGTTCAGCCAAGAGATCGGACTGGCTTCTCTGGGAGCCCCTGACGAGTACATT |
| | GAGAAGCTGGCCACCATCTACTGGTTCACCGTGGAATTCGGCCTGTGCAAAGAGGGCGAC |
| | AGCATCAAGGCTTATGGCGCTGGACTGCTGTCTAGCTTCGGCGAGCTGCAGTACTGTCTG |
| | AGCGACAAGCCTAAGCTGCTGCCCCTGGAACTGGAAAAGACCGCCTGCCAAGAGTACACA |
| | GTGACCGAGTTCCAGCCTCTGTACTACGTGGCCGAGAGCTTCAACGACGCCAAAGAAAAA |
| | GTGCGGACCTTCGCCGCCACCATTCCTCGGCCTTTTAGCGTCAGATACGACCCCTACACA |
| | CAGCGCGTGGAAGTGCTGGACAACACACAGCAGCTGAAGATTCTGGCCGACTCCATCAAC |
| | AGCGAAGTGGGCATTCTGTGTCACGCCCTGCAGAAGATCAAGAGCTGA |
| WPRE | TCATAATCAACCTCTGGATTACAAAATTTGTGAAAGATTGACTGGTATTCTTAACTATGT |
| (mutated | TGCTCCTTTTACGCTATGTGGATACGCTGCTTTAATGCCTTTGTATCATGCTATTGCTTC |
| woodchuck | CCGTATGGCTTTCATTTTCTCCTCCTTGTATAAATCCTGGTTGCTGTCTCTTTATGAGGA |
| hepatitis virus | GTTGTGGCCCGTTGTCAGGCAACGTGGCGTGGTGTGCACTGTGTTTGCTGACGCAACCCC |
| post- | CACTGGTTGGGGCATTGCCACCACCTGTCAGCTCCTTTCCGGGACTTTCGCTTTCCCCCT |
| transcriptional | CCCTATTGCCACGGCGGAACTCATCGCCGCCTGCCTTGCCCGCTGCTGGACAGGGGCTCG |
| regulatory | GCTGTTGGGCACTGACAATTCCGTGGTGTTGTCGGGGAAATCATCGTCCTTTCCTTGGCT |
| element) | GCTCGCCTGTGTTGCCACCTGGATTCTGCGCGGGACGTCCTTCTGCTACGTCCCTTCGGC |
| (SEQ ID | CCTCAATCCAGCGGACCTTCCTTCCCGCGGCCTGCTGCCGGCTCTGCGGCCTCTTCCGCG |
| NO: 120) | TCTTCGCCTTCGCCCTCAGACGAGTCGGATCTCCCTTTGGGCCGCCTCCCCGCTG |
| bGHpA | CGACTGTGCCTTCTAGTTGCCAGCCATCTGTTGTTTGCCCCTCCCCCGTGCCTTCCTTGA |
| (bovine growth | CCCTGGAAGGTGCCACTCCCACTGTCCTTTCCTAATAAAATGAGGAAATTGCATCGCATT |
| hormone | GTCTGAGTAGGTGTCATTCTATTCTGGGGGGTGGGGTGGGGCAGGACAGCAAGGGGGAGG |
| polyadenylation | ATTGGGAAGACAATAGCAGGCATGCTGGGGA |
| signal) (SEQ | |
| ID NO: 122) | |
| 3′ ITR inverted | AAAATTTAAAAGAAGACACCAAATCAGATGCCGCCGGTCGCCGCCGGTAGGCGGGACTTC |
| terminal | CGGTACAAGATGGCGGACAATTACGTCATTTCCTGTGACGTCATTTCCTGTGACGTCACT |
| repeat (SEQ | TCCGGTGGGCGGGACTTCCGGAATTAGGGTTGGCTCTGGGCCAGCGCTTGGGGTTGACGT |
| ID NO: 181) | GCCACTAAGATCAAGCGGCGCGCCGCTTGTCTTAGTGTCAAGGCAACCCCAAGCAAGCTG |
| | GCCCAGAG |

Full-length Sequence (SEQ ID NO: 197)
CTCTGGGCCAGCTTGCTTGGGGTTGCCTTGACACTAAGACAAGCGGCGCGCCGCTTGATCTTAGTGGCACGTC
AACCCCAAGCGCTGGCCCAGAGCCAACCCTAATTCCGGAAGTCCCGCCCACCGGAAGTGACGTCACAGGAAAT
GACGTCACAGGAAATGACGTAATTGTCCGCCATCTTGTACCGGAAGTCCCGCCTACCGGCGGCGACCGGCGGC
ATCTGATTTGGTGTCTTCTTTTAAATTTTGCGGCAATTCAGTCGATAACTATAACGGTCCTAAGGTAGCGATT
TAAATACGCGCTCTCTTAAGGTAGCCCCGGGACGCGTCAATTGAGATCTGGATCCGGTACCGAATTCGCGGCC
GCCTCGACGACTAGCGTTTAGTAATGAGACGCACAAACTAATATCACAAACTGGAAATGTCTATCAATATATA
GTTGCTCTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATT
ACCATGCATGGTCGAGGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGGGGGCGCGCGCCAGGCG
GGGCGGGGCGGGGCGAGGGGCGGGGCGGGGCGAGGCGGAGAGGTGCGGCGGCAGCCAATCAGAGCGGCGCGCT
CCGAAAGTTTCCTTTTATGGCGAGGCGGCGGCGGCGGCGGCCCTATAAAAAGCGAAGCGCGCGGCGGGCGGGA
GTCGCTGCGCGCTGCCTTCGCCCCGTGCCCCGCTCCGCCGCCGCCTCGCGCCGCCCGCCCCGGCTCTGACTGA
CCGCGTTACTCCCACAGGTGAGCGGGCGGGACGGCCCTTCTCCTTCGGGCTGTAATTAGCGCTTGGTTTAATG
ACGGCTTGTTTCTTTTCTGTGGCTGCGTGAAAGCCTTGAGGGGCTCCGGGAGGGCCCTTTGTGCGGGGGGAGC
GGCTCGGGGCTGTCCGCGGGGGGACGGCTGCCTTCGGGGGGGACGGGGCAGGGCGGGGTTCGGCTTCTGGCGT
GTGACCGGCGGCTCTAGAGCCTCTGCTAACCATGTTCATGCCTTCTTCTTTTTCCTACAGCTCCTGGGCAACG
TGCTGGTTATTGTGCTGTCTCATCATTTTGGCAAAGAATTGGATCGCGAAGCCGCCACCATGGACTACAAAGA
CCATGACGGTGATTATAAAGATCATGATATCGATTACAAGGATGACGATGACAAGGCTGCTGTGGTTCTGGAA
AATGGCGTGCTGAGCCGGAAGCTGAGCGACTTCGGACAAGAGACAAGCTACATCGAGGACAACAGCAACCAGA
ATGGCGCCGTGTCTCTGATCTTCAGCCTGAAAGAAGAAGTGGGCGCCCTGGCCAAGGTGCTGAGACTGTTCGA
GGAAAACGAGATCAATCTGACCCACATCGAGAGCAGACCCAGCAGACTGAACAAGGACGAGTACGAGTTCTTC
ACCTACCTGGACAAGCGGAGCAAGCCTGTGCTGGGCAGCATCATCAAGAGCCTGAGAAACGACATCGGCGCCA
CCGTGCACGAGCTGAGCAGAGACAAAGAAAAGAACACCGTGCCATGGTTCCCCAGGACCATCCAAGAGCTGGA
CAGATTCGCCAACCAGATCCTGAGCTATGGCGCCGAGCTGGACGCTGATCACCCTGGCTTTAAGGACCCCGTG
TACCGGGCCAGAAGAAAGCAGTTTGCCGATATCGCCTACAACTACCGGCACGGCCAGCCTATTCCTCGGGTCG
AGTACACCGAGGAAGAGAGAAAGACCTGGGGCACCGTGTTCAGAACCCTGAAGGCCCTGTACAAGACCCACGC
CTGCTACGAGCACAACCACATCTTCCCACTGCTGGAAAAGTACTGCGGCTTCCGCGAGGACAATATCCCTCAG
CTCGAAGACGTGTCCCAGTTCCTGCAGACCTGCACCGGCTTTAGACTGAGGCCTGTTGCCGGACTGCTGAGCA
GCAGAGATTTTCTCGGCGGCCTGGCCTTCAGAGTGTTCCACTGTACCCAGTACATCAGACACGGCAGCAAGCC
CATGTACACCCCTGAGCCTGATATCTGCCACGAGCTGCTGGGACATGTGCCCCTGTTCAGCGATAGAAGCTTC
GCCCAGTTCAGCCAAGAGATCGGACTGGCTTCTCTGGGAGCCCCTGACGAGTACATTGAGAAGCTGGCCACCA
TCTACTGGTTCACCGTGGAATTCGGCCTGTGCAAAGAGGGCGACAGCATCAAGGCTTATGGCGCTGGACTGCT
GTCTAGCTTCGGCGAGCTGCAGTACTGTCTGAGCGACAAGCCTAAGCTGCTGCCCCTGGAACTGGAAAAGACC
GCCTGCCAAGAGTACACAGTGACCGAGTTCCAGCCTCTGTACTACGTGGCCGAGAGCTTCAACGACGCCAAAG
AAAAAGTGCGGACCTTCGCCGCCACCATTCCTCGGCCTTTTAGCGTCAGATACGACCCCTACACACAGCGCGT
GGAAGTGCTGGACAACACACAGCAGCTGAAGATTCTGGCCGACTCCATCAACAGCGAAGTGGGCATTCTGTGT
CACGCCCTGCAGAAGATCAAGAGCTGAGCAAGTAATGAGCGCTGATCATAATCAACCTCTGGATTACAAAATT
TGTGAAAGATTGACTGGTATTCTTAACTATGTTGCTCCTTTTACGCTATGTGGATACGCTGCTTTAATGCCTT
TGTATCATGCTATTGCTTCCCGTATGGCTTTCATTTTCTCCTCCTTGTATAAATCCTGGTTGCTGTCTCTTTA
TGAGGAGTTGTGGCCCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTATTGCCACGGCGGAAC
TCATCGCCGCCTGCCTTGCCCGCTGCTGGACAGGGGCTCGGCTGTTGGGCACTGACAATTCCGTGGTGTTGTC
GGGGAAATCATCGTCCTTTCCTTGGCTGCTCGCCTGTGTTGCCACCTGGATTCTGCGCGGGACGTCCTTCTGC
TACGTCCCTTCGGCCCTCAATCCAGCGGACCTTCCTTCCCGCGGCCTGCTGCCGGCTCTGCGGCCTCTTCCGC
GTCTTCGCCTTCGCCCTCAGACGAGTCGGATCTCCCTTTGGGCCGCCTCCCCGCTGATCAGCCTCGACTGTGC
CTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGGGATGCGGTGGGCTCTATGG
CTTCTGAGGCGGAAAGAACGGGCTCGAGAAGCTTCTAGATATCCTCTCTTAAGGTAGCATCGAGATTTAAATT
AGGGATAACAGGGTAATGGCGCGGGCCGCAAAATTTAAAAGAAGACACCAAATCAGATGCCGCCGGTCGCCGC
CGGTAGGCGGGACTTCCGGTACAAGATGGCGGACAATTACGTCATTTCCTGTGACGTCATTTCCTGTGACGTC
ACTTCCGGTGGGCGGGACTTCCGGAATTAGGGTTGGCTCTGGGCCAGCGCTTGGGGTTGACGTGCCACTAAGA
TCAAGCGGCGCGCCGCTTGTCTTAGTGTCAAGGCAACCCCAAGCAAGCTGGCCCAGAG

TABLE 10B
GPV-PAH construct bearing GPVd162 ITRs (nucleotides 1-4214; SEQ ID NO: 198)

| Description | Sequence |
|---|---|
| 5′ ITR (SEQ ID | CGGTGACGTGTTTCCGGCTGTTAGGTTGACCACGCGCATGCCGCGCGGTCAGCCCAAT |
| NO: 183) | AGTTAAGCCGGAAACACGTCACCGGAAGTCACATGACCGGAAGTCACGTGACCGGAAA |
| | CACGTGACAGGAAGCACGTGACCGGAACTACGTCACCGGATGTGCGTCACCGGAAGCA |
| | TGTGACCGGAACTTGCGTCACTTCCCCCTCCCCTGATTGGCTGGTTCGAACGAACGAA |
| | CCCTCCAATGAGACTCAAGGACAAGAGGATATTTTGCGCGCCAGGAAGTG |
| CAGp promoter | CTAGTTATTAATAGTAATCAATTACGGGGTCATTAGTTCATAGCCCATATATGGAGTT |
| (SEQ ID NO: 195) | CCGCGTTACATAACTTACGGTAAATGGCCCGCCTGGCTGACCGCCCAACGACCCCCGC |
| | CCATTGACGTCAATAATGACGTATGTTCCCATAGTAACGCCAATAGGGACTTTCCATT |
| | GACGTCAATGGGTGGAGTATTTACGGTAAACTGCCCACTTGGCAGTACATCAAGTGTA |
| | TCATATGCCAAGTACGCCCCCTATTGACGTCAATGACGGTAAATGGCCCGCCTGGCAT |
| | TATGCCCAGTACATGACCTTATGGGACTTTCCTACTTGGCAGTACATCTACGTATTAG |
| | TCATCGCTATTACCATGCATGGTCGAGGTGAGCCCCACGTTCTGCTTCACTCTCCCCA |
| | TCTCCCCCCCCTCCCCACCCCCAATTTTGTATTTATTTATTTTTTAATTATTTTGTGC |
| | AGCGATGGGGGCGGGGGGGGGGGGGGGGCGCGCGCCAGGCGGGGCGGGGGGGGGCGAG |
| | GGGCGGGGCGGGGCGAGGCGGAGAGGTGCGGCGGCAGCCAATCAGAGCGGCGCGCTCC |
| | GAAAGTTTCCTTTTATGGCGAGGCGGCGGCGGCGGCGGCCCTATAAAAAGCGAAGCGC |
| | GCGGCGGGCG |
| Synthetic Intron | GTGAGCGGGCGGGACGGCCCTTCTCCTTCGGGCTGTAATTAGCGCTTGGTTTAATGAC |
| (SEQ ID NO: 192) | GGCTTGTTTCTTTTCTGTGGCTGCGTGAAAGCCTTGAGGGGCTCCGGGAGGGCCCTTT |
| | GTGCGGGGGGAGCGGCTCGGGGCTGTCCGCGGGGGGACGGCTGCCTTCGGGGGGGACG |
| | GGGCAGGGCGGGGTTCGGCTTCTGGCGTGTGACCGGCGGCTCTAGAGCCTCTGCTAAC |
| | CATGTTCATGCCTTCTTCTTTTTCCTACAG |
| PAH sequence | ATGGACTACAAAGACCATGACGGTGATTATAAAGATCATGATATCGATTACAAGGATG |
| (SEQ ID NO: 196) | ACGATGACAAGGCTGCTGTGGTTCTGGAAAATGGCGTGCTGAGCCGGAAGCTGAGCGA |
| | CTTCGGACAAGAGACAAGCTACATCGAGGACAACAGCAACCAGAATGGCGCCGTGTCT |
| | CTGATCTTCAGCCTGAAAGAAGAAGTGGGCGCCCTGGCCAAGGTGCTGAGACTGTTCG |
| | AGGAAAACGAGATCAATCTGACCCACATCGAGAGCAGACCCAGCAGACTGAACAAGGA |
| | CGAGTACGAGTTCTTCACCTACCTGGACAAGCGGAGCAAGCCTGTGCTGGGCAGCATC |
| | ATCAAGAGCCTGAGAAACGACATCGGCGCCACCGTGCACGAGCTGAGCAGAGACAAAG |
| | AAAAGAACACCGTGCCATGGTTCCCCAGGACCATCCAAGAGCTGGACAGATTCGCCAA |
| | CCAGATCCTGAGCTATGGCGCCGAGCTGGACGCTGATCACCCTGGCTTTAAGGACCCC |
| | GTGTACCGGGCCAGAAGAAAGCAGTTTGCCGATATCGCCTACAACTACCGGCACGGCC |
| | AGCCTATTCCTCGGGTCGAGTACACCGAGGAAGAGAGAAAGACCTGGGGCACCGTGTT |
| | CAGAACCCTGAAGGCCCTGTACAAGACCCACGCCTGCTACGAGCACAACCACATCTTC |
| | CCACTGCTGGAAAAGTACTGCGGCTTCCGCGAGGACAATATCCCTCAGCTCGAAGACG |
| | TGTCCCAGTTCCTGCAGACCTGCACCGGCTTTAGACTGAGGCCTGTTGCCGGACTGCT |
| | GAGCAGCAGAGATTTTCTCGGCGGCCTGGCCTTCAGAGTGTTCCACTGTACCCAGTAC |
| | ATCAGACACGGCAGCAAGCCCATGTACACCCCTGAGCCTGATATCTGCCACGAGCTGC |
| | TGGGACATGTGCCCCTGTTCAGCGATAGAAGCTTCGCCCAGTTCAGCCAAGAGATCGG |
| | ACTGGCTTCTCTGGGAGCCCCTGACGAGTACATTGAGAAGCTGGCCACCATCTACTGG |
| | TTCACCGTGGAATTCGGCCTGTGCAAAGAGGGCGACAGCATCAAGGCTTATGGCGCTG |
| | GACTGCTGTCTAGCTTCGGCGAGCTGCAGTACTGTCTGAGCGACAAGCCTAAGCTGCT |
| | GCCCCTGGAACTGGAAAAGACCGCCTGCCAAGAGTACACAGTGACCGAGTTCCAGCCT |
| | CTGTACTACGTGGCCGAGAGCTTCAACGACGCCAAAGAAAAAGTGCGGACCTTCGCCG |
| | CCACCATTCCTCGGCCTTTTAGCGTCAGATACGACCCCTACACACAGCGCGTGGAAGT |
| | GCTGGACAACACACAGCAGCTGAAGATTCTGGCCGACTCCATCAACAGCGAAGTGGGC |
| | ATTCTGTGTCACGCCCTGCAGAAGATCAAGAGCTGA |
| WPRE (mutated | TCATAATCAACCTCTGGATTACAAAATTTGTGAAAGATTGACTGGTATTCTTAACTAT |
| woodchuck | GTTGCTCCTTTTACGCTATGTGGATACGCTGCTTTAATGCCTTTGTATCATGCTATTG |
| hepatitis virus | CTTCCCGTATGGCTTTCATTTTCTCCTCCTTGTATAAATCCTGGTTGCTGTCTCTTTA |
| post- | TGAGGAGTTGTGGCCCGTTGTCAGGCAACGTGGCGTGGTGTGCACTGTGTTTGCTGAC |
| transcriptional | GCAACCCCCACTGGTTGGGGCATTGCCACCACCTGTCAGCTCCTTTCCGGGACTTTCG |
| regulatory | CTTTCCCCCTCCCTATTGCCACGGCGGAACTCATCGCCGCCTGCCTTGCCCGCTGCTG |
| element) (SEQ | GACAGGGGCTCGGCTGTTGGGCACTGACAATTCCGTGGTGTTGTCGGGGAAATCATCG |
| ID NO: 120) | TCCTTTCCTTGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCGGCCTGCTGCCGGC |
| | TCTGCGGCCTCTTCCGCGTCTTCGCCTTCGCCCTCAGACGAGTCGGATCTCCCTTTGG |
| | GCCGCCTCCCCGCTG |
| bGHpA (bovine | CGACTGTGCCTTCTAGTTGCCAGCCATCTGTTGTTTGCCCCTCCCCCGTGCCTTCCTT |
| growth hormone | GACCCTGGAAGGTGCCACTCCCACTGTCCTTTCCTAATAAAATGAGGAAATTGCATCG |
| polyadenylation | CATTGTCTGAGTAGGTGTCATTCTATTCTGGGGGGTGGGGTGGGGCAGGACAGCAAGG |
| signal) (SEQ ID | GGGAGGATTGGGAAGACAATAGCAGGCATGCTGGGGA |
| NO: 122) | |
| 3′ ITR inverted | CACTTCCTGGCGCGCAAAATATCCTCTTGTCCTTGAGTCTCATTGGAGGGTTCGTTCG |
| terminal repeat | TTCGAACCAGCCAATCAGGGGAGGGGGAAGTGACGCAAGTTCCGGTCACATGCTTCCG |
| (SEQ ID NO: | GTGACGCACATCCGGTGACGTAGTTCCGGTCACGTGCTTCCTGTCACGTGTTTCCGGT |
| 184) | CACGTGACTTCCGGTCATGTGACTTCCGGTGACGTGTTTCCGGCTTAACTATTGGGCT |
| | GACCGCGCGGCATGCGCGTGGTCAACCTAACAGCCGGAAACACGTCACCG |

Full-length Sequence (SEQ ID NO: 198)
CGGTGACGTGTTTCCGGCTGTTAGGTTGACCACGCGCATGCCGCGCGGTCAGCCCAATAGTTAAGCCGGAAAC
ACGTCACCGGAAGTCACATGACCGGAAGTCACGTGACCGGAAACACGTGACAGGAAGCACGTGACCGGAACTA
CGTCACCGGATGTGCGTCACCGGAAGCATGTGACCGGAACTTGCGTCACTTCCCCCTCCCCTGATTGGCTGGT
TCGAACGAACGAACCCTCCAATGAGACTCAAGGACAAGAGGATATTTTGCGCGCCAGGAAGTGGCGGCAATTC
AGTCGATAACTATAACGGTCCTAAGGTAGCGATTTAAATACGCGCTCTCTTAAGGTAGCCCCGGGACGCGTCA
ATTGAGATCTGGATCCGGTACCGAATTCGCGGCCGCCTCGACGACTAGCGTTTAGTAATGAGACGCACAAACT
AATATCACAAACTGGAAATGTCTATCAATATATAGTTGCTCTAGTTATTAATAGTAATCAATTACGGGGTCAT
TAGTTCATAGCCCATATATGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATAGGGACTTTCCATTGACGT
CAATGGGTGGAGTATTTACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCTTATGGGACTTTCCTAC
TTGGCAGTACATCTACGTATTAGTCATCGCTATTACCATGCATGGTCGAGGTGAGCCCCACGTTCTGCTTCAC
TCTCCCCATCTCCCCCCCCTCCCCACCCCCAATTTTGTATTTATTTATTTTTTAATTATTTTGTGCAGCGATG
GGGGCGGGGGGGGGGGGGGGGCGCGCGCCAGGCGGGGCGGGGCGGGGCGAGGGGCGGGGGGGGGCGAGGCGGA
GAGGTGCGGCGGCAGCCAATCAGAGCGGCGCGCTCCGAAAGTTTCCTTTTATGGCGAGGCGGCGGCGGCGGCG
GCCCTATAAAAAGCGAAGCGCGCGGCGGGCGGGAGTCGCTGCGCGCTGCCTTCGCCCCGTGCCCCGCTCCGCC
GCCGCCTCGCGCCGCCCGCCCCGGCTCTGACTGACCGCGTTACTCCCACAGGTGAGCGGGCGGGACGGCCCTT
CTCCTTCGGGCTGTAATTAGCGCTTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGCTGTCCGCGGGGGGACGGCTGCCTTCGGGG
GGGACGGGGCAGGGCGGGGTTCGGCTTCTGGCGTGTGACCGGCGGCTCTAGAGCCTCTGCTAACCATGTTCAT
GCCTTCTTCTTTTTCCTACAGCTCCTGGGCAACGTGCTGGTTATTGTGCTGTCTCATCATTTTGGCAAAGAAT
TGGATCGCGAAGCCGCCACCATGGACTACAAAGACCATGACGGTGATTATAAAGATCATGATATCGATTACAA
GGATGACGATGACAAGGCTGCTGTGGTTCTGGAAAATGGCGTGCTGAGCCGGAAGCTGAGCGACTTCGGACAA
GAGACAAGCTACATCGAGGACAACAGCAACCAGAATGGCGCCGTGTCTCTGATCTTCAGCCTGAAAGAAGAAG
TGGGCGCCCTGGCCAAGGTGCTGAGACTGTTCGAGGAAAACGAGATCAATCTGACCCACATCGAGAGCAGACC
CAGCAGACTGAACAAGGACGAGTACGAGTTCTTCACCTACCTGGACAAGCGGAGCAAGCCTGTGCTGGGCAGC
ATCATCAAGAGCCTGAGAAACGACATCGGCGCCACCGTGCACGAGCTGAGCAGAGACAAAGAAAAGAACACCG
TGCCATGGTTCCCCAGGACCATCCAAGAGCTGGACAGATTCGCCAACCAGATCCTGAGCTATGGCGCCGAGCT
GGACGCTGATCACCCTGGCTTTAAGGACCCCGTGTACCGGGCCAGAAGAAAGCAGTTTGCCGATATCGCCTAC
AACTACCGGCACGGCCAGCCTATTCCTCGGGTCGAGTACACCGAGGAAGAGAGAAAGACCTGGGGCACCGTGT
TCAGAACCCTGAAGGCCCTGTACAAGACCCACGCCTGCTACGAGCACAACCACATCTTCCCACTGCTGGAAAA
GTACTGCGGCTTCCGCGAGGACAATATCCCTCAGCTCGAAGACGTGTCCCAGTTCCTGCAGACCTGCACCGGC
TTTAGACTGAGGCCTGTTGCCGGACTGCTGAGCAGCAGAGATTTTCTCGGCGGCCTGGCCTTCAGAGTGTTCC
ACTGTACCCAGTACATCAGACACGGCAGCAAGCCCATGTACACCCCTGAGCCTGATATCTGCCACGAGCTGCT
GGGACATGTGCCCCTGTTCAGCGATAGAAGCTTCGCCCAGTTCAGCCAAGAGATCGGACTGGCTTCTCTGGGA
GCCCCTGACGAGTACATTGAGAAGCTGGCCACCATCTACTGGTTCACCGTGGAATTCGGCCTGTGCAAAGAGG
GCGACAGCATCAAGGCTTATGGCGCTGGACTGCTGTCTAGCTTCGGCGAGCTGCAGTACTGTCTGAGCGACAA
GCCTAAGCTGCTGCCCCTGGAACTGGAAAAGACCGCCTGCCAAGAGTACACAGTGACCGAGTTCCAGCCTCTG
TACTACGTGGCCGAGAGCTTCAACGACGCCAAAGAAAAAGTGCGGACCTTCGCCGCCACCATTCCTCGGCCTT
TTAGCGTCAGATACGACCCCTACACACAGCGCGTGGAAGTGCTGGACAACACACAGCAGCTGAAGATTCTGGC
CGACTCCATCAACAGCGAAGTGGGCATTCTGTGTCACGCCCTGCAGAAGATCAAGAGCTGAGCAAGTAATGAG
CGCTGATCATAATCAACCTCTGGATTACAAAATTTGTGAAAGATTGACTGGTATTCTTAACTATGTTGCTCCT
TTTACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCGTGGT
GTGCACTGTGTTTGCTGACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCTGCTGGACAGGGGCTC
GGCTGTTGGGCACTGACAATTCCGTGGTGTTGTCGGGGAAATCATCGTCCTTTCCTTGGCTGCTCGCCTGTGT
TGCCACCTGGATTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCGGATCTCCCTTT
GGGCCGCCTCCCCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGAAGACAAT
AGCAGGCATGCTGGGGATGCGGTGGGCTCTATGGCTTCTGAGGCGGAAAGAACGGGCTCGAGAAGCTTCTAGA
TATCCTCTCTTAAGGTAGCATCGAGATTTAAATTAGGGATAACAGGGTAATGGCGCGGGCCGCCACTTCCTGG
CGCGCAAAATATCCTCTTGTCCTTGAGTCTCATTGGAGGGTTCGTTCGTTCGAACCAGCCAATCAGGGGAGGG
GGAAGTGACGCAAGTTCCGGTCACATGCTTCCGGTGACGCACATCCGGTGACGTAGTTCCGGTCACGTGCTTC
CTGTCACGTGTTTCCGGTCACGTGACTTCCGGTCATGTGACTTCCGGTGACGTGTTTCCGGCTTAACTATTGG
GCTGACCGCGCGGCATGCGCGTGGTCAACCTAACAGCCGGAAACACGTCACCG

### Example 7b. Preparation of ssDNA Reporter Genetic Constructs Bearing ITRs of B19 or GPV Origin

ssDNA reporter/PAH constructs will be prepared as described in Example 1c. Briefly, plasmids will be digested with Lgul. ssDNA fragments with formed hairpin ITR structures will be generated by denaturing the double-stranded DNA fragment products (reporter expression cassette and plasmid backbone) of Lgul digestion at 95° C. and then cooling down at 4° C. to allow the palindromic ITR sequences to fold (FIG. 1A). The resulting ssDNA constructs will be tested in mice for the ability to establish persistent transduction of liver, muscle tissue, photoreceptors in the eye, and central nervous system (CNS).

### Example 7c. In Vivo Evaluation of ssDNA-Mediated Reporter Expression

To validate the ability of the ssDNA reporter constructs described in Example 7b to mediate persistent transgene expression in vivo, 5-12-week old mice (4 animals/group) will be injected with 5, 10, or 20 μg/mouse of reporter ssDNA systemically, locally to target muscle tissue and CNS cells, and/or subretinally to target photoreceptor cells.

To evaluate expression of PAH from B19 and GPV ITR-based expression constructs, a relevant disease mouse model will be used. These genetic constructs will be delivered systemically by HDI to target the liver.

1-76. (canceled)

77. A method of expressing a clotting factor in a subject in need thereof, or treating a clotting factor deficiency in a subject in need thereof, comprising administering to the subject a nucleic acid molecule comprising a first inverted terminal repeat (ITR) and a second ITR flanking a genetic cassette comprising a heterologous polynucleotide sequence, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof,
wherein the heterologous polynucleotide sequence encodes a clotting factor.

78. A method of treating a disease or disorder in a subject in need thereof, or expressing a heterologous polynucleotide sequence in a subject in need thereof, comprising administering to the subject a nucleic acid molecule comprising a first inverted terminal repeat (ITR) and a second ITR flanking a genetic cassette comprising a heterologous polynucleotide sequence, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

79-80. (canceled)

81. The method of claim 77, wherein the nucleic acid molecule is administered intravenously, transdermally, intradermally, subcutaneously, orally, pulmonarily, or any combination thereof.

82-89. (canceled)

90. The method of claim 78, wherein the disease or disorder comprises a bleeding disorder.

91. (canceled)

92. A method of treating a metabolic disorder of the liver in a subject in need thereof, comprising administering to the subject a nucleic acid molecule comprising a first inverted terminal repeat (ITR) and a second ITR flanking a genetic cassette comprising a heterologous polynucleotide sequence encoding a liver-associated metabolic enzyme that is deficient in the subject, wherein the first ITR and/or second ITR are an ITR of a non-adeno-associated virus (non-AAV).

93. The method of claim 92, wherein the first ITR and/or second ITR comprises a nucleotide sequence at least about 75%, at least about 80%, at least about 85%, at least about 90%, at least about 95%, at least about 96%, at least about 97%, at least about 98%, at least about 99%, or 100% identical to a nucleotide sequence set forth in SEQ ID NO: 180, 181, 183, 184, 185, 186, 187 or 188, or a functional derivative thereof.

94-123. (canceled)

124. The method of claim 78, wherein:
the first ITR comprises the nucleotide sequence set forth in SEQ ID NO: 180 and the second ITR comprises the nucleotide sequence set forth in SEQ ID NO: 181;
the first ITR comprises the nucleotide sequence set forth in SEQ ID NO: 183 and the second ITR comprises the nucleotide sequence set forth in SEQ ID NO: 184;
the first ITR comprises the nucleotide sequence set forth in SEQ ID NO: 185 and the second ITR comprises the nucleotide sequence set forth in SEQ ID NO: 186;
the first ITR comprises the nucleotide sequence set forth in SEQ ID NO: 187 and the second ITR comprises the nucleotide sequence set forth in SEQ ID NO: 188; and/or
the first ITR and the second ITR are reverse complements of each other.

125. The method of claim 78, further comprising a promoter.

126. The method of claim 78, wherein the heterologous polynucleotide sequence further comprises an intronic sequence and/or the heterologous polynucleotide sequence encodes a clotting factor, a growth factor, a hormone, a cytokine, an antibody, a fragment thereof, or any combination thereof.

127. The method of claim 78, wherein the genetic cassette further comprises a post-transcriptional regulatory element, a 3′UTR poly(A) tail sequence, and/or an enhancer sequence.

128. The method of claim 78, wherein the genetic cassette comprises a single stranded nucleic acid; and/or the genetic cassette comprises a double stranded nucleic acid.

129. The method of claim 78, wherein the nucleic acid molecule comprises from 5′ to 3′: the first ITR, the genetic cassette, and the second ITR; wherein the genetic cassette comprises a tissue-specific promoter sequence, an intronic sequence, the heterologous polynucleotide sequence, a post-transcriptional regulatory element, and a 3′UTR poly(A) tail sequence.

130. The method of claim 78, wherein the nucleic acid molecule is formulated with a delivery agent.

131. the method of claim 78, wherein the nucleic acid molecule is formulated for intravenous, transdermal, intradermal, subcutaneous, pulmonary, or oral delivery, or any combination thereof.

132. The method of claim 77, wherein the clotting factor is selected from the group consisting of factor I (FI), factor II (FII), factor III (FIII), factor IV (FVI), factor V (FV), factor VI (FVI), factor VII (FVII), factor VIII (FVIII), factor IX (FIX), factor X (FX), factor XI (FXI), factor XII (FXII), factor XIII (FVIII), Von Willebrand factor (VWF), prekallikrein, high-molecular weight kininogen, fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI-1), plasminogen activator inhibitor-2 (PAI2), and any combination thereof.

133. The method of claim 132, wherein the clotting factor is FVIII.

134. The method of claim 90, wherein the bleeding disorder is hemophilia A.

135. The method of claim 92, wherein the metabolic disorder of the liver is selected from the group consisting of phenylketonuria (PKU), a urea cycle disease, a lysosomal storage disorder, and a glycogen storage disease.

136. The method of claim 92, wherein the heterologous polynucleotide sequence encodes a gene selected from dystrophin X-linked, MTM1 (myotubularin), tyrosine hydroxylase, AADC, cyclohydrolase, SMN1, FXN (frataxin), GUCY2D, RS1, CFH, HTRA, ARMS, CFB/CC2, CNGA/CNGB, Prf65, ARSA, PSAP, IDUA (MPS I), IDS (MPS II), PAH, GAA (acid alpha-glucosidase), and any combination thereof.

137. The method of claim 78, wherein the nucleic acid molecule comprises from 5′ to 3′: the first ITR, the genetic cassette, and the second ITR; wherein the genetic cassette comprises a tissue-specific promoter sequence, an intronic sequence, the heterologous polynucleotide sequence, a post-transcriptional regulatory element, and a 3′UTR poly(A) tail sequence.
